SEQUENCE LISTING (94 sequences)

SEQ ID NO: 1 - protein, 404 aa, Pyrococcus furiosus
MKRMIMYLSTVLLIAVVSGCISEQTQTQTLESNSPTQTTTTTSPQITVTFIVSVPEYTPENDSIYIAGDFNNWNPKDERYKLVKLPDGRWKITLTFPYGKTIQFKFTRGSWETVEKGINGEEIPNRRFTFTKSGTYEFKVHNWRDFVEKNVKHTITGNVITFEMFIPQLNTTRRIWIYLPPDYNYSTKRYPVLYMFDGQNLFDAATSFAGEWGVDEALEKLYKEKNFSIIVVGIDNGGDRRIDEYAPWVNRDYRRGGLGNATVKFIVETLKPYIDAHYRTDPEKTGIMGSSLGGLMAIYAGFSYPEVFRYVGAMSSAFWFNPEIYDFVREAKKGPEKIYIDWGTNEGRNPKAFSESNEKMVKILKEKGYREEFNLKVVIDKGGLHNEYYWGKRFPQAVLWLFEE

SEQ ID NO: 2 - protein, 290 aa, Neurospora crassa
MAGLHSRLTTFLLLLLSALPAIAAAAPSSGCGKGPTLRNGQTVTTNINGKSRRYTVRLPDNYNQNNPYRLIFLWHPLGSSMQKIIQGEDPNRGGVLPYYGLPPLDTSKSAIYVVPDGLNAGWANQNGEDVSFFDNILQTVSDGLCIDTNLVFSTGFSYGGGMSFSLACSRANKVRAVAVISGAQLSGCAGGNDPVAYYAQHGTSDGVLNVAMGRQLRDRFVRNNGCQPANGEVQPGSGGRSTRVEYQGCQQGKDVVWVVHGGDHNPSQRDPGQNDPFAPRNTWEFFSRFN

SEQ ID NO: 3 - protein, 566 aa, Bifidobacterium longum
MTTHNSQYSAETTHPDKQESSPAPTAAGTTASNVSTTGNATTPDASIALNADATPVADVPPRLFGSFVEHLGRCVYGGIYEPSHPTADENGFRQDVLDLVKELGVTCVRYPGGNFVSNYNWEDGIGPRENRPMRRDLAWHCTETNEMGIDDFYRWSQKAGTEIMLAVNMGTRGLKAALDELEYVNGAPGTAWADQRVANGIEEPMDIKMWCIGNEMDGPWQVGHMSPEEYAGAVDKVAHAMKLAESGLELVACGSSGAYMPTFGTWEKTVLTKAYENLDFVSCHAYYFDRGHKTRAAASMQDFLASSEDMTKFIATVSDAADQAREANNGTKDIALSFDEWGVWYSDKWNEQEDQWKAEAAQGLHHEPWPKSPHLLEDIYTAADAVVEGSLMITLLKHCDRVRSASRAQLVNVIAPIMAEEHGPAWRQTTFYPFAEAALHARGQAYAPAISSPTIHTEAYGDVPAIDAVVTWDEQARTGLLLAVNRDANTPHTLTIDLSGLPGLPGLGTLALGKAQLLHEDDPYRTNTAEAPEAVTPQPLDIAMNATGTCTATLPAISWISVEFHG

SEQ ID NO: 4 - protein, 521 aa, Sphingomonas sp.
MISYLRRATAALLLATSALAAPAIADTDGTPTSATIHADTPGPVYDRRIFTQFAEHLGNGIYGGLWVGNDKSIPNTNGFRNDVVAALRNLSVPVIRWPGGCFADEYHWREGVGPKAKRPVKVNTHWGGVTEPNSVGTDEFFELLRQVGAEAYVAGNVGNGTPQEMAEWVEYMTAPAGTLAEERAKNGHKEPYAVPYFGIGNELWGCGGNMRAEYAADVTRRYATFIKAPRGTKILKIAAGANVDDYNWTETMMRVAADQLDALSLHYYTLPQGGWPPKADPVNFGETEWADTLAKAVHMDELITKHVAIMDKYDPKKRVFLAVDEWGTWYAQDPGTHPGFLRQQNTLRDALVASVHLDIFAKHADRVRMTAIAQMVNVLQAMILTDGKKMVLTPTYHVFEMYKPWQDATVLPIELDTPWYGKGQFTMPAVSGSAVRGKDGKVHVGLSNLDPNQPNTVTVKLDGLNAATVAGRILTASAMNAHNSFDAPETIKPAPFTGAQVSGGTLSVTLPPKSVVVLDLQ

SEQ ID NO: 5 - protein, 441 aa, Sorangium cellulosum
MITPFDLHRQTLTPSRSLLRLSALGCVLAALAGCASDTGDDQPSSGGTGGSENPTTSASSTTGAGAGASTSASGTGGSGPGTSTSTSTSSGSDTGTGGDPTSGAGGSGGDPGDGGGGAGAGTGAGGSPVTCDLATSFKWKSGPPVINPKSAAGRNFVSIKDPTIVFHDGKYHVFATVYDTAGNGGWSSVYLNFTDFSQAASAQQHHMANWPTGGTVAPQVFFFRPHNKWYLIYQWNGRYSTNDDISNVNGWSRPQALLKGEPGQMGNTLGALDFWNICDDKNCHLFFSRDDGKLYRSKVSIDKFPAFDGYETVMTAPSAGLLFEASNVYKVDGTNKYLLLVEAFDNSPRFFRSWTSESIDGPWAPLADTKQKPFAGPANVTFEGGKWSDDISHGEMVRSGSDERMTINACNMQFLYQGRDPNAGGAYERLPYKLGLITLEK

SEQ ID NO: 6 - protein, 540 aa, Sorangium cellulosum
MMRIRFRRWSLLTTIAATAACVSAEQLDEDGHEFDELAESVTIDTAATYTIVGVQSGKCVEVAGGSTADAAALQIASCNGSTRQQFRMESAGGGYYRIRNVNSNRCMDVAGASTSDGARIQQYSCWSGENQQWSFTDVASGVVRLTARNSGKSLDVYGRGTADGTAVIQWASNGGTNQQFRITPVSSGTGGTGSGGTGGSGGTGGTGGTGGTGGSGGSGGSGGGEGCGLPTTFRWQSSSALVSPKSDATHNIVSIKDPTVSFFNDRWHIYATTANTAGNWQMTYLNFTDWSQAASASHYYMDRTPGFSGYRCAPQMFFFRPQNKWYLIYQSQPPQFSTTSDPSRPDTWTRPQNFFASTPAGMPSLPIDYWVICDSANCYLFFTGDDGRMYRSQTTLQNFPNGFGPVSIALQDSNRNNLFEGSSTYKIKGMNKYLTLIEAIGPTGARFYRSFTADRLDGAWTPLAHTWNAPFAGQNNVTYAPGVADWSDDVSHGELVRDGNDETATIDTCNLQFLYQGRNPSSGGEYSQLPYRLGLLKAVR

SEQ ID NO: 7 - protein, 419 aa, Sorangium cellulosum
MITHLDLPSHALAPCRSLLRLSALGCVLAALAGCSGGTTDDQPSPDGTGGSENPVTGASSASSTTGTGGSTGTSSSVGSGGSGTTGTGGSTSASGSGGDPGDGGGGAGGSPPTCDLPTTFKWKAGPPVISPKPPAGRSWASVKDPTIVFFENKYHVFATVFDTTSGNGGWQSMYSNFTDIPQANAAEQHYMANWPTGSTVAPQVFFFQPHNKWYLIYQWNGRYSTNDDINNMNGWSRPQGLLRGEPNGALDFWNICDDKNCHLFFSRDDGKLYRSKVSIDKFPAFDGYETVMSAPSASLLFEASNVYKVDGSNKYLLMVEAYDNSPRFFRSWTSESLDGPWAPLADTKQNPFAGPANVTYEGQDWSDDISHGELIRSGHDEKMTIDPCDLRFLYQGRDPKVGGDYGKLPYRLGMLTLQK

SEQ ID NO: 8 - protein, 376 aa, Pseudomonas fluorescens
MKKKILAATAILLAAIANTGVADNTPFYVGADLSYVNEMESCGATYRDQGKKVDPFQLFADKGADLVRVRLWHNATWTKYSDLKDVSKTLKRAKNAGMKTLLDFHYSDTWTDPEKQFIPKAWAHITDTKELAKALYDYTTDTLASLDQQQLLPNLVQVGNETNIEILQAEDTLVHGIPNWQRNATLLNSGVNAVRDYSKKTGKPIQVVLHIAQPENALWWFKQAKENGVIDYDVIGLSYYPQWSEYSLPQLPDAIAELQNTYHKPVMIVETAYPWTLHNFDQAGNVLGEKAVQPEFPASPRGQLTYLLTLTQLVKSAGGMGVIYWEPAWVSTRCRTLWGKGSHWENASFFDATRKNNALPAFLFFKADYQASAQAE

SEQ ID NO: 9 - protein, 298 aa, Rhodopirellula baltica
MVSSPFHSKGPKMPFNLPRLLASVLCLPLLSTLALPSIGVAQEENPPSADTSETAQLPPTGLHLFLLAGQSNMAGRGKIADEDLQPHPRVLVFNKAGEWAPAIAPLHFDKPRIAGVGLGRTFAIEYAENNPQATVGLIPCAVGGSSLDVWQPGGFHESTNTHPYDDCMKRMQQAIVAGELKGILWHQGESDSNPALSKTYQSKLNELFERFRTEFGSPNVPIVIGQLGQFTEKPWDESRKLVDQAHRTLPDRMTNTVFVHSDGLGHKGDQTHFSAEAYREFGHRYFLAYQQLTGSSNE

SEQ ID NO: 10 - protein, 252 aa, Solibacter usitatus
MKLFLLTLCAAFLLKGQPHEIFLLIGQSNMAGRGVVEEQDRQPIPRVFMLNKAMEWVPAIDPVHFDKPDIAGVGLARTFGKVLAAADPNASIGLVPAAFGGTSLEEWKVGGKLYEEAVRRAKFAMSSGKLRGILWHQGEADAGKKELASSYRQRFSAMITQLRADLGEPDVPVVVGQLGEFLSESATPRSPFASVVDEQLATVPLTVPHSAFVSSNGLTSNADHLHFDARSQREFGRRYALAFLSIDASWAH

SEQ ID NO: 11 - protein, 539 aa, Fibrobacter succinogenes
MSVEMSFKKLMGIAGVAAGLSMFAVMGANAAPDPNFHIYIAYGQSNMEGNARNFTDVDKKEHPRVKMFATTSCPSLGRPTVGEMYPAVPPMFKCGEGLSVADWFGRHMADSLPNVTIGIIPVAQGGTSIRLFDPDDYKNYLNSAESWLKNGAKAYGDDGNAMGRIIEVAKKAQEKGVIKGIIFHQGETDGGMSNWEQIVKKTYEYMLKQLGLNAEETPFVAGEMVDGGSCAGFSSRVRGLSKYIANFGVASSKGYGSKGDGLHFTVEGYRGMGLRYAQQMLKLINVAPVDPVPQEPFKGAPIAIPGKVEVEDFDKPGIGKNEDGTSNASYSDEDSENHGDSDYRKDTGVDLYKAGDGVALGYTQTGEWLEYTVDVKADGEYNIDASVAAGNSTSAFKLYIDEKAITDDVSVPQTADNSWDTYKTISVKEKVTLKAGKHVLKLEITANYVNIDWIQFSEPKKEDPPSAIAKVRFDMTEAESNFSVYSMQGQKLGTFTAKGMADAMNLVKTDAKLRKQAKGVFFVRKEGAKLMSKKVVVFE

SEQ ID NO: 12 - protein, 346 aa, Sorangium cellulosum
MTQMNRTLRGTARFLLLPLLAMLAASGCGESSSPGATGDTDNTGGTGPGTGGGAASSTTAGTGGGAASSTTAGTGGDAASSTTAGTGGGATSSTTAGTGSDSSGAGTGGAPNSRPTFHIFMLMGQSNMAGVAAKQASDQNSDQRLKVLGGCNQPAGQWNLANPPLSDCPGESRINLSTSVDPGIWFGKTLLGKLREGDTIGLIGTAESGESINTFISGGSHHQTILNKIAKAKTAENARFAGIIFHQGETDTGQSSWPGKVVQLYNEMKAAWGVDYDVPFILGELPAGGCCSVHNNLVHQAADMLPDGYWISQEGTKVMDQYHFDHASVVLMGTRYGEKMIEALKW

SEQ ID NO: 13 - protein, 754 aa, Sulfolobus solfataricus
MTAIKSLLNQMSIEEKIAQLQAIPIDALMEGKEFSEEKARKYLKLGIGQITRVAGSRLGLKPKEVVKLVNKVQKFLVENTRLKIPAIIHEECLSGLMGYSSTAFPQAIGLASTWNPELLTNVASTIRSQGRLIGVNQCLSPVLDVCRDPRWGRCEETYGEDPYLVASMGLAYITGLQGETQLVATAKHFAAHGFPEGGRNIAQVHVGNRELRETFLFPFEVAVKIGKVMSIMPAYHEIDGVPCHGNPQLLTNILRQEWGFDGIVVSDYDGIRQLEAIHKVASNKMEAAILALESGVDIEFPTIDCYGEPLVTAIKEGLVSEAIIDRAVERVLRIKERLGLLDNPFVDESAVPERLDDRKSRELALKAARESIVLLKNENNMLPLSKNINKIAVIGPNANDPRNMLGDYTYTGHLNIDSGIEIVTVLQGIAKKVGEGKVLYAKGCDIAGESKEGFSEAIEIAKQADVIIAVMGEKSGLPLSWTDIPSEEEFKKYQAVTGEGNDRASLRLLGVQEELLKELYKTGKPIILVLINGRPLVLSPIINYVKAIIEAWFPGEEGGNAIADIIFGDYNPSGRLPITFPMDTGQIPLYYSRKPSSFRPYVMLHSSPLFTFGYGLSYTQFEYSNLEVTPKEVGPLSYITILLDVKNVGNMEGDEVVQLYISKSFSSVARPVKELKGFAKVHLKPGEKRRVKFALPMEALAFYDNFMRLVVEKGEYQILIGNSSENIILKDTFRIKETKPIMERRIFLSNVQIE

SEQ ID NO: 14 - protein, 778 aa, Thermotoga neapolitana
MELYRDPSQPVEVRVKDLLSRMTLEEKIAQLGSVWGYELIDERGKFKREKAKDLLKNGIGQITRPGGSTNLEPQEAAELVNEIQRFLVEETRLGIPAMIHEECLTGYMGLGGTNFPQAIAMASTWDPDLIEKMTAAIREDMRKLGAHQGLAPVLDVARDPRWGRTEETFGESPYLVARMGVSYVKGLQGENIKEGVVATVKHFAGYSASEGGKNWAPTNIPEREFREVFLFPFEAAVKEARVLSVMNSYSEIDGVPCAANRRLLTDILRKDWGFEGIVVSDYFAVNMLGEYHRIAKDKSESARLALEAGIDVELPKTDCYQHLKDLVEKGIVPESLIDEAVSRVLKLKFMLGLFENPYVDVEKAKIESHRDLALEIARKSIILLKNDGTLPLQKNKKVALIGPNAGEVRNLLGDYMYLAHIRALLDNIDDVFGNPQIPRENYERLKKSIEEHMKSIPSVLDAFKEEGIDFEYAKGCEVTGEDRSGFKEAIEVAKRSDVAIVVVGDRSGLTLDCTTGESRDMANLKLPGVQEELVLEIAKTGKPVVLVLITGRPYSLKNLVDRVNAILQVWLPGEAGGRAIVDVIYGKVNPSGKLPISFPRSAGQIPVFHYVKPSGGRSHWHGDYVDESTKPLFPFGHGLSYTRFEYSNLRIEPKEVPSAGEVVIKVDVENVGDMDGDEVVQLYIGREFASVTRPVKELKGFKRVSLKAKEKKTVVFRLHTDVLAYYDRDMKLVVEPGEFRVMVGSSSEDIRLTGSFSVTGSKREVVGKRKFFTEVYEE

SEQ ID NO: 15 - protein, 715 aa, Clostridium stercorarium
MENKPVYLDPSYSFEERAKDLVSRMTIEEKVSQMLYNSPAIERLGIPAYNWWNEALHGVARAGTATMFPQAIGMAATFDEELIYKVADVISTEGRAKYHASSKKGDRGIYKGLTFWSPNINIFRDPRWGRGQETYGEDPYLTARLGVAFVKGLQGNHPKYLKAGGMCKNILPFTVVPESLRHEFNAVVSKKDLYETYLPAFKALVQEAKVESVMGAYNRTNGEPCCGSKTLLSDILRGEWGFKGHVVSDCWAIRDFHMHHHVTATAPESAALAVRNGCDLNCGNMFGNLLIALKEGLITEEEIDRAVTRLMITRMKLGMFDPEDQVPYASISSFVDCKEHRELALDVAKKSIVLLKNDGLLPLDRKKIRSIAVIGPNADSRQALIGNYEGTASEYVTVLDGIREMAGDDVRIYYSVGCHLYKDRVENLGEPGDRIAEAVTCAEHADVVIMCLGLDSTIEGEEMHESNIYGSGDKPDLNLPGQQQELLEAVYATGKPIVLVLLTGSALAVTWADEHIPAILNAWYPGALGGRAIASVLFGETNPSGKLPVTFYRTTEELPDFTDYSMENRTYRFMKNEALYPFGFGLSYTTFDYSDLKLSKDTIRAGEGFNVSVKVTNTGKMAGEEVVQVYIKDLEASWRVPNWQLSGMKRVRLESGETAEITFEIRPEQLAVVTDEGKSVIEPGEFEIYVGGSQPDARSVRLMGKAPLKAVLRVQ

SEQ ID NO: 16 - protein, 841 aa, Clostridium thermocellum
MVKKFTSKIKAAVFAAVVAATAIFGPAISSQAVTSVPYKWDNVVIGGGGGFMPGIVFNETEKDLIYARADIGGAYRWDPSTETWIPLLDHFQMDEYSYYGVESIATDPVDPNRVYIVAGMYTNDWLPNMGAILRSTDRGETWEKTILPFKMGGNMPGRSMGERLAIDPNDNRILYLGTRCGNGLWRSTDYGVTWSKVESFPNPGTYIYDPNFDYTKDIIGVVWVVFDKSSSTPGNPTKTIYVGVADKNESIYRSTDGGVTWKAVPGQPKGLLPHHGVLASNGMLYITYGDTCGPYDGNGKGQVWKFNTRTGEWIDITPIPYSSSDNRFCFAGLAVDRQNPDIIMVTSMNAWWPDEYIFRSTDGGATWKNIWEWGMYPERILHYEIDISAAPWLDWGTEKQLPEINPKLGWMIGDIEIDPFNSDRMMYVTGATIYGCDNLTDWDRGGKVKIEVKATGIEECAVLDLVSPPEGAPLVSAVGDLVGFVHDDLKVGPKKMHVPSYSSGTGIDYAELVPNFMALVAKADLYDVKKISFSYDGGRNWFQPPNEAPNSVGGGSVAVAADAKSVIWTPENASPAVTTDNGNSWKVCTNLGMGAVVASDRVNGKKFYAFYNGKFISTDGGLTFTDTKAPQLPKSVNKIKAVPGKEGHVWLAAREGGLWRSTDGGYTFEKLSNVDTAHVVGFGKAAPGQDYMAIYITGKIDNVLGFFRSDDAGKTWVRINDDEHGYGAVDTAITGDPRVYGRVYIATNGRGIVYGEPASDEPVPTPPQVDKGLVGDLNGDNRINSTDLTLMKRYILKSIEDLPVEDDLWAADINGDGKINSTDYTYLKKYLLQAIPELPKK

SEQ ID NO: 17 - protein, 388 aa, Bacillus halodurans
MKKTTEGAFYTREYRNLFKEFGYSEAEIQERVKDTWEQLFGDNPETKIYYEVGDDLGYLLDTGNLDVRTEGMSYGMMMAVQMDRKDIFDRIWNWTMKNMYMTEGVHAGYFAWSCQPDGTKNSWGPAPDGEEYFALALFFASHRWGDGDEQPFNYSEQARKLLHTCVHNGEGGPGHPMWNRDNKLIKFIPEVEFSDPSYHLPHFYELFSLWANEEDRVFWKEAAEASREYLKIACHPETGLAPEYAYYDGTPNDEKGYGHFFSDSYRVAANIGLDAEWFGGSEWSAEEINKIQAFFADKEPEDYRRYKIDGEPFEEKSLHPVGLIATNAMGSLASVDGPYAKANVDLFWNTPVRTGNRRYYDNCLYLFAMLALSGNFKIWFPEGQEEEH

SEQ ID NO: 18 - protein, 313 aa, Geobacillus thermodenitrificans
MVHFHPFGNVNFYEMDWSLKGDLWAHDPVIAKEGSRWYVFHTGSGIQIKTSEDGVHWENMGRVFPSLPDWCKQYVPEKDEDHLWAPDICFYNGIYYLYYSVSTFGKNTSVIGLATNRTLDPRDPDYEWKDMGPVIHSTASDNYNAIDPNVVFDQEGQPWLSFGSFWSGIQLIQLDTETMKPAAQAELLTIASRGEEPNAIEAPFIVCRNGYYYLFVSFDFCCRGIESTYKIAVGRSKDITGPYVDKNGVSMMQGGGTILDAGNDRWIGPGHCAVYFSGVSAILVNHAYDALKNGEPTLQIRPLYWDDEGWPYL

SEQ ID NO: 19 - protein, 382 aa, Sitophilus oryzae
MKIIVLLLLAVVLASADQTAPGTASRPILTASESNYFTTATYLQGWSPPSISTSKADYTVGNGYNTIQAAVNAAINAGGTTRKYIKINAGTYQEVVYIPNTKVPLTIYGGGSSPSDTLITLNMPAQTTPSAYKSLVGSLFNSADPAYSMYNSWRSKSGAIGTSCSTVFWGKAPAVQIVNLSIENSAKNTGDQQAVALQTNSDQIQIHNARLLGYQDTLYAGSGSSSVERSYYTNTYIEGDIDFVFGGGSAIFESCTFYVKADRRSDTSVVFAPDTDPHKMYGYFVYKSTITGDSAWSSSKKAYLGRAWDSAVSSSSAYVPGTSPNGQLIIKESTIDGIINTSGPWTTATSGRTYSGNNANSRDLNNDNYNRFWEYNNSGNGA

SEQ ID NO: 20 - protein, 433 aa, Erwinia chrysanthemi
MSLTHYSGLAAAVSMSLILTACGGQTPNSARFQPVFPGTVSRPVLSAQEAGRFTPQHYFAHGGEYAKPVADGWTPTPIDTSRVTAAYVVGPRAGVAGATHTSIQQAVNAALRQHPGQTRVYIKLLPGTYTGTVYVPEGAPPLTLFGAGDRPEQVVVSLALDSMMSPADYRARVNPHGQYQPADPAWYMYNACATKAGATINTTCSAVMWSQSNDFQLKNLTVVNALLDTVDSGTHQAVALRTDGESGATGKCPPAQPSDTFFVNTSDRQNSYVTDHYSRAYIKDSYIEGDVDYVFGRATAVFDRVRFHTVSSRGSKEAYVFAPDSIPSVKYGFLVINSQLTGDNGYRGAQKAKLGRAWDQGAKQTGYLPGKTANGQLVIRDSTIDSSYDLANPWGAAATTDRPFKGNISPQRDLDDIHFNRLWEYNTQVLLHE

SEQ ID NO: 21 - protein, 368 aa, Erwinia carotovora
MINASHLGKTLTLAMLISSPWALAQAADYNALVSANVTDAKAYKTITEAIASAPADSSPFVIYVKNGVYHERLTVTRPNIHLQGESRDGTVITATTAAGMLKPDGSKWGTYGSNTVKVDAPDFSARSLTISNDFDYPANQAKADEDPTKLKDSQAVALLVAENSDRAWFHDVSLTGYQDTLYVKGGRSFFSKCRISGTVDFIFGNGTALFDDCDIVARNRTDVKDQPLGYLTAPSTDIKQKYGLVIINSRVIKEKDVPAKSYGLGRPWHPTTTFEDGRYADPNAIGQTVFLNTSMDDHIYGWDKMSGKDKQGEKIWFHPQDSRFFEYKSSGTGTEKNDQRRQLSEAEAAEYTADKVLAGWVPTAPKGK

SEQ ID NO: 22 - protein, 462 aa, Caldivirga maquilingensis
MINSLPSGRTYNVVEYGADPKGLDDSTGAINEAITQASETRGIVYIPPGNYLSRNIILRSNVMLLIDKGAVVKFSTDYKSYPIIETRREGVHHCGVMPLIFGKDVRNVRIIGEGVFDGQGYAWWPIRRFRVTEDYWRRLVESGGVVGDDGKTWWPTRNAMEGAEAFRKITSEGGKPSTEDCERYREFFRPQLLQLYNAENVTIEGVTFKDSPMWTIHILYSRHVTLINTSSIAPDYSPNTDGVVVDSSSDVEVRGCMIDVGDDCLVIKSGRDEEGRRIGIPSENIHASGCLMKRGHGGFVIGSEMSGGVRNVSIQDSVFDGTERGVRIKTTRGRGGLIENVYVNNIYMRNIIHEAVVVDMFYEKRPVEPVSERTPKIRGVVIRNTSCDGADQAVLINGLPEMPIEDIIIENTRITSNKGIHIENASSIRLSNVKVNSRAIPVITMSNVRNITLDDVSGLSME

SEQ ID NO: 23 - protein, 402 aa, Erwinia carotovorum
MEYQSGKRVLSLSLGLIGLFSASAFASDSRTVSEPKAPSSCTVLKADSSTATSTIQKALNNCGQGKAVKLSAGSSSVFLSGPLSLPSGVSLLIDKGVTLRAVNNAKSFENAPSSCGVVDTNGKGCDAFITATSTTNSGIYGPGTIDGQGGVKLQDKKVSWWDLAADAKVKKLKQNTPRLIQINKSKNFTLYNVSLINSPNFHVVFSDGDGFTAWKTTIKTPSTARNTDGIDPMSSKNITIAHSNISTGDDNVAIKAYKGRSETRNISILHNEFGTGHGMSIGSETMGVYNVTVDDLIMTGTTNGLRIKSDKSAAGVVNGVRYSNVVMKNVAKPIVIDTVYEKKEGSNVPDWSDITFKDITSQTKGVVVLNGENAKKPIEVTMKNVKLTSDSTWQIKNVTVKK

SEQ ID NO: 24 - protein, 402 aa, Erwinia carotovorum
MEYQSGKRVLSLSLGLIGLFSASAWASDSRTVSEPKTPSSCTTLKADSSTATSTIQKALNNCDQGKAVRLSAGSTSVFLSGPLSLPSGVSLLIDKGVTLRAVNNAKSFENAPSSCGVVDKNGKGCDAFITAVSTTNSGIYGPGTIDGQGGVKLQDKKVSWWELAADAKVKKLKQNTPRLIQINKSKNFTLYNVSLINSPNFHVVFSDGDGFTAWKTTIKTPSTARNTDGIDPMSSKNITIAYSNIATGDDNVAIKAYKGRAETRNISILHNDFGTGHGMSIGSETMGVYNVTVDDLKMNGTTNGLRIKSDKSAAGVVNGVRYSNVVMKNVAKPIVIDTVYEKKEGSNVPDWSDITFKDVTSETKGVVVLNGENAKKPIEVTMKNVKLTSDSTWQIKNVNVKK

SEQ ID NO: 25 - protein, 978 aa, Bacillus sp.
MKSLKVNGVLFLILLLVFSSFSGAVYAKSEGSPNAPSSPVNLQIPGLAFDDDSITLVWEKPKHYNDIVDFNIYMNKKKIGSALEDNSGPAKAYIDNFYENIDKDNFHEKILIHNFKANNLKPNKSYEFYVTSVNAEGTESAPSNKIVGKTTKVPEIFNIVDYGAIPDDDSKDTEAIQAAIDAATPGSKVLIPDGKFITGELWLKSDMTLQVDGYLLGSPDAEDYSTNFWLYDYSTDERSYSLINAHTYDYGSLKNIRIVGTGIIDGNGWKYDKNHPTRDELGNELPRYVAGNNSKVTGNVKVENGKMSPLDLNSENTLGILAANQSYAAQEMGMDAKSAYAARSNLITVRGVDGMYYEGITQLNPANHGIVNLHSKNIVINGTISKTYDGNNADGYEFGDSQNIMVFNNFVDTGDDAINFASGMGQAAAKSEPTGNAWIFNNYIREGHGGVVTGSHTGGWIQDFLVEDNIMYKTDVGLRSKTNTPMGGGAKNILFRNNALEGIDGDGPFVFTSAYTDANAAIQYEPAEVISQFRDMEIVDTTVRNQGGSNKQAILVNGNNSAGEVYHENITFKNVKFDNVYSVNMDYAKDFKFINVSFTNVKDNGGNPWRIKNSTFVFENTTTAPIDATQKPEWAEDTIINAGSSPDGKNVTLTWSEATDNVGVSGYTIYKDREKLGQDYTTTNLTSFTVDGLAPATEYTFKVEATDATGNRTSNGPEIKVMTNGEADQTAPVLPKNTKISESTTKIPSSDTFSGKNVNVVYTGFTWTSITWDAASDDTGIAGYNVYANGELNGFATSNKYTLTRLEPGTKYNIEVEAVDIAGNTAPYNSVLEFETARPYPIGAPSDGGLDAKINSDGTSVTLSWNAAKALNQDVIGYRVYVNGQPMKSEGAPFTPINSEMTTSDTNYTVTGLKQGKRYTFKVEAVGHASKYSKRERLSDVLPNGLLEVSGYRWSGFGPSVDVHLIPGKAKSEQAKSK

SEQ ID NO: 26 - protein, 448 aa, Thermotoga maritima
MIMEELAKKIEEEILNHVREPQIPDREVNLLDFGARGDGRTDCSESFKRAIEELSKQGGGRLIVPEGVFLTGPIHLKSNIELHVKGTIKFIPDPERYLPVVLTRFEGIELYNYSPLVYALDCENVAITGSGVLDGSADNEHWWPWKGKKDFGWKEGLPNQQEDVKKLKEMAERGTPVEERVFGKGHYLRPSFVQFYRCRNVLVEGVKIINSPMWCVHPVLSENVIIRNIEISSTGPNNDGIDPESCKYMLIEKCRFDTGDDSVVIKSGRDADGRRIGVPSEYILVRDNLVISQASHGGLVIGSEMSGGVRNVVARNNVYMNVERALRLKTNSRRGGYMENIFFIDNVAVNVSEEVIRINLRYDNEEGEYLPVVRSVFVKNLKATGGKYAVRIEGLENDYVKDILISDTIIEGAKISVLLEFGQLGMENVIMNGSRFEKLYIEGKALLK

SEQ ID NO: 27 - protein, 602 aa, Erwinia chrysanthemi
METITFSRRPALASIVAACLISTPALAATAQAPQKLQIPTLSYDDHSVALVWDAPEDTSNITDYQIYQNGQLIGLASQNNDKNSPAKPYISAFYKNDTGNFHRRVVIQNAKIDGLKANTDYQFTVRTVYADGSTSADSNAVTATTAATPQVINITQYGAKGDGTTLNTTAIQKAIDACQTGCRVDIPAGVFKTGALWLKSDMTLNLLQGATLLGSDNAADYPEAYKIYSYSSQVRPASLINAIDKTTSAVGTFKNIRIIGKGVIDGNGWKRSADAKDELGNSLPQYVKSDSSKVSKDGILAKNQVAAAVAKGMDTKTAYSQRRSSLVTLRGVKNVYIADVTIRNPANHGVMFLESQNVVENGVIHQTFDANNGDGVEFGNSQNIMVFNSVFDTGDDSINFAAGMGQDAQSQEPSQNAWLFNNYFRRGHGAVVMGSHTGAGIIDVLAENNVISQNDVGLRAKSAPAIGGGAHGIVFRNSAMKNLAKQAVIVTLSYSDSNGTIDYTPAKVPARFYDFTVKNVTVQDSTGSSPVIEITGDSGKGIWHSQFTFSNMKLSGVTPASISDLSDSQFNNLTFSKLRSGSSPWKFGTVKNVSVDGKIVTP

SEQ ID NO: 28 - protein, 324 aa, Bacillus subtilis
MLKNKKTWKRFFHLSSAALAAGLIFTSAAPAEAAFWGASNELLHDPTMIKEGSSWYALGTGLNEERGLRVLKSSDAKNWTVQKSIFSTPLSWWSNYVPNYEKNQWAPDIQYYNGKYWLYYSVSSFGNNTSAIGLASSTSISSGNWEDEGLVIRSTSSNNYNAIDPELTFDKDGNPWLAFGSFWSGIKLTKLDKSTMKPTGSPYSIAARPNNNGALEAPTLTYQNGYYYLMVSFDKCCNGVNSTYKIAYGRSKSITGPYLDKSGKSMLDGGGTILDSGNDQWKGPGGQDIVNGNILVRHAYDANDNGTPKLLINDLNWSSGWPSY

SEQ ID NO: 29 - protein, 290 aa, Erwinia carotovorum
MAYPTTNLTGIIGFAKAANVTGGTGGKVVTVNSLADFKSAVSGSAKTIVVLGLSLKASALTKVVFGSNKTIVGSFGGYANVLTNIHLRAESNSSNVIFQNLVFKHDVAIKDNDDIQLYLNYGKGYWVDHCSWPGHTWSDNDGSLDKLIYIGEKADYITISNCLFSNHKYGCIFGHPADDNNSAYNGYPRLTICHNYYENIQVRAPGLMRYGYFHVFNQPTSINSTWPLQLRRNANLISERNVFGTGAENKGMVDDKGNGSTLRIMAVHRLRWRANRLRRNGRRHLTIHTV

SEQ ID NO: 30 - protein, 345 aa, Bacillus subtilis
MKRFCLWFAVFSLLLVLLPGKAFGAVDFPNTSTNGLLGFAGNAKNEKGISKASTTGGKNGQIVYIQSVNDLKTHLGGSTPKILVLQNDISASSKTTVTIGSNKTLVGSYAKKTLKNIYLTTSSASGNVIFQNLTFEHSPQINGNNDIQLYLDSGINYWIDHVTFSGHSYSASGSDLDKLLYVGKSADYITISNSKFANHKYGLILGYPDDSQHQYDGYPHMTIANNYFENLYVRGPGLMRYGYFHVKNNYSNNFNQAITIATKAKIYSEYNYFGKGSEKGGILDDKGTGYFKDTGSYPSLNKQTSPLTSWNPGSNYSYRVQTPQYTKDFVTKYAGSQSTTLVFGY

SEQ ID NO: 31 - protein, 392 aa, Erwinia chrysanthemi
MNKVSGRSFTRTSTCLLATLIAGVMTSGVSAAELVNSKALESAPAAGWASQNGSTTGGAAATSDNIYVVTNISEFTSALSAGAVAKIIQITGTVDISGGTPYKDFADQKARSQINIPANTTVIGIGTDAKFINGSLIIDGTDGTNNVIIRNVYIQTPIDVEPHYEKGDGWNAEWDGMNITNGAHHVWVDHVTISDGSFTDDMYTTKDGETYVQHDGALDIKRGSDYVTISNSLFDQHDKTMLIGHSDTNSAQDKGKLHVTLFNNVFNRVTERAPRVRYGSIHSFNNVFNGDVKDPVYRYLYSFGIGTSGSVLSEGNSFTIANLSASKACKVVKKFNGSIFSDNGSVLNGSAADLSGCGFSAYTSAIPYVYAVQPMTTELAQSITDHAGSGKL

SEQ ID NO: 32 - protein, 380 aa, Pseudomonas marginalis [sequence omitted]

SEQ ID NO: 33 - protein, 578 aa, Erwinia chrysanthemi
MHMNKPLQAWRTPLLTLIFVLPLTATGAVKLTLDGMNSTLDNGLLKVRFGADGSAKEVWKGGTNLISRLSGAARDPDKNRSFYLDYYSGGVNEFVPERLEVIKQTPDQVHLAYIDDQNGKLRLEYHLIMTRDVSGLYSYVVAANTGSAPVTVSELRNVYRFDATRLDTLFNSIRRGTPLLYDELEQLPKVQDETWRLPDGSVYSKYDFAGYQRESRYWGVMGNGYGAWMVPASGEYYSGDALKQELLVHQDAIILNYLTGSHFGTPDMVAQPGFEKLYGPWLLYINQGNDRELVADVSRRAEHERASWPYRWLDDARYPRQRATVSGRLRTEAPHATVVLNSSAENFDIQTTGYLFSARTNRDGRFSLSNVPPGEYRLSAYADGGTQIGLLAQQTVRVEGKKTRLGQIDARQPAPLAWAIGQADRRADEFRFGDKPRQYRWQTEVPADLTFEIGKSRERKDWYYAQTQPGSWHILFNTRTPEQPYTLNIAIAAASNNGMTTPASSPQLAVKLNGQLLTTLKYDNDKSIYRGAMQSGRYHEAHIPLPAGALQQGGNRITLELLGGMVMYDAITLTETPQ

SEQ ID NO: 34 - protein, 567 aa, Xanthomonas oryzae
MLEVRTVRTFSVSDARLASRAGIATKSCFRTVTTMPRHRLHTFACALLLYAGVSAPALAEFGCTRSGDRVIVDSGAELVFSVDTHDGDIVSMRYRDNELQTTEPKGSQIASGLGSASVDARIAGGTIIVSAKAGDLIQYYIVRKGRNAIYMATYAPTLPPVGELRFVARLNVSKLPDAQQEPDSNVGTAIEGNDVFLLPDGRTSSKFYSARRMMDDQVHGVSGPGVAVFMLMGNREHSAGGPFFKDIATQKTRVTHELYNYMYSDHTQTEAFRGGLHGVYGLLFTDGSAPSDAQLNTDFVDATLGLSDYLPASGRGAVGGQVSGVLPDQPAVIGLCNAQAQYWATADGSGEYQVTGVRPGRYRMTLYQNELEVAWRDIEVFANDTAHATLQAVALPGTLKWQIGIPDGTPAGFGYADLLPHAHPSDARMRWSATTYTVGSSGQSSFPAVQWRGINTPSRIDFTLAADEVRDYRLRIFVPLAQGSARPQISVNARWNGPMPDAPLQPKTRGITRGTTRGNNALYEMDIPASALQAGSNCIEIGIASGSPDNGFLSPAIVFDSIQLVAL

SEQ ID NO: 35 - protein, 894 aa, Caldivirga maquilingensis
MVHGLRIIDARVEFTVNPLGIDESKPRFSWILEHEERGQYQSAYRVIVSSSLENAVKGIGDVWDSGKVNSRDQVIKYNGPPLSSFTKYYWRVKAWDSNGVEGDWSDVQWFETAVLKPEEWSGKWIGGGQLLRRSFRVEGSVIEAKAYVTGLGYYELRINGERVGDRVLDPPWSEYDKTVYYSVYDVTNLVKSGENVIGLILGRGRYGPVSPNRAQIPGLKYYDEPKASAMIRIRLSDGSVITINTDESWKCLVKGPILYDDIYNGYRYDARLEPYGWDKAGFDDSNWVQCSVVKPPGGRLRSTAAVPGTKVKGTLKPREYYNPRPGVYVFDFGQNITGWVRLRVRGSSGVEVKVRHSEVINSDGSLNVENIRGAEATDTYILSGRDVEVLEPRFTYHGFRYAEVTGYPGVPSIDDVEAVIVQTDFESTGSIATSSKIINDIHRITWWSLRANLLNGIQTDCPQRDERMGWLGDAWLSSDSAVFNFNMVKYYEKFIRDIIDSQRDDGSIPDTVPPYWNTYPADPAWGTALIYIPWLLYVHYGDVEILEEAYEAMKKWWSFLNSRVKDNVLYFSKYGEWVPPGRVFSAEYCPPEILSTWILYRDTLTLAQIAKVLGRGEDASFFTKRAEEIRDAFNRVFLTERGYYSKYTAPDGSVRMLGGSQTCNALPLYLDMVPGNRVNDIVKALAHNIEADWDRHLVVGIFGAKYVPEVLVKYGYVDLAYRAVTQESYPGWGYMIKEGATTLWERWEKLTGAGMNSHNHHMFGSIDAWFYRDLAGLMTLEPGFSRIMIKPNIPSELRYCSASLYTVRGLTSVEWSRVNDELVVTVTVPVNSTAEVHLPKLGESTVVREGDKVLWSGGKVVEVSPGVLSVKDAGDRIVVEVGSGRFIFTIKTIN

SEQ ID NO: 36 - protein, 932 aa, Thermomicrobia bacterium
MLRIDRVKVERSRDGLGLGTGRPRLCWRVETDIRDWRQAAYEVELYDGSGQLVGSTGRVESGESVWVAWPFEALGSRQRAGVRVRVWGEDGSESDWSDLQWLEVGLLARDDWQGAFITPDWEEDTSVANPCPYLRKTFSLPGGVRRARLYVTGLGVYEVELNGQRVGDHVLSPGWTSYRHRLLYETFDVTGLLREGDNCLGAILGDGWYRGRLGFGGGRRNLYGERLALLAQLEVELEDGSRQVVVTDGSWRAHRGPILESGIYDGEVYDARLEMPGWSTPEYDDSEWAGTRELGWPTESLEPLEVPARRTQEVAPREILRSFSGKTIVDFGQNLVGRVRLRVSGPRGQRVRLRHAEVLEGGELCTRTLRTARATDEYVLRGDGEEEWEPRFTFHGFRYVEVEGWPGELRAEDLVAVVCHSDMERIGWFGCSDPLVERLHENVVWSMRGNFLHIPTDCPQRDERLGWTGDIQVFSPAACFIYDASGFLTSWLRDVALDQDESGAVPFVVPNALGGQVIPAAAWGDAAVIVPWVLYQRYGDAGVLEAQWPSMRAWVDCIKTIAGPARLWNKGFQFGDWLDPAAPPDNPAAARTDPYIVASAYFARSAEIVGLSAQVLGMQDMAEEYLGLASEVREAFNREYVTPNGRVVSDAQTAYSLAIGFALLPTQEQRQHAGERLAELVRAEGYKIGTGFVGTPLICDALCATGHHDVAYRLLMSRECPSWLYPVTMGATTIWERWDSLRPDGSVNPGEMTSFNHYALGAVADWLHRVVGGLAPAEPGYRKLRIQPVPGGGLSYARARHVTPYGTAECSWRTEGGEIEVRVVVPPNTSAQVVLPGSGREVEVGSGEHVWRYAFEAHRYPPVTLDTPLKEILEDAEAWEVLTRHFPEVASMPPRRLERIGTIRDLAASVVAFNERVGRLERELQALSRERS

SEQ ID NO: 37 - protein, 954 aa, Thermomicrobia bacterium
MQWQASWIWLEGEPSPRNDWVCFRKSFELDRSASPLEEAKLSITADSRYVLYVNGQLVGRGPVRSWPFEQSYDTYDLRHLLHPGRNCLAVLVTHFGVSTFSYVRGRGGLLAQLELSSGDDRTTIGTDGSWKVHRHLGYSRRTTRISPQQGFVEQLDARAWSSEWKDLMYDDSGWEDAMIVGPVGTPPWEQLVPRDIPFLTEEVLHPTRVVSLHSTVPPKIAVAVDMRAIMMPDSADHAEQVQYAGFLATILRTDGEGTARLLLSKPWVGDGIAASINGQVYGAELMSRTPTGRELEVELSAGDNLLLVYVCGSDHADPLRLALDSDLGLELVSPTGGESAFVAIGPLASRVVRNFDFSQPLEYDETAVRRISSCASVADLRAWSHLPRSVPPELVSPADVFTLCTWPRQRTELTTGKELEAMVFPSKDPGLVPILRAGDTELVLDFGQEVSGYLFLDVEASEGTLIDLYGFEFMEDDYRQDTVGLDNTLRYTCREGRQHYVSPQRRGLRYLMLTVREARAPLRVHGVGVVQSTYPVSQVGTFRCSDPLLNDIWEISRLTTKLCMEDTFVDCPAYEQTFWVGDSRNEALTAYYLFGAEELVRRCLRLVPGSRRYTPLYMDQVPSGWVSVIPNWTFLWVMACREYYERTGDLAFVQDIWPDIQYTLDHYLQHINDDGLLEISAWNLLDWAPIDQPNSGVVTHQNCFFVRALKDADELGQSAGDETAGRYAERARELAAAINTHLWSDEHKAYIDSIHADSTRSSVISMQTQVVALLTGVAEGDRAEVVRSHIASPPAGWVQIGSPFMSFFLYEAMVRQGMYAQMLEDIRQKYGLMLEHGATTCWETFPGALGARYTRSHCHAWSAAPGYFLGAYVLGVRPGGPGWHRVIVAPQPCDLAWARGSVPLPRGDRVDVSWRREGQKLLLRVERPQEVELEVVPPEEYELELDERVRQTTQ

SEQ ID NO: 38 - protein, 551 aa, Erwinia chrysanthemi
MLTTTWNRAFFLGSLLCLPISFAQAEGTVTETNASPTSPVLNVVTLAPNTSISGRVAYRDIRFPATLLIKDQHGVERSVKTDIQGRFYVDVSSLVTPLRLSAIEAGGQNCLLSNQLRAVCLGALVPELRDGHENRININPLTDRILSEVAVSAGYIGPQQLIDAATLPSLSTTVWETAYREFHVGFDDALKQAGIADPSQFDPLTYSDTMTPAFTKILQVINHTRGYNNNNGQASHTVLTDIKFRPIAGLNASGSYEPLDLTSANQHRKALEQSHTRIFIVSDSTAATYEKARFPRMGWGQVFEQQFRPGGDVTVVNGARAGRSSRDFYYEGWFRQMEPFMRPGDYLFIGMGHNDQNCDSQKALRGAADVANLCTYPNSADGRPQYPQGKPDMSFQISLERYIRYAQAHRMIPVLLTPTARVKNAEGKNGTPAVHSHLTKQNKAGGYAYIGDYTQTIRDTASKNKVPLLDVETATLALANQGDGQQWQQYWLAVDPDRYPYYRDQAGSLTQPDTTHFQQKGAQAVAAIVADQIKATPSLRELAGKLQAANR

SEQ ID NO: 39 - protein, 322 aa, Erwinia chrysanthemi
MSLRRVIAGTLMMSVSGFTLADTIFPIWPQGEAPGAATSSVQQQVVERSKDPTLPDRAVTGIRSPEITVYTPEKPNGTALLITPGGSYQRVVLDKEGSDLAPFFTRQGYTLFVMTYRMPGDGHQEGADAPLADAQRAIRTLRAHAAQWQIDPQRIGIMGFSAGGHVAASLGTRFAQTVYPAQDEIDHISARPDFMVLMYPVISMQENIAHAGSRKALIGSHPSDAQIQRYSAEKQVSAQTPPTFLVHAIDDPSVSVDNSLVMLAALRAHQIPAEIHLFEQGKHGFGIRGTVGLPAAIWPQLLDNWLTSLPLKKNTANQPDKK

SEQ ID NO: 40 - protein, 455 aa, Talaromyces emersonii
MLRRALLLSSSAILAVKAQQAGTATAENHPPLTWQECTAPGSCTTQNGAVVLDANWRWVHDVNGYTNCYTGNTWDPTYCPDDETCAQNCALDGADYEGTYGVTSSGSSLKLNFVTGSNVGSRLYLLQDDSTYQIFKLLNREFSFDVDVSNLPCGLNGALYFVAMDADGGVSKYPNNKAGAKYGTGYCDSQCPRDLKFIDGEANVEGWQPSSNNANTGIGDHGSCCAEMDVWEANSISNAVTPHPCDTPGQTMCSGDDCGGTYSNDRYAGTCDPDGCDFNPYRMGNTSFYGPGKIIDTTKPFTVVTQFLTDDGTDTGTLSEIKRFYIQNSNVIPQPNSDISGVTGNSITTEFCTAQKQAFGDTDDFSQHGGLAKMGAAMQQGMVLVMSLWDDYAAQMLWLDSDYPTDADPTTPGIARGTCPTDSGVPSDVESQSPNSYVTYSNIKFGPINSTFTAS

SEQ ID NO: 41 - protein, 257 aa, Thermotoga maritime
MVLMTKPGTSDFVWNGIPLSMELNLWNIKEYSGSVAMKFDGEKITFDADIQNLSPKEPERYVLGYPEFYYGYKPWENHTAEGSKLPVPVSSMKSFSVEVSFDIHHEPSLPLNFAMETWLTREKYQTEASIGDVEIMVWFYFNNLTPGGEKIEEFTIPFVLNGESVEGTWELWLAEWGWDYLAFRLKDPVKKGRVKFDVRHFLDAAGKALSSSARVKDFEDLYFTVWEIGTEFGSPETKSAQFGWKFENFSIDLEVRE

SEQ ID NO: 42 - protein, 297 aa, Pyrococcus furiosus
MKKEALLFLSLIFLVFVSGCIHHSTNQQLSSKQQVPEVIEIDGKQWRLIWHDEFEGSEVNKEYWTFEKGNGIAYGIPGWGNGELEYYTENNTYIVNGTLVIEARKEIITDPNEGTFLYTSSRLKTEGKVEFSPPVVVEARIKLPKGKGLWPAFWMLGSNIREVGWPNCGEIDIMEFLGHEPRTIHGTVHGPGYSGSKGITRAYTLPEGVPDFTEDFHVFGIVWYPDKIKWYVDGTFYHEVTKEQVEAMGYEWVFDKPFYIILNLAVGGYWPGNPDATTPFPAKMVVDYVRVYSFVSG

SEQ ID NO: 43 - protein, 276 aa, Rhodothermus marinus
MMQRVAFILCSLLFGCSILDGDQPIRLPHWELVWSDEFDYNGLPDPAKWDYDVGGHGWGNQELQYYTRARIENARVGGGVLIIEARRESYEGREYTSARLVTRGKASWTYGRFEIRARLPSGRGTWPAIWMLPDRQTYGSAYWPDNGEIDIMEHVGFNPDVVHGTVHTKAYNHLLGTQRGGSIRVPTARTDFHVYAIEWTPEEIRWFVDDSLYYRFPNERLTNPEADWRHWPFDQPFHLIMNIAVGGTWGGQQGVDPEAFPAQLVVDYVRVYRWVE

SEQ ID NO: 44 - protein, 646 aa, Thermotoga neapolitana
MKKLVLVLLLFPVFILAQNILHNGSFDAPILIAGVDIEPPAADGSINTQNNWVFFTNSNGEGEARVENGVLVVEITNGGDHTWSVQIIQSPIRVEKLHKYRVFFKAKASVQRNIGVKIGGTAGRGWAAYNPGTDESGGMVFELGTDWKTYEFEFVMRQETDENARFEFQLGKSTGTVWIDTVWIDDVVMEDVGTLEVSGEENEIYTEEDEDKVEDWQLVWSQEFDDGVIDPNVWNFEIGNGHAKGIPGWGNAELEYYTDKNAFVENGCLVIEARKEQVSDEYGTYDYTSARITTEGKFEIKYGKIEIRAKLPKGKGIWPALWMLGNNIGEVGWPTCGEIDIMEMLGHDTRTVLRTAHGPGYSGGASIGVAYHLPEEVPDFSEDFHVFSIEWDENEVEWYVDGQLYHVLSKDELAELGLEWVFDHPFFLILNVAMGGYWPGYPDETTQFPQRMYIDYIRVYKDMNPETITGEVDDCEYEQSQQQTGPEVTYEQINNGTFDEPIVNDQANNPDEWFIWQAGDYGISGARVSDYGVTDGYAYITIEDSGTDTWHIQFNQWIGLYKGKTYTISFRAKADTPRPINVKILQNHDPWINYFAQTVNLTTEWQTFTFTYTHPDDADEVVQISFELGKEAPTTIYFDDVSVSPQ

SEQ ID NO: 45 - protein, 565 aa, Pseudomonas sp.
MTIKYSHPKTLLSAALCASAILCSHASLAARFQAEDYTAFADTSAGNTGGAYRSDDVDIEATSDEGGGYNVGWVETGEWLTYASLNIPANGRYVVRARVASDTGGAMSVDLNAGSILLGELAIPATGGWQSWQTVEREVDLSAGTYNLGVYASTGGWNFNWIEVEPVGNTGGGGSSVTFEAEDYDNASDTTPGNTGGAYRSGDVDIEATSDQGGGYNVGWTESGEWLAYNDFNVPTAGNYRFEVRVASGSGGVLSLDLNGGSTSLGEVAIPVTGGWQTWQTVTLDAYVPAGNHSLGVYATTGGWNLNWIKATPTGGGGNPNPNPTVTWSDEFDSIDLNTWNFETGGNGWGNNELQYYTNGNNASIQYDPQAGSNVLVLEARQETGGACWFGGNCGYTSTRMNTRNKKSFKYGRMEARLKLPKAQGIWPAFWMLGDNFNTQGWPQGGELDIMEHVGTNNITSGALHGPGYSGNTPITGHLDHATPIEQSYKTYAVEWDANGIRWYVDDINFYSVSRAQVEQYGQWVYDQPFWFLLNVAVGGNWPGDPDHANFSTQRMYVDYVRVYQ

SEQ ID NO: 46 - protein, 269 aa, Sinorhizobium meliloti
MTIDRYRRFARLAFIATLPLAGLATAAAAQEGANGKSFKDDFDTLDTRVWFVSDGWNNGGHQNCTWSKKQVKTVDGILELTFEEKKVKERNFACGEIQTRKRFGYGTYEARIKAADGSGLNSAFFTYIGPADKKPHDEIDFEVLGKNTAKVQINQYVSAKGGNEFLADVPGGANQGFNDYAFVWEKNRIRYYVNGELVHEVTDPAKIPVNAQKIFFSLWGTDTLTDWMGTFSYKEPTKLQVDRVAFTAAGDECQFAESVACQLERAQSE

SEQ ID NO: 47 - protein, 418 aa, Thermococcus sp.
MFRFPDGFLLGTATSSYQIEGDNVWSDWWYWAEKGKLPPAGKACNSWELYEKDLELMAGLGYAAYRFSIEWGRVFPEEGRPNEEALMRYQGIIDLLRENGITPMLTLHHFTLPAWFALRGGFEREENLEHWRGYVELIADNIEGVELVATFNEPMVYVVASYVEGTWPPFRKNPLKAEKVAANLIRAHAIAYEILHGKFRVGIVKNRPHFIPASDSERDRKATDEIDYTFNRSLLDGILTGRFKGFMRTFDVPASGLDWLGMNYYNIMKVRAVRNPLRRFAVEDAGVSRKTDMGWSVYPKGIYDGLRAFAEYGLPLYVTENGIATLDDEWRVEFIVQHLQYVHKALKEGIDVRGYFYWSLVDNYEWAEGFRPRFGLVEVDYETFERKPRKSAHIYGEIAKKGEIRGELLEGYGLGEKL

SEQ ID NO: 48 - protein, 448 aa, Clostridium thermocellum
MSKITFPKDFIWGSATAAYQIEGAYNEDGKGESIWDRFSHTPGNIADGHTGDVACDHYHRYEEDIKIMKEIGIKSYRFSISWPRIFPEGTGKLNQKGLDFYKRLTNLLLENGIMPAITLYHWDLPQKLQDKGGWKNRDTTDYFTEYSEVIFKNLGDIVPIWFTHNEPGVVSLLGHFLGIHAPGIKDLRTSLEVSHNLLLSHGKAVKLFREMNIDAQIGIALNLSYHYPASEKAEDIEAAELSFSLAGRWYLDPVLKGRYPENALKLYKKKGIELSFPEDDLKLISQPIDFIAFNNYSSEFIKYDPSSESGFSPANSILEKFEKTDMGWIIYPEGLYDLLMLLDRDYGKPNIVISENGAAFKDEIGSNGKIEDTKRIQYLKDYLTQAHRAIQDGVNLKAYYLWSLLDNFEWAYGYNKRFGIVHVNFDTLERKIKDSGYWYKEVIKNNGF

SEQ ID NO: 49 - protein, 374 aa, Sorangium cellulosum
MPTLPEPSGLTEVNRKLPDPFTFFNGTKVTTKEQWECRRKEILAMAAKYLYGPVPPEPDEVTGTVSGGTVSITAKAGGKTETFSASISGSGSVIALKLSGGIFPSGHKTLSFGSGFEGKIRNLFGLSEVNTNIANGWMIDRVMDVLEQNPGSGHDPTKVMVSGCSGCGKGAYLAGVFSRAPVVVIVESGGGGVANLRQAEWFRHGEGGSVWQCSDAKPQSIDNLEDNGICGPWVTSAARWLRSDPSKVYNLPFDTHMLLATIAPRHLVHFTNANGRNSWCHLGGTCEALSAWAAKPVWKALGVPERMGFQMYSANHCGASGSQTALAGEMFKRAFEGNTSANTDVMGILDNGVQQPVSEWEDMWIDWDMDTVLQ

SEQ ID NO: 50 - protein, 505 aa, Sorangium cellulosum
MRLRTARPTISLALFAVLPWMLAACGSEGGSEDPSGSGGSPAASTGGVGASGSGTGGTPTGTGGPSSSSGTPTGTGGDATTSEASTGGGGPAGTGGAPGTGGTGGSGDGGNAGSAEWGEVENPGAGCTVGPMPSVASLTANSKLPDPFKKMDGSRIASKSEWACRREEILQQAYKFIYGDKPVPAKGSVSGTVSTSRITVEVKDGGGSGSFNLTVNMNGATAPAPAIIGYGGLSGMPVPSGVATITFTAIESTGTSGAKNGPFYSVYGSDHPAGYLTAQAWQISRVLDVLEQNPGVIDPRRVGVTGCSRWGKGAFVAGVLDNRIALTIPVESGLGGTIGLRLVEVLDSYSGSEWPYHGISYVRWLSEVALGQFTTGNNAGADNTNKLPVDMHEMMGLIAPRGLYIVDNPSTMYNGLDRNSAWVTANVGKMIFEALGVGNHIAYTGAGGSHCSWRSQYTASLNAMVDKFLKGNNAAATGNFATDLPNKPNHMDHIDWTPPTLAGEL

SEQ ID NO: 51 - protein, 488 aa, Sorangium cellulosum
MRTLATRTARAALGLCLTAAACGQSQPNLSGQGGAGGGSDGSGGESATSSGDTTSSSSSGSGTASSSSSSGGTTSSSSSGVDTTSSSSSGTGPDDTPVENASADCEVAALPEASALPKVSKLPDPFTKLDGTSVSTKAEWHCRRQEIRKQAEKYIYGEKPTPDVVTGTVTENKISVHVEAQGKKIDFSADIVLPSKGEAPFPAIINVGGKGGFGGITLGESRILDQGVAVIYYNHNEIGREGTAEQSRGKPNPGKFYDIYGGDHSAGLLMAWAWGASRILDVIQASGGDIIDPTGIGVTGCSRNGKGAFAIGVFDDRIALTIPHETSTAGVPAYRIADVLGKERTDHNYFGLNWLSNNFEPFVFKNNASNAVKLPIDTHALIAMMAPRGLLVLENPHQAQMGAPAGHTATAAGAAVYKALGVEKNVSYHSKVAETAHCSYKNEYTDVLAKSIARFLKHEGEAPGEFVVGSGGSLSMADWVDWQAPTLE

SEQ ID NO: 52 - protein, 600 aa, Sorangium cellulosum
MRITRLLGCVSASFAFGLLACAVEPIEEEDLDTLDGALDSADGSMSADIAIQSDWGNGYCANVRVTNKSRSPATTGWNVGVRLNGSTLANAWNVTSVSSNGQFTATNVTHNAAIKEKGWVEWGFCANGSGRPAVASVAGSGGTIVGTGASSSSSSSASSSSSSSSSSTSSSSSSSSSGAGGSGGAGGSGGAGGSGAGGSGGSGGSGGSGGSGGSTGAVEDSGASCPKPTLPAASSLPVFDTHHDPFLSLSGSRITKKSEWACRRAEIKSQVETYESGSKPVVSKDNVTGQFSANRLTVSVNDAGKSASFSINISRPSGAPAGPIPLVIGIGGNNLDTSVFTQNGVAMATFDNNAMGAQNGGGSRGTGTFYNLYGSNHSASSMIAWAWGVSRIIDALEKTPGANIDPKRIAVTGCSRNGKGALTVGAFDERIVLTIPQESGAGGSASWRVSQAGANAGENVQTLSSAASEQPWFRANFGSTFGNRVTSLPFDHHMVMGLVAPRALLVIDNRIDWLGINSTFTAGSIAQQIWKGLGVPDKMGYWQTAAHAHCAFPSSQRAALDAYVKKFLVGGGTADTNLLKGDGATADLNRWMKWTAPTLQ

SEQ ID NO: 53 - protein, 647 aa, Thermoplasma volcanium
METAKDFYRKGFRLGVNFWPRLANIKMWKEWNEQEILDDLKEAKNIGCDFLRVFILDEDFVNAYGEINVKSMAYMTRFLDMCSSLHLKVFITFIVGHMSGRNWVIPWAPDNNIYESKAIMNFSKFVEHFVNEYKTHPAIEGWLMSNEITLVKRPSSPEQAMVLESVFYGIVKNLDPDHTVSSGDVLSFLQQPPNIRNHSDYAGLHLYFYDNDLLRQRYSYGSLLNIFSNDGSVPVFLEEFGFSTNQGTEKSQGEFIYSTLWTALANESMGGLVWCFSDFIGEEDPPYDWRPLEINFGLIRADGTRKYSAEKFLQFSIELKELENMMFFQSFQRIYHEISVIVPFYAYADYTSVSEAYSDYLFNRIPNPILTSLLLCKMASLQPTVFYENDLEDHINGKKLLIIPSVPTMRATTWNRLLKASVDSDIHIMASTFRGSEGSVPLTSFHDSFTHIWEKLFGVKTITELGSKGIPYSGNIEIIFTKEFGPFKKGQHINMQAFSNTYYCYSIEATKAQIIAVDKDNRPVFTYNEETRAYLFSIPFELVLTVDDTGKYSKPFMDIYREIARRSGIKSLSTSTHPAIEVADFSNGTKNICITINHSTDTVQSTIKCYGINPMMKMGNAKYVKNCREGIVIIYPPGGVALIESSL

SEQ ID NO: 54 - protein, 640 aa, Thermofilum pendens
MDEAFKFLLGVNYWPRLYNVKMWKEWDEESLKKDIEKMKELGVRVVRIFLRDIDFADERGIPIEESLQKLQRFLDLLHEKNLQAFVTLLVGHMSGKNFPIPWTSFDSLYTPSSVEKTATFARKIAERLASHPALAGWILSNELSLVKRATTREDALRLLEAFTKTMKSVDPNHIVSSGDIPDSFMQETPNVRHLVDYVGPHLYLYDTDLVRLGYFYGAMLELFSNAGDLPVILEEFGFSTLQFSEESHARFVEEILYTSLAHEASGAFIWCFSDFTEESGEPYDWRPLELGFGLLKKDGSEKLAADSYRNFSHVVERIEKLGLHSKYKRLSSTFVVYPFYLFRDYEFIWYKESLGFWESIKPHLMSYSLLSASSVPSRMVYELDLKKILKSAKLVVLPSVVATLASTWRNLLEYVELGGTLYSSVIRGAGAFKALHDAPTHLWNELFGVENVLEAGSMGRKIFGVVKLKFVRKFGNLSEGDELLLKVPESIYTFKAQSTDSDVIALDDEGEPVIFFSRRGRGKTILSLIPIEVILQAQENAQWHEGTIFYEQLAFVSEVERRYASKDPRVELQVYTGEKDDLLIVINHSNENVETSITSATRIVEAQVIGGKARLLPESKREMRAVFPPKSGSIIRVVKT

SEQ ID NO: 55 - protein, 425 aa, Thermus caldophilus
MRWVSLALLSLLLALGGCAAQKGAEGSPPPKGTGQTVPLYASRPDGVYKNGVPLPLYGVNWFGLETCDRAPHGLWSGRSVADFLAQLKGFGFNALRLPVAPEVLRDQGTVASWAQGGDPAYPTSPLAGLRYVLEKAQGLGFYVLLDFHTFRCDLIGGRLPGRPFDPSRGYTKDDWLADLRRLAGLSLEFPNVFGIDLANEPYDLTWAEWKALAQEGARAVLGVNPRVLVAVEGVGNLSPNGGYNAFWGENLAEARDDLGLGDRLLYLPHVYGPSVYDQPYFSDSTFPNNMPAVWDAHFGHLSGRGLPWGIGEFGGKYTGQDRVWQEAFVDYLRSKGVRVWFYWALNPNSGDTGGLLEEDWKTPVWDKIRLLERLMAPGGGLAFDFLPATFEVPNPERGFAEDSYYPDEPSLDAPALVAEARGKGY

SEQ ID NO: 56 - protein, 317 aa, Thermotoga maritima [sequence omitted]

SEQ ID NO: 57 - protein, 596 aa, Thermobifida fusca
MSKVRATNRRSWMRRGLAAASGLALGASMVAFAAPANAAGCSVDYTVNSWGTGFTANVTITNLGSAINGWTLEWDFPGNQQVTNLWNGTYTQSGQHVSVSNAPYNASIPANGTVEFGFNGSYSGSNDIPSSFKLNGVTCDGSDDPDPEPSPSPSPSPSPTDPDEPGGPTNPPTNPGEKVDNPFEGAKLYVNPVWSAKAAAEPGGSAVANESTAVWLDRIGAIEGNDSPTTGSMGLRDHLEEAVRQSGGDPLTIQVVIYNLPGRDCAALASNGELGPDELDRYKSEYIDPIADIMWDFADYENLRIVAIIEIDSLPNLVTNVGGNGGTELCAYMKQNGGYVNGVGYALRKLGEIPNVYNYIDAAHHGWIGWDSNFGPSVDIFYEAANASGSTVDYVHGFISNTANYSATVEPYLDVNGTVNGQLIRQSKWVDWNQYVDELSFVQDLRQALIAKGFRSDIGMLIDTSRNGWGGPNRPTGPSSSTDLNTYVDESRIDRRIHPGNWCNQAGAGLGERPTVNPAPGVDAYVWVKPPGESDGASEEIPNDEGKGFDRMCDPTYQGNARNGNNPSGALPNAPISGHWFSAQFRELLANAYPPL

SEQ ID NO: 58 - protein, 453 aa, Streptomyces sp.
MSRSRTAMLAALTLAAGSMTLALAAGPASAGPAAPTARVDNPYVGATMYVNPEWSALAASEPGGDRVADQPTAVWLDRIATIEGVDGKMGLREHLDEALQQKGSGELVVQLVIYDLPGRDCAALASNGELGPDELDRYKSEYIDPIADILSDSKYEGLRIVTVIEPDSLPNLVTNAGGTDTTTEACTTMKANGNYEKGVSYALSKLGAIPNVYNYIDAAHHGWLGWDTNLGPSVQEFYKVATSNGASVDDVAGFAVNTANYSPTVEPYFTVSDTVNGQTVRQSKWVDWNQYVDEQSYAQALRNEAVAAGFNSDIGVIIDTSRNGWGGSDRPSGPGPQTSVDAYVDGSRIDRRVHVGNWCNQSGAGLGERPTAAPASGIDAYTWIKPPGESDGNSAPVDNDEGKGFDQMCDPSYQGNARNGYNPSGALPDAPLSGQWFSAQFRELMQNAYPPLS

SEQ ID NO: 59 - protein, 516 aa, Phanerochaete chrysosporium
MFRTATLLAFTMAAMVFGQQVGTNTAENHRTLTSQKCTKSGGCSNLNTKIVLDANWRWLHSTSGYTNCYTGNQWDATLCPDGKTCAANCALDGADYTGTYGITASGSSLKLQFVTGSNVGSRVYLMADDTHYQMFQLLNQEFTFDVDMSNLPCGLNGALYLSAMDADGGMAKYPTNKAGAKYGTGYCDSQCPRDIKFINGEANVEGWNATSANAGTGNYGTCCTEMDIWEANNDAAAYTPHPCTTNAQTRCSGSDCTRDTGLCDADGCDFNSFRMGDQTFLGKGLTVDTSKPFTVVTQFITNDGTSAGTLTEIRRLYVQNGKVIQNSSVKIPGIDPVNSITDNFCSQQKTAFGDTNYFAQHGGLKQVGEALRTGMVLALSIWDDYAANMLWLDSNYPTNKDPSTPGVARGTCATTSGVPAQIEAQSPNAYVVFSNIKFGDLNTTYTGTVSSSSVSSSHSSTSTSSSHSSSSTPPTQPTGVTVPQWGQCGGIGYTGSTTCASPYTCHVLNPYYSQCY

SEQ ID NO: 60 - protein, 506 aa, Agaricus bisporus
MFPRSILLALSLTAVALGQQVGTNMAENHPSLTWQRCTSSGCQNVNGKVTLDANWRWTHRINDFTNCYTGNEWDTSICPDGVTCAENCALDGADYAGTYGVTSSGTALTLKFVTESQQKNIGSRLYLMADDSNYEIFNLLNKEFTFDVDVSKLPCGLNGALYFSEMAADGGMSSTNTAGAKYGTGYCDSQCPRDIKFIDGEANSEGWEGSPNDVNAGTGNFGACCGEMDIWEANSISSAYTPHPCREPGLQRCEGNTCSVNDRYATECDPDGCDFNSFRMGDKSFYGPGMTVDTNQPITVVTQFITDNGSDNGNLQEIRRIYVQNGQVIQNSNVNIPGIDSGNSISAEFCDQAKEAFGDERSFQDRGGLSGMGSALDRGMVLVLSIWDDHAVNMLWLDSDYPLDASPSQPGISRGTCSRDSGKPEDVEANAGGVQVVYSNIKFGDINSTFNNNGGGGGNPSPTTTRPNSPAQTMWGQCGGQGWTGPTACQSPSTCHVINDFYSQCF

SEQ ID NO: 61 - protein, 741 aa, Clostridium thermocellum
MVKSRKISILLAVAMLVSIMIPTTAFAGPTKAPTKDGTSYKDLFLELYGKIKDPKNGYFSPDEGIPYHSIETLIVEAPDYGHVTTSEAFSYYVWLEAMYGNLTGNWSGVETAWKVMEDWIIPDSTEQPGMSSYNPNSPATYADEYEDPSYYPSELKFDTVRVGSDPVHNDLVSAYGPNMYLMHWLMDVDNWYGFGTGTRATFINTFQRGEQESTWETIPHPSIEEFKYGGPNGFLDLFTKDRSYAKQWRYTNAPDAEGRAIQAVYWANKWAKEQGKGSAVASVVSKAAKMGDFLRNDMFDKYFMKIGAQDKTPATGYDSAHYLMAWYTAWGGGIGASWAWKIGCSHAHFGYQNPFQGWVSATQSDFAPKSSNGKRDWTTSYKRQLEFYQWLQSAEGGIAGGATNSWNGRYEKYPAGTSTFYGMAYVPHPVYADPGSNQWFGFQAWSMQRVMEYYLETGDSSVKNLIKKWVDWVMSEIKLYDDGTFAIPSDLEWSGQPDTWTGTYTGNPNLHVRVTSYGTDLGVAGSLANALATYAAATERWEGKLDTKARDMAAELVNRAWYNFYCSEGKGVVTEEARADYKRFFEQEVYVPAGWSGTMPNGDKIQPGIKFIDIRTKYRQDPYYDIVYQAYLRGEAPVLNYHRFWHEVDLAVAMGVLATYFPDMTYKVPGTPSTKLYGDVNDDGKVNSTDAVALKRYVLRSGISINTDNADLNEDGRVNSTDLGILKRYILKEIDTLPYKN

SEQ ID NO: 62 - protein, 619 aa, Clostridium thermocellum
MLKKKLLTLLTVFALLTVGICGSFLPLPKASAAALIYDDFETGLNGWGPRGPETVELTTEEAYSGRYSLKVSGRTSTWNGPMVDKTDVLTLGESYKLGVYVKFVGDSYSNEQRFSLQLQYNDGAGDVYQNIKTATVYKGTWTLLEGQLTVPSHAKDVKIYVETEFKNSPSPQDLMDFYIDDFTATPANLPEIEKDIPSLKDVFAGYFKVGGAATVAELAPKPAKELFLKHYNSLTFGNELKPESVLDYDATIAYMEANGGDQVNPQITLRAARPLLEFAKEHNIPVRGHTLVWHSQTPDWFFRENYSQDENAPWASKEVMLQRLENYIKNLMEALATEYPTVKFYAWDVVNEAVDPNTSDGMRTPGSNNKNPGSSLWMQTVGRDFIVKAFEYARKYAPADCKLFYNDYNEYEDRKCDFIIEILTELKAKGLVDGMGMQSHWVMDYPSISMFEKSIRRYAALGLEIQLTELDIRNPDNSQWALERQANRYKELVTKLVDLKKEGINITALVFWGITDATSWLGGYPLLFDAEYKAKPAFYAIVNSVPPLPTEPPVQVIPGDVNGDGRVNSSDLTLMKRYLLKSISDFPTPEGKIAADLNEDGKVNSTDLLALKKLVLREL

SEQ ID NO: 63 - protein, 760 aa, Clostridium thermocellum
MIVGKVLDMDEKTAIIMTDDFAFLNVVRTSEMAVGKKVKVLDSDIIKPKNSLRRYLPVAAVAACFVIVLSFVLMFINGNTARKNIYAYVGIDINPSIELWINYNNKIAEAKALNGDAETVLEGLELKEKTVAEAVNEIVQKSMELGFISREKENIILISTACDLKAGEGSENKDVQNKIGQLFDDVNKAVSDLKNSGITTRILNLTLEERESSKEENISMGRYAVYLKAKEQNVNLTIDEIKDADLLELIAKVGIDNENVPEDIVTEDKDNLDAINTGPAESAVPEVTETLPATSTPGRTEGNTATGSVDSTPALSKNETPGKTETPGRTFNTPAKSSLGQSSTPKPVSPVQTATATKGIGTLTPRNSPTPVIPSTGIQWIDQANERINEIRKRNVQIKVVDSSNKPIENAYVEAVLTNHAFGFGTAITRRAMYDSNYTKFIKDHFNWAVFENESKWYTNEPSMGIITYDDADYLYEFCRSNGIKVRGHCIFWEAEEWQPAWVRSLDPFTLRFAVDNRLNSAVGHFKGKFEHWDVNNEMIHGNFFKSRLGESIWPYMFNRAREIDPNAKYFVNNNITTLKEADDCVALVNWLRSQGVRVDGVGVHGHFGDSVDRNLLKGILDKLSVLNLPIWITEYDSVTPDEYRRADNLENLYRTAFSHPSVEGIVMWGFWERVHWRGRDASIVNDNWTLNEAGRRFESLMNEWTTRAYGSTDGSGSFGFRGFYGTYRITVTVPGKGKYNYTLNLNRGSGTLQTTYRIP

SEQ ID NO: 64 - protein, 576 aa, Vibrio sp.
MKRTYLSLIAAGVMSLSVSAWSLDGVLVPESGILVSVGQDVDSVNDYASALGTIPAGVTNYVGIVNLDGLNSDADAGAGRNNIAELANAYPTSALVVGVSMNGEVDAVASGRYNANIDTLLNTLAGYDRPVYLRWAYEVDGPWNGHSPSGIVTSFQYVHDRIIALGHQAKISLVWQVASYCPTPGGQLDQWWPGSEYVDWVGLSYFAPQDCNWDRVNEAAQFARSKGKPLFLNESTPQRYQVADLTYSADPAKGTNRQSKTSQQLWDEWFAPYFQFMSDNSDIVKGFTYINADWDSQWRWAAPYNEGYWGDSRVQANALIKSNWQQEIAKGQYINHSETLFETLGYGSTGGGDNGGGDNGGTNPPEPCNEEFGYRYVSDSTIEVFHKNNGWSAEWNYVCLNGLCLQGEIKNGEYVKQFDAQLGSTYGIEFKVADGESQFITDKSVTFENKQCGSTGTPGGGDNGSGGDNGGDNGSGGDNGSGGGTDPSQCSADFGYNYRSDTEIEVFHKDLGWSASWNYICLDDYCVPGDKSGDSYNRSFNATLGSDYKITFKVEDSASQFITEKNITFVNTSCAQ

SEQ ID NO: 65 - protein, 469 aa, Alcaligenes sp.
MKKLAKMISIATLGACAFSAHALDGKLVPNEGVLVSVGQDVDSVNDYSSAMSTTPAGVTNYVGIVNLDGLASNADAGAGRNNVVELANLYPTSALIVGVSMNGQIQNVAQGQYNANIDTLIQTLGELDRPVYLRWAYEVDGPWNGHNTEDLKQSFRNVYQRIRELGYGDNISMIWQVASYCPTAPGQLSSWWPGDDVVDWVGLSYFAPQDCNWDRVNEAAQWARSHNKPLFINESSPQRYQLADRTYSSDPAKGTNRQSKTEQQIWSEWFAPYFQFMEDNKDILKGFTYINADWDSQWRWAAPYNEGYWGDSRVQVLPYIKQQWQDTLENPKFINHSSDLFAKLGYVADGGDNGGDNGGDNGGDNGGDNGGDNGGTEPPENCQDDFNFNYVSDQEIEVYHVDKGWSAGWNYVCLNDYCLPGNKSNGAFRKTFNAVLGQDYKLTFKVEDRYGQGQQILDRNITFTTQVCN

SEQ ID NO: 66 - protein, 398 aa, Dictyoglomus thermophilum
MHELIIGYAAPYGYKENSLYVNGEFQTNVKFPQSQKFTTVYAGLIPLKNGKNTISIVKSWGWFLLDYFKIKKAEIPTMNPTNKLVTPNPSKEAQKLMDYLVSIYGKYTLSGQMGYKDAFWIWNITDKFPAICGFDMMDYSPSRVERGASSRDVEDAIDWWNMGGIVQFQWHWNAPKGLYDTPGKEWWRGFYTNATSFDIEYALNHPESEDYKLIIRDIDAIAVQLKRLQEAKVPILWRPLHEAEGRWFWWGAKGPEACKKLWRLLFDRLVNYHKINNLIWVWTTTDSPDALKWYPGDEYVDIVGADIYLKDKDYSPSTGMFYNIVKLFGGKKLVALTENGIIPDPDLMKEQKAYWVWFMTWSGFENDPNKNEISHIKKVFNHPFVITKDELPNLKVEE

SEQ ID NO: 67 - protein, 329 aa, Thermotoga maritima
MNNTIPRWRGFNLLEAFSIKSTGNFKEEDFLWMAQWDFNFVRIPMCHLLWSDRGNPFIIREDFFEKIDRVIFWGEKYGIHICISLHRAPGYSVNKEVEEKTNLWKDETAQEAFIHHWSFIARRYKGISSTHLSFNLINEPPFPDPQIMSVEDHNSLIKRTITEIRKIDPERLIIIDGLGYGNIPVDDLTIENTVQSCRGYIPFSVTHYKAEWVDSKDFPVPEWPNGWHFGEYWNREKLLEHYLTWIKLRQKGIEVFCGEMGAYNKTPHDVVLKWLEDLLEIFKTLNIGFALWNFRGPFGILDSERKDVEYEEWYGHKLDRKMLELLRKY

SEQ ID NO: 68 - protein, 694 aa, Geobacillus stearothermophilus
MNKKWSYTFIALLVSIVCAVVPIFFSQNNVHAKTKREPATPTKDNEFVYRKGDKLMIGNKEFRFVGTNNYYLHYKSNQMIDDVIESAKKMGIKVIRLWGFFDGMTSENQAHNTYMQYEMGKYMGEGPIPKELEGAQNGFERLDYTIYKAKQEGIRLVIVLTNNWNNFGGMMQYVNWIGETNHDLFYTDERIKTAYKNYVHYLINRKNQYTGIIYKNEPTIMAWELANEPRNDSDPTGDTLVRWADEMSTYIKSIDPHHLVAVGDEGFFRRSSGGFNGEGSYMYTGYNGVDWDRLIALKNIDYGTFHLYPEHWGISPENVEKWGEQYILDHLAAGKKAKKPVVLEEYGISATGVQNREMIYDTWNRTMFEHGGTGAMFWLLTGIDDNPESADENGYYPDYDGFRIVNDHSSVTNLLKTYAKLFNGDRHVEKEPKVYFAFPAKPQDVRGTYRVKVKVASDQHKVQKVQLQLSSHDEAYTMKYNASFDYYEFDWDTTKEIEDSTVTLKATATLTNKQTIASDEVTVNIQNASAYEIIKQFSFDSDMNNVYADGTWQANFGIPAISTPKTRCLRVNVDLPGNADWEEVKVKISPISELSETSRISFDLLLPRVDVNGALRPYIALNPGWIKIGVDQYHVNVNDLTTVTIHNQQYKLLHVNVEFNAMPNVNELFLNIVGNKLAYKGPIYIDNVTLFKKI

SEQ ID NO: 69 - protein, 569 aa, Ruminococcus albus
MKQNGVNLYAISVQNEPDYAKDWTAWTPDETTDFIANYGDQITSTKLMSPESFQYGAYNNGKDYYSKILNNSKAYANCDIFGTHFYGTPRSKMDFPALENCGKQLWMTEVYVPDSNVDSNIWPDNLKQAVSIHDSLVVGGMQAYVVWPLRRNYSILREDTHKISKRGYAFAQYSKFVRPGDVRVDVTEQPSSNVFVSAYKNNKNQVTIVAINNSSSGYSQQFSLNGKTIIDVDRWRTSGSENLAETDNLTIDNGTSFWAQLPAQSVSTFVCTLSGGSSSGNNGSSNTELDSDGYYFHDTFEDDLTWQAHGGTELLKSGRTPYKGSEAVVVTNRTSAWMGAERTLPSSVVPGKTYSFSVNVTELDGEDTETFYLKLNYTDSSGTAHYPTIAEGVCPKGKYLQLSNTNYTIPSDAVDPVIYVETKDTTSNFYIDEAICAPAGKSLPGAGIPEIPSNNNNNNNNNNNNNNNNNNNNQNNSVYPVVSSIDYNVTYHQFRISWNSVPNAQAYGIAYYAAGKWRVYTQSISVNTTSWISPKLTAGKTYTMVIAAKVGGKWDTSNLSSRAINVTVK

SEQ ID NO: 70 - protein, 1217 aa, Cytophaga hutchinsonii
MKKLFTVLFYLSTCLVWAQTSTVNLTSEKQYIRGFGGINHPEWAGDMTAVQRTTAFGNGAGEMGLTVLRIFVNDDKTQWNKALATALRAQQLGATIFATPWNPPASMCETITRNNRQEKRLKPGSYSAYAQHLIDFNNYMKNNGVNLYAMSFANEPDWGFDWTWYSADEVYNFTKNIAGTLRVNGIKVITAESFSYNKSYYDKVLNDPTALSNIDIIGCHLYGSDANSPVSVFNYPLADSKAPTKERWMTEHYTNSDANSSDLWPSANDVSYEIYRCMVEGQMSVYTWWYIRRQYGPMNENGTISKRGYCMAQYSKFIRPGYKRVDATKNPATGVYISAYKKGDDVVVVAINRSTSSQTITLSVPGTKVTTWEKYVTSGSKSLAKEANINSSTGSFQITLDPQSTTSFVGTAPVITTPSPVVSLTAPVNNTVYTEGDNITINATATITSGSISKVEFYNGTTLLGTDASSPYSYTITAAAAGTYPVTAKATSAANAVTTSTAINIQVAKPIYQTGSAPTIDGTVDGLWSNFPSTGITKNNTGTISSGTDLSGNWKAMWDASNLYVLVQVTDDVKRNDGGTDVYNDDGVEVYIDLGNTKATTYGTNDQQYTFRWNDVTAAYEINGHPVTGITKGISNTATGYIVEVSIPWSTIGGTASLNSFQGFEVMINDDDDGGAREGKLAWVASTDDTWSNPALMGTVVLKGLNCTVPAAAITASTATTFCSGGSVVLNAGTGTGYSYVWKNGAATIAGATNSGYTATASGSYTVTVTNPGGCSATSAGTTVTVNALPVLTQYAQVDGGTWNQVSGATVCAGSSVVLGPQPTVNTGWSWTGPNGYSASARELRLTSVQTNQGGVYTASYTDGNTCKSTSVFTLTVTALPAAAITTSTPTTFCAGGSTTLTAGSGASYKWMNGTVAITGATAQTYTATAAGSYTVEVTNAGNCKATSAATVVTVTALPTATITATGSTTIPQGGSVALQANAGSALTYKWFNGTVAITGATAQTYTATTAGSYTVEVTNAGNCKATSAAATVSVVANQPSVITITSPAPNAAVTGAIDISVNITDADGSITLVEFLAGDDVIGTAAAAPYTYTWDTPTAGSHTITVRVTDSNGGVTTSAPVTVTSESITTGVQALNTLNAAVYPNPSNGIVFIDTDADLSDASFTLIDVLGKEGTVFSTATGNGAMIDVSSLAGGTYVLIVKKDTSVIRKKITVIR

SEQ ID NO: 71 - protein, 536 aa, Piromyces equi
MKTSIVLSIVALFLTSKASADCWSERLGWPCCSDSNAEVIYVDDDGDWGVENNDWCGIQKEEENNNSWDMGDWNQGGNQGGGMPWGDFGGNQGGGMQWGDFGGNQGGGMPWGDFGGNQGGGMPWGDFGGNQGGNQGGGMPWGDFGGNQGGNQGGGMPWGDFGGNQGGGMQWGDFGGNQGGNQGGGMPWGDFGGNQGGGMQWGDFGGNQGGNQGGGMPWGDFGGNQGGGMQWGDFGGNQGGGMQWGDFGGNQGGNQDWGNQGGNSGPTVEYSTDVDCSGKTLKSNTNLNINGRKVIVKFPSGFTGDKAAPLLINYHPIMGSASQWESGSQTAKAALNDGAIVAFMDGAQGPMGQAWNVGPCCTDADDVQFTRNFIKEITSKACVDPKRIYAAGFSMGGGMSNYAGCQLADVIAAAAPSAFDLAKEIVDGGKCKPARPFPILNFRGTQDNVVMYNGGLSQVVQGKPITFMGAKNNFKEWAKMNGCTGEPKQNTPGNNCEMYENCKGGVKVGLCTINGGGHAEGDGKMGWDFVKQFSLP

SEQ ID NO: 72 - protein, 558 aa, Fusarium oxysporum
MLFASLVLVLGFIPQVLSDTSTDICLPQDNMRPTFLLFSGLGACAAAGKGDDFAAKCAGFKTSLKLPNTKVWFTEHVPAGKHITFPDNHPTCTPKSTITDVEICRVAMFVTTGPKSNLTLEAWLPSNWTGRFLSTGNGGMAGCIQYDDVAYGAGFGFATVGANNGHNGTSAVSMYKNSGVVEDYWYRSVHTGTVLGKELTKKFYGKKHTKSYYLGCSTGGRQGWKEAQSFPDDFDGIVAGAPAMRFNGLQSRSGSFWGITGPPGAPTHLSPEEWAMVQKNVLVQCDEPLDGVADGILEDPNLCQYRPEALVCSKGQTKNCLTGPQIETVRKVFGPLYGNNGTYIYPRIPPGADQGFGFAIGEQPFPYSTEWFQYVIWNDTKWDPNTIGPNDYQKASEVNPFNVETWEGDLSKFRKRGSKIIHWHGLEDGLISSDNSMEYYNHVSATMGLSNTELDEFYRYFRVSGCGHCSGGIGANRIGNNRANLGGKEAKNNVLLALVKWVEEDQAPETITGVRYVNGATTGKVEVERRHCRYPYRNVWDRKGNYKNPDSWKCELPK

SEQ ID NO: 73 - protein, 280 aa, Penicillium chrysogenum
MKISAPRALALSVAVGHALAAVTKGVSDNIYNRLVDMATISQAAYADLCKIPATITTVEKIYNAQTDINGWVLRDDSRQEIIVVFRGTAGDTNLQLDTNYTLAPFDTLPKCIGCAVHGGYYLGWTSVQDQVESLVQQQAGQYPEYALTVTGHSLGASMAAITASQLSATYEHVTLYTFGEPRTGNLAYASYMNENFEATSPETTRFFRVTHGNDGIPNLPPAEQGYVHSGIEYWSVDPHRPGSTSVCTGNEVQCCEAQGGQGVNDDHITYFGMASGACSW

SEQ ID NO: 74 - protein, 353 aa, Penicillium funiculosum
MAIPLVLVLAWLLPVVLAASLTQVNNFGDNPGSLQMYIYVPNKLASKPAIIVAMHPCGGSATEYYGMYDYHSPADQYGYILIYPSATRDYNCFDAYSSASLTHNGGSDSLSIVNMVKYVISTYGADSSKVYMTGSSSGAIMTNVLAGAYPDVFAAGSAFSGMPYACLYGAGAADPIMSNQTCSQGQIQHTGQQWAAYVHNGYPGYTGQYPRLQMWHGTADNVISYADLGQEISQWTTIMGLSFTGNQTNTPLSGYTKMVYGDGSKFQAYSAAGVGHFVPTDVSVVLDWFGITSGTTTTTTPTTTPTTSTSPSSTGGCTAAHWAQCGGIGYSGCTACASPYTCQKANDYYSQCL

SEQ ID NO: 75 - protein, 292 aa, Neurospora crassa
MLPRTLLGLALTAATGLCASLQQVTNWGSNPTNIRMHTYVPDKLATKPAIIVALHGCGGTAPSWYSGTRLPSYADQYGFILIYPGTPNMSNCWGVNDPASLTHGAGGDSLGIVAMVNYTIAKYNADASRVYVMGTSSGGMMTNVMAATYPEVFEAGAAYSGVAHACFAGAASATPFSPNQTCARGLQHTPEEWGNFVRNSYPGYTGRRPRMQICHGLADNLVYPRCAMEALKQWSNVLGVEFSRNVSGVPSQAYTQIVYGDGSKLVGYMGAGVGHVAPTNEQVMLKFFGLIN

SEQ ID NO: 76 - protein, 767 aa, Saccharophagus degradans
MKSINVCGRRLKQALAAIATAAATLWFTPVDAQTLTSNQTGTHGGYYYSFWTDSAGTVSMTLGNGGNYSSSWSNTGNWVGGKGWQTGGRKTVNYSGTFNPSGNGYLTLYGWTQNPLIEYYIIESWGTYRPGESGTYYGTVNTDGGTYDIYRTQRVNQPSIEGTATFYQYWSVRQQKRVGGTITTGNHFDAWASHGLNLGTHNYMVMATEGYQSSGNSNITVSEGSGSSSTSSSSSSTGGPSGTNIVVRAQGVSGQEHINLIIGGNVVADWTLSTSMQDYTYTGNAAGDLQVEYDNDASGRDVELDYVYVNGEIRQAEDMEYNTATYSGECGGGSYSQTMHCSGVIGFGDTSDCFSGNCNGASSTSSSSSSSSTSSSTSSGGNNNSGITVRARGTNGDEHINLIVGGNIVGNWTLTTSNQNYVYNGNASGDVEVQFDNDANGRDVILDYVIVNGETRQAEDMEYNTATYSGSCGGGSYSETMHCSGEIGFGHTDDCFSGNCTSSSGTTGSSGGTSSNNGTSSCNGYVGITFDDGPGNNTATLINLLQQNNLTPVTWFNTGQNIAANTGQFAQQKSVGEIQNHSYTHSHMLNWSYQQVRDELASTNQAIVNAGGATPTLFRPPYGETNSTINQAAQDLGLRVITWDVDSRDWDGASASAIANSANQLQNGQVILMHDASYNNTNGAISQFAANLRARGLCAGKIDPSTGRAVAPSTNTGGNTGSNTGNGGNGGMCNWYGTSIPLCQTTNDGWGWENSQSCVSQNTCNSQ

SEQ ID NO: 77 - protein, 382 aa, Penicillium purpurogenum
MKSLSFSFLVTLFLYLTLSSARTLGKDVNKRVTAGSLQQVTGFGDNASGTLMYIYVPKNLATNPGIVVAIHYCTGTAQAYYTGSPYAQLAEQYGFIVIYPQSPYSGTCWDVSSQAALTHNGGGDSNSIANMVTWTISQYNANTAKVFVTGSSSGAMMTNVMAATYPELFAAATVYSGVGAGCFYSSSNQADAWNSSCATGSVISTPAVWGGIAKNMYSGYSGSRPRMQIYHGSADTTLYPQNYYETCKQWAGVFGYNYDSPQSTLANTPDANYQTTNWGPNLQGIYATGVGHTVPIHGAKDMEWFGFSGSGSSSTTTASATKTSTTSTTSTKTTSSTSSTTTSSTGVAAHWGQCGGSGWTGPTVCESGYTCTYSNAWYSQCL

SEQ ID NO: 78 - protein, 413 aa, Erwinia chrysanthemi
MNGNVSLWVRHCLHAALFVSATAGSFSVYADTVKIDANVNYQIIQGFGGMSGVGWINDLTTEQINTAYGSGVGQIGLSIMRVRIDPDSSKWNIQLPSARQAVSLGAKIMATPWSPPAYMKSNNSLINGGRLLPANYSAYTSHLLDFSKYMQTNGAPLYAISIQNEPDWKPDYESCEWSGDEFKSYLKSQGSKFGSLKVIVAESLGFNPALTDPVLKDSDASKYVSIIGGHLYGTTPKPYPLAQNAGKQLWMTEHYVDSKQSANNWTSAIEVGTELNASMVSNYSAYVWWYIRRSYGLLTEDGKVSKRGYVMSQYARFVRPGALRIQATENPQSNVHLTAYKNTDGKMVIVAVNTNDSDQMLSLNISNANVTKFEKYSTSASLNVEYGGSSQVDSSGKATVWLNPLSVTTFVSK

SEQ ID NO: 79 - protein, 1467 aa, Paenibacillus sp. JDR-2
MSRSLKKFVSILLAAALLIPIGRLAPVAEAAENPTIVYHEDFAIDKGKAIQSGGASLTQVTGKVFDGNNDGSALYVSNRANTWDAADFKFADIGLQNGKTYTVTVKGYVDQDATVPSGAQAFLQAVDSNNYGFLASANFAAGTAFTLTKEFTVDTSVSTQLRVQSSEEGKAVPFYIGDILITANPTTTTNTVYHEDFATDKGKAVQSGGANLAQVADKVFDGNDDGKALYVSNRANTWDAADFKFADIGLQNGKTYTVTVKGYVDQDATVPSGAQAFLQAVDSNNYGFLASANFAARSAFTLTKEFTVDTSVTTQLRVQSSEEGKAVPFYIGDILITETVNSGGGQEDPPRPPALPFNTITFEDQTAGGFTGRAGTETLTVTNESNHTADGSYSLKVEGRTTSWHGPSLRVEKYVDKGYEYKVTAWVKLLSPETSTKLELASQVGDGGSANYPTPTTQAWQARRLPAADGWVQLQGNYRYNSVGGEYLTIYVQSSNATASYYIDDISFESTGSGPVGIQKDLAPLKDVYKNDFLIGNAISAEDLEGTRLELLKMHHDVVTAGNAMKPDALQPTKGNFTFTAADAMIDKVLAEGMKMHGHVLVWHQQSPAWLNTKKDDNNNTVPLGRDEALDNLRTHIQTVMKHFGNKVISWDVVNEAMNDNPSNPADYKASLRQTPWYQAIGSDYVEQAFLAAREVLDENPSWNIKLYYNDYNEDNQNKATAIYNMVKDINDRYAAAHNGKLLIDGVGMQGHYNINTNPDNVKLSLEKFISLGVEVSVSELDVTAGNNYTLPENLAVGQAYLYAQLFKLYKEHADHIARVTFWGMDDNTSWRAENNPLLFDKNLQAKPAYYGVIDPDKYMEEHAPESKDANQAEAQYGTPVIDGTVDSIWSNAQAMPVNRYQMAWQGATGTAKALWDDQNLYVLIQVSDSQLNKANENAWEQDSVEVFLDQNNGKTTFYQNDDGQYRVNFDNETSFSPASIAAGFESQTKKTANSYTVELKIPLTAVTPANQKKLGFDVQINDATDGARTSVAAWNDTTGNGYQDTSVYGELTLAGKGTGGTGTVGTTVPQTGNVVKNPDGSTTLKPEVKTTNGNAVGTVTGDDLKKALDQAAPAAGGKKQVIIDVPLQANAATYAVQLPTQSLKSQDGYQLTAKIANAFIQIPSNMLANTNVTTDQVSIRVAKASLDNVDAATRELIGNRPVIDLSLVAGGNVIAWNNPTAPVTVAVPYAPTAEELKHPEHILIWYIDGSGKATPVPNSRYDAALGAVVFQTTHFSTYAAVSVFTTFGDLAKVPWAKEAIDAMASRGVIKGTGENTFSPAASIKRADFIALLVRALELHGTGTTDTAMFSDVPANAYYYNELAVAKQLGIATGFEDNTFKPDSSISRQDMMVLTTRALAVLGKQLPAGGSLNAFSDAASVAGYAQDSVAALVKAGVVQGSGSKLAPNDQLTRAEAAVILYRIWKLQ

SEQ ID NO: 80 - protein, 444 aa, Thermotoga neapolitana
MKKFPEGFLWGVATASYQIEGSPLADGAGMSIWHTFSHTPGNVKNGDTGDVACDHYNRWKEDIEIIEKIGAKAYRFSISWPRILPEGTGKVNQKGLDFYNRIIDTLLEKNITPFITIYHWDLPFSLQLKGGWANRDIADWFAEYSRVLFENFGDRVKHWITLNEPWVVAIVGHLYGVHAPGMKDIYVAFHTVHNLLRAHAKSVKVFRETVKDGKIGIVFNNGYFEPASEREEDIRAARFMHQFNNYPLFLNPIYRGEYPDLVLEFAREYLPRNYEDDMEEIKQEIDFVGLNYYSGHMVKYDPNSPARVSFVERNLPKTAMGWEIVPEGIYWILKGVKEEYNPQEVYITENGAAFDDVVSEGGKVHDQNRIDYLRAHIEQVWRAIQDGVPLKGYFVWSLLDNFEWAEGYSKRFGIVYVDYNTQKRIIKDSGYWYSNGIKNNGLTD

SEQ ID NO: 81 - protein, 720 aa, Fibrobacter succinogenes
MRIYKLSPIFSAAVLLSAGVASAETKFFYNQVGYDVDQPISVIVQSENLADGAEFSVMSGGTAVKTGKLSTGSNPDNWLNSGKFYVADLTGLKAGKYTLQVSENGQPQKSGEFTVGENALAANTLASVLNYFYDDRADDPTVEGWDKQMPVYKSDKKLDVHGGWYDASGDVSKYLSHLSYANYLNPQQIPLTVWSLAFASERIPKLLGSTSTKAKTADEAAYGADFLVRMLDEQGFFYMTVFDNWGSPMGKREICAFSGSDGIKSTDYQTAFREGGGMAIAALASAARLKLKGDFTSEQYLAAAEKAYKHLSEKQSVGGDCAYCDDHKENIIDDYTALLAATELYAATKKQEYLEDAYDRAEHLSSRVSKDGYFWSDDAKTRPFWHASDAGLPLVALARYSEVVGAIDEDAGIKVHGRPFPYWGCVTMIGGGCVNESIDNVRNAIRSHFDWLVKITNKVDNPFGYARQTYKTQDKIKDGFFIPHDNESNYWWQGEDARLASLSAAIMYANRIIDGEYRNVTTSDVQKYATDQLDWILGKNPYATCMMYGKGTKNPQKYDGQSKYDATLEGGIANGISGKNQDGSGIAWTDDGVAAVGFDSEKESWQVWRWDEQWLPHSTWYLMALVERYDELTKPVEFSVGLSKSTVAAKASVSLVGKMLSLNLPRSVVGKSVKVLDVRGNVLMQKTVQGVSETMDVSTLNRGLYLVQIQGFAAKKFVVK

SEQ ID NO: 82 - protein, 925 aa, Thermobifida fusca
MTATAQRTPPPPTPRRRGIIARALTCIAAAATVAAVGLVHSAAAPASATTGYTWRNVEIVGGGFVPGIVFNQSEPDLIYARTDIGGAYRWDPATERWIPLLDHVGWDDWGHSGVVSIATDPVDPDRVYAAVGTYTNDWDPNNGAIKRSTDRGETWETTELPFKLGGNMPGRGMGERLAIDPNDNSVLYLGAPSGHGLWKSTDYGKTWQKVTSFPNPGNYVADPSDVGGYLGDNQGVVWVVFDPTSSSPGHVTKDIYVGVADKQNTVYRSTDGGQTWERIPGQPTGFLAQKGVFDHVNGLLYIATSDTGGPYDGSDGEVWRYDTTTGTWTDITPADPDGFEYGFSGLTIDRQNPDTIMVVSQILWWPDIQIWRSTDRGETWSRIWEFSGYPDRTLRYNHDISAAPWLDFNRQDNPPEVSPKLGWMTQAFEIDPFNSDRMLYGTGATIYGSDNLTNWDEGKKIDIKVRAQGIEETAVQDLIAPPGDTELVSALGDIGGFVHDDITVVPDAMFDSPFHGNTRSIDFAELNPSVMARVGEAVDGEVDSHIGISTSGGSHWWAGQEPSGVTGAGTVAVNADGSRIVWSPDGTGVHYSTTLGSSWTPSQGVPAGARVEADRVNPDKFYAFANGTFYTSTDGGATFTKSSAAGLPTKGNIRFAAVPGHEGDIWLAGGETNSTYGMWRSTDSGATFTRITAVDEGDVVGFGKPAPGRSYPAVYTSSKINGVRGIFRSDDAGTTWVRINDDQHQWAWTGAAITGDPDVYGRVYIGTNGRGVIVGDLDGPPPQPTEEPTEEPSTPPTEEPTEEPTEEPSTPPTEEPPGDAACAVSYQVLNEWGGGFQGEVTITNTGDTPINGWELTWTFPDNQQITQAWNTQLTQSGAKVTARDAGWNSTIAPGGTASFGFLGSPAPGSKPTEFTLNGTPCSAAG

SEQ ID NO: 83 - protein, 540 aa, Phanerochaete chrysosporium
MFRAAALLAFTCLAMVSGQQAGTNTAENHPQLQSQQCTTSGGCKPLSTKVVLDSNWRWVHSTSGYTNCYTGNEWDTSLCPDGKTCAANCALDGADYSGTYGITSTGTALTLKFVTGSNVGSRVYLMADDTHYQLLKLLNQEFTFDVDMSNLPCGLNGALYLSAMDADGGMSKYPGNKAGAKYGTGYCDSQCPKDIKFINGEANVGNWTETGSNTGTGSYGTCCSEMDIWEANNDAAAFTPHPCTTTGQTRCSGDDCARNTGLCDGDGCDFNSFRMGDKTFLGKGMTVDTSKPFTVVTQFLTNDNTSTGTLSEIRRIYIQNGKVIQNSVANIPGVDPVNSITDNFCAQQKTAFGDTNWFAQKGGLKQMGEALGNGMVLALSIWDDHAANMLWLDSDYPTDKDPSAPGVARGTCATTSGVPSDVESQVPNSQVVFSNIKFGDIGSTFSGTSSPNPPGGSTTSSPVTTSPTPPPTGPTVPQWGQCGGIGYSGSTTCASPYTCHVLNPCESILSLQRSSNADQYLQTTRSATKRRLDTALQPRK

SEQ ID NO: 84 - protein, 837 aa, Clostridium thermocellum
MSRKLFSVLLVGLMLMTSLLVTISSTSAASLPTMPPSGYDQVRNGVPRGQVVNISYFSTATNSTRPARVYLPPGYSKDKKYSVLYLLHGIGGSENDWFEGGGRANVIADNLIAEGKIKPLIIVTPNTNAAGPGIADGYENFTKDLLNSLIPYIESNYSVYTDREHRAIAGLSMGGGQSFNIGLTNLDKFAYIGPISAAPNTYPNERLFPDGGKAAREKLKLLFIACGTNDSLIGFGQRVHEYCVANNINHVYWLIQGGGHDFNVWKPGLWNFLQMADEAGLTRDGNTPVPTPSPKPANTRIEAEDYDGINSSSIEIIGVPPEGGRGIGYITSGDYLVYKSIDFGNGATSFKAKVANANTSNIELRLNGPNGTLIGTLSVKSTGDWNTYEEQTCSISKVTGINDLYLVFKGPVNIDWFTFGVESSSTGLGDLNGDGNINSSDLQALKRHLLGISPLTGEALLRADVNRSGKVDSTDYSVLKRYILRIITEFPGQGDVQTPNPSVTPTQTPIPTISGNALRDYAEARGIKIGTCVNYPFYNNSDPTYNSILQREFSMVVCENEMKFDALQPRQNVFDFSKGDQLLAFAERNGMQMRGHTLIWHNQNPSWLTNGNWNRDSLLAVMKNHITTVMTHYKGKIVEWDVANECMDDSGNGLRSSIWRNVIGQDYLDYAFRYAREADPDALLFYNDYNIEDLGPKSNAVFNMIKSMKERGVPIDGVGFQCHFINGMSPEYLASIDQNIKRYAEIGVIVSFTEIDIRIPQSENPATAFQVQANNYKELMKICLANPNCNTFVMWGFTDKYTWIPGTFPGYGNPLIYDSNYNPKPAYNAIKEALMGY

SEQ ID NO: 85 - DNA, 1330 bp, Talaromyces emersonii
ccatggatccacagcaagcgggtacggccaccgcggagaaccatcccccccttacgtggcaagaatgcaccgcccccggatcgtgcactactcaaaatggcgctgtggttctcgatgctaactggcggtgggttcacgatgttaatggttacactaactgctatacaggcaatacatgggacccgacctactgccctgacgacgagacttgcgcccagaactgcgcacttgatggtgcggattatgaaggaacgtacggagtcacctcctccggctcttcccttaagcttaatttcgtgacaggcagcaatgtgggatcaaggctctatctgctccaggacgattctacctaccaaatattcaagctcctcaacagagaattttccttcgacgtcgacgtttctaatctcccttgtggcctcaatggtgcactctatttcgtagccatggacgcagacggcggagtctcgaaatacccaaacaacaaggctggtgctaagtatggtacgggatactgcgatagccagtgtccacgcgatcttaaatttattgacggtgaagcaaacgtagaaggttggcagccatcatctaacaacgcaaacacaggtatcggcgatcacggcagctgttgtgctgaaatggacgtctgggaagcaaactcaatatccaatgcggttaccccccatccttgcgataccccaggtcagacgatgtgctctggagacgattgtggtggaacctactcgaatgaccgctatgccggcacctgcgatccagatggatgcgacttcaatccctaccgcatgggtaatacctcattctacggccccggaaaaataattgacaccacgaagcctttcactgtagtaactcaatttttgactgacgacggaacagacaccggtaccctgtccgagatcaaaagattctacatccagaattcaaacgtcatccctcaacctaatagcgacatatcaggcgtgaccggtaactcgataacaactgagttttgcacagcccagaaacaagcgttcggcgacacagacgatttctcccaacacggaggcctggcaaaaatgggagctgcgatgcaacaaggcatggtactcgtgatgagtctttgggatgattatgctgcgcaaatgctttggctggattccgattatccgacagatgcagacccaacaaccccaggaatagctagaggcacctgcccaactgattcaggcgtaccgagcgatgtcgaaagccagtctcctaattcttacgttacatactccaatattaagttcggaccaattaactctacattcacggcctcaggagatct

SEQ ID NO: 86 - DNA, 823 bp, Neurospora crassa
ccatggatccagctccctcctccggctgcggaaaaggaccaactctgcgcaacggccaaacggtgacaacaaatattaacggcaagagtaggagatacaccgtgaggttgccggataactacaatcagaacaacccataccgcctgatattcctctggcatccgctcggatcttccatgcagaagatcatccagggcgaggaccccaacagaggcggcgtcctgccttactacggcctgccgccgctcgatacatccaagtcagccatctatgtggttccggatggattgaacgcgggctgggcgaatcagaacggagaggacgtctcattctttgataacatcttgcaaaccgtgtcagacggtctgtgtatcgacacaaatcttgtgttcagcaccggcttcagctacggagggggcatgtctttctcccttgcctgcagccgcgcgaacaaggtgcgcgctgtcgccgtgattagtggtgcacagctctccgggtgcgcaggcggaaacgacccggtggcgtactacgctcagcacggtaccagcgacggcgtccttaatgtggcgatgggccgccagctccgggacaggttcgtcaggaacaacggctgccagcccgccaatggcgaggtgcagccaggcagtggaggaaggagcacccgcgtcgaataccaaggttgtcagcaaggcaaggatgtggtgtgggtcgttcacggcggggaccacaacccatcccaaagggaccccggtcagaatgacccgttcgctcctaggaacacctgggaatttttcagtcgcttcaactaaggcgcgccagatct

SEQ ID NO: 87 - DNA, 1180 bp, Pyrococcus furiosus
ccatggatccagagcagacccaaacacagacacttgagtcgaacagcccgactcaaaccacaaccacgaccagccctcaaatcactgtgactttcattgtctcagtccccgaatacacccctgagaatgactctatctatatcgcgggcgacttcaacaactggaatccgaaggatgaaagatacaagctggtgaagctgccggacgggaggtggaagattactctcaccttcccttacggtaagaccatccagttcaagttcacgcgcggctcctgggagacggtggagaagggcatcaacggcgaggagatcccgaaccgcagatttacgttcacgaagagcggcacctatgaatttaaggttcacaattggagagattttgtggaaaagaatgtgaagcacacaatcaccggcaacgtgatcactttcgagatgttcatcccacagctcaacaccacaaggagaatctggatctatctcccaccggactacaactactcaaccaagcgctacccggtgctctacatgttcgatggccagaatctgttcgatgcggcaacatctttcgctggggagtggggagtggacgaagcgcttgagaagctttacaaggaaaagaatttctccattattgttgtcggcattgataacggcggcgacaggcgcattgatgagtatgccccttgggttaaccgggattacagaaggggtggactgggaaacgccaccgtcaagttcatagtcgagacgctgaagccttacattgacgcgcactacaggacagaccccgaaaagaccggtatcatgggaagcagcctgggaggcctgatggctatatatgccggtttctcttatccggaagtgttcaggtacgtaggcgccatgtcgagtgccttctggtttaacccggaaatttatgatttcgttcgcgaggccaagaagggcccagagaagatttatatcgactggggtaccaacgaaggccgcaacccgaaggcgttcagcgagagtaacgagaaaatggtcaagatcctcaaagagaaggggtaccgcgaggagttcaacctcaaggtcgtgatcgataaaggagggctgcacaacgagtattactggggaaagagattccctcaggccgtgttgtggctcttcgaggagtaaggcgcgccagatctgagctc

SEQ ID NO: 88 - DNA, 1363 bp, Thermotoga neapolitana
ccatggatcctaagaagttcccggagggctttctctggggcgtggcgaccgccagctaccagatcgagggctccccactcgccgatggcgcaggcatgtccatctggcacaccttcagtcacacgccgggcaatgtcaagaacggtgacaccggcgacgtggcttgcgaccactacaaccgctggaaggaggacatcgagatcatagagaagatcggcgccaaggcctacaggttttccatctcctggccaaggatactcccggagggaaccggcaaggtcaaccagaagggcctcgacttttacaaccggatcattgacaccctcctggagaagaacatcaccccgttcatcaccatctaccactgggatctccccttttcccttcagctcaagggcggctgggccaacagggacatcgctgattggttcgccgagtattcccgcgtgctcttcgagaacttcggcgacagagtgaagcactggatcaccctcaacgagccgtgggtggtggccatcgttggccacctctacggcgtgcacgccccaggcatgaaggatatatacgtggctttccacaccgtgcacaatctccttagggcccacgcgaagagcgtgaaggtgtttagggaaaccgtgaaggacggcaagatcggcattgtgttcaacaatggctacttcgagccggcttccgagagggaagaggacatcagggccgccaggtttatgcaccagttcaataactacccgctgtttctcaacccgatatacaggggcgagtacccggacctcgtgcttgagttcgccagggaatacctgcccaggaactacgaggatgacatggaggaaatcaagcaggagattgacttcgtgggcctcaactactacagtggccacatggtgaagtacgatccgaactccccagccagggtgtccttcgtggagaggaacctcccaaagaccgctatgggctgggagatcgttccggagggcatatactggattctcaagggcgtgaaggaggagtacaacccgcaggaggtgtatatcaccgagaacggcgctgccttcgacgatgttgtgtccgagggcggtaaagtgcacgaccagaacaggatcgactacttgcgagcccatattgagcaggtctggagggcaattcaggatggcgttccgctcaaggggtacttcgtgtggtccctgctcgacaattttgagtgggccgagggctactccaagaggttcggcatcgtttacgtggactacaacacccagaagaggatcattaaggactccggctactggtacagtaacggcatcaaaaacaacggcctcaccgactaaggcgcgccagatctgagctc

SEQ ID NO: 89 - DNA, 1253 bp, Erwinia chrysanthemi
ccatggcaaacggcaacgtgtccctctgggtgaggcactgcctccacgcagcactcttcgtgtccgcaaccgcaggctccttctccgtgtacgccgacaccgtgaagatcgacgccaacgtgaactaccagatcatccagggcttcggcggcatgtccggcgtgggctggatcaacgacctcaccaccgagcagatcaacaccgcctacggctccggcgtgggccagatcggcctctccatcatgagggtgaggatcgacccggactcctccaagtggaacatccagctcccgtccgccaggcaggccgtgtccctcggagcaaagatcatggcaaccccgtggtccccaccagcctacatgaagtccaacaactccctcatcaacggcggcaggctcctcccggccaactactccgcctacacctcccacctcctcgacttctccaagtacatgcagaccaacggcgccccgctctacgccatctccatccagaacgagccggactggaagccggactacgagtcctgcgagtggtccggcgacgagttcaagtcctacctcaagtcccagggctccaagttcggctccctcaaggtcatcgtggcagagtccctcggcttcaacccagcactcaccgacccggtgctcaaggactccgacgcctccaagtatgtgagcattatcggaggacacctctacggaaccaccccaaagccatacccactcgcacagaacgcaggcaagcagctctggatgaccgagcactacgtggactccaagcagtccgccaacaactggacctccgccatcgaagtgggcaccgagctgaacgccagcatggtgtccaactactccgcctacgtgtggtggtacatcaggaggtcctatggcctcctcaccgaggacggcaaggtgtccaagaggggctacgtgatgtcccagtacgccaggttcgtgaggccgggcgccctcagaatccaggccaccgagaacccgcagtccaacgtgcacctcaccgcctacaagaacaccgacggaaagatggtcatcgtggccgtgaacaccaacgactccgaccagatgctctccctcaacatctccaacgccaacgtgaccaagttcgagaagtactccacctccgcctccctcaacgtggagtacggaggctcctcccaggtggactcctccggcaaggcaaccgtgtggctcaacccactctccgtgaccaccttcgtgtccaagtcagatctc

SEQ ID NO: 90 - DNA, 1544 bp, Fibrobacter succinogenes
ccatggcagccccggacccgaacttccacatctacatcgcctacggccagtccaacatggagggcaacgccaggaacttcaccgacgtggacaagaaggagcacccgagggtgaagatgttcgcaaccacctcctgcccgtccctcggaaggccaaccgtgggagagatgtacccagcagtgccaccaatgttcaagtgcggagagggactctccgtggcagactggttcggaaggcacatggcagactccctcccaaacgtgaccatcggcatcatcccagtggcacagggaggcacctccatcaggctcttcgacccggacgactacaagaactacctcaactccgccgagtcctggctcaagaacggcgccaaggcctacggcgacgacggcaacgctatgggaaggatcatcgaggtggccaagaaggcccaggagaagggcgtgatcaagggcatcatcttccaccagggcgagaccgacggcggcatgtccaactgggagcagatcgtgaagaagacctacgagtacatgctcaagcagctcggcctcaacgcagaggagaccccattcgtggcaggagagatggtggacggaggctcctgcgcaggcttctcctccagggtgaggggcctctccaagtacatcgccaacttcggcgtggcctcctccaagggctacggctccaagggcgacggcctccacttcaccgtggagggctacaggggcatgggcctccgctacgcccagcagatgctcaagctcatcaacgtggcaccagtggacccggtgccacaggagccgttcaagggtgctccaatcgcaatcccaggcaaggtggaggtggaggacttcgacaagccgggcatcggcaagaacgaggacggcacctccaacgcctcctactccgacgaggactccgagaaccacggcgactccgactacaggaaggacaccggagtggacctctacaaggcaggcgacggagtggcactcggatacacccagaccggagagtggctggagtacaccgtggacgtgaaggccgacggcgagtacaacatcgacgcctccgtggccgccggcaactccacctccgccttcaagctctacatcgacgagaaggccatcaccgacgacgtgtccgtgccgcagaccgccgacaactcctgggacacctacaagaccatctccgtgaaggagaaggtgaccctcaaggccggcaagcacgtgctcaagctggagatcaccgccaactacgtgaacatcgactggattcagttctccgagccgaagaaggaggacccgccgtccgccatcgccaaggtgaggttcgacatgaccgaggccgagtccaacttctccgtgtactccatgcagggccagaagctcggcaccttcaccgccaagggcatggccgacgccatgaacctcgtgaagaccgacgccaagctcaggaagcaggccaagggcgtgttcttcgtgaggaaggagggcgccaagctcatgtccaagaaggtggtggtgttcgagtcagatctc

SEQ ID NO: 91 - protein, 15 aa, Artificial sequence
  Portion obtained from neurospora crassa ferulic acid esterasesequence
CNPSQRDPGQNDPFA

SEQ ID NO: 92 - protein, 17 aa, Artificial sequence
  Portion obtained from neurospora crassa ferulic acid esterasesequence
CRYTVRLPDNYNQNNPY

SEQ ID NO: 93 - protein, 15 aa, Artificial sequence
  Portion obtained from Talaromyces emersonii exoglucanase sequence
CYPNNKAGAKYGTGY

SEQ ID NO: 94 - protein, 16 aa, Artificial sequence
  Portion obtained from Talaromyces emersonii exoglucanase sequence
CNPYRMGNTSFYGPGK

(19) United States
(12) Patent Application Publication
(10) Pub. No.: US 2010/0017916 A1
(43) Pub. Date: Jan. 21, 2010

(54) Systems for reducing biomass recalcitrance

(75) Inventors: Kirk PAPPAN, Abilene, KS (US); Ramesh NAIR, Manhattan, KS (US); Forrest CHUMLEY, Manhattan, KS (US)

Correspondence Address: CHOATE, HALL & STEWART LLP, TWO INTERNATIONAL PLACE, BOSTON, MA, 02110, US

(73) Assignee: EDENSPACE SYSTEMS CORPORATION, Manhattan, KS (US)

(21) Appl. No.: 12/476,247

(22) Filed: Jun. 1, 2009

Related U.S. Application Data
(60) Provisional application No. 61/057,756, filed on May 30, 2008.

(51) Int. Cl. A01H 5/00; C12N 9/14; C12N 9/24; C12N 9/16; C12N 9/88; C12N 9/42; C12N 1/20; C12N 5/04; C12P 19/14; C07K 16/40; C40B 40/08

(52) U.S. Cl. 800/298; 435/195; 435/200; 435/183; 435/196; 435/232; 435/209; 435/320.1; 435/419; 435/99; 530/387.9; 506/17

(57) ABSTRACT

Improved systems and methods for reducing costs and increasing yields of cellulosic ethanol including compositions of matter comprising plant biomass and cell wall-modifying enzyme polypeptides and transgenic plants expression cell wall-modifying enzyme polypeptides.

### RELATED APPLICATION INFORMATION

The present application claims benefit of, and priority to, U.S. provisional application Ser. No. 61/057,756, filed on May 30, 2008, the contents of which are herein incorporated by reference in their entirety.

### GOVERNMENT SUPPORT

This invention was made with U.S. government support under the United States Department of Agriculture and Department of Energy Biomass Grant No. DE-PS36-06GO96002F. The government of the United States of America has certain rights in the invention.

### BACKGROUND

Resistance of cell wall components to degradation is a key source of strength and pathogen defense for plants, but this resistance, commonly referred to as biomass recalcitrance, also represents a significant barrier in the conversion of lignocellulosic mass into simple sugars for fuel ethanol production and for improvement of forage and silage digestibility. Conversion of glucan (i.e., cellulose) to fermentable sugars is accomplished by a series of enzymes known as cellulases. However, before cellulases can efficiently hydrolyze cellulose to simpler sugars, the surrounding matrix of hemicellulose, lignin, beta-glucans, homogalacturonans and rhamnogalacturonans should be partially or completely removed to expose the cellulose. Hemicellulose, lignin, and pectin are cell wall structural polymers that provide additional strength to cell wall through extensive networks of cross-links with one another. Side chains of hemicellulose and pectin provide sites for covalent cross-linking and these side chains can also limit the accessibility of the polysaccharide backbone to enzymatic hydrolysis. Mixed (1,3),(1,4)-beta-D-glucans also embed within cellulosic microfibrils and act as an additional barrier to cellulose.

### SUMMARY

The present invention encompasses the understanding that several distinct classes of enzyme polypeptides would be advantageous for the breakdown of lignocellulosic biomass, given the diversity of chemical components and bonds involved in the matrix surrounding cellulose.

In one aspect, provided are enzyme polypeptides that modify plant cell wall (“cell wall-modifying enzyme polypeptides”) that may be used alone or in conjunction with other enzymes to break down lignocellulosic biomass. In some embodiments, provided are compositions of matter comprising plant biomass and an enzyme polypeptide having at least 85% amino acid sequence identity to at least one of SEQ ID NO: 1 to 84.

In one aspect, provided are plants that transgenically express microbial, plant or animal genes encoding cell wall-modifying enzyme polypeptides. In some embodiments, provided are transgenic plants, the genomes of which are augmented with: a recombinant polynucleotide encoding at least one enzyme polypeptide operably linked to a promoter sequence, wherein the polynucleotide is optimized for expression in the plant, wherein the at least one enzyme polypeptide has at least 85% sequence identity to at least one of SEQ ID NO.: 1 to 84.

In various aspects, provided are expression vectors and transformed cells useful in methods and systems of the invention. In some embodiments, provided are expression vectors comprising a nucleic acid encoding an enzyme polypeptide having at least 85% amino acid sequence identity to at least one of SEQ ID NO: 1 to 84. In some embodiments, provided are expression vectors comprising a nucleic acid encoding an enzyme polypeptide having at least 85% amino acid sequence identity to at least one of SEQ ID NO: 1 to 84.

In one aspect, provided are methods for cost-effective processing of lignocellullosic biomass. In some embodiments, provided are methods comprising steps of: pretreating a plant part under conditions to promote accessibility of celluloses within the lignocellulosic biomass; and treating the pretreated plant part under conditions that promote hydrolysis of cellulose to fermentable sugars, wherein the plant part is obtained from at least one transgenic plant, the genome of which is augmented with: a recombinant polynucleotide encoding at least one enzyme polypeptide operably linked to a promoter sequence, wherein the polynucleotide is optimized for expression in the plant and wherein the at least one enzyme polypeptide has at least 85% sequence identity to at least one of SEQ ID NO.: 1 to 84.

In various aspects, provided are antibodies, gene arrays, and plates useful for testing, screening, and/or characterizing transgenic plants of the invention. In various embodiments, provided are an isolated antibody to a feruloyl esterase polypeptide and an isolated antibody to an exoglucanase polypeptide. In some embodiments, provided are arrays comprising a solid substrate, the substrate having a surface, and a plurality of genetic probes wherein each genetic probe is immobilized to a discrete spot on the surface of the substrate to form an array, and wherein the plurality of genetic probes comprises at least ten different oligonucleotides, each oligonucleotide comprising at least ten consecutive nucleotides from a nucleic acid encoding a polypeptide have a sequence of one of SEQ ID NO: 1 to 84. 65. In some embodiments, provided are plates comprising a solid substrate, the substrate having a surface, and a peptide immobilized to the surface, wherein the peptide comprises at least six consecutive amino acids from a polypeptide having a sequence of one of SEQ ID NO: 1 to 84.

These and other objects, advantages and features of the present invention will become apparent to those of ordinary skill in the art having read the following detailed description.

### BRIEF DESCRIPTION OF THE DRAWING

FIGS. 1-6 are maps of bacterial expression plasmids for expressing fusion proteins that are tagged cell wall-modifying enzyme polypeptides.

FIG. 1 depicts a plasmid map for expressing a HAT-tagged exoglucanase, CBH-E.

FIG. 2 depicts a plasmid map for expressing a HAT-tagged cellulase, TnGGH.

FIG. 3 depicts a plasmid map for expressing a HAT-tagged feruloyl esterase from Neurospora crassa, NcFAE.

FIG. 4 depicts a plasmid map for expressing a HAT-tagged feruloyl esterase from Pyrococcus furiosis, PfFAE.

FIG. 5 depicts a plasmid map for expressing a HAT-tagged glucuronoxylanase from Erwinia chrysanthemi, EcGXX.

FIG. 6 depicts a plasmid map for expressing a HAT-tagged acetyl xylan esterase from, Fibrobacter succinogene, FsAXE.

FIG. 7 depicts production and purification of HAT-tagged CBH-E by cobalt metal ion affinity chromatography column. Fractions were run on 10% SDS-PAGE gels, which were either blotted to a membrane for Western Blot analysis using an anti-HAT tag antibody (upper panel) or stained by Coomassie to visualize protein (lower panel). Clar.=Clarified original bacterial extract; Sup.=unbound proteins in column flow-through; Wash 1&2=proteins rinsed away in the column washes; Elut.1-4=Serial fractions collected after exposing the column to elution buffer containing imidazole. HAT-positive bands migrating at the expected size for HAT-CBH-E were detected in all four fractions containing imidazole (Elut.1 through Elut.4).

FIG. 8 depicts production and purification of HAT-tagged GGH by cobalt metal ion affinity chromatography column. Fractions were run on 10% SDS-PAGE gels, which were either blotted to a membrane for Western Blot analysis using an anti-HAT tag antibody (upper panel) or stained by Coomassie to visualize protein (lower panel). Clar.=Clarified original bacterial extract; Sup.=unbound proteins in column flow-through; Wash 1&2=proteins rinsed away in the column washes; Elut.1-4=Serial fractions collected after exposing the column to elution buffer containing imidazole. HAT-positive bands migrating at the expected size for HAT-GGH were detected in all four fractions containing imidazole (Elut.1 through Elut.4).

FIG. 9 is a graph depicting results from cellulase activity assays of purified recombinant HAT-CBH-E. HAT-CBH-E was incubated with substrate (4-methylumbelliferyl cellobioside (MUC). Absorbance at 405 nm was taken as a measure of cleavage of the substrate and therefore a measure of cellulase activity. pHAT12=control vector; Elut.1-4=Serial fractions collected after exposing the column to elution buffer containing imidazole.

FIG. 10 is a graph depicting results from cellulase activity assays of purified recombinant HAT-GGH. HAT-GGH was incubated with substrate (p-nitrophenyl-α-D-glucopyranoside (pNPG)). Absorbance at 405 nm was measured to detect release of p-nitrophenyl, indicating cleavage of the substrate and therefore of cellulase activity. pHAT12=control vector; Elut.1-4=Serial fractions collected after exposing the column to elution buffer containing imidazole.

FIGS. 11-17 depicts maps of plasmids containing constructs for expressing cell wall-modifying enzymes in plants. D35S=CaMV Double 35S promoter; NPTII=neomycin phosphotransferase II minigene; 35S 3′UTR=CaMV 35S 3′ UTR terminator; OsAct1=rice actin promoter; SP=signal peptide for apoplast targeting; NOS=nopaline synthase terminator.

FIG. 11 depicts a map of pEDEN132, which is designed for expression of NcFAE in plants.

FIG. 12 depicts a map of pEDEN122, which is designed for expression of CBH-E in plants.

FIG. 13 depicts a map of pEDEN140, which is designed for expression of GGH in plants.

FIG. 14 depicts a map of pEDEN163, which is designed for expression of EcGXX in plants.

FIG. 15 depicts a map of pEDEN164, which is designed for expression of FsAXE in plants.

FIG. 16 depicts a map of pEDEN129, which is designed for expression of CBH-E in plants.

FIG. 17 depicts a map of pEDEN130, which is designed for expression NcFAE in plants.

FIG. 18 is a schematic of a plant transformation process for corn.

FIG. 19 is a schematic of a plant transformation process for poplar.

FIG. 20 is a graph depicting results from cellulase activity assays of tobacco leaf extracts from leaves infiltrated with media (control) or Agrobacterium containing pEDEN140. Tobacco leaf extracts were incubated with substrate (4-methylumbelliferyl cellobioside (MUC) and the amount of 4-MU released was taken as a measure of enzyme activity.

FIG. 21 depicts agarose gels that show results from a PCR analysis to screen corn plants regenerated from immature embryos transformed with pEDEN132, an expression plasmid for NcFAE. Presence of NcFAE and of the selectable marker was detected using primers for each gene, shown in Table 6.

FIG. 22 is a graph depicting results from feruloyl esterase activity assays of corn leaf extracts from corn plants transformed with pEDEN132, an expression vector encoding NcFAE. Extracts were incubated with substrate (4-methylumbelliferyl p-trimethylammonio-cinnamate chloride (MUTMAC)) and the amount of 4-MU release was taken as an indication of MUTMAC hydrolysis and enzyme activity. 13C, 13D, 5A, 7C, 10D, 12A, and 12F refer to samples from corn plants generated from different transformation events. PCR screening indicated that samples from events 13A and 13D contained the selectable marker but lacked the NcFAE. Events 5A, 7C, 10D, 12A, and 12F contained both the selectable marker and NcFAE.

FIG. 23 depicts results from experiments characterizing the effects of feruloyl esterase expression on corn biomass digestibility. Upper panel—soluble reducing sugars released into the media during enzyme hydrolysis by Celluclast 1.5 L and β-glucosidase as measured using the DNS reagent. Lower panel—digestibility of biomass samples following enzyme hydrolysis by Celluclast 1.5 L and β-glucosidase. The “tempered” group refers to samples that were incubated at 37° C. for 24 h before digestion; the “not treated” group did not undergo such treatment.

FIG. 24 depicts results from experiments demonstrating synergistic effects between feruloyl esterase expressed in corn biomass and exogenously added xylanase. Sugar yields from feruloyl esterase-expressing and control corn biomass incubated with or without xylanase were measured.

FIG. 25 depicts agarose gels that show results from a PCR analysis to screen corn plants regenerated from immature embryos transformed with pEDEN122, an expression plasmid for CBH-E. Presence of CBH-E (“exocellulase”) and of the selectable marker was detected using primers for each gene, shown in Table X.

FIG. 26 shows effects of in planta exoglucanase (in this case CBH-E) expression on enzyme dosage requirements and digestibility in corn biomass. The “pretreated” group of samples was pretreated with dilute sulfuric acid. Samples were incubated with low (0.4 mg/g) or high (8 mg/g) concentration of commercial cellulase cocktail (Novozymes Celluclast 1.5 L).

FIG. 27 is a graph depicting results from exoglucanase activity assays of poplar leaf extracts from plants transformed with pEDEN129, an expression vector encoding CBH-E. Extracts were incubated with substrate (4-methylumbelliferyl cellobioside; MUC) and the amount of 4-MU release was taken as an indication of MUC hydrolysis and enzyme activity. Labels on the x-axis refer to samples from poplar plants generated from independent transformation events.

FIG. 28 is a graph depicting results from feruloyl esterase activity assays of poplar leaf extracts from plants transformed with pEDEN130, an expression vector encoding NcFAE. Extracts were incubated with substrate (4-methylumbelliferyl p-trimethylammonio-cinnamate chloride (MUTMAC)) and the amount of 4-MU release was taken as an indication of MUTMAC hydrolysis and enzyme activity. Labels on the x-axis refer to samples from poplar plants generated from independent transformation events.

FIG. 29 depicts results from Western Blot analysis of CBH-E expression in poplar leaf extracts from plants transformed with pEDEN129. “CBH-E(+)” denotes HAT-tagged recombinant CBH-E protein, used as a positive control.

### DEFINITIONS

Throughout the specification, several terms are employed that are defined in the following paragraphs.

As used herein, the terms “about” and “approximately,” in reference to a number, is used herein to include numbers that fall within a range of 20%, 10%, 5%, or 1% in either direction (greater than or less than) the number unless otherwise stated or otherwise evident from the context (except where such number would exceed 100% of a possible value).

As used herein, the phrase “binary vector” refers to cloning vectors that are capable of replicating in both E. coli and Agrobacterium tumefaciens. In a binary vector system, two different plasmids are employed for generating transgenic plants. In many embodiments, the first plasmid is a small vector known as disarmed Ti plasmid has an origin of replication (ori) that permits the maintenance of the plasmid in a wide range of bacteria including E. coli and Agrobacterium. In many embodiments, the small vector contains foreign DNA in place of T-DNA, the left and right T-DNA borders (or at least the right T-border), markers for selection and maintenance in both E. coli and A. tumefaciens, and a selectable marker for plants. In many embodiments, the second plasmid is known as helper Ti plasmid, harbored in A. tumefaciens, which lacks the entire T-DNA region but contains an intact vir region essential for transfer of the T-DNA from Agrobacterium to plant cells.

As used herein, the phrase “cell wall-modifying enzyme polypeptide” refers to a polypeptide that modifies at least one component (e.g., xylans, xylan side chains, glucuronoarabinoxylans, xyloglucans, mixed-linkage glucans, pectins, pectates, rhamnogalacturonans, rhamnogalacturonan side chains, lignin, cellulose, mannans, galactans, arabinans, oligosaccharides derived from cell wall polysaccharides, and combinations thereof) or interaction (e.g., covalent linkage, ionic bond interaction, hydrogen bond interaction, and combinations thereof) in plant cell wall. In some embodiments, cell wall-modifying enzyme polypeptides have at least 50%, 60%, 70%, 80% or more overall sequence identity with a polypeptide whose amino acid sequence is set forth in Table 1 (which shows sequences listed as SEQ ID NO. 1 to 84). Alternatively or additionally, in some embodiments, cell wall-modifying enzyme polypeptide shows at least 90%, 95%, 96%, 97%, 98%, 99%, or greater identity with at least one sequence element found in a polypeptide whose amino acid sequence is set forth in Table 1, which sequence element is at least 4, 5, 6, 7, 8, 9, 10, 11, 12, 13, 14, 15, 16, 17, 18, 19, 20 or more amino acids long. In some embodiments, a provided cell wall-modifying enzyme polypeptide disrupts a linkage selected from the group consisting of hemicellulose-cellulose-lignin, hemicellulose-cellulose-pectin, hemicellulosediferululate-hemicellulose, hemicellulose-ferulate-lignin, mixed beta-D-glucan-cellulose, mixed-beta-D-glucan-hemicellulose, pectin-ferulate-lignin linkages, and combinations thereof.

It will be appreciated that the present invention describes use of cell wall-modifying enzyme polypeptides generally, but also of particular cell wall-modifying enzyme polypeptides (e.g., those listed in Table 1).

As used herein, the phrase “externally applied,” when used to describe enzyme polypeptides used in the processing of biomass, refers to enzyme polypeptides that are not produced by the organism whose biomass is being processed. “Externally applied” enzyme polypeptides as used herein does not include enzyme polypeptides that are expressed (whether endogenously or transgenically) by the organism (e.g., plant) from which the biomass is obtained.

As used herein, the term “extract,” when used as noun, refers to a preparation from a biological material (such as lignocellulosic biomass) in which a substantial portion of proteins are in solution. In some embodiments of the invention, the extract is a crude extract, e.g., an extract that is prepared by disrupting cells such that proteins are solubilized and optionally removing debris, but not performing further purification steps. In some embodiments of the invention, the extract is further purified in that certain substances, molecules, or combinations thereof are removed.

As used herein, the term “gene” refers to a discrete nucleic acid sequence responsible for a discrete cellular product and/or performing one or more intracellular or extracellular functions. More specifically, the term “gene” refers to a nucleic acid that includes a portion encoding a protein and optionally encompasses regulatory sequences, such as promoters, enhancers, terminators, and the like, which are involved in the regulation of expression of the protein encoded by the gene of interest. The gene and regulatory sequences may be derived from the same natural source, or may be heterologous to one another. The definition can also include nucleic acids that do not encode proteins but rather provide templates for transcription of functional RNA molecules such as tRNAs, rRNAs, etc. Alternatively, a gene may define a genomic location for a particular event/function, such as the binding of proteins and/or nucleic acids.

As used herein, the term “gene expression” refers to the conversion of the information, contained in a gene, into a gene product. A gene product can be the direct transcriptional product of a gene (e.g., mRNA, tRNA, rRNA, antisense RNA, ribozyme structural RNA or any other type of RNA) or a protein produced by translation of an mRNA. Gene products also include RNAs that are modified by processes such as capping, polyadenylation, methylation, and editing, proteins post-translationally modified, and proteins modified by, for example, methylation, acetylation, phosphorylation, ubiquitination, ADP-ribosylation, myristilation, and glycosylation.

As used herein, the term “gene expression array” refers to an array comprising a plurality of genetic probes immobilized on a substrate surface that can be used for quantitation of mRNA expression levels. In the context of the present invention, the term “array-based gene expression analysis” is used to refer to methods of gene expression analysis that use gene-expression arrays.

The terms “genetically modified” and “transgenic” are used herein interchangeably. A transgenic or genetically modified organism is one that has a genetic background which is at least partially due to manipulation by the hand of man through the use of genetic engineering. For example, the term “transgenic cell”, as used herein, refers to a cell whose DNA contains an exogenous nucleic acid not originally present in the non-transgenic cell. A transgenic cell may be derived or regenerated from a transformed cell or derived from a transgenic cell. Exemplary transgenic cells in the context of the present invention include plant calli derived from a stably transformed plant cell and particular cells (such as leaf, root, stem, or reproductive cells) obtained from a transgenic plant. A “transgenic plant” is any plant in which one or more of the cells of the plant contain heterologous nucleic acid sequences introduced by way of human intervention. Transgenic plants typically express DNA sequences, which confer the plants with characters different from that of native, non-transgenic plants of the same strain. The progeny from such a plant or from crosses involving such a plant in the form of plants, seeds, tissue cultures and isolated tissue and cells, which carry at least part of the modification originally introduced by genetic engineering, are comprised by the definition.

As used herein, the term “genetic probe” refers to a nucleic acid molecule of known sequence, which has its origin in a defined region of the genome and can be a short DNA sequence (or oligonucleotide), a PCR product, or mRNA isolate. Genetic probes are gene-specific DNA sequences to which nucleic acids from a sample (e.g., RNA from a plant extract) are hybridized. Genetic probes specifically bind (or specifically hybridize) to nucleic acid of complementary or substantially complementary sequence through one or more types of chemical bonds, usually through hydrogen bond formation.

As used herein, the term “lignocellulolytic enzyme polypeptide” refers to a polypeptide that disrupts or degrades lignocellulose, which comprises cellulose, hemicellulose, and lignin. The term “lignocelluloytic enzyme polypeptide” encompasses, but is not limited to cellobiohydrolases, endoglucanases, β-D-glucosidases, xylanases, arabinofuranosidases, acetyl xylan esterases, glucuronidases, mannanases, galactanases, arabinases, lignin peroxidases, manganese-dependent peroxidases, hybrid peroxidases, laccases, ferulic acid esterases and related polypeptides. In some embodiments, disruption or degradation of lignocellulose by a lignocellulolytic enzyme polypeptide leads to the formation of substances including monosaccharides, disaccharides, polysaccharides, and phenols. In some embodiments, a lignocellulolytic enzyme polypeptide shares at least 50%, 60%, 70%, 80% or more overall sequence identity with a polypeptide whose amino acid sequence is set forth in Table 3. Alternatively or additionally, in some embodiments, a lignocellulolytic enzyme polypeptide shows at least 90%, 95%, 96%, 97%, 98%, 99%, or greater identity with at least one sequence element found in a polypeptide whose amino acid sequence is set forth in Table 3, which sequence element is at least 4, 5, 6, 7, 8, 9, 10, 11, 12, 13, 14, 15, 16, 17, 18, 19, 20 or more amino acids long. It will be appreciated that the present invention describes use of lignocellulolytic enzyme polypeptides generally, but also of particular lignocellulolytic enzyme polypeptides (e.g., Acidothermus cellulolyticus E1 endo-1,4-O-glucanase polypeptide, Acidothermus cellulolyticus xylE polypeptide, Acidothermus cellulolyticus gux1 polypeptide, Acidothermus cellulolyticus aviIII polypeptide, and Talaromyces emersonii cbhE polypeptide).

As used herein, the term “mixed linkage glucans” refer to non-cellulosic glucans present in plants and often enriched in seed bran. β-D-glucan residues of mixed-linkage glucans are unbranched but contain both (1→3) and (1→4)-linkages. In some embodiments, enzymes that modify mixed-linkage glucans include laminarinase (E.C. 3.2.1.39), licheninase (E.C. 3.2.1.73/74). In some embodiments, some cellulases can hydrolyze certain (1→4)-linkages.

As used herein, the term “nucleic acid construct” refers to a polynucleotide or oligonucleotide comprising nucleic acid sequences not normally associated in nature. A nucleic acid construct of the present invention is prepared, isolated, or manipulated by the hand of man. The terms “nucleic acid”, “polynucleotide” and “oligonucleotide” are used herein interchangeably and refer to a deoxyribonucleotide (DNA) or ribonucleotide (RNA) polymer either in single- or double-stranded form. For the purposes of the present invention, these terms are not to be construed as limited with respect to the length of the polymer and should also be understood to encompass analogs of DNA or RNA polymers made from analogs of natural nucleotides and/or from nucleotides that are modified in the base, sugar and/or phosphate moieties.

As used herein, the term “operably linked” refers to a relationship between two nucleic acid sequences wherein the expression of one of the nucleic acid sequences is controlled by, regulated by or modulated by the other nucleic acid sequence. Preferably, a nucleic acid sequence that is operably linked to a second nucleic acid sequence is covalently linked, either directly or indirectly, to such second sequence, although any effective three-dimensional association is acceptable. A single nucleic acid sequence can be operably linked to multiple other sequences. For example, a single promoter can direct transcription of multiple RNA species.

As will be clear from the context, the term “plant”, as used herein, can refer to a whole plant, plant parts (e.g., cuttings, tubers, pollen), plant organs (e.g., leaves, stems, flowers, roots, fruits, branches, etc.), individual plant cells, groups of plant cells (e.g., cultured plant cells), protoplasts, plant extracts, seeds, and progeny thereof. The class of plants which can be used in the methods of the present invention is as broad as the class of higher plants amenable to transformation techniques, including both monocotyledonous and dicotyledonous plants, as well as certain lower plants such as algae. The term includes plants of a variety of a ploidy levels, including polyploid, diploid and haploid. In certain embodiments of the invention, plants are green field plants. In other embodiments, plants are grown specifically for “biomass energy”. For example, suitable plants include, but are not limited to, alfalfa, bamboo, barley, canola, corn, cotton, cottonwood (e.g. Populus deltoides), eucalyptus, miscanthus, poplar, pine (pinus sp.), potato, rape, rice, soy, sorghum, sugar beet, sugarcane, sunflower, sweetgum, switchgrass, tobacco, turf grass, wheat, and willow. Using transformation methods, genetically modified plants, plant cells, plant tissue, seeds, and the like can be obtained.

As used herein, “plant biomass” refers to biomass that includes a plurality of components found in plant, such as lignin, cellulose, hemicellulose, beta-glucans, homogalacturonans, and rhamnogalacturonans. Plant biomass may be obtained, for example, from a transgenic plant expressing at least one cell wall-modifying enzyme polypeptide as described herein. Plant biomass may be obtained from any part of a plant, including, but not limited to, leaves, stems, seeds, and combinations thereof.

The term “polypeptide”, as used herein, generally has its art-recognized meaning of a polymer of at least three amino acids. However, the term is also used to refer to specific functional classes of polypeptides, such as, for example, lignocellulolytic enzyme polypeptides (including, for example, Acidothermus cellulolyticus E1 endo-1,4-O-glucanase polypeptide, Acidothermus cellulolyticus xylE polypeptide, Acidothermus cellulolyticus gux1 polypeptide, Acidothermus cellulolyticus aviIII polypeptide, and Talaromyces emersonii cbhE polypeptide). For each such class, the present specification provides specific examples of known sequences of such polypeptides. Those of ordinary skill in the art will appreciate, however, that the term “polypeptide” is intended to be sufficiently general as to encompass not only polypeptides having the complete sequence recited herein (or in a reference or database specifically mentioned herein), but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides presented herein.

As used herein, the term “pretreatment” refers to a thermo-chemical process to remove lignin and hemicellulose bound to cellulose in plant biomass, thereby increasing accessibility of the cellulose to cellulases for hydrolysis. Common methods of pretreatment involve using dilute acid (such as, for example, sulfuric acid), ammonia fiber expansion (AFEX), steam explosion, lime, and combinations thereof.

As used herein, the terms “promoter” and “promoter element” refer to a polynucleotide that regulates expression of a selected polynucleotide sequence operably linked to the promoter, and which effects expression of the selected polynucleotide sequence in cells. The term “plant promoter”, as used herein, refers to a promoter that functions in a plant. In some embodiments of the invention, the promoter is a constitutive promoter, i.e., an unregulated promoter that allows continual expression of a gene associated with it. A constitutive promoter may in some embodiments allow expression of an associated gene throughout the life of the plant. Examples of constitutive plant promoters include, but are not limited to, rice act1 promoter, Cauliflower mosaic virus (CaMV) 35S promoter, and nopaline synthase promoter from Agrobacterium tumefaciens. In some embodiments of the invention, the promoter is a tissue-specific promoter that selectively functions in a part of a plant body, such as a flower. In some embodiments of the invention, the promoter is a developmentally specific promoter. In some embodiments of the invention, the promoter is an inducible promoter. In some embodiments of the invention, the promoter is a senescence promoter, i.e., a promoter that allows transcription to be initiated upon a certain event relating to the age of the organism.

As used herein, the term “protoplast” refers to an isolated plant cell without cell walls which has the potency for regeneration into cell culture or a whole plant.

As used herein, the term “regeneration” refers to the process of growing a plant from a plant cell (e.g., plant protoplast, plant callus or plant explant).

As used herein, the term “stably transformed”, when applied to a plant cell, callus or protoplast refers to a cell, callus or protoplast in which an inserted exogenous nucleic acid molecule is capable of replication either as an autonomously replicating plasmid or as part of the host chromosome. The stability is demonstrated by the ability of the transformed cells to establish cell lines or clones comprised of a population of daughter cells containing the exogenous nucleic acid molecule.

As used herein, the term “tempering” refers to a process to condition lignocellulosic biomass prior to pretreatment so as to favor improved yield from hydrolysis and/or allow use of less severe pretreatment conditions without sacrificing yield. In some embodiments, the lignocellulosic biomass transgenically expresses a lignocellulolytic enzyme polypeptide and tempering facilitates activation of the lignocellulolytic enzyme polypeptide. In some embodiments, tempering facilitates improved yield from subsequent hydrolysis as compared to yield obtained from processing without tempering. In some embodiments, tempering facilitates comparable or improved yield from subsequent hydrolysis using less severe pretreatment conditions than would be required without tempering. In some embodiments, tempering comprises a process selected from the group consisting of ensilement, grinding, pelleting, forming a warm water suspension and/or slurry, incubating at a specific temperature, incubating at a specific pH, and combinations thereof. In some embodiments, tempering comprises separating liquid from a slurry that contains soluble sugars and crude enzyme extracts and re-addition of the separated liquid back to the solid biomass after pretreatment. Specific conditions for tempering may depend on specific traits (such as, e.g., traits of the transgene) of the biomass.

As used herein, the term “transformation” refers to a process by which an exogenous nucleic acid molecule (e.g., a vector or recombinant DNA molecule) is introduced into a recipient cell, callus or protoplast. The exogenous nucleic acid molecule may or may not be integrated into (i.e., covalently linked to) chromosomal DNA making up the genome of the host cell, callus or protoplast. For example, the exogenous polynucleotide may be maintained on an episomal element, such as a plasmid. Alternatively, the exogenous polynucleotide may become integrated into a chromosome so that it is inherited by daughter cells through chromosome replication. Methods for transformation include, but are not limited to, electroporation, magnetoporation, Ca²⁺ treatment, injection, particle bombardment, retroviral infection, and lipofection.

The term “transgene”, as used herein, refers to an exogenous gene which, when introduced into a host cell through the hand of man, for example, using a process such as transformation, electroporation, particle bombardment, and the like, is expressed by the host cell and integrated into the cell's DNA such that the trait or traits produced by the expression of the transgene is inherited by the progeny of the transformed cell. A transgene may be partly or entirely heterologous (i.e., foreign to the cell into which it is introduced). Alternatively, a transgene may be homologous to an endogenous gene of the cell into which it is introduced, but is designed to be inserted (or is inserted) into the cell's genome in such a way as to alter the genome of the cell (e.g., it is inserted at a location which differs from that of the natural gene or its insertion results in a knockout). A transgene can also be present in a cell in the form of an episome. A transgene can include one or more transcriptional regulatory sequences and other nucleic acids, such as introns. Alternatively or additionally, a transgene is one that is not naturally associated with the vector sequences with which it is associated according to the present invention.

### DETAILED DESCRIPTION OF CERTAIN EMBODIMENTS OF THE INVENTION

As mentioned above, the present invention relates to improved systems and strategies for reducing costs and increasing yields of ethanol production from lignocellulosic biomass. In some embodiments, provided are enzyme polypeptides that attack the backbone and sidechains of hemicellulose and pectin and feruloyl ester cross-links as a means of releasing fermentable sugars from cellulose, hemicellulose and pectin. Such enzyme activity may improve forage and silage digestibility for livestock and expose cellulose to direct hydrolytic attack by cellulases. Provided are methods of using such enzyme polypeptides that may allow improved plant fiber processing. In some embodiments, provided are plants that transgenically express microbial, plant or animal genes encoding enzyme polypeptides that hydrolyze or otherwise modify components of the plant cell wall, including feruloyl ester linkages, xylans, xylan side chains, glucuronoarabinoxylans, xyloglucans, mixed-linkage glucans, pectins, pectates, rhamnogalacturonans, rhamnogalcturonan side chains, lignin, cellulose, mannans, galactans, arabinans, and oligosaccharides derived from cell wall polysaccharides.

### I. Cell Wall-Modifying Enzyme Polypeptides

In some aspects of the invention, provided are cell wall-modifying enzyme polypeptides of the invention that may be used, alone or in conjunction with other enzymes, to break down lignocellulosic biomass. In some embodiments, cell wall-modifying enzyme polypeptides are lignocellulolytic enzyme polypeptides (described below).

Lignocellulosic biomass is a complex substrate in which crystalline cellulose is embedded within a matrix of hemicellulose and lignin. Lignocellulose represents approximately 90% of the dry weight of most plant material with cellulose making up between 30% to 50% of the dry weight of lignocellulose and hemicellulose making up between 20% and 50% of the dry weight of lignocellulose.

Disruption and degradation (e.g., hydrolysis) of lignocellulose by lignocellulolytic enzyme polypeptides (including those enzyme polypeptides described further below) leads to the formation of substances including monosaccharides, disaccharides, polysaccharides and phenols. In some embodiments, cell wall-modifying enzyme polypeptides provided herein are characterized by and/or are employed under conditions and/or according to a protocol that achieves enhanced disruption and/or degradation of lignocellulose. In some embodiments, cell wall-modifying enzyme polypeptides are used in combination with other lignocellulolytic enzyme polypeptides (as described below) and/or as described in U.S. patent application publication US 2007-0250961 A1, the contents of which are incorporated by reference herein in their entirety.

Cell wall-modifying enzyme polypeptides useful in accordance with the present invention include those having at least 50%, 60%, 70%, 80% or more overall sequence identity with a polypeptide whose amino acid sequence is set forth in Table 1 (which shows sequences listed as SEQ ID NO. 1 to 84). Alternatively or additionally, in some embodiments, cell wall-modifying enzyme polypeptide shows at least 90%, 95%, 96%, 97%, 98%, 99%, or greater identity with at least one sequence element found in a polypeptide whose amino acid sequence is set forth in Table 1, which sequence element is at least 4, 5, 6, 7, 8, 9, 10, 11, 12, 13, 14, 15, 16, 17, 18, 19, 20 or more amino acids long. It will be appreciated that the present invention describes use of cell wall-modifying enzyme polypeptides generally, but also of particular cell wall-modifying enzyme polypeptides (e.g., those listed in Table 1).

TABLE 1
Sequences of certain provided cell wall-modifying
enzyme polypeptides

Sequence ID: 1
Sequence Length: 404
Sequence Type: Protein
Organism: Pyrococcus furiosus
Class of Enzyme: Ferulic acid esterase
CAzy Family:
Enzyme Classifcation Number:
Definition of Activity:
Accession Number:
MKRMIMYLSTVLLIAVVSGCISEQTQTQTLESNSPTQTTTTTSPQITVTF
IVSVPEYTPENDSIYIAGDFNNWNPKDERYKLVKLPDGRWKITLTFPYGK
TIQFKFTRGSWETVEKGINGEEIPNRRFTFTKSGTYEFKVHNWRDFVEKN
VKHTITGNVITFEMFIPQLNTTRRIWIYLPPDYNYSTKRYPVLYMFDGQN
LFDAATSFAGEWGVDEALEKLYKEKNFSIIVVGIDNGGDRRIDEYAPWVN
RDYRRGGLGNATVKFIVETLKPYIDAHYRTDPEKTGIMGSSLGGLMAIYA
GFSYPEVFRYVGAMSSAFWFNPEIYDFVREAKKGPEKIYIDWGTNEGRNP
KAFSESNEKMVKILKEKGYREEFNLKVVIDKGGLHNEYYWGKRFPQAVLW
LFEE
--------------------------------------------------
Sequence ID: 2
Sequence Length: 290
Sequence Type: Protein
Organism: Neurospora crassa
Class of Enzyme: Ferulic acid esterase
CAzy Family:
Enzyme Classifcation Number:
Definition of Activity:
Accession Number:
MAGLHSRLTTFLLLLLSALPAIAAAAPSSGCGKGPTLRNGQTVTTNINGK
SRRYTVRLPDNYNQNNPYRLIFLWHPLGSSMQKIIQGEDPNRGGVLPYYG
LPPLDTSKSAIYVVPDGLNAGWANQNGEDVSFFDNILQTVSDGLCIDTNL
VFSTGFSYGGGMSFSLACSRANKVRAVAVISGAQLSGCAGGNDPVAYYAQ
HGTSDGVLNVAMGRQLRDRFVRNNGCQPANGEVQPGSGGRSTRVEYQGCQ
QGKDVVWVVHGGDHNPSQRDPGQNDPFAPRNTWEFFSRFN
--------------------------------------------------
Sequence ID: 3
Sequence Length: 566
Sequence Type: Protein
Organism: Bifidobacterium longum
Class of Enzyme: Alpha-L-arabinofuranosidase
CAzy Family: GH51
Enzyme Classifcation Number: EC 3.2.1.55
Definition of Activity:
Accession Number: AAO84266
MTTHNSQYSAETTHPDKQESSPAPTAAGTTASNVSTTGNATTPDASIALN
ADATPVADVPPRLFGSFVEHLGRCVYGGIYEPSHPTADENGFRQDVLDLV
KELGVTCVRYPGGNFVSNYNWEDGIGPRENRPMRRDLAWHCTETNEMGID
DFYRWSQKAGTEIMLAVNMGTRGLKAALDELEYVNGAPGTAWADQRVANG
IEEPMDIKMWCIGNEMDGPWQVGHMSPEEYAGAVDKVAHAMKLAESGLEL
VACGSSGAYMPTFGTWEKTVLTKAYENLDFVSCHAYYFDRGHKTRAAASM
QDFLASSEDMTKFIATVSDAADQAREANNGTKDIALSFDEWGVWYSDKWN
EQEDQWKAEAAQGLHHEPWPKSPHLLEDIYTAADAVVEGSLMITLLKHCD
RVRSASRAQLVNVIAPIMAEEHGPAWRQTTFYPFAEAALHARGQAYAPAI
SSPTIHTEAYGDVPAIDAVVTWDEQARTGLLLAVNRDANTPHTLTIDLSG
LPGLPGLGTLALGKAQLLHEDDPYRTNTAEAPEAVTPQPLDIAMNATGTC
TATLPAISWISVEFHG
--------------------------------------------------
Sequence ID: 4
Sequence Length: 521
Sequence Type: Protein
Organism: Sphingomonas sp.
Class of Enzyme: Alpha-L-arabinofuranosidase
CAzy Family:
Enzyme Classifcation Number: EC3.2.1.55
Definition of Activity:
Accession Number: ZP_01302570
MISYLRRATAALLLATSALAAPAIADTDGTPTSATIHADTPGPVYDRRIF
TQFAEHLGNGIYGGLWVGNDKSIPNTNGFRNDVVAALRNLSVPVIRWPGG
CFADEYHWREGVGPKAKRPVKVNTHWGGVTEPNSVGTDEFFELLRQVGAE
AYVAGNVGNGTPQEMAEWVEYMTAPAGTLAEERAKNGHKEPYAVPYFGIG
NELWGCGGNMRAEYAADVTRRYATFIKAPRGTKILKIAAGANVDDYNWTE
TMMRVAADQLDALSLHYYTLPQGGWPPKADPVNFGETEWADTLAKAVHMD
ELITKHVAIMDKYDPKKRVFLAVDEWGTWYAQDPGTHPGFLRQQNTLRDA
LVASVHLDIFAKHADRVRMTAIAQMVNVLQAMILTDGKKMVLTPTYHVFE
MYKPWQDATVLPIELDTPWYGKGQFTMPAVSGSAVRGKDGKVHVGLSNLD
PNQPNTVTVKLDGLNAATVAGRILTASAMNAHNSFDAPETIKPAPFTGAQ
VSGGTLSVTLPPKSVVVLDLQ
--------------------------------------------------
Sequence ID: 5
Sequence Length: 441
Sequence Type: Protein
Organism: Sorangium cellulosum
Class of Enzyme: Alpha-L-arabinofuranosidase
CAzy Family: GH62
Enzyme Classifcation Number: EC 3.2.1.55
Definition of Activity:
Accession Number: CAN98023
MITPFDLHRQTLTPSRSLLRLSALGCVLAALAGCASDTGDDQPSSGGTGG
SENPTTSASSTTGAGAGASTSASGTGGSGPGTSTSTSTSSGSDTGTGGDP
TSGAGGSGGDPGDGGGGAGAGTGAGGSPVTCDLATSFKWKSGPPVINPKS
AAGRNFVSIKDPTIVFHDGKYHVFATVYDTAGNGGWSSVYLNFTDFSQAA
SAQQHHMANWPTGGTVAPQVFFFRPHNKWYLIYQWNGRYSTNDDISNVNG
WSRPQALLKGEPGQMGNTLGALDFWNICDDKNCHLFFSRDDGKLYRSKVS
IDKFPAFDGYETVMTAPSAGLLFEASNVYKVDGTNKYLLLVEAFDNSPRF
FRSWTSESIDGPWAPLADTKQKPFAGPANVTFEGGKWSDDISHGEMVRSG
SDERMTINACNMQFLYQGRDPNAGGAYERLPYKLGLITLEK
--------------------------------------------------
Sequence ID: 6
Sequence Length: 540
Sequence Type: Protein
Organism: Sorangium cellulosum
Class of Enzyme: Alpha-L-arabinofuranosidase
CAzy Family: GH62
Enzyme Classifcation Number: ED 3.2.1.55
Definition of Activity:
Accession Number: CAN94755
MMRIRFRRWSLLTTIAATAACVSAEQLDEDGHEFDELAESVTIDTAATYT
IVGVQSGKCVEVAGGSTADAAALQIASCNGSTRQQFRMESAGGGYYRIRN
VNSNRCMDVAGASTSDGARIQQYSCWSGENQQWSFTDVASGVVRLTARNS
GKSLDVYGRGTADGTAVIQWASNGGTNQQFRITPVSSGTGGTGSGGTGGS
GGTGGTGGTGGTGGSGGSGGSGGGEGCGLPTTFRWQSSSALVSPKSDATH
NIVSIKDPTVSFFNDRWHIYATTANTAGNWQMTYLNFTDWSQAASASHYY
MDRTPGFSGYRCAPQMFFFRPQNKWYLIYQSQPPQFSTTSDPSRPDTWTR
PQNFFASTPAGMPSLPIDYWVICDSANCYLFFTGDDGRMYRSQTTLQNFP
NGFGPVSIALQDSNRNNLFEGSSTYKIKGMNKYLTLIEAIGPTGARFYRS
FTADRLDGAWTPLAHTWNAPFAGQNNVTYAPGVADWSDDVSHGELVRDGN
DETATIDTCNLQFLYQGRNPSSGGEYSQLPYRLGLLKAVR
--------------------------------------------------
Sequence ID: 7
Sequence Length: 419
Sequence Type: Protein
Organism: Sorangium cellulosum
Class of Enzyme: Alpha-L-arabinofuranosidase
CAzy Family: GH62
Enzyme Classifcation Number: EC 3.2.1.55
Definition of Activity: CAN98020
Accession Number:
MITHLDLPSHALAPCRSLLRLSALGCVLAALAGCSGGTTDDQPSPDGTGG
SENPVTGASSASSTTGTGGSTGTSSSVGSGGSGTTGTGGSTSASGSGGDP
GDGGGGAGGSPPTCDLPTTFKWKAGPPVISPKPPAGRSWASVKDPTIVFF
ENKYHVFATVFDTTSGNGGWQSMYSNFTDIPQANAAEQHYMANWPTGSTV
APQVFFFQPHNKWYLIYQWNGRYSTNDDINNMNGWSRPQGLLRGEPNGAL
DFWNICDDKNCHLFFSRDDGKLYRSKVSIDKFPAFDGYETVMSAPSASLL
FEASNVYKVDGSNKYLLMVEAYDNSPRFFRSWTSESLDGPWAPLADTKQN
PFAGPANVTYEGQDWSDDISHGELIRSGHDEKMTIDPCDLRFLYQGRDPK
VGGDYGKLPYRLGMLTLQK
--------------------------------------------------
Sequence ID: 8
Sequence Length: 376
Sequence Type: Protein
Organism: Pseudomonas fluorescens
Class of Enzyme: Endogalactanase
CAzy Family: GH53
Enzyme Classifcation Number: EC 3.2.1.89
Definition of Activity: Endohydrolysis of 1,4-
beta-D-galactosidic linkages in arabinogalactanans
Accession Number: CAA62990
MKKKILAATAILLAAIANTGVADNTPFYVGADLSYVNEMESCGATYRDQG
KKVDPFQLFADKGADLVRVRLWHNATWTKYSDLKDVSKTLKRAKNAGMKT
LLDFHYSDTWTDPEKQFIPKAWAHITDTKELAKALYDYTTDTLASLDQQQ
LLPNLVQVGNETNIEILQAEDTLVHGIPNWQRNATLLNSGVNAVRDYSKK
TGKPIQVVLHIAQPENALWWFKQAKENGVIDYDVIGLSYYPQWSEYSLPQ
LPDAIAELQNTYHKPVMIVETAYPWTLHNFDQAGNVLGEKAVQPEFPASP
RGQLTYLLTLTQLVKSAGGMGVIYWEPAWVSTRCRTLWGKGSHWENASFF
DATRKNNALPAFLFFKADYQASAQAE
--------------------------------------------------
Sequence ID: 9
Sequence Length: 298
Sequence Type: Protein
Organism: Rhodopirellula baltica
Class of Enzyme: Acetylxylan esterase
CAzy Family: CE6
Enzyme Classifcation Number: EC3.1.1.1.72
Definition of Activity: Deacetylation of xylans
and oligoxylans
Accession Number: CAD78234
MVSSPFHSKGPKMPFNLPRLLASVLCLPLLSTLALPSIGVAQEENPPSAD
TSETAQLPPTGLHLFLLAGQSNMAGRGKIADEDLQPHPRVLVFNKAGEWA
PAIAPLHFDKPRIAGVGLGRTFAIEYAENNPQATVGLIPCAVGGSSLDVW
QPGGFHESTNTHPYDDCMKRMQQAIVAGELKGILWHQGESDSNPALSKTY
QSKLNELFERFRTEFGSPNVPIVIGQLGQFTEKPWDESRKLVDQAHRTLP
DRMTNTVFVHSDGLGHKGDQTHFSAEAYREFGHRYFLAYQQLTGSSNE
--------------------------------------------------
Sequence ID: 10
Sequence Length: 232
Sequence Type: Protein
Organism: Solibacter usitatus
Class of Enzyme: Acetylxylan esterase
CAzy Family: CE6
Enzyme Classifcation Number: EC 3.1.1.72
Definition of Activity: Deacetylation of xylans
and olagoxylans
Accession Number: ABJ86882
MKLFLLTLCAAFLLKGQPHEIFLLIGQSNMAGRGVVEEQDRQPIPRVFML
NKAMEWVPAIDPVHFDKPDIAGVGLARTFGKVLAAADPNASIGLVPAAFG
GTSLEEWKVGGKLYEEAVRRAKFAMSSGKLRGILWHQGEADAGKKELASS
YRQRFSAMITQLRADLGEPDVPVVVGQLGEFLSESATPRSPFASVVDEQL
ATVPLTVPHSAFVSSNGLTSNADHLHFDARSQREFGRRYALAFLSIDASW
AH
--------------------------------------------------
Sequence ID: 11
Sequence Length: 539
Sequence Type: Protein
Organism: Fibrobacter succinogenes
Class of Enzyme: Acetylzylan esterase
CAzy Family: CE6
Enzyme Ciassifcation Number: 3.1.1.72
Definition of Activity: Deacetylation of xylans
and oligoxylans
Accession Number: AAG36766
MSVEMSFKKLMGIAGVAAGLSMFAVMGANAAPDPNFHIYIAYGQSNMEGN
ARNFTDVDKKEHPRVKMFATTSCPSLGRPTVGEMYPAVPPMFKCGEGLSV
ADWFGRHMADSLPNVTIGIIPVAQGGTSIRLFDPDDYKNYLNSAESWLKN
GAKAYGDDGNAMGRIIEVAKKAQEKGVIKGIIFHQGETDGGMSNWEQIVK
KTYEYMLKQLGLNAEETPFVAGEMVDGGSCAGFSSRVRGLSKYIANFGVA
SSKGYGSKGDGLHFTVEGYRGMGLRYAQQMLKLINVAPVDPVPQEPFKGA
PIAIPGKVEVEDFDKPGIGKNEDGTSNASYSDEDSENHGDSDYRKDTGVD
LYKAGDGVALGYTQTGEWLEYTVDVKADGEYNIDASVAAGNSTSAFKLYI
DEKAITDDVSVPQTADNSWDTYKTISVKEKVTLKAGKHVLKLEITANYVN
IDWIQFSEPKKEDPPSAIAKVRFDMTEAESNFSVYSMQGQKLGTFTAKGM
ADAMNLVKTDAKLRKQAKGVFFVRKEGAKLMSKKVVVFE
--------------------------------------------------
Sequence ID: 12
Sequence Length: 346
Sequence Type: Protein
Organism: Sorangium cellulosum
Class of Enzyme: Acetylxylan esterase
CAzy Family: CE6
Enzyme Classifcation Number: EC 3.1.1.72
Definition of Activity:
Accession Number: CAN99484
MTQMNRTLRGTARFLLLPLLAMLAASGCGESSSPGATGDTDNTGGTGPGT
GGGAASSTTAGTGGGAASSTTAGTGGDAASSTTAGTGGGATSSTTAGTGS
DSSGAGTGGAPNSRPTFHIFMLMGQSNMAGVAAKQASDQNSDQRLKVLGG
CNQPAGQWNLANPPLSDCPGESRINLSTSVDPGIWFGKTLLGKLREGDTI
GLIGTAESGESINTFISGGSHHQTILNKIAKAKTAENARFAGIIFHQGET
DTGQSSWPGKVVQLYNEMKAAWGVDYDVPFILGELPAGGCCSVHNNLVHQ
AADMLPDGYWISQEGTKVMDQYHFDHASVVLMGTRYGEKMIEALKW
Sequence ID: 13
Sequence Length: 754
Sequence Type: Protein
Organism: Sulfolobus solfataricus
Class of Enzyme: Beta-xylosidase/Alpha-L-arabino-
furanosidase
CAzy Family: GH3
Enzyme Classifcation Number: EC 3.2.1.37/3.2.1.55
Definition of Activity:
Accession Number: AAK43134
MTAIKSLLNQMSIEEKIAQLQAIPIDALMEGKEFSEEKARKYLKLGIGQI
TRVAGSRLGLKPKEVVKLVNKVQKFLVENTRLKIPAIIHEECLSGLMGYS
STAFPQAIGLASTWNPELLTNVASTIRSQGRLIGVNQCLSPVLDVCRDPR
WGRCEETYGEDPYLVASMGLAYITGLQGETQLVATAKHFAAHGFPEGGRN
IAQVHVGNRELRETFLFPFEVAVKIGKVMSIMPAYHEIDGVPCHGNPQLL
TNILRQEWGFDGIVVSDYDGIRQLEAIHKVASNKMEAAILALESGVDIEF
PTIDCYGEPLVTAIKEGLVSEAIIDRAVERVLRIKERLGLLDNPFVDESA
VPERLDDRKSRELALKAARESIVLLKNENNMLPLSKNINKIAVIGPNAND
PRNMLGDYTYTGHLNIDSGIEIVTVLQGIAKKVGEGKVLYAKGCDIAGES
KEGFSEAIEIAKQADVIIAVMGEKSGLPLSWTDIPSEEEFKKYQAVTGEG
NDRASLRLLGVQEELLKELYKTGKPIILVLINGRPLVLSPIINYVKAIIE
AWFPGEEGGNAIADIIFGDYNPSGRLPITFPMDTGQIPLYYSRKPSSFRP
YVMLHSSPLFTFGYGLSYTQFEYSNLEVTPKEVGPLSYITILLDVKNVGN
MEGDEVVQLYISKSFSSVARPVKELKGFAKVHLKPGEKRRVKFALPMEAL
AFYDNFMRLVVEKGEYQILIGNSSENIILKDTFRIKETKPIMERRIFLSN
VQIF
--------------------------------------------------
Sequence ID: 14
Sequence Length: 778
Sequence Type: Protein
Organism: Thermotoga neapolitana
Class of Enzyme: Beta-xylosidase
CAzy Family: GH3
Enzyme Classifcation Number: EC 3.2.1.37
Definition of Activity:
Accession Number: AAB70867
MELYRDPSQPVEVRVKDLLSRMTLEEKIAQLGSVWGYELIDERGKFKREK
AKDLLKNGIGQITRPGGSTNLEPQEAAELVNEIQRFLVEETRLGIPAMIH
EECLTGYMGLGGTNFPQAIAMASTWDPDLIEKMTAAIREDMRKLGAHQGL
APVLDVARDPRWGRTEETFGESPYLVARMGVSYVKGLQGENIKEGVVATV
KHFAGYSASEGGKNWAPTNIPEREFREVFLFPFEAAVKEARVLSVMNSYS
EIDGVPCAANRRLLTDILRKDWGFEGIVVSDYFAVNMLGEYHRIAKDKSE
SARLALEAGIDVELPKTDCYQHLKDLVEKGIVPESLIDEAVSRVLKLKFM
LGLFENPYVDVEKAKIESHRDLALEIARKSIILLKNDGTLPLQKNKKVAL
IGPNAGEVRNLLGDYMYLAHIRALLDNIDDVFGNPQIPRENYERLKKSIE
EHMKSIPSVLDAFKEEGIDFEYAKGCEVTGEDRSGFKEAIEVAKRSDVAI
VVVGDRSGLTLDCTTGESRDMANLKLPGVQEELVLEIAKTGKPVVLVLIT
GRPYSLKNLVDRVNAILQVWLPGEAGGRAIVDVIYGKVNPSGKLPISFPR
SAGQIPVFHYVKPSGGRSHWHGDYVDESTKPLFPFGHGLSYTRFEYSNLR
IEPKEVPSAGEVVIKVDVENVGDMDGDEVVQLYIGREFASVTRPVKELKG
FKRVSLKAKEKKTVVFRLHTDVLAYYDRDMKLVVEPGEFRVMVGSSSEDI
RLTGSFSVTGSKREVVGKRKFFTEVYEE
--------------------------------------------------
Sequence ID: 15
Sequence Length: 715
Sequence Type: Protein
Organism: Clostridium stercorarium
Class of Enzyme: Beta-xylosidase
CAzy Family: GH3
Enzyme Classifcation Number: EC 3.2.1.37
Definition of Activity:
Accession Number: CAD4B309
MENKPVYLDPSYSFEERAKDLVSRMTIEEKVSQMLYNSPAIERLGIPAYN
WWNEALHGVARAGTATMFPQAIGMAATFDEELIYKVADVISTEGRAKYHA
SSKKGDRGIYKGLTFWSPNINIFRDPRWGRGQETYGEDPYLTARLGVAFV
KGLQGNHPKYLKAGGMCKNILPFTVVPESLRHEFNAVVSKKDLYETYLPA
FKALVQEAKVESVMGAYNRTNGEPCCGSKTLLSDILRGEWGFKGHVVSDC
WAIRDFHMHHHVTATAPESAALAVRNGCDLNCGNMFGNLLIALKEGLITE
EEIDRAVTRLMITRMKLGMFDPEDQVPYASISSFVDCKEHRELALDVAKK
SIVLLKNDGLLPLDRKKIRSIAVIGPNADSRQALIGNYEGTASEYVTVLD
GIREMAGDDVRIYYSVGCHLYKDRVENLGEPGDRIAEAVTCAEHADVVIM
CLGLDSTIEGEEMHESNIYGSGDKPDLNLPGQQQELLEAVYATGKPIVLV
LLTGSALAVTWADEHIPAILNAWYPGALGGRAIASVLFGETNPSGKLPVT
FYRTTEELPDFTDYSMENRTYRFMKNEALYPFGFGLSYTTFDYSDLKLSK
DTIRAGEGFNVSVKVTNTGKMAGEEVVQVYIKDLEASWRVPNWQLSGMKR
VRLESGETAEITFEIRPEQLAVVTDEGKSVIEPGEFEIYVGGSQPDARSV
RLMGKAPLKAVLRVQ
--------------------------------------------------
Sequence ID: 16
Sequence Length: 842
Sequence Type: Protein
Organism: Clostridium thermocellum
Class of Enzyme: Endoxyloglucanase
CAzy Family: GH74
Enzyme Classifcation Number: EC 3.2.1.151
Definition of Activity:
Accession Number: CAE51306
MVKKFTSKIKAAVFAAVVAATAIFGPAISSQAVTSVPYKWDNVVIGGGGG
FMPGIVFNETEKDLIYARADIGGAYRWDPSTETWIPLLDHFQMDEYSYYG
VESIATDPVDPNRVYIVAGMYTNDWLPNMGAILRSTDRGETWEKTILPFK
MGGNMPGRSMGERLAIDPNDNRILYLGTRCGNGLWRSTDYGVTWSKVESF
PNPGTYIYDPNFDYTKDIIGVVWVVFDKSSSTPGNPTKTIYVGVADKNES
IYRSTDGGVTWKAVPGQPKGLLPHHGVLASNGMLYITYGDTCGPYDGNGK
GQVWKFNTRTGEWIDITPIPYSSSDNRFCFAGLAVDRQNPDIIMVTSMNA
WWPDEYIFRSTDGGATWKNIWEWGMYPERILHYEIDISAAPWLDWGTEKQ
LPEINPKLGWMIGDIEIDPFNSDRMMYVTGATIYGCDNLTDWDRGGKVKI
EVKATGIEECAVLDLVSPPEGAPLVSAVGDLVGFVHDDLKVGPKKMHVPS
YSSGTGIDYAELVPNFMALVAKADLYDVKKISFSYDGGRNWFQPPNEAPN
SVGGGSVAVAADAKSVIWTPENASPAVTTDNGNSWKVCTNLGMGAVVASD
RVNGKKFYAFYNGKFISTDGGLTFTDTKAPQLPKSVNKIKAVPGKEGHVW
LAAREGGLWRSTDGGYTFEKLSNVDTAHVVGFGKAAPGQDYMAIYITGKI
DNVLGFFRSDDAGKTWVRINDDEHGYGAVDTAITGDPRVYGRVYIATNGR
GIVYGEPASDEPVPTPPQVDKGLVGDLNGDNRINSTDLTLMKRYILKSIE
DLPVEDDLWAADINGDGKINSTDYTYLKKYLLQAIPELPKK
--------------------------------------------------
Sequence ID: 17
Sequence Length: 388
Sequence Type: Protein
Organism: Bacillus halodurans
Class of Enzyme: Reducing end exooligoxylanase
CAzy Family: GH8
Enzyme Classifcation Number: EC 3.2.1.156
Definition of Activity: Hydrolysis of 1,4-beta-D-
xylose residues from the reducing end of oligo-
saccharides
Accession Number: BAB05824
MKKTTEGAFYTREYRNLFKEFGYSEAEIQERVKDTWEQLFGDNPETKIYY
EVGDDLGYLLDTGNLDVRTEGMSYGMMMAVQMDRKDIFDRIWNWTMKNMY
MTEGVHAGYFAWSCQPDGTKNSWGPAPDGEEYFALALFFASHRWGDGDEQ
PFNYSEQARKLLHTCVHNGEGGPGHPMWNRDNKLIKFIPEVEFSDPSYHL
PHFYELFSLWANEEDRVFWKEAAEASREYLKIACHPETGLAPEYAYYDGT
PNDEKGYGHFFSDSYRVAANIGLDAEWFGGSEWSAEEINKIQAFFADKEP
EDYRRYKIDGEPFEEKSLHPVGLIATNAMGSLASVDGPYAKANVDLFWNT
PVRTGNRRYYDNCLYLFAMLALSGNFKIWFPEGQEEEH
--------------------------------------------------
Sequence ID: 18
Sequence Length: 313
Sequence Type: Protein
Organism: Bacillus sp. (Geobacillus thermodeni-
trificans)
Class of Enzyme: Endoarabinase
CAzy Family: GH43
Enzyme Classifcation Number: EC 3.2.1.99
Definition of Activity: Endohydrolysis of 1,5-
alpha-arabinofuranosidic linkages in 1,5 arabinans
Accession Number: BAB64339
MVHFHPFGNVNFYEMDWSLKGDLWAHDPVIAKEGSRWYVFHTGSGIQIKT
SEDGVHWENMGRVFPSLPDWCKQYVPEKDEDHLWAPDICFYNGIYYLYYS
VSTFGKNTSVIGLATNRTLDPRDPDYEWKDMGPVIHSTASDNYNAIDPNV
VFDQEGQPWLSFGSFWSGIQLIQLDTETMKPAAQAELLTIASRGEEPNAI
EAPFIVCRNGYYYLFVSFDFCCRGIESTYKIAVGRSKDITGPYVDKNGVS
MMQGGGTILDAGNDRWIGPGHCAVYFSGVSAILVNHAYDALKNGEPTLQI
RPLYWDDEGWPYL
--------------------------------------------------
Sequence ID: 19
Sequence Length: 382
Sequence Type: Protein
Organism: Sitophilus oryzae
Class of Enzyme: Pectin methylesterase
CAzy Family: CE8
Enzyme Classifcation Number: EC 3.1.1.11
Definition of Activity:
Accession Number: AAW28928
MKIIVLLLLAVVLASADQTAPGTASRPILTASESNYFTTATYLQGWSPPS
ISTSKADYTVGNGYNTIQAAVNAAINAGGTTRKYIKINAGTYQEVVYIPN
TKVPLTIYGGGSSPSDTLITLNMPAQTTPSAYKSLVGSLFNSADPAYSMY
NSWRSKSGAIGTSCSTVFWGKAPAVQIVNLSIENSAKNTGDQQAVALQTN
SDQIQIHNARLLGYQDTLYAGSGSSSVERSYYTNTYIEGDIDFVFGGGSA
IFESCTFYVKADRRSDTSVVFAPDTDPHKMYGYFVYKSTITGDSAWSSSK
KAYLGRAWDSAVSSSSAYVPGTSPNGQLIIKESTIDGIINTSGPWTTATS
GRTYSGNNANSRDLNNDNYNRFWEYNNSGNGA
--------------------------------------------------
Sequence ID: 20
Sequence Length: 433
Sequence Type: Protein
Organism: Erwinia chrysanthemi
Class of Enzyme: Pectin methylesterase
CAzy Family: CE8
Enzyme Classifcation Number: EC 3.1.1.11
Definition of Activity:
Accession Number: CAA59151
MSLTHYSGLAAAVSMSLILTACGGQTPNSARFQPVFPGTVSRPVLSAQEA
GRFTPQHYFAHGGEYAKPVADGWTPTPIDTSRVTAAYVVGPRAGVAGATH
TSIQQAVNAALRQHPGQTRVYIKLLPGTYTGTVYVPEGAPPLTLFGAGDR
PEQVVVSLALDSMMSPADYRARVNPHGQYQPADPAWYMYNACATKAGATI
NTTCSAVMWSQSNDFQLKNLTVVNALLDTVDSGTHQAVALRTDGESGATG
KCPPAQPSDTFFVNTSDRQNSYVTDHYSRAYIKDSYIEGDVDYVFGRATA
VFDRVRFHTVSSRGSKEAYVFAPDSIPSVKYGFLVINSQLTGDNGYRGAQ
KAKLGRAWDQGAKQTGYLPGKTANGQLVIRDSTIDSSYDLANPWGAAATT
DRPFKGNISPQRDLDDIHFNRLWEYNTQVLLHE
--------------------------------------------------
Sequence ID: 21
Sequence Length: 358
Sequence Type: Protein
Organism: Erwinia carotovora
Class of Enzyme: Pectin methylesterase
CAzy Family: CE8
Enzyme Classifcation Number: EC 3.1.1.11
Definition of Activity:
Accession Number: CAG76151
MINASHLGKTLTLAMLISSPWALAQAADYNALVSANVTDAKAYKTITEAI
ASAPADSSPFVIYVKNGVYHERLTVTRPNIHLQGESRDGTVITATTAAGM
LKPDGSKWGTYGSNTVKVDAPDFSARSLTISNDFDYPANQAKADEDPTKL
KDSQAVALLVAENSDRAWFHDVSLTGYQDTLYVKGGRSFFSKCRISGTVD
FIFGNGTALFDDCDIVARNRTDVKDQPLGYLTAPSTDIKQKYGLVIINSR
VIKEKDVPAKSYGLGRPWHPTTTFEDGRYADPNAIGQTVFLNTSMDDHIY
GWDKMSGKDKQGEKIWFHPQDSRFFEYKSSGTGTEKNDQRRQLSEAEAAE
YTADKVLAGWVPTAPKGK
--------------------------------------------------
Sequence ID: 22
Sequence Length: 452
Sequence Type: Protein
Organism: Caldivirga maquilingensis
Class of Enzyme: Exopolygalacturonase
CAzy Family: GH28
Enzyme Classifcaticn Number: EC 3.2.1.-
Definition of Activity:
Accession Number: ABW01078
MINSLPSGRTYNVVEYGADPKGLDDSTGAINEAITQASETRGIVYIPPGN
YLSRNIILRSNVMLLIDKGAVVKFSTDYKSYPIIETRREGVHHCGVMPLI
FGKDVRNVRIIGEGVFDGQGYAWWPIRRFRVTEDYWRRLVESGGVVGDDG
KTWWPTRNAMEGAEAFRKITSEGGKPSTEDCERYREFFRPQLLQLYNAEN
VTIEGVTFKDSPMWTIHILYSRHVTLINTSSIAPDYSPNTDGVVVDSSSD
VEVRGCMIDVGDDCLVIKSGRDEEGRRIGIPSENIHASGCLMKRGHGGFV
IGSEMSGGVRNVSIQDSVFDGTERGVRIKTTRGRGGLIENVYVNNIYMRN
IIHEAVVVDMFYEKRPVEPVSERTPKIRGVVIRNTSCDGADQAVLINGLP
EMPIEDIIIENTRITSNKGIHIENASSIRLSNVKVNSRAIPVITMSNVRN
ITLDDVSGLSME
--------------------------------------------------
Sequence ID: 23
Sequence Length: 402
Sequence Type: Protein
Organism: Erwinia carotovorum
Class of Enzyme: Endopolygalacturonase
CAzy Family: GH28
Enzyme Classifcation Number: EC 3.2.1.15
Definition of Activity: Random hydrolysis of 1,4-
galactosiduronic linkages in pectate or other
galacturonans
Accession Number: CAA37119
MEYQSGKRVLSLSLGLIGLFSASAFASDSRTVSEPKAPSSCTVLKADSST
ATSTIQKALNNCGQGKAVKLSAGSSSVFLSGPLSLPSGVSLLIDKGVTLR
AVNNAKSFENAPSSCGVVDTNGKGCDAFITATSTTNSGIYGPGTIDGQGG
VKLQDKKVSWWDLAADAKVKKLKQNTPRLIQINKSKNFTLYNVSLINSPN
FHVVFSDGDGFTAWKTTIKTPSTARNTDGIDPMSSKNITIAHSNISTGDD
NVAIKAYKGRSETRNISILHNEFGTGHGMSIGSETMGVYNVTVDDLIMTG
TTNGLRIKSDKSAAGVVNGVRYSNVVMKNVAKPIVIDTVYEKKEGSNVPD
WSDITFKDITSQTKGVVVLNGENAKKPIEVTMKNVKLTSDSTWQIKNVTV
KK
--------------------------------------------------
Sequence ID: 24
Sequence Length: 402
Sequence Type: Protein
Organism: Erwinia carotovorum
Class of Enzyme: Endopolygalacturonase
CAzy Family: GH28
Enzyme Classifcation Number: EC 3.2.1.15
Definition of Activity: Random hydrolysis of 1,4-
galactosiduronic linkages in pectate or other
galacturonans
Accession Number: CAA35998
MEYQSGKRVLSLSLGLIGLFSASAWASDSRTVSEPKTPSSCTTLKADSST
ATSTIQKALNNCDQGKAVRLSAGSTSVFLSGPLSLPSGVSLLIDKGVTLR
AVNNAKSFENAPSSCGVVDKNGKGCDAFITAVSTTNSGIYGPGTIDGQGG
VKLQDKKVSWWELAADAKVKKLKQNTPRLIQINKSKNFTLYNVSLINSPN
FHVVFSDGDGFTAWKTTIKTPSTARNTDGIDPMSSKNITIAYSNIATGDD
NVAIKAYKGRAETRNISILHNDFGTGHGMSIGSETMGVYNVTVDDLKMNG
TTNGLRIKSDKSAAGVVNGVRYSNVVMKNVAKPIVIDTVYEKKEGSNVPD
WSDITFKDVTSETKGVVVLNGENAKKPIEVTMKNVKLTSDSTWQIKNVNV
KK
--------------------------------------------------
Sequence ID: 25
Sequence Length: 980
Sequence Type: Protein
Organism: Bacillus sp.
Class of Enzyme: Exopolygalacturonase
CAzy Family: GH28
Enzyme Classifcation Number: EC 3.2.1.82
Definition of Activity: Hydrolysis of pectate from
the non-reducing end, releasing digalacturonate
Accession Number: BAB85762
MKSLKVNGVLFLILLLVFSSFSGAVYAKSEGSPNAPSSPVNLQIPGLAFD
DDSITLWVEKPKHYNDIVDFNIYMNKKKIGSALEDNSGPAKAYIDNFYEN
IDKDNFHEKILIHNFKANNLKPNKSYEFYVTSVNAEGTESAPSNKIVGKT
TKVPEIFNIVDYGAIPDDDSKDTEAIQAAIDAATPGSKVLIPDGKFITGE
LWLKSDMTLQVDGYLLGSPDAEDYSTNFWLYDYSTDERSYSLINAHTYDY
GSLKNIRIVGTGIIDGNGWKYDKNHPTRDELGNELPRYVAGNNSKVTGNV
KVENGKMSPLDLNSENTLGILAANQSYAAQEMGMDAKSAYAARSNLITVR
GVDGMYYEGITQLNPANHGIVNLHSKNIVINGTISKTYDGNNADGYEFGD
SQNIMVFNNFVDTGDDAINFASGMGQAAAKSEPTGNAWIFNNYIREGHGG
VVTGSHTGGWIQDFLVEDNIMYKTDVGLRSKTNTPMGGGAKNILFRNNAL
EGIDGDGPFVFTSAYTDANAAIQYEPAEVISQFRDMEIVDTTVRNQGGSN
KQAILVNGNNSAGEVYHENITFKNVKFDNVYSVNMDYAKDFKFINVSFTN
VKDNGGNPWRIKNSTFVFENTTTAPIDATQKPEWAEDTIINAGSSPDGKN
VTLTWSEATDNVGVSGYTIYKDREKLGQDYTTTNLTSFTVDGLAPATEYT
FKVEATDATGNRTSNGPEIKVMTNGEADQTAPVLPKNTKISESTTKIPSS
DTFSGKNVNVVYTGFTWTSITWDAASDDTGIAGYNVYANGELNGFATSNK
YTLTRLEPGTKYNIEVEAVDIAGNTAPYNSVLEFETARPYPIGAPSDGGL
DAKINSDGTSVTLSWNAAKALNQDVIGYRVYVNGQPMKSEGAPFTPINSE
MTTSDTNYTVTGLKQGKRYTFKVEAVGHASKYSKRERLSDVLPNGLLEVS
GYRWSGFGPSVDVHLIPGKAKSEQAKSK
--------------------------------------------------
Sequence ID: 26
Sequence Length: 448
Sequence Type: Protein
Organism: Thermotoga maritima
Class of Enzyme: Exopolygalacturonase
CAzy Family: GH28
Enzyme Classifcation Number: EC 3.2.1.67
Definition of Activity: Hydrolysis of 1,4-alpha-D-
galactosiduronic linkages from pectate and other
galacturonans, releasing D-galacturonate
Accession Number: AAD35522
MIMEELAKKIEEEILNHVREPQIPDREVNLLDFGARGDGRTDCSESFKRA
IEELSKQGGGRLIVPEGVFLTGPIHLKSNIELHVKGTIKFIPDPERYLPV
VLTRFEGIELYNYSPLVYALDCENVAITGSGVLDGSADNEHWWPWKGKKD
FGWKEGLPNQQEDVKKLKEMAERGTPVEERVFGKGHYLRPSFVQFYRCRN
VLVEGVKIINSPMWCVHPVLSENVIIRNIEISSTGPNNDGIDPESCKYML
IEKCRFDTGDDSVVIKSGRDADGRRIGVPSEYILVRDNLVISQASHGGLV
IGSEMSGGVRNVVARNNVYMNVERALRLKTNSRRGGYMENIFFIDNVAVN
VSEEVIRINLRYDNEEGEYLPVVRSVFVKNLKATGGKYAVRIEGLENDYV
KDILISDTIIEGAKISVLLEFGQLGMENVIMNGSRFEKLYIEGKALLK
--------------------------------------------------
Sequence ID: 27
Sequence Length: 602
Sequence Type: Protein
Organism: Erwinia chrysanthemi
Class of Enzyme: Exo-poly-alpha-D-galacturonase
CAzy Family: GH28
Enzyme Classifcation Number: EC 3.2.1.82
Definition of Activity:
Accession Number: CAB99320
METITFSRRPALASIVAACLISTPALAATAQAPQKLQIPTLSYDDHSVAL
VWDAPEDTSNITDYQIYQNGQLIGLASQNNDKNSPAKPYISAFYKNDTGN
FHRRVVIQNAKIDGLKANTDYQFTVRTVYADGSTSADSNAVTATTAATPQ
VINITQYGAKGDGTTLNTTAIQKAIDACQTGCRVDIPAGVFKTGALWLKS
DMTLNLLQGATLLGSDNAADYPEAYKIYSYSSQVRPASLINAIDKTTSAV
GTFKNIRIIGKGVIDGNGWKRSADAKDELGNSLPQYVKSDSSKVSKDGIL
AKNQVAAAVAKGMDTKTAYSQRRSSLVTLRGVKNVYIADVTIRNPANHGV
MFLESQNVVENGVIHQTFDANNGDGVEFGNSQNIMVFNSVFDTGDDSINF
AAGMGQDAQSQEPSQNAWLFNNYFRRGHGAVVMGSHTGAGIIDVLAENNV
ISQNDVGLRAKSAPAIGGGAHGIVFRNSAMKNLAKQAVIVTLSYSDSNGT
IDYTPAKVPARFYDFTVKNVTVQDSTGSSPVIEITGDSGKGIWHSQFTFS
NMKLSGVTPASISDLSDSQFNNLTFSKLRSGSSPWKFGTVKNVSVDGKIV
TP
--------------------------------------------------
Sequence ID: 28
Sequence Length: 324
Sequence Type: Protein
Organism: Bacillus subtilis
Class of Enzyme: Endoarabinase
CAzy Family: GH43
Enzyme Classifcation Number: EC 3.2.1.99
Definition of Activity:
Accession Number: BAA20372
MLKNKKTWKRFFHLSSAALAAGLIFTSAAPAEAAFWGASNELLHDPTMIK
EGSSWYALGTGLNEERGLRVLKSSDAKNWTVQKSIFSTPLSWWSNYVPNY
EKNQWAPDIQYYNGKYWLYYSVSSFGNNTSAIGLASSTSISSGNWEDEGL
VIRSTSSNNYNAIDPELTFDKDGNPWLAFGSFWSGIKLTKLDKSTMKPTG
SPYSIAARPNNNGALEAPTLTYQNGYYYLMVSFDKCCNGVNSTYKIAYGR
SKSITGPYLDKSGKSMLDGGGTILDSGNDQWKGPGGQDIVNGNILVRHAY
DANDNGTPKLLINDLNWSSGWPSY
--------------------------------------------------
Sequence ID: 29
Sequence Length: 290
Sequence Type: Protein
Organism: Erwinia carotovorum
Class of Enzyme: Pectin lyase
CAzy Family: PL1
Enzyme Classifcation Number: EC 4.2.2.10
Definition of Activity: Eliminative cleavage of
(1,4)-alpha-D-galacturonan methyl ester to give
oligosaccharides with 4-deoxy-6-O-methyl-alpha-D-
galact-4-enuronosyl groups at their reducing ends
Accession Number: AAA24856
MAYPTTNLTGIIGFAKAANVTGGTGGKVVTVNSLADFKSAVSGSAKTIVV
LGLSLKASALTKVVFGSNKTIVGSFGGYANVLTNIHLRAESNSSNVIFQN
LVFKHDVAIKDNDDIQLYLNYGKGYWVDHCSWPGHTWSDNDGSLDKLIYI
GEKADYITISNCLFSNHKYGCIFGHPADDNNSAYNGYPRLTICHNYYENI
QVRAPGLMRYGYFHVFNQPTSINSTWPLQLRRNANLISERNVFGTGAENK
GMVDDKGNGSTLRIMAVHRLRWRANRLRRNGRRHLTIHTV
--------------------------------------------------
Sequence ID: 30
Sequence Length: 345
Sequence Type: Protein
Organism: Bacillus subtilis
Class of Enzyme: Pectin lyase
CAzy Family: PL1
Enzyme Classifcation Number: EC 4.2.2.10
Definition of Activity: Eliminative cleavage of
(1,4)-alpha-D-galacturonan methyl ester to give
oligosaccharides with 4-deoxy-6-O-methyl-alpha-D-
galact-4-enuronosyl groups at their reducing ends
Accession Number: BAA12119
MKRFCLWFAVFSLLLVLLPGKAFGAVDFPNTSTNGLLGFAGNAKNEKGIS
KASTTGGKNGQIVYIQSVNDLKTHLGGSTPKILVLQNDISASSKTTVTIG
SNKTLVGSYAKKTLKNIYLTTSSASGNVIFQNLTFEHSPQINGNNDIQLY
LDSGINYWIDHVTFSGHSYSASGSDLDKLLYVGKSADYITISNSKFANHK
YGLILGYPDDSQHQYDGYPHMTIANNYFENLYVRGPGLMRYGYFHVKNNY
SNNFNQAITIATKAKIYSEYNYFGKGSEKGGILDDKGTGYFKDTGSYPSL
NKQTSPLTSWNPGSNYSYRVQTPQYTKDFVTKYAGSQSTTLVFGY
--------------------------------------------------
Sequence ID: 31
Sequence Length: 392
Sequence Type: Protein
Organism: Erwinia chrysanthemi
Class of Enzyme: Pectace lyase
CAzy Family: PL1
Enzyme Classifcation Number: EC 4.2.2.2
Definition of Activity: Eliminative cleavage of
(1,4)-alpha-D-galacturonan to give oligo-
saccharides with 4-deoxy-alpha-D-galact-4-
enuronosyl groups at their non-reducing ends
Accession Number: AAA24846
MNKVSGRSFTRTSTCLLATLIAGVMTSGVSAAELVNSKALESAPAAGWAS
QNGSTTGGAAATSDNIYVVTNISEFTSALSAGAVAKIIQITGTVDISGGT
PYKDFADQKARSQINIPANTTVIGIGTDAKFINGSLIIDGTDGTNNVIIR
NVYIQTPIDVEPHYEKGDGWNAEWDGMNITNGAHHVWVDHVTISDGSFTD
DMYTTKDGETYVQHDGALDIKRGSDYVTISNSLFDQHDKTMLIGHSDTNS
AQDKGKLHVTLFNNVFNRVTERAPRVRYGSIHSFNNVFNGDVKDPVYRYL
YSFGIGTSGSVLSEGNSFTIANLSASKACKVVKKFNGSIFSDNGSVLNGS
AADLSGCGFSAYTSAIPYVYAVQPMTTELAQSITDHAGSGKL
--------------------------------------------------
Sequence ID: 32
Sequence Length: 390
Sequence Type: Protein
Organism: Pseudomonas marginalis
Class of Enzyme: Pectate lyase
CAzy Family: PL1
Enzyme Classifcation Number: EC 4.2.2.2
Definition of Activity: of (1,4)-alpha-D-galactu-
ronan to give oligosaccharides with 4-deoxy-alpha-
D-galact-4-enuronosyl groups at their non-reducing
ends
Accession Number: AAC60448
MTKPSTFTACKLASAVFGALLFSSVPAHAADIWLDVATTGWATQNGGTKG
GSRAAANDIYTVKNAAELKKALSASAGSNGRIIKITGIIDVSEGKVYTKT
ADMKVRGRLDIPGKTTIVGIGSNAEIREGFFYAKENDVIIRNITVENPWD
PEPIFDKDDGADGNWNSEYDGLTVEGANNVWVDHVTFTDGRRTDDQNGTE
HERPKQHHDGALDVKNGANFVTISYSVFKSHEKNNLIGSSDSRTTDDGKL
KVTIHNTLFENISARAPRVRYGQVHLYNNYHVGSTSHKVYPFSYAHGVGK
NSKIFSERNAFEIAGISGCDKIAGDYGGSVYRDTGSTLNGSALSCSWSSS
IGWTPPYSYTPLAADKVAADVKAKAGAGKL
--------------------------------------------------
Sequence ID: 33
Sequence Length: 578
Sequence Type: Protein
Organism: Erwinia chrysanthemi
Class of Enzyme: Rhamnogalacturonan lyase
CAzy Family: PL4
Enzyme Ciassifcation Number: EC 4.2.2.-
Definition of Activity:
Accession Number: CAD27359
MHMNKPLQAWRTPLLTLIFVLPLTATGAVKLTLDGMNSTLDNGLLKVRFG
ADGSAKEVWKGGTNLISRLSGAARDPDKNRSFYLDYYSGGVNEFVPERLE
VIKQTPDQVHLAYIDDQNGKLRLEYHLIMTRDVSGLYSYVVAANTGSAPV
TVSELRNVYRFDATRLDTLFNSIRRGTPLLYDELEQLPKVQDETWRLPDG
SVYSKYDFAGYQRESRYWGVMGNGYGAWMVPASGEYYSGDALKQELLVHQ
DAIILNYLTGSHFGTPDMVAQPGFEKLYGPWLLYINQGNDRELVADVSRR
AEHERASWPYRWLDDARYPRQRATVSGRLRTEAPHATVVLNSSAENFDIQ
TTGYLFSARTNRDGRFSLSNVPPGEYRLSAYADGGTQIGLLAQQTVRVEG
KKTRLGQIDARQPAPLAWAIGQADRRADEFRFGDKPRQYRWQTEVPADLT
FEIGKSRERKDWYYAQTQPGSWHILFNTRTPEQPYTLNIAIAAASNNGMT
TPASSPQLAVKLNGQLLTTLKYDNDKSIYRGAMQSGRYHEAHIPLPAGAL
QQGGNRITLELLGGMVMYDAITLTETPQ
--------------------------------------------------
Sequence ID: 34
Sequence Length: 567
Sequence Type: Protein
Organism: Xanthomonas oryzae
Class of Enzyme: Rhamnogalacturonan lyase
CAzy Family: PL4
Enzyme Classifcation Number: EC 4.2.2.-
Definition of Activity:
Accession Number: AAW74332
MLEVRTVRTFSVSDARLASRAGIATKSCFRTVTTMPRHRLHTFACALLLY
AGVSAPALAEFGCTRSGDRVIVDSGAELVFSVDTHDGDIVSMRYRDNELQ
TTEPKGSQIASGLGSASVDARIAGGTIIVSAKAGDLIQYYIVRKGRNAIY
MATYAPTLPPVGELRFVARLNVSKLPDAQQEPDSNVGTAIEGNDVFLLPD
GRTSSKFYSARRMMDDQVHGVSGPGVAVFMLMGNREHSAGGPFFKDIATQ
KTRVTHELYNYMYSDHTQTEAFRGGLHGVYGLLFTDGSAPSDAQLNTDFV
DATLGLSDYLPASGRGAVGGQVSGVLPDQPAVIGLCNAQAQYWATADGSG
EYQVTGVRPGRYRMTLYQNELEVAWRDIEVFANDTAHATLQAVALPGTLK
WQIGIPDGTPAGFGYADLLPHAHPSDARMRWSATTYTVGSSGQSSFPAVQ
WRGINTPSRIDFTLAADEVRDYRLRIFVPLAQGSARPQISVNARWNGPMP
DAPLQPKTRGITRGTTRGNNALYEMDIPASALQAGSNCIEIGIASGSPDN
GFLSPAIVFDSIQLVAL
--------------------------------------------------
Sequence ID: 35
Sequence Length: 894
Sequence Type: Protein
Organism: Caldivirga maquilingensis
Class of Enzyme: Alpha-L-rhamnosidase
CAzy Family: GH 78
Enzyme Classifcation Number: EC 3.2.1.40
Definition of Activity: Hydrolysis of terminal
non-reducing alpha-L-rhamnose residues in alpha-L-
rhamnosides
Accession Number: ABW01021
MVHGLRIIDARVEFTVNPLGIDESKPRFSWILEHEERGQYQSAYRVIVSS
SLENAVKGIGDVWDSGKVNSRDQVIKYNGPPLSSFTKYYWRVKAWDSNGV
EGDWSDVQWFETAVLKPEEWSGKWIGGGQLLRRSFRVEGSVIEAKAYVTG
LGYYELRINGERVGDRVLDPPWSEYDKTVYYSVYDVTNLVKSGENVIGLI
LGRGRYGPVSPNRAQIPGLKYYDEPKASAMIRIRLSDGSVITINTDESWK
CLVKGPILYDDIYNGYRYDARLEPYGWDKAGFDDSNWVQCSVVKPPGGRL
RSTAAVPGTKVKGTLKPREYYNPRPGVYVFDFGQNITGWVRLRVRGSSGV
EVKVRHSEVINSDGSLNVENIRGAEATDTYILSGRDVEVLEPRFTYHGFR
YAEVTGYPGVPSIDDVEAVIVQTDFESTGSIATSSKIINDIHRITWWSLR
ANLLNGIQTDCPQRDERMGWLGDAWLSSDSAVFNFNMVKYYEKFIRDIID
SQRDDGSIPDTVPPYWNTYPADPAWGTALIYIPWLLYVHYGDVEILEEAY
EAMKKWWSFLNSRVKDNVLYFSKYGEWVPPGRVFSAEYCPPEILSTWILY
RDTLTLAQIAKVLGRGEDASFFTKRAEEIRDAFNRVFLTERGYYSKYTAP
DGSVRMLGGSQTCNALPLYLDMVPGNRVNDIVKALAHNIEADWDRHLVVG
IFGAKYVPEVLVKYGYVDLAYRAVTQESYPGWGYMIKEGATTLWERWEKL
TGAGMNSHNHHMFGSIDAWFYRDLAGLMTLEPGFSRIMIKPNIPSELRYC
SASLYTVRGLTSVEWSRVNDELVVTVTVPVNSTAEVHLPKLGESTVVREG
DKVLWSGGKVVEVSPGVLSVKDAGDRIVVEVGSGRFIFTIKTIN
--------------------------------------------------
Sequence ID: 36
Sequence Length: 932
Sequence Type: Protein
Organism: Thermomicrobia bacterium
Class of Enzyme: Alpha-L-rhamnosidase
CAzy Family: GH78
Enzyme Classifcation Number: EC 3.2.1.40
Definition of Activity: Hydrolysis of terminal
non-reducing alpha-L-rhamnose residues in alpha-L-
rhamnosides
Accession Number: AAR96046
MLRIDRVKVERSRDGLGLGTGRPRLCWRVETDIRDWRQAAYEVELYDGSG
QLVGSTGRVESGESVWVAWPFEALGSRQRAGVRVRVWGEDGSESDWSDLQ
WLEVGLLARDDWQGAFITPDWEEDTSVANPCPYLRKTFSLPGGVRRARLY
VTGLGVYEVELNGQRVGDHVLSPGWTSYRHRLLYETFDVTGLLREGDNCL
GAILGDGWYRGRLGFGGGRRNLYGERLALLAQLEVELEDGSRQVVVTDGS
WRAHRGPILESPIYDGEVYDARLEMPGWSTPEYDDSEWAGTRELGWPTES
LEPLEVPARRTQEVAPREILRSFSGKTIVDFGQNLVGRVRLRVSGPRGQR
VRLRHAEVLEGGELCTRTLRTARATDEYVLRGDGEEEWEPRFTFHGFRYV
EVEGWPGELRAEDLVAVVCHSDMERIGWFGCSDPLVERLHENVVWSMRGN
FLHIPTDCPQRDERLGWTGDIQVFSPAACFIYDASGFLTSWLRDVALDQD
ESGAVPFVVPNALGGQVIPAAAWGDAAVIVPWVLYQRYGDAGVLEAQWPS
MRAWVDCIKTIAGPARLWNKGFQFGDWLDPAAPPDNPAAARTDPYIVASA
YFARSAEIVGLSAQVLGMQDMAEEYLGLASEVREAFNREYVTPNGRVVSD
AQTAYSLAIGFALLPTQEQRQHAGERLAELVRAEGYKIGTGFVGTPLICD
ALCATGHHDVAYRLLMSRECPSWLYPVTMGATTIWERWDSLRPDGSVNPG
EMTSFNHYALGAVADWLHRVVGGLAPAEPGYRKLRIQPVPGGGLSYARAR
HVTPYGTAECSWRTEGGEIEVRVVVPPNTSAQVVLPGSGREVEVGSGEHV
WRYAFEAHRYPPVTLDTPLKEILEDAEAWEVLTRHFPEVASMPPRRLERI
GTIRDLAASVVAFNERVGRLERELQALSRERS
--------------------------------------------------
Sequence ID: 37
Sequence Length: 954
Sequence Type: Protein
Organism: Thermomicrobia bacterium
Class of Enzyme: Alpha-L-rhamnosidase
CAzy Family: GH78
Enzyme Classifcation Number: EC 3.2.1.40
Definition of Activity: Hydrolysis of terminal
non-reducing alpha-L-rhamnose residues in alpha-L-
rhamnosides
Accession Number: AAR96047
MQWQASWIWLEGEPSPRNDWVCFRKSFELDRSASPLEEAKLSITADSRYV
LYVNGQLVGRGPVRSWPFEQSYDTYDLRHLLHPGRNCLAVLVTHFGVSTF
SYVRGRGGLLAQLELSSGDDRTTIGTDGSWKVHRHLGYSRRTTRISPQQG
FVEQLDARAWSSEWKDLMYDDSGWEDAMIVGPVGTPPWEQLVPRDIPFLT
EEVLHPTRVVSLHSTVPPKIAVAVDMRAIMMPDSADHAEQVQYAGFLATI
LRTDGEGTARLLLSKPWVGDGIAASINGQVYGAELMSRTPTGRELEVELS
AGDNLLLVYVCGSDHADPLRLALDSDLGLELVSPTGGESAFVAIGPLASR
VVRNFDFSQPLEYDETAVRRISSCASVADLRAWSHLPRSVPPELVSPADV
FTLCTWPRQRTELTTGKELEANVFPSKDPGLVPILRAGDTELVLDFGQEV
SGYLFLDVEASEGTLIDLYGFEFMEDDYRQDTVGLDNTLRYTCREGRQHY
VSPQRRGLRYLMLTVREARAPLRVHGVGVVQSTYPVSQVGTFRCSDPLLN
DIWEISRLTTKLCMEDTFVDCPAYEQTFWVGDSRNEALTAYYLFGAEELV
RRCLRLVPGSRRYTPLYMDQVPSGWVSVIPNWTFLWVMACREYYERTGDL
AFVQDIWPDIQYTLDHYLQHINDDGLLEISAWNLLDWAPIDQPNSGVVTH
QNCFFVRALKDADELGQSAGDETAGRYAERARELAAAINTHLWSDEHKAY
IDSIHADSTRSSVISMQTQVVALLTGVAEGDRAEVVRSHIASPPAGWVQI
GSPFMSFFLYEAMVRQGMYAQMLEDIRQKYGLMLEHGATTCWETFPGALG
ARYTRSHCHAWSAAPGYFLGAYVLGVRPGGPGWHRVIVAPQPCDLAWARG
SVPLPRGDRVDVSWRREGQKLLLRVERPQEVELEVVPPEEYELELDERVR
QTTQ
--------------------------------------------------
Sequence ID: 38
Sequence Length: 551
Sequence Type: Protein
Organism: Erwinia chrysanthemi
Class of Enzyme: Pectin acetylesterase
CAzy Family: CE12
Enzyme Classifcation Number: 3.1.1.-
Definition of Activity:
Accession Number: CAA70971
MLTTTWNRAFFLGSLLCLPISFAQAEGTVTETNASPTSPVLNVVTLAPNT
SISGRVAYRDIRFPATLLIKDQHGVERSVKTDIQGRFYVDVSSLVTPLRL
SAIEAGGQNCLLSNQLRAVCLGALVPELRDGHENRININPLTDRILSEVA
VSAGYIGPQQLIDAATLPSLSTTVWETAYREFHVGFDDALKQAGIADPSQ
FDPLTYSDTMTPAFTKILQVINHTRGYNNNNGQASHTVLTDIKFRPIAGL
NASGSYEPLDLTSANQHRKALEQSHTRIFIVSDSTAATYEKARFPRMGWG
QVFEQQFRPGGDVTVVNGARAGRSSRDFYYEGWFRQMEPFMRPGDYLFIG
MGHNDQNCDSQKALRGAADVANLCTYPNSADGRPQYPQGKPDMSFQISLE
RYIRYAQAHRMIPVLLTPTARVKNAEGKNGTPAVHSHLTKQNKAGGYAYI
GDYTQTIRDTASKNKVPLLDVETATLALANQGDGQQWQQYWLAVDPDRYP
YYRDQAGSLTQPDTTHFQQKGAQAVAAIVADQIKATPSLRELAGKLQAAN
R
--------------------------------------------------
Sequence ID: 39
Sequence Length: 322
Sequence Type: Protein
Organism: Erwinia chrysanthemi
Class of Enzyme: Pectin acetylesterase
CAzy Family: CE10
Enzyme Classifcation Number: 3.1.1.-
Definition of Activity:
Accession Number: CAD45188
MSLRRVIAGTLMMSVSGFTLADTIFPIWPQGEAPGAATSSVQQQVVERSK
DPTLPDRAVTGIRSPEITVYTPEKPNGTALLITPGGSYQRVVLDKEGSDL
APFFTRQGYTLFVMTYRMPGDGHQEGADAPLADAQRAIRTLRAHAAQWQI
DPQRIGIMGFSAGGHVAASLGTRFAQTVYPAQDEIDHISARPDFMVLMYP
VISMQENIAHAGSRKALIGSHPSDAQIQRYSAEKQVSAQTPPTFLVHAID
DPSVSVDNSLVMLAALRAHQIPAEIHLFEQGKHGFGIRGTVGLPAAIWPQ
LLDNWLTSLPLKKNTANQPDKK
--------------------------------------------------
Sequence ID: 40
Sequence Length: 455
Sequence Type: Protein
Organism: Talaromyces emersonii
Class of Enzyme: Reducing end exoglucanase
CAzy Family: GH7
Enzyme Classifcation Number:
Definition of Activity:
Accession Number: AAL33603
MLRRALLLSSSAILAVKAQQAGTATAENHPPLTWQECTAPGSCTTQNGAV
VLDANWRWVHDVNGYTNCYTGNTWDPTYCPDDETCAQNCALDGADYEGTY
GVTSSGSSLKLNFVTGSNVGSRLYLLQDDSTYQIFKLLNREFSFDVDVSN
LPCGLNGALYFVAMDADGGVSKYPNNKAGAKYGTGYCDSQCPRDLKFIDG
EANVEGWQPSSNNANTGIGDHGSCCAEMDVWEANSISNAVTPHPCDTPGQ
TMCSGDDCGGTYSNDRYAGTCDPDGCDFNPYRMGNTSFYGPGKIIDTTKP
FTVVTQFLTDDGTDTGTLSEIKRFYIQNSNVIPQPNSDISGVTGNSITTE
FCTAQKQAFGDTDDFSQHGGLAKMGAAMQQGMVLVMSLWDDYAAQMLWLD
SDYPTDADPTTPGIARGTCPTDSGVPSDVESQSPNSYVTYSNIKFGPINS
TFTAS
--------------------------------------------------
Sequence ID: 41
Sequence Length: 257
Sequence Type: Protein
Organism: Thermotoga maritime
Class of Enzyme: Licheninase
CAzy Family: GH12
Enzyme Classifcation Number: EC 3.2.1.4
Definition of Activity: Endohydrolysis of beta-
1,4-D-glucosidic linkages in cellulose,
licheninan, and cereal beta-D-glucans
Accession Number: CAA93273
MVLMTKPGTSDFVWNGIPLSMELNLWNIKEYSGSVAMKFDGEKITFDADI
QNLSPKEPERYVLGYPEFYYGYKPWENHTAEGSKLPVPVSSMKSFSVEVS
FDIHHEPSLPLNFAMETWLTREKYQTEASIGDVEIMVWFYFNNLTPGGEK
IEEFTIPFVLNGESVEGTWELWLAEWGWDYLAFRLKDPVKKGRVKFDVRH
FLDAAGKALSSSARVKDFEDLYFTVWEIGTEFGSPETKSAQFGWKFENFS
IDLEVRE
--------------------------------------------------
Sequence ID: 42
Sequence Length: 297
Sequence Type: Protein
Organism: Pyrococcus furiosus
Class of Enzyme: Laminarinase
CAzy Family: GH16
Enzyme Classification Number: EC 3.2.1.39
Definition of Activity: Hydrolysis of 1,3-beta-D-
glucosidic linkages in 1,3-beta-D-glucans
Accession Number: AAL80200
MKKEALLFLSLIFLVFVSGCIHHSTNQQLSSKQQVPEVIEIDGKQWRLIW
HDEFEGSEVNKEYWTFEKGNGIAYGIPGWGNGELEYYTENNTYIVNGTLV
IEARKEIITDPNEGTFLYTSSRLKTEGKVEFSPPVVVEARIKLPKGKGLW
PAFWMLGSNIREVGWPNCGEIDIMEFLGHEPRTIHGTVHGPGYSGSKGIT
RAYTLPEGVPDFTEDFHVFGIVWYPDKIKWYVDGTFYHEVTKEQVEAMGY
EWVFDKPFYIILNLAVGGYWPGNPDATTPFPAKMVVDYVRVYSFVSG
--------------------------------------------------
Sequence ID: 43
Sequence Length: 276
Sequence Type: Protein
Organism: Rhodothermus marinus
Class of Enzyme: Laminarinase
CAzy Family: GH16
Enzyme Classifcation Number: EC 3.2.1.39
Definition of Activity: Hydrolysis of 1,3-beta-D-
glucosidic linkages in 1,3-beta-D-glucans
Accession Number: AAC69707
MMQRVAFILCSLLFGCSILDGDQPIRLPHWELVWSDEFDYNGLPDPAKWD
YDVGGHGWGNQELQYYTRARIENARVGGGVLIIEARRESYEGREYTSARL
VTRGKASWTYGRFEIRARLPSGRGTWPAIWMLPDRQTYGSAYWPDNGEID
IMEHVGFNPDVVHGTVHTKAYNHLLGTQRGGSIRVPTARTDFHVYAIEWT
PEEIRWFVDDSLYYRFPNERLTNPEADWRHWPFDQPFHLIMNIAVGGTWG
GQQGVDPEAFPAQLVVDYVRVYRWVE
--------------------------------------------------
Sequence ID: 44
Sequence Length: 646
Sequence Type: Protein
Organism: Thermotoga neapolitana
Class of Enzyme: Laminarinase
CAzy Family: GH16
Enzyme Classifcation Number: Ec 3.2.1.39
Definition of Activity: Hydrolysis of 1,3-beta-D-
glucosidic linkages in 1,3-beta-D-glucans
Accession Number: CAA88008
MKKLVLVLLLFPVFILAQNILHNGSFDAPILIAGVDIEPPAADGSINTQN
NWVFFTNSNGEGEARVENGVLVVEITNGGDHTWSVQIIQSPIRVEKLHKY
RVFFKAKASVQRNIGVKIGGTAGRGWAAYNPGTDESGGMVFELGTDWKTY
EFEFVMRQETDENARFEFQLGKSTGTVWIDTVWIDDVVMEDVGTLEVSGE
ENEIYTEEDEDKVEDWQLVWSQEFDDGVIDPNVWNFEIGNGHAKGIPGWG
NAELEYYTDKNAFVENGCLVIEARKEQVSDEYGTYDYTSARITTEGKFEI
KYGKIEIRAKLPKGKGIWPALWMLGNNIGEVGWPTCGEIDIMEMLGHDTR
TVLRTAHGPGYSGGASIGVAYHLPEEVPDFSEDFHVFSIEWDENEVEWYV
DGQLYHVLSKDELAELGLEWVFDHPFFLILNVAMGGYWPGYPDETTQFPQ
RMYIDYIRVYKDMNPETITGEVDDCEYEQSQQQTGPEVTYEQINNGTFDE
PIVNDQANNPDEWFIWQAGDYGISGARVSDYGVTDGYAYITIEDSGTDTW
HIQFNQWIGLYKGKTYTISFRAKADTPRPINVKILQNHDPWINYFAQTVN
LTTEWQTFTFTYTHPDDADEVVQISFELGKEAPTTIYFDDVSVSPQ
--------------------------------------------------
Sequence ID: 45
Sequence Length: 565
Sequence Type: Protein
Organism: Pseudomonas sp.
Class of Enzyme: Beta-1,3(4)-glucanase
CAzy Family: GH 16
Enzyme Classifcation Number: EC 3.2.1.6
Definition of Activity: Endohydrolysis of 1,3 or
1,4-linkages in beta-D-glucans when the glucose
residue whose reducing group is involved in the
linkage to be hydrolyzed is itself substituted at
C-3
Accession Number: BAC16332
MTIKYSHPKTLLSAALCASAILCSHASLAARFQAEDYTAFADTSAGNTGG
AYRSDDVDIEATSDEGGGYNVGWVETGEWLTYASLNIPANGRYVVRARVA
SDTGGAMSVDLNAGSILLGELAIPATGGWQSWQTVEREVDLSAGTYNLGV
YASTGGWNFNWIEVEPVGNTGGGGSSVTFEAEDYDNASDTTPGNTGGAYR
SGDVDIEATSDQGGGYNVGWTESGEWLAYNDFNVPTAGNYRFEVRVASGS
GGVLSLDLNGGSTSLGEVAIPVTGGWQTWQTVTLDAYVPAGNHSLGVYAT
TGGWNLNWIKATPTGGGGNPNPNPTVTWSDEFDSIDLNTWNFETGGNGWG
NNELQYYTNGNNASIQYDPQAGSNVLVLEARQETGGACWFGGNCGYTSTR
MNTRNKKSFKYGRMEARLKLPKAQGIWPAFWMLGDNFNTQGWPQGGELDI
MEHVGTNNITSGALHGPGYSGNTPITGHLDHATPIEQSYKTYAVEWDANG
IRWYVDDINFYSVSRAQVEQYGQWVYDQPFWFLLNVAVGGNWPGDPDHAN
FSTQRMYVDYVRVYQ
--------------------------------------------------
Sequence ID: 46
Sequence Length: 269
Sequence Type: Protein
Organism: Sinorhizobium meliloti
Class of Enzyme: Licheninase
CAzy Family: GH16
Enzyme Classifcation Number: EC 3.2.1.73
Definition of Activity: Hydrolysis of 1,4-beta-D-
glucosidic linkages in beta-D-glucans containing
1,3 and 1,4-bonds
Accession Number: CAC49480
MTIDRYRRFARLAFIATLPLAGLATAAAAQEGANGKSFKDDFDTLDTRVW
FVSDGWNNGGHQNCTWSKKQVKTVDGILELTFEEKKVKERNFACGEIQTR
KRFGYGTYEARIKAADGSGLNSAFFTYIGPADKKPHDEIDFEVLGKNTAK
VQINQYVSAKGGNEFLADVPGGANQGFNDYAFVWEKNRIRYYVNGELVHE
VTDPAKIPVNAQKIFFSLWGTDTLTDWMGTFSYKEPTKLQVDRVAFTAAG
DECQFAESVACQLERAQSE
--------------------------------------------------
Sequence ID: 47
Sequence Length: 418
Sequence Type: Protein
Organism: Thermococcus sp.
Class of Enzyme: Beta-glucosidase
CAzy Family: GH1
Enzyme Classifcation Number: EC 3.2.1.21
Definition of Activity: Hydrolysis of terminal
non-reducing beta-D-glucan residues with release
of beta-D-glucose
Accession Number: CAA94187
MFRFPDGFLLGTATSSYQIEGDNVWSDWWYWAEKGKLPPAGKACNSWELY
EKDLELMAGLGYAAYRFSIEWGRVFPEEGRPNEEALMRYQGIIDLLRENG
ITPMLTLHHFTLPAWFALRGGFEREENLEHWRGYVELIADNIEGVELVAT
FNEPMVYVVASYVEGTWPPFRKNPLKAEKVAANLIRAHAIAYFILHGKFR
VGIVKNRPHFIPASDSERDRKATDEIDYTFNRSLLDGILTGRFKGFMRTF
DVPASGLDWLGMNYYNIMKVRAVRNPLRRFAVEDAGVSRKTDMGWSVYPK
GIYDGLRAFAEYGLPLYVTENGIATLDDEWRVEFIVQHLQYVHKALKEGI
DVRGYFYWSLVDNYEWAEGFRPRFGLVEVDYETFERKPRKSAHIYGEIAK
KGEIRGELLEGYGLGEKL
--------------------------------------------------
Sequence ID: 48
Sequence Length: 448
Sequence Type: Protein
Organism: Clostridium thermocellum
Class of Enzyme: Beta-glucosidase
CAzy Family: GH1
Enzyme Classifcation Number: 3.2.1.21
Definition of Activity: Hydrolysis of terminal
non-reducing beta-D-glucan residues with release
of beta-D-glucose
Accession Number: CAA42814
MSKITFPKDFIWGSATAAYQIEGAYNEDGKGESIWDRFSHTPGNIADGHT
GDVACDHYHRYEEDIKIMKEIGIKSYRFSISWPRIFPEGTGKLNQKGLDF
YKRLTNLLLENGIMPAITLYHWDLPQKLQDKGGWKNRDTTDYFTEYSEVI
FKNLGDIVPIWFTHNEPGVVSLLGHFLGIHAPGIKDLRTSLEVSHNLLLS
HGKAVKLFREMNIDAQIGIALNLSYHYPASEKAEDIEAAELSFSLAGRWY
LDPVLKGRYPENALKLYKKKGIELSFPEDDLKLISQPIDFIAFNNYSSEF
IKYDPSSESGFSPANSILEKFEKTDMGWIIYPEGLYDLLMLLDRDYGKPN
IVISENGAAFKDEIGSNGKIEDTKRIQYLKDYLTQAHRAIQDGVNLKAYY
LWSLLDNFEWAYGYNKRFGIVHVNFDTLERKIKDSGYWYKEVIKNNGF
--------------------------------------------------
Sequence ID: 49
Sequence Length: 374
Sequence Type: Protein
Organism: Sorangium cellulosum
Class of Enzyme: Glucuronyl esterase 4-O-methyl-
glucuronyl esterse
CAzy Family: CE15
Enzyme Classifcation Number: EC 3.1.1.-
Definition of Activity: Hydrolysis of xylan-bound
4-O-methyl-D-glucuronic acid and lignin
Accession Number: CAN95599
MPTLPEPSGLTEVNRKLPDPFTFFNGTKVTTKEQWECRRKEILAMAAKYL
YGPVPPEPDEVTGTVSGGTVSITAKAGGKTETFSASISGSGSVIALKLSG
GIFPSGHKTLSFGSGFEGKIRNLFGLSEVNTNIANGWMIDRVMDVLEQNP
GSGHDPTKVMVSGCSGCGKGAYLAGVFSRAPVVVIVESGGGGVANLRQAE
WFRHGEGGSVWQCSDAKPQSIDNLEDNGICGPWVTSAARWLRSDPSKVYN
LPFDTHMLLATIAPRHLVHFTNANGRNSWCHLGGTCEALSAWAAKPVWKA
LGVPERMGFQMYSANHCGASGSQTALAGEMFKRAFEGNTSANTDVMGILD
NGVQQPVSEWEDMWIDWDMDTVLQ
--------------------------------------------------
Sequence ID: 50
Sequence Length: 505
Sequence Type: Protein
Organism: Sorangium cellulosum
Class of Enzyme: Glucuronyl esterase 4-O-methyl-
glucuronyl esterase
CAzy Family: CE15
Enzyme Classifcation Number: EC 3.1.1.-
Definition of Activity: Hydrolysis of xylan-bound
4-O-methyl-D-glucuronic acid and lignin
Accession Number: YP_001612814
MRLRTARPTISLALFAVLPWMLAACGSEGGSEDPSGSGGSPAASTGGVGA
SGSGTGGTPTGTGGPSSSSGTPTGTGGDATTSEASTGGGGPAGTGGAPGT
GGTGGSGDGGNAGSAEWGEVENPGAGCTVGPMPSVASLTANSKLPDPFKK
MDGSRIASKSEWACRREEILQQAYKFIYGDKPVPAKGSVSGTVSTSRITV
EVKDGGGSGSFNLTVNMNGATAPAPAIIGYGGLSGMPVPSGVATITFTAI
ESTGTSGAKNGPFYSVYGSDHPAGYLTAQAWQISRVLDVLEQNPGVIDPR
RVGVTGCSRWGKGAFVAGVLDNRIALTIPVESGLGGTIGLRLVEVLDSYS
GSEWPYHGISYVRWLSEVALGQFTTGNNAGADNTNKLPVDMHEMMGLIAP
RGLYIVDNPSTMYNGLDRNSAWVTANVGKMIFEALGVGNHIAYTGAGGSH
CSWRSQYTASLNAMVDKFLKGNNAAATGNFATDLPNKPNHMDHIDWTPPT
LAGEL
--------------------------------------------------
Sequence ID: 51
Sequence Length: 488
Sequence Type: Protein
Organism: Sorangium cellulosum
Class of Enzyme: Glucuronyl esterase 4-O-methyl-
glucuronyl esterase
CAzy Family: CE15
Enzyme Classifcation Number: EC 3.1.1.-
Definition of Activity: Hydrolysis of xylan-bound
4-O--methyl-D-glucuronic acid and lignin
Accession Number: CAN92371
MRTLATRTARAALGLCLTAAACGQSQPNLSGQGGAGGGSDGSGGESATSS
GDTTSSSSSGSGTASSSSSSGGTTSSSSSGVDTTSSSSSGTGPDDTPVEN
ASADCEVAALPEASALPKVSKLPDPFTKLDGTSVSTKAEWHCRRQEIRKQ
AEKYIYGEKPTPDVVTGTVTENKISVHVEAQGKKIDFSADIVLPSKGEAP
FPAIINVGGKGGFGGITLGESRILDQGVAVIYYNHNEIGREGTAEQSRGK
PNPGKFYDIYGGDHSAGLLMAWAWGASRILDVIQASGGDIIDPTGIGVTG
CSRNGKGAFAIGVFDDRIALTIPHETSTAGVPAYRIADVLGKERTDHNYF
GLNWLSNNFEPFVFKNNASNAVKLPIDTHALIAMMAPRGLLVLENPHQAQ
MGAPAGHTATAAGAAVYKALGVEKNVSYHSKVAETAHCSYKNEYTDVLAK
SIARFLKHEGEAPGEFVVGSGGSLSMADWVDWQAPTLE
--------------------------------------------------
Sequence ID: 52
Sequence Length: 600
Sequence Type: Protein
Organism: Sorangium cellulosum
Class of Enzyme: Glucuronyl esterase 4-O-methy-
glucuronyl esterase
CAzy Family: CE15
Enzyme Classifcation Number: EC 3.1.1.-
Definition of Activity: Hydrolysis of xylan-bound
4-O-methyl-D-glucuronic acid and lignin
Accession Number: YP_001611635
MRITRLLGCVSASFAFGLLACAVEPIEEEDLDTLDGALDSADGSMSADIA
IQSDWGNGYCANVRVTNKSRSPATTGWNVGVRLNGSTLANAWNVTSVSSN
GQFTATNVTHNAAIKEKGWVEWGFCANGSGRPAVASVAGSGGTIVGTGAS
SSSSSSASSSSSSSSSSTSSSSSSSSSGAGGSGGAGGSGGAGGSGAGGSG
GSGGSGGSGGSGGSTGAVEDSGASCPKPTLPAASSLPVFDTHHDPFLSLS
GSRITKKSEWACRRAEIKSQVETYESGSKPVVSKDNVTGQFSANRLTVSV
NDAGKSASFSINISRPSGAPAGPIPLVIGIGGNNLDTSVFTQNGVAMATF
DNNAMGAQNGGGSRGTGTFYNLYGSNHSASSMIAWAWGVSRIIDALEKTP
GANIDPKRIAVTGCSRNGKGALTVGAFDERIVLTIPQESGAGGSASWRVS
QAGANAGENVQTLSSAASEQPWFRANFGSTFGNRVTSLPFDHHMVMGLVA
PRALLVIDNRIDWLGINSTFTAGSIAQQIWKGLGVPDKMGYWQTAAHAHC
AFPSSQRAALDAYVKKFLVGGGTADTNLLKGDGATADLNRWMKWTAPTLQ
--------------------------------------------------
Sequence ID: 53
Sequence Length: 647
Sequence Type: Protein
Organism: Thermoplasma volcanium
Class of Enzyme: Endoglucanase
CAzy Family: GH5
Enzyme Classifcation Number: EC 3.2.1.4
Definition of Activity:
Accession Number: BAB59829
METAKDFYRKGFRLGVNFWPRLANIKMWKEWNEQEILDDLKEAKNIGCDF
LRVFILDEDFVNAYGEINVKSMAYMTRFLDMCSSLHLKVFITFIVGHMSG
RNWVIPWAPDNNIYESKAIMNFSKFVEHFVNEYKTHPAIEGWLMSNEITL
VKRPSSPEQAMVLESVFYGIVKNLDPDHTVSSGDVLSFLQQPPNIRNHSD
YAGLHLYFYDNDLLRQRYSYGSLLNIFSNDGSVPVFLEEFGFSTNQGTEK
SQGEFIYSTLWTALANESMGGLVWCFSDFIGEEDPPYDWRPLEINFGLIR
ADGTRKYSAEKFLQFSIELKELENMMFFQSFQRIYHEISVIVPFYAYADY
TSVSEAYSDYLFNRIPNPILTSLLLCKMASLQPTVFYENDLEDHINGKKL
LIIPSVPTMRATTWNRLLKASVDSDIHIMASTFRGSEGSVPLTSFHDSFT
HIWEKLFGVKTITELGSKGIPYSGNIEIIFTKEFGPFKKGQHINMQAFSN
TYYCYSIEATKAQIIAVDKDNRPVFTYNEETRAYLFSIPFELVLTVDDTG
KYSKPFMDIYREIARRSGIKSLSTSTHPAIEVADFSNGTKNICITINHST
DTVQSTIKCYGINPMMKMGNAKYVKNCREGIVIIYPPGGVALIESSL
--------------------------------------------------
Sequence ID: 54
Sequence Length: 640
Sequence Type: Protein
Organism: Thermofilum pendens
Class of Enzyme: Endoglucanase
CAzy Family: GH5
Enzyme Classifcation Number: EC 3.2.1.4
Definition of Activity:
Accession Number: ABL79067
MDEAFKFLLGVNYWPRLYNVKMWKEWDEESLKKDIEKMKELGVRVVRIFL
RDIDFADERGIPIEESLQKLQRFLDLLHEKNLQAFVTLLVGHMSGKNFPI
PWTSFDSLYTPSSVEKTATFARKIAERLASHPALAGWILSNELSLVKRAT
TREDALRLLEAFTKTMKSVDPNHIVSSGDIPDSFMQETPNVRHLVDYVGP
HLYLYDTDLVRLGYFYGAMLELFSNAGDLPVILEEFGFSTLQFSEESHAR
FVEEILYTSLAHEASGAFIWCFSDFTEESGEPYDWRPLELGFGLLKKDGS
EKLAADSYRNFSHVVERIEKLGLHSKYKRLSSTFVVYPFYLFRDYEFIWY
KESLGFWESIKPHLMSYSLLSASSVPSRMVYELDLKKILKSAKLVVLPSV
VATLASTWRNLLEYVELGGTLYSSVIRGAGAFKALHDAPTHLWNELFGVE
NVLEAGSMGRKIFGVVKLKFVRKFGNLSEGDELLLKVPESIYTFKAQSTD
SDVIALDDEGEPVIFFSRRGRGKTILSLIPIEVILQAQENAQWHEGTIFY
EQLAFVSEVERRYASKDPRVELQVYTGEKDDLLIVINHSNENVETSITSA
TRIVEAQVIGGKARLLPESKREMRAVFPPKSGSIIRVVKT
--------------------------------------------------
Sequence ID: 55
Sequence Length: 425
Sequence Type: Protein
Organism: Thermus caldophilus
Class of Enzyme: Endoglucanase
CAzy Family: GH5
Enzyme Classifcation Number: EC 3.2.1.4
Definition of Activity:
Accession Number: AAK60011
MRWVSLALLSLLLALGGCAAQKGAEGSPPPKGTGQTVPLYASRPDGVYKN
GVPLPLYGVNWFGLETCDRAPHGLWSGRSVADFLAQLKGFGFNALRLPVA
PEVLRDQGTVASWAQGGDPAYPTSPLAGLRYVLEKAQGLGFYVLLDFHTF
RCDLIGGRLPGRPFDPSRGYTKDDWLADLRRLAGLSLEFPNVFGIDLANE
PYDLTWAEWKALAQEGARAVLGVNPRVLVAVEGVGNLSPNGGYNAFWGEN
LAEARDDLGLGDRLLYLPHVYGPSVYDQPYFSDSTFPNNMPAVWDAHFGH
LSGRGLPWGIGEFGGKYTGQDRVWQEAFVDYLRSKGVRVWFYWALNPNSG
DTGGLLEEDWKTPVWDKIRLLERLMAPGGGLAFDFLPATFEVPNPERGFA
EDSYYPDEPSLDAPALVAEARGKGY
Sequence ID: 56
Sequence Length: 317
Sequence Type: Protein
Organism: Thermotoga maritima
Class of Enzyme: Endoglucanase
CAzy Family: GH5
Enzyme Classifcation Nuitber: Ec 3.2.1.4
Definition of Activity:
Accession Number: AAD36816
MGVDPFERNKILGRGINIGNALEAPNEGDWGVVIKDEFFDIIKEAGFSHV
RIPIRWSTHAYAFPPYKIMDRFFKRVDEVINGALKRGLAVVINIHHYEEL
MNDPEEHKERFLALWKQIADRYKDYPETLFFEILNEPHGNLTPEKWNELL
EEALKVIRSIDKKHTIIIGTAEWGGISALEKLSVPKWEKNSIVTIHYYNP
FEFTHQGAEWVEGSEKWLGRKWGSPDDQKHLIEEFNFIEEWSKKNKRPIY
IGEFGAYRKADLESRIKWTSFVVREMEKRRWSWAYWEFCSGFGVYDTLRK
TWNKDLLEALIGGDSIE
--------------------------------------------------
Sequence ID: 57
Sequence Length: 596
Sequence Type: Protein
Organism: Thermobifida fusca
Class of Enzyme: beta-1,4-exocellulase
CAzy Family: GH6
Enzyme Classifcation Number: EC 3.2.1.91
Definition of Activity:
Accession Number: AAA62211
MSKVRATNRRSWMRRGLAAASGLALGASMVAFAAPANAAGCSVDYTVNSW
GTGFTANVTITNLGSAINGWTLEWDFPGNQQVTNLWNGTYTQSGQHVSVS
NAPYNASIPANGTVEFGFNGSYSGSNDIPSSFKLNGVTCDGSDDPDPEPS
PSPSPSPSPTDPDEPGGPTNPPTNPGEKVDNPFEGAKLYVNPVWSAKAAA
EPGGSAVANESTAVWLDRIGAIEGNDSPTTGSMGLRDHLEEAVRQSGGDP
LTIQVVIYNLPGRDCAALASNGELGPDELDRYKSEYIDPIADIMWDFADY
ENLRIVAIIEIDSLPNLVTNVGGNGGTELCAYMKQNGGYVNGVGYALRKL
GEIPNVYNYIDAAHHGWIGWDSNFGPSVDIFYEAANASGSTVDYVHGFIS
NTANYSATVEPYLDVNGTVNGQLIRQSKWVDWNQYVDELSFVQDLRQALI
AKGFRSDICMLIDTSRNGWGGPNRPTGPSSSTDLNTYVDESRIDRRIHPG
NWCNQAGAGLGERPTVNPAPGVDAYVWVKPPGESDGASEEIPNDEGKGFD
RMCDPTYQGNARNGNNPSGALPNAPISGHWFSAQFRELLANAYPPL
--------------------------------------------------
Sequence ID: 58
Sequence Length: 453
Sequence Type: Protein
Organism: Streptomyces sp.
Class of Enzyme: beta-1,4-exocellulase
CAzy Family: GH6
Enzyme Classifcation Number: EC 3.2.1.91
Definition of Activity:
Accession Number: BAB83928
MSRSRTAMLAALTLAAGSMTLALAAGPASAGPAAPTARVDNPYVGATMYV
NPEWSALAASEPGGDRVADQPTAVWLDRIATIEGVDGKMGLREHLDEALQ
QKGSGELVVQLVIYDLPGRDCAALASNGELGPDELDRYKSEYIDPIADIL
SDSKYEGLRIVTVIEPDSLPNLVTNAGGTDTTTEACTTMKANGNYEKGVS
YALSKLGAIPNVYNYIDAAHHGWLGWDTNLGPSVQEFYKVATSNGASVDD
VAGFAVNTANYSPTVEPYFTVSDTVNGQTVRQSKWVDWNQYVDEQSYAQA
LRNEAVAAGFNSDIGVIIDTSRNGWGGSDRPSGPGPQTSVDAYVDGSRID
RRVHVGNWCNQSGAGLGERPTAAPASGIDAYTWIKPPGESDGNSAPVDND
EGKGFDQMCDPSYQGNARNGYNPSGALPDAPLSGQWFSAQFRELMQNAYP
PLS
--------------------------------------------------
Sequence ID: 59
Sequence Length: 516
Sequence Type: Protein
Orqanisrn: Phanerochaete chrysosporium
Class of Enzyme: exo-cellobiohydrolase I
CAzy Family: GH7
Enzyme Classifcation Number: EC 3.2.1.-
Definition of Activity:
Accession Number: AAB46373
MFRTATLLAFTMAAMVFGQQVGTNTAENHRTLTSQKCTKSGGCSNLNTKI
VLDANWRWLHSTSGYTNCYTGNQWDATLCPDGKTCAANCALDGADYTGTY
GITASGSSLKLQFVTGSNVGSRVYLMADDTHYQMFQLLNQEFTFDVDMSN
LPCGLNGALYLSAMDADGGMAKYPTNKAGAKYGTGYCDSQCPRDIKFING
EANVEGWNATSANAGTGNYGTCCTEMDIWEANNDAAAYTPHPCTTNAQTR
CSGSDCTRDTGLCDADGCDFNSFRMGDQTFLGKGLTVDTSKPFTVVTQFI
TNDGTSAGTLTEIRRLYVQNGKVIQNSSVKIPGIDPVNSITDNFCSQQKT
AFGDTNYFAQHGGLKQVGEALRTGMVLALSIWDDYAANMLWLDSNYPTNK
DPSTPGVARGTCATTSGVPAQIEAQSPNAYVVFSNIKFGDLNTTYTGTVS
SSSVSSSHSSTSTSSSHSSSSTPPTQPTGVTVPQWGQCGGIGYTGSTTCA
SPYTCHVLNPYYSQCY
--------------------------------------------------
Sequence ID: 60
Sequence Length: 506
Sequence Type: Protein
Organism: Agaricus bisporus
Class of Enzyme: exo-cellobiohydrolase I
CAzy Family: GH7
Enzyme Classifcation Number: EC 3.2.1.-
Definition of Activity:
Accession Number: CAA90422
MFPRSILLALSLTAVALGQQVGTNMAENHPSLTWQRCTSSGCQNVNGKVT
LDANWRWTHRINDFTNCYTGNEWDTSICPDGVTCAENCALDGADYAGTYG
VTSSGTALTLKFVTESQQKNIGSRLYLMADDSNYEIFNLLNKEFTFDVDV
SKLPCGLNGALYFSEMAADGGMSSTNTAGAKYGTGYCDSQCPRDIKFIDG
EANSEGWEGSPNDVNAGTGNFGACCGEMDIWEANSISSAYTPHPCREPGL
QRCEGNTCSVNDRYATECDPDGCDFNSFRMGDKSFYGPGMTVDTNQPITV
VTQFITDNGSDNGNLQEIRRIYVQNGQVIQNSNVNIPGIDSGNSISAEFC
DQAKEAFGDERSFQDRGGLSGMGSALDRGMVLVLSIWDDHAVNMLWLDSD
YPLDASPSQPGISRGTCSRDSGKPEDVEANAGGVQVVYSNIKFGDINSTF
NNNGGGGGNPSPTTTRPNSPAQTMWGQCGGQGWTGPTACQSPSTCHVIND
FYSQCF
--------------------------------------------------
Sequence ID: 61
Sequence Length: 741
Sequence Type: Protein
Organism: Clostridium thermocellum
Class of Enzyme: exo-cellobiohydrolase I
CAzy Family: GH7
Enzyme Classifcation Number: EC 3.2.1.-
Definition of Activity:
Accession Nunber: AAA23226
MVKSRKISILLAVAMLVSIMIPTTAFAGPTKAPTKDGTSYKDLFLELYGK
IKDPKNGYFSPDEGIPYHSIETLIVEAPDYGHVTTSEAFSYYVWLEAMYG
NLTGNWSGVETAWKVMEDWIIPDSTEQPGMSSYNPNSPATYADEYEDPSY
YPSELKFDTVRVGSDPVHNDLVSAYGPNMYLMHWLMDVDNWYGFGTGTRA
TFINTFQRGEQESTWETIPHPSIEEFKYGGPNGFLDLFTKDRSYAKQWRY
TNAPDAEGRAIQAVYWANKWAKEQGKGSAVASVVSKAAKMGDFLRNDMFD
KYFMKIGAQDKTPATGYDSAHYLMAWYTAWGGGIGASWAWKIGCSHAHFG
YQNPFQGWVSATQSDFAPKSSNGKRDWTTSYKRQLEFYQWLQSAEGGIAG
GATNSWNGRYEKYPAGTSTFYGMAYVPHPVYADPGSNQWFGFQAWSMQRV
MEYYLETGDSSVKNLIKKWVDWVMSEIKLYDDGTFAIPSDLEWSGQPDTW
TGTYTGNPNLHVRVTSYGTDLGVAGSLANALATYAAATERWEGKLDTKAR
DMAAELVNRAWYNFYCSEGKGVVTEEARADYKRFFEQEVYVPAGWSGTMP
NGDKIQPGIKFIDIRTKYRQDPYYDIVYQAYLRGEAPVLNYHRFWHEVDL
AVAMGVLATYFPDMTYKVPGTPSTKLYGDVNDDGKVNSTDAVALKRYVLR
SGISINTDNADLNEDGRVNSTDLGILKRYILKEIDTLPYKN
--------------------------------------------------
Sequence ID: 62
Sequence Length: 619
Sequence Type: Protein
Organism: Clostridium thermocellum
Class of Enzyme: xylanase
CAzy Family: GH10
Enzyme Classifcation Number: EC 3.2.1.8
Definition of Activity:
Accession Number: BAA21516
MLKKKLLTLLTVFALLTVGICGSFLPLPKASAAALIYDDFETGLNGWGPR
GPETVELTTEEAYSGRYSLKVSGRTSTWNGPMVDKTDVLTLGESYKLGVY
VKFVGDSYSNEQRFSLQLQYNDGAGDVYQNIKTATVYKGTWTLLEGQLTV
PSHAKDVKIYVETEFKNSPSPQDLMDFYIDDFTATPANLPEIEKDIPSLK
DVFAGYFKVGGAATVAELAPKPAKELFLKHYNSLTFGNELKPESVLDYDA
TIAYMEANGGDQVNPQITLRAARPLLEFAKEHNIPVRGHTLVWHSQTPDW
FFRENYSQDENAPWASKEVMLQRLENYIKNLMEALATEYPTVKFYAWDVV
NEAVDPNTSDGMRTPGSNNKNPGSSLWMQTVGRDFIVKAFEYARKYAPAD
CKLFYNDYNEYEDRKCDFIIEILTELKAKGLVDGMGMQSHWVMDYPSISM
FEKSIRRYAALGLEIQLTELDIRNPDNSQWALERQANRYKELVTKLVDLK
KEGINITALVFWGITDATSWLGGYPLLFDAEYKAKPAFYAIVNSVPPLPT
EPPVQVIPGDVNGDGRVNSSDLTLMKRYLLKSISDFPTPEGKIAADLNED
GKVNSTDLLALKKLVLREL
--------------------------------------------------
Sequence ID: 63
Sequence Length: 760
Sequence Type: Protein
Organism: Clostridium thermocellum
Class of Enzyme: xylanase
CAzy Family: GH10
Enzyme Classifcation Number: EC 3.2.1.8
Definition of Activity:
Accession Number: ABN53326
MIVGKVLDMDEKTAIIMTDDFAFLNVVRTSEMAVGKKVKVLDSDIIKPKN
SLRRYLPVAAVAACFVIVLSFVLMFINGNTARKNIYAYVGIDINPSIELW
INYNNKIAEAKALNGDAETVLEGLELKEKTVAEAVNEIVQKSMELGFISR
EKENIILISTACDLKAGEGSENKDVQNKIGQLFDDVNKAVSDLKNSGITT
RILNLTLEERESSKEENISMGRYAVYLKAKEQNVNLTIDEIKDADLLELI
AKVGIDNENVPEDIVTEDKDNLDAINTGPAESAVPEVTETLPATSTPGRT
EGNTATGSVDSTPALSKNETPGKTETPGRTFNTPAKSSLGQSSTPKPVSP
VQTATATKGIGTLTPRNSPTPVIPSTGIQWIDQANERINEIRKRNVQIKV
VDSSNKPIENAYVEAVLTNHAFGFGTAITRRAMYDSNYTKFIKDHFNWAV
FENESKWYTNEPSMGIITYDDADYLYEFCRSNGIKVRGHCIFWEAEEWQP
AWVRSLDPFTLRFAVDNRLNSAVGHFKGKFEHWDVNNEMIHGNFFKSRLG
ESIWPYMFNRAREIDPNAKYFVNNNITTLKEADDCVALVNWLRSQGVRVD
GVGVHGHFGDSVDRNLLKGILDKLSVLNLPIWITEYDSVTPDEYRRADNL
ENLYRTAFSHPSVEGIVMWGFWERVHWRGRDASIVNDNWTLNEAGRRFES
LMNEWTTRAYGSTDGSGSFGFRGFYGTYRITVTVPGKGKYNYTLNLNRGS
GTLQTTYRIP
--------------------------------------------------
Sequence ID: 64
Sequence Length: 576
Sequence Type: Protein
Organism: Vibrio sp.
Class of Enzyme: beta-1,3-xylanase
CAzy Family: GH26
Enzyme Classifcation Number: EC 3.2.1.32
Definition of Activity:
Accession Number: BAD51934
MKRTYLSLIAAGVMSLSVSAWSLDGVLVPESGILVSVGQDVDSVNDYASA
LGTIPAGVTNYVGIVNLDGLNSDADAGAGRNNIAELANAYPTSALVVGVS
MNGEVDAVASGRYNANIDTLLNTLAGYDRPVYLRWAYEVDGPWNGHSPSG
IVTSFQYVHDRIIALGHQAKISLVWQVASYCPTPGGQLDQWWPGSEYVDW
VGLSYFAPQDCNWDRVNEAAQFARSKGKPLFLNESTPQRYQVADLTYSAD
PAKGTNRQSKTSQQLWDEWFAPYFQFMSDNSDIVKGFTYINADWDSQWRW
AAPYNEGYWGDSRVQANALIKSNWQQEIAKGQYINHSETLFETLGYGSTG
GGDNGGGDNGGTNPPEPCNEEFGYRYVSDSTIEVFHKNNGWSAEWNYVCL
NGLCLQGEIKNGEYVKQFDAQLGSTYGIEFKVADGESQFITDKSVTFENK
QCGSTGTPGGGDNGSGGDNGGDNGSGGDNGSGGGTDPSQCSADFGYNYRS
DTEIEVFHKDLGWSASWNYICLDDYCVPGDKSGDSYNRSFNATLGSDYKI
TFKVEDSASQFITEKNITFVNTSCAQ
--------------------------------------------------
Sequence ID: 65
Sequence Length: 469
Sequence Type: Protein
Organism: Alcaligenes sp.
Class of Enzyme: beta-1,3-xylanse
CAzy Family: GH26
Enzyme Classifcation Number: EC 3.2.1.32
Definition of Activity:
Accession Number: BAB88993
MKKLAKMISIATLGACAFSAHALDGKLVPNEGVLVSVGQDVDSVNDYSSA
MSTTPAGVTNYVGIVNLDGLASNADAGAGRNNVVELANLYPTSALIVGVS
MNGQIQNVAQGQYNANIDTLIQTLGELDRPVYLRWAYEVDGPWNGHNTED
LKQSFRNVYQRIRELGYGDNISMIWQVASYCPTAPGQLSSWWPGDDVVDW
VGLSYFAPQDCNWDRVNEAAQWARSHNKPLFINESSPQRYQLADRTYSSD
PAKGTNRQSKTEQQIWSEWFAPYFQFMEDNKDILKGFTYINADWDSQWRW
AAPYNEGYWGDSRVQVLPYIKQQWQDTLENPKFINHSSDLFAKLGYVADG
GDNGGDNGGDNGGDNGGDNGGDNGGTEPPENCQDDFNFNYVSDQEIEVYH
VDKGWSAGWNYVCLNDYCLPGNKSNGAFRKTFNAVLGQDYKLTFKVEDRY
GQGQQILDRNITFTTQVCN
--------------------------------------------------
Sequence ID: 66
Sequence Length: 398
Sequence Type: Protein
Organism: Dictyoglomus thermophilum
Class of Enzyme: beta-mannanase
CAzy Family: GH26
Enzyme Classifcation Number: EC 3.2.1.78
Definition of Activity:
Accession Number: AAB82454
MHELIIGYAAPYGYKENSLYVNGEFQTNVKFPQSQKFTTVYAGLIPLKNG
KNTISIVKSWGWFLLDYFKIKKAEIPTMNPTNKLVTPNPSKEAQKLMDYL
VSIYGKYTLSGQMGYKDAFWIWNITDKFPAICGFDMMDYSPSRVERGASS
RDVEDAIDWWNMGGIVQFQWHWNAPKGLYDTPGKEWWRGFYTNATSFDIE
YALNHPESEDYKLIIRDIDAIAVQLKRLQEAKVPILWRPLHEAEGRWFWW
GAKGPEACKKLWRLLFDRLVNYHKINNLIWVWTTTDSPDALKWYPGDEYV
DIVGADIYLKDKDYSPSTGMFYNIVKLFGGKKLVALTENGIIPDPDLMKE
QKAYWVWFMTWSGFENDPNKNEISHIKKVFNHPFVITKDELPNLKVEE
--------------------------------------------------
Sequence ID: 67
Sequence Length: 329
Sequence Type: Protein
Organism: Thermotoga maritima
Class of Enzyme: beta-mannanase
CAzy Family: GH5
Enzyme Classifcation Number: EC 3.2.1.78
Definition of Activity:
Accession Number: AAD36817
MNNTIPRWRGFNLLEAFSIKSTGNFKEEDFLWMAQWDFNFVRIPMCHLLW
SDRGNPFIIREDFFEKIDRVIFWGEKYGIHICISLHRAPGYSVNKEVEEK
TNLWKDETAQEAFIHHWSFIARRYKGISSTHLSFNLINEPPFPDPQIMSV
EDHNSLIKRTITEIRKIDPERLIIIDGLGYGNIPVDDLTIENTVQSCRGY
IPFSVTHYKAEWVDSKDFPVPEWPNGWHFGEYWNREKLLEHYLTWIKLRQ
KGIEVFCGEMGAYNKTPHDVVLKWLEDLLEIFKTLNIGFALWNFRGPFGI
LDSERKDVEYEEWYGHKLDRKMLELLRKY
--------------------------------------------------
Sequence ID: 68
Sequence Length: 694
Sequence Type: Protein
Organism: Geobacillus stearothermophilus
Class of Enzyme: beta-mannanase
CAzy Family: GH5
Enzyme Classifcation Number: EC 3.2.1.78
Definition of Activity:
Accession Number: AAC71692
MNKKWSYTFIALLVSIVCAVVPIFFSQNNVHAKTKREPATPTKDNEFVYR
KGDKLMIGNKEFRFVGTNNYYLHYKSNQMIDDVIESAKKMGIKVIRLWGF
FDGMTSENQAHNTYMQYEMGKYMGEGPIPKELEGAQNGFERLDYTIYKAK
QEGIRLVIVLTNNWNNFGGMMQYVNWIGETNHDLFYTDERIKTAYKNYVH
YLINRKNQYTGIIYKNEPTIMAWELANEPRNDSDPTGDTLVRWADEMSTY
IKSIDPHHLVAVGDEGFFRRSSGGFNGEGSYMYTGYNGVDWDRLIALKNI
DYGTFHLYPEHWGISPENVEKWGEQYILDHLAAGKKAKKPVVLEEYGISA
TGVQNREMIYDTWNRTMFEHGGTGAMFWLLTGIDDNPESADENGYYPDYD
GFRIVNDHSSVTNLLKTYAKLFNGDRHVEKEPKVYFAFPAKPQDVRGTYR
VKVKVASDQHKVQKVQLQLSSHDEAYTMKYNASFDYYEFDWDTTKEIEDS
TVTLKATATLTNKQTIASDEVTVNIQNASAYEIIKQFSFDSDMNNVYADG
TWQANFGIPAISTPKTRCLRVNVDLPGNADWEEVKVKISPISELSETSRI
SFDLLLPRVDVNGALRPYIALNPGWIKIGVDQYHVNVNDLTTVTIHNQQY
KLLHVNVEFNAMPNVNELFLNIVGNKLAYKGPIYIDNVTLFKKI
--------------------------------------------------
Sequence ID: 69
Sequence Length: 569
Sequence Type: Protein
Organism: Ruminococcus albus
Class of Enzyme: xylanase (possible fexeranase)
CAzy Family: GH5
Enzyme Classifcation Number: EC 3.2.1.8/3.2.1.136
Definition of Activity:
Accession Number: BAB39495
MKQNGVNLYAISVQNEPDYAKDWTAWTPDETTDFIANYGDQITSTKLMSP
ESFQYGAYNNGKDYYSKILNNSKAYANCDIFGTHFYGTPRSKMDFPALEN
CGKQLWMTEVYVPDSNVDSNIWPDNLKQAVSIHDSLVVGGMQAYVVWPLR
RNYSILREDTHKISKRGYAFAQYSKFVRPGDVRVDVTEQPSSNVFVSAYK
NNKNQVTIVAINNSSSGYSQQFSLNGKTIIDVDRWRTSGSENLAETDNLT
IDNGTSFWAQLPAQSVSTFVCTLSGGSSSGNNGSSNTELDSDGYYFHDTF
EDDLTWQAHGGTELLKSGRTPYKGSEAVVVTNRTSAWMGAERTLPSSVVP
GKTYSFSVNVTELDGEDTETFYLKLNYTDSSGTAHYPTIAEGVCPKGKYL
QLSNTNYTIPSDAVDPVIYVETKDTTSNFYIDEAICAPAGKSLPGAGIPE
IPSNNNNNNNNNNNNNNNNNNNNQNNSVYPVVSSIDYNVTYHQFRISWNS
VPNAQAYGIAYYAAGKWRVYTQSISVNTTSWISPKLTAGKTYTMVIAAKV
GGKWDTSNLSSRAINVTVK
--------------------------------------------------
Sequence ID: 70
Sequence Length: 1217
Sequence Type: Protein
Organism: Cytophaga hutchinsonii
Class of Enzyme: xylanase (possible fexeranase)
CAzy Family: GH5
Enzyme Classifcation Number: EC 3.2.1.8/3.2.1.136
Definition of Activity:
Accession Number: YP_678647
MKKLFTVLFYLSTCLVWAQTSTVNLTSEKQYIRGFGGINHPEWAGDMTAV
QRTTAFGNGAGEMGLTVLRIFVNDDKTQWNKALATALRAQQLGATIFATP
WNPPASMCETITRNNRQEKRLKPGSYSAYAQHLIDFNNYMKNNGVNLYAN
SFANEPDWGFDWTWYSADEVYNFTKNIAGTLRVNGIKVITAESFSYNKSY
YDKVLNDPTALSNIDIIGCHLYGSDANSPVSVFNYPLADSKAPTKERWMT
EHYTNSDANSSDLWPSANDVSYEIYRCMVEGQMSVYTWWYIRRQYGPMNE
NGTISKRGYCMAQYSKFIRPGYKRVDATKNPATGVYISAYKKGDDVVVVA
INRSTSSQTITLSVPGTKVTTWEKYVTSGSKSLAKEANINSSTGSFQITL
DPQSTTSFVGTAPVITTPSPVVSLTAPVNNTVYTEGDNITINATATITSG
SISKVEFYNGTTLLGTDASSPYSYTITAAAAGTYPVTAKATSAANAVTTS
TAINIQVAKPIYQTGSAPTIDGTVDGLWSNFPSTGITKNNTGTISSGTDL
SGNWKAMWDASNLYVLVQVTDDVKRNDGGTDVYNDDGVEVYIDLGNTKAT
TYGTNDQQYTFRWNDVTAAYEINGHPVTGITKGISNTATGYIVEVSIPWS
TIGGTASLNSFQGFEVMINDDDDGGAREGKLAWVASTDDTWSNPALMGTV
VLKGLNCTVPAAAITASTATTFCSGGSVVLNAGTGTGYSYVWKNGAATIA
GATNSGYTATASGSYTVTVTNPGGCSATSAGTTVTVNALPVLTQYAQVDG
GTWNQVSGATVCAGSSVVLGPQPTVNTGWSWTGPNGYSASARELRLTSVQ
TNQGGVYTASYTDGNTCKSTSVFTLTVTALPAAAITTSTPTTFCAGGSTT
LTAGSGASYKWMNGTVAITGATAQTYTATAAGSYTVEVTNAGNCKATSAA
TVVTVTALPTATITATGSTTIPQGGSVALQANAGSALTYKWFNGTVAITG
ATAQTYTATTAGSYTVEVTNAGNCKATSAAATVSVVANQPSVITITSPAP
NAAVTGAIDISVNITDADGSITLVEFLAGDDVIGTAAAAPYTYTWDTPTA
GSHTITVRVTDSNGGVTTSAPVTVTSESITTGVQALNTLNAAVYPNPSNG
IVFIDTDADLSDASFTLIDVLGKEGTVFSTATGNGAMIDVSSLAGGTYVL
IVKKDTSVIRKKITVIR
--------------------------------------------------
Sequence ID: 71
Sequence Length: 536
Sequence Type: Protein
Organism: Piromyces equi
Class of Enzyme: Ferulic acid esterase
CAZy Family: CE1
Enzyme Classification Number: 3.1.1.73
Accession Number: AAD45376
MKTSIVLSIVALFLTSKASADCWSERLGWPCCSDSNAEVIYVDDDGDWGV
ENNDWCGIQKEEENNNSWDMGDWNQGGNQGGGMPWGDFGGNQGGGMQWGD
FGGNQGGGMPWGDFGGNQGGGMPWGDFGGNQGGNQGGGMPWGDFGGNQGG
NQGGGMPWGDFGGNQGGGMQWGDFGGNQGGNQGGGMPWGDFGGNQGGGMQ
WGDFGGNQGGNQGGGMPWGDFGGNQGGGMQWGDFGGNQGGGMQWGDFGGN
QGGNQDWGNQGGNSGPTVEYSTDVDCSGKTLKSNTNLNINGRKVIVKFPS
GFTGDKAAPLLINYHPIMGSASQWESGSQTAKAALNDGAIVAFMDGAQGP
MGQAWNVGPCCTDADDVQFTRNFIKEITSKACVDPKRIYAAGFSMGGGMS
NYAGCQLADVIAAAAPSAFDLAKEIVDGGKCKPARPFPILNFRGTQDNVV
MYNGGLSQVVQGKPITFMGAKNNFKEWAKMNGCTGEPKQNTPGNNCEMYE
NCKGGVKVGLCTINGGGHAEGDGKMGWDFVKQFSLP
--------------------------------------------------
Sequence ID: 72
Sequence Length: 558
Sequence Type: Protein
Organism: Fusarium oxysporum
Class of Enzyme: Ferulic acid esterase
CAZy Family:
Enzyme Classification Number:
Accession Number:
MLFASLVLVLGFIPQVLSDTSTDICLPQDNMRPTFLLFSGLGACAPAGKG
DDFAAKCAGFKTSLKLPNTKVWFTEHVPAGKHITFPDNHPTCTPKSTITD
VEICRVAMFVTTGPKSNLTLEAWLPSNWTGRFLSTGNGGMAGCIQYDDVA
YGAGFGFATVGANNGHNGTSAVSMYKNSGVVEDYWYRSVHTGTVLGKELT
KKFYGKKHTKSYYLGCSTGGRQGWKEAQSFPDDFDGIVAGAPAMRFNGLQ
SRSGSFWGITGPPGAPTHLSPEEWAMVQKNVLVQCDEPLDGVADGILEDP
NLCQYRPEALVCSKGQTKNCLTGPQIETVRKVFGPLYGNNGTYIYPRIPP
GADQGFGFAIGEQPFPYSTEWFQYVIWNDTKWDPNTIGPNDYQKASEVNP
FNVETWEGDLSKFRKRGSKIIHWHGLEDGLISSDNSMEYYNHVSATMGLS
NTELDEFYRYFRVSGCGHCSGGIGANRIGNNRANLGGKEAKNNVLLALVK
WVEEDQAPETITGVRYVNGATTGKVEVERRHCRYPYRNVWDRKGNYKNPD
SWKCELPK
--------------------------------------------------
Sequence ID: 73
Sequence Length: 280
Sequence Type: Protein
Organism: Penicillium chrysogenum
Class of Enzyme: Ferulic acid esterase
CAZy Family: CE1
Enzyme Classification Number: 3.1.1.73
Accession Number: CAP86030
MKISAPRALALSVAVGHALAAVTKGVSDNIYNRLVDMATISQAAYADLCK
IPATITTVEKIYNAQTDINGWVLRDDSRQEIIVVFRGTAGDTNLQLDTNY
TLAPFDTLPKCIGCAVHGGYYLGWTSVQDQVESLVQQQAGQYPEYALTVT
GHSLGASMAAITASQLSATYEHVTLYTFGEPRTGNLAYASYMNENFEATS
PETTRFFRVTHGNDGIPNLPPAEQGYVHSGIEYWSVDPHRPGSTSVCTGN
EVQCCEAQGGQGVNDDHITYFGMASGACSW
--------------------------------------------------
Sequence ID: 74
Sequence Length: 353
Sequence Type: Protein
Organism: Penicillium funiculosum
Class of Enzyme: Ferulic acid esterase
CAZy Family: CE1
Enzyme Classification Number: 3.1.1.73
Accession Number: CAC14144
MAIPLVLVLAWLLPVVLAASLTQVNNFGDNPGSLQMYIYVPNKLASKPAI
IVAMHPCGGSATEYYGMYDYHSPADQYGYILIYPSATRDYNCFDAYSSAS
LTHNGGSDSLSIVNMVKYVISTYGADSSKVYMTGSSSGAIMTNVLAGAYP
DVFAAGSAFSGMPYACLYGAGAADPIMSNQTCSQGQIQHTGQQWAAYVHN
GYPGYTGQYPRLQMWHGTADNVISYADLGQEISQWTTIMGLSFTGNQTNT
PLSGYTKMVYGDGSKFQAYSAAGVGHFVPTDVSVVLDWFGITSGTTTTTT
PTTTPTTSTSPSSTGGCTAAHWAQCGGIGYSGCTACASPYTCQKANDYYS
QCL
--------------------------------------------------
Sequence ID: 75
Sequence Length: 292
Sequence Type: Protein
Organism: Neurospora crassa
Class of Enzyme: Ferulic acid esterase
CAZy Family: CE1
Enzyme Classification Number: 3.1.1.73
Accession Number: CAC05587
MLPRTLLGLALTAATGLCASLQQVTNWGSNPTNIRMHTYVPDKLATKPAI
IVALHGCGGTAPSWYSGTRLPSYADQYGFILIYPGTPNMSNCWGVNDPAS
LTHGAGGDSLGIVAMVNYTIAKYNADASRVYVMGTSSGGMMTNVMAATYP
EVFEAGAAYSGVAHACFAGAASATPFSPNQTCARGLQHTPEEWGNFVRNS
YPGYTGRRPRMQICHGLADNLVYPRCAMEALKQWSNVLGVEFSRNVSGVP
SQAYTQIVYGDGSKLVGYMGAGVGHVAPTNEQVMLKFFGLIN
--------------------------------------------------
Sequence ID: 76
Sequence Length: 767
Sequence Type: Protein
Organism: Saccharophagus degradans
Class of Enzyme: Acetylxylan esterase
CAZy Family:
Enzyme Classification Number: 3.1.1.72
Accession Number: ABD82318
MKSINVCGRRLKQALAAIATAAATLWFTPVDAQTLTSNQTGTHGGYYYSF
WTDSAGTVSMTLGNGGNYSSSWSNTGNWVGGKGWQTGGRKTVNYSGTFNP
SGNGYLTLYGWTQNPLIEYYIIESWGTYRPGESGTYYGTVNTDGGTYDIY
RTQRVNQPSIEGTATFYQYWSVRQQKRVGGTITTGNHFDAWASHGLNLGT
HNYMVMATEGYQSSGNSNITVSEGSGSSSTSSSSSSTGGPSGTNIVVRAQ
GVSGQEHINLIIGGNVVADWTLSTSMQDYTYTGNAAGDLQVEYDNDASGR
DVELDYVYVNGEIRQAEDMEYNTATYSGECGGGSYSQTMHCSGVIGFGDT
SDCFSGNCNGASSTSSSSSSSSTSSSTSSGGNNNSGITVRARGTNGDEHI
NLIVGGNIVGNWTLTTSNQNYVYNGNASGDVEVQFDNDANGRDVILDYVI
VNGETRQAEDMEYNTATYSGSCGGGSYSETMHCSGEIGFGHTDDCFSGNC
TSSSGTTGSSGGTSSNNGTSSCNGYVGITFDDGPGNNTATLINLLQQNNL
TPVTWFNTGQNIAANTGQFAQQKSVGEIQNHSYTHSHMLNWSYQQVRDEL
ASTNQAIVNAGGATPTLFRPPYGETNSTINQAAQDLGLRVITWDVDSRDW
DGASASAIANSANQLQNGQVILMHDASYNNTNGAISQFAANLRARGLCAG
KIDPSTGRAVAPSTNTGGNTGSNTGNGGNGGMCNWYGTSIPLCQTTNDGW
GWENSQSCVSQNTCNSQ
--------------------------------------------------
Sequence ID: 77
Sequence Length: 382
Sequence Type: Protein
Organism: Penicillium purpurogenum
Class of Enzyme: Acetylxylan esterase
CAZy Family: CE1
Enzyme Classification Number: 3.1.1.72
Accession Number: AAM93261
MKSLSFSFLVTLFLYLTLSSARTLGKDVNKRVTAGSLQQVTGFGDNASGT
LMYIYVPKNLATNPGIVVAIHYCTGTAQAYYTGSPYAQLAEQYGFIVIYP
QSPYSGTCWDVSSQAALTHNGGGDSNSIANMVTWTISQYNANTAKVFVTG
SSSGAMMTNVMAATYPELFAAATVYSGVGAGCFYSSSNQADAWNSSCATG
SVISTPAVWGGIAKNMYSGYSGSRPRMQIYHGSADTTLYPQNYYETCKQW
AGVFGYNYDSPQSTLANTPDANYQTTNWGPNLQGIYATGVGHTVPIHGAK
DMEWFGFSGSGSSSTTTASATKTSTTSTTSTKTTSSTSSTTTSSTGVAAH
WGQCGGSGWTGPTVCESGYTCTYSNAWYSQCL
--------------------------------------------------
Sequence ID: 78
Sequence Length: 413
Sequence Type: Protein
Organism: Erwinia chrysanthemi
Class of Enzyme: Glucuronoxylan xylanase
CAZy Family: GH5
Enzyme Classification Number: 3.2.1.136
Accession Number: AAB53151
MNGNVSLWVRHCLHAALFVSATAGSFSVYADTVKIDANVNYQIIQGFGGM
SGVGWINDLTTEQINTAYGSGVGQIGLSIMRVRIDPDSSKWNIQLPSARQ
AVSLGAKIMATPWSPPAYMKSNNSLINGGRLLPANYSAYTSHLLDFSKYM
QTNGAPLYAISIQNEPDWKPDYESCEWSGDEFKSYLKSQGSKFGSLKVIV
AESLGFNPALTDPVLKDSDASKYVSIIGGHLYGTTPKPYPLAQNAGKQLW
MTEHYVDSKQSANNWTSAIEVGTELNASMVSNYSAYVWWYIRRSYGLLTE
DGKVSKRGYVMSQYARFVRPGALRIQATENPQSNVHLTAYKNTDGKMVIV
AVNTNDSDQMLSLNISNANVTKFEKYSTSASLNVEYGGSSQVDSSGKATV
WLNPLSVTTFVSK
--------------------------------------------------
Sequence ID: 79
Sequence Length: 1467
Sequence Type: Protein
Organism: Paenibacillus sp. JDR-2
Class of Enzyme: Glucuronoxylan xylanase
CAZy Family: GH5
Enzyme Classification Number: 3.2.1.136
Accession Number: AJ938162
MSRSLKKFVSILLAAALLIPIGRLAPVAEAAENPTIVYHEDFAIDKGKAI
QSGGASLTQVTGKVFDGNNDGSALYVSNRANTWDAADFKFADIGLQNGKT
YTVTVKGYVDQDATVPSGAQAFLQAVDSNNYGFLASANFAAGTAFTLTKE
FTVDTSVSTQLRVQSSEEGKAVPFYIGDILITANPTTTTNTVYHEDFATD
KGKAVQSGGANLAQVADKVFDGNDDGKALYVSNRANTWDAADFKFADIGL
QNGKTYTVTVKGYVDQDATVPSGAQAFLQAVDSNNYGFLASANFAARSAF
TLTKEFTVDTSVTTQLRVQSSEEGKAVPFYIGDILITETVNSGGGQEDPP
RPPALPFNTITFEDQTAGGFTGRAGTETLTVTNESNHTADGSYSLKVEGR
TTSWHGPSLRVEKYVDKGYEYKVTAWVKLLSPETSTKLELASQVGDGGSA
NYPTPTTQAWQARRLPAADGWVQLQGNYRYNSVGGEYLTIYVQSSNATAS
YYIDDISFESTGSGPVGIQKDLAPLKDVYKNDFLIGNAISAEDLEGTRLE
LLKMHHDVVTAGNAMKPDALQPTKGNFTFTAADAMIDKVLAEGMKMHGHV
LVWHQQSPAWLNTKKDDNNNTVPLGRDEALDNLRTHIQTVMKHFGNKVIS
WDVVNEAMNDNPSNPADYKASLRQTPWYQAIGSDYVEQAFLAAREVLDEN
PSWNIKLYYNDYNEDNQNKATAIYNMVKDINDRYAAAHNGKLLIDGVGMQ
GHYNINTNPDNVKLSLEKFISLGVEVSVSELDVTAGNNYTLPENLAVGQA
YLYAQLFKLYKEHADHIARVTFWGMDDNTSWRAENNPLLFDKNLQAKPAY
YGVIDPDKYMEEHAPESKDANQAEAQYGTPVIDGTVDSIWSNAQAMPVNR
YQMAWQGATGTAKALWDDQNLYVLIQVSDSQLNKANENAWEQDSVEVFLD
QNNGKTTFYQNDDGQYRVNFDNETSFSPASIAAGFESQTKKTANSYTVEL
KIPLTAVTPANQKKLGFDVQINDATDGARTSVAAWNDTTGNGYQDTSVYG
ELTLAGKGTGGTGTVGTTVPQTGNVVKNPDGSTTLKPEVKTTNGNAVGTV
TGDDLKKALDQAAPAAGGKKQVIIDVPLQANAATYAVQLPTQSLKSQDGY
QLTAKIANAFIQIPSNMLANTNVTTDQVSIRVAKASLDNVDAATRELIGN
RPVIDLSLVAGGNVIAWNNPTAPVTVAVPYAPTAEELKHPEHILIWYIDG
SGKATPVPNSRYDAALGAVVFQTTHFSTYAAVSVFTTFGDLAKVPWAKEA
IDAMASRGVIKGTGENTFSPAASIKRADFIALLVRALELHGTGTTDTAMF
SDVPANAYYYNELAVAKQLGIATGFEDNTFKPDSSISRQDMMVLTTRALA
VLGKQLPAGGSLNAFSDAASVAGYAQDSVAALVKAGVVQGSGSKLAPNDQ
LTRAEAAVILYRIWKLQ
--------------------------------------------------
Sequence ID: 80
Sequence Length: 444
Sequence Type: Protein
Organism: Thermotoga neapolitana
Class of Enzyme: Beta-glucan glucohydrolase
CAZy Family: GH1
Enzyme Classification Number: 3.2.1.74
Accession Number: AAB95492
MKKFPEGFLWGVATASYQIEGSPLADGAGMSIWHTFSHTPGNVKNGDTGD
VACDHYNRWKEDIEIIEKIGAKAYRFSISWPRILPEGTGKVNQKGLDFYN
RIIDTLLEKNITPFITIYHWDLPFSLQLKGGWANRDIADWFAEYSRVLFE
NFGDRVKHWITLNEPWVVAIVGHLYGVHAPGMKDIYVAFHTVHNLLRAHA
KSVKVFRETVKDGKIGIVFNNGYFEPASEREEDIRAARFMHQFNNYPLFL
NPIYRGEYPDLVLEFAREYLPRNYEDDMEEIKQEIDFVGLNYYSGHMVKY
DPNSPARVSFVERNLPKTAMGWEIVPEGIYWILKGVKEEYNPQEVYITEN
GAAFDDVVSEGGKVHDQNRIDYLRAHIEQVWRAIQDGVPLKGYFVWSLLD
NFEWAEGYSKRFGIVYVDYNTQKRIIKDSGYWYSNGIKNNGLTD
--------------------------------------------------
Sequence ID: 81
Sequence Length: 720
Sequence Type: Protein
Organism: Fibrobacter succinogenes
Class of Enzyme: Beta-glucan glucohydrolase
CAZy Family: GH1
Enzyme Classification Number: 3.2.1.74
Accession Number: ABY60376
MRIYKLSPIFSAAVLLSAGVASAETKFFYNQVGYDVDQPISVIVQSENLA
DGAEFSVMSGGTAVKTGKLSTGSNPDNWLNSGKFYVADLTGLKAGKYTLQ
VSENGQPQKSGEFTVGENALAANTLASVLNYFYDDRADDPTVEGWDKQMP
VYKSDKKLDVHGGWYDASGDVSKYLSHLSYANYLNPQQIPLTVWSLAFAS
ERIPKLLGSTSTKAKTADEAAYGADFLVRMLDEQGFFYMTVFDNWGSPMG
KREICAFSGSDGIKSTDYQTAFREGGGMAIAALASAARLKLKGDFTSEQY
LAAAEKAYKHLSEKQSVGGDCAYCDDHKENIIDDYTALLAATELYAATKK
QEYLEDAYDRAEHLSSRVSKDGYFWSDDAKTRPFWHASDAGLPLVALARY
SEVVGAIDEDAGIKVHGRPFPYWGCVTMIGGGCVNESIDNVRNAIRSHFD
WLVKITNKVDNPFGYARQTYKTQDKIKDGFFIPHDNESNYWWQGEDARLA
SLSAAIMYANRIIDGEYRNVTTSDVQKYATDQLDWILGKNPYATCMMYGK
GTKNPQKYDGQSKYDATLEGGIANGISGKNQDGSGIAWTDDGVAAVGFDS
EKESWQVWRWDEQWLPHSTWYLMALVERYDELTKPVEFSVGLSKSTVAAK
ASVSLVGKMLSLNLPRSVVGKSVKVLDVRGNVLMQKTVQGVSETMDVSTL
NRGLYLVQIQGFAAKKFVVK
--------------------------------------------------
Sequence ID: 82
Sequence Length: 925
Sequence Type: Protein
Organism: Thermobifida fusca
Class of Enzyme: Xyloglucanase
CAZy Family: GH74
Enzyme Classification Number: 3.2.1.151
Accession Number: YP_289670
MTATAQRTPPPPTPRRRGIIARALTCIAPAATVAAVGLVHSAAAPASATT
GYTWRNVEIVGGGFVPGIVFNQSEPDLIYARTDIGGAYRWDPATERWIPL
LDHVGWDDWGHSGVVSIATDPVDPDRVYAAVGTYTNDWDPNNGAIKRSTD
RGETWETTELPFKLGGNMPGRGMGERLAIDPNDNSVLYLGAPSGHGLWKS
TDYGKTWQKVTSFPNPGNYVADPSDVGGYLGDNQGVVWVVFDPTSSSPGH
VTKDIYVGVADKQNTVYRSTDGGQTWERIPGQPTGFLAQKGVFDHVNGLL
YIATSDTGGPYDGSDGEVWRYDTTTGTWTDITPADPDGFEYGFSGLTIDR
QNPDTIMVVSQILWWPDIQIWRSTDRGETWSRIWEFSGYPDRTLRYNHDI
SAAPWLDFNRQDNPPEVSPKLGWMTQAFEIDPFNSDRMLYGTGATIYGSD
NLTNWDEGKKIDIKVRAQGIEETAVQDLIAPPGDTELVSALGDIGGFVHD
DITVVPDAMFDSPFHGNTRSIDFAELNPSVMARVGEAVDGEVDSHIGIST
SGGSHWWAGQEPSGVTGAGTVAVNADGSRIVWSPDGTGVHYSTTLGSSWT
PSQGVPAGARVEADRVNPDKFYAFANGTFYTSTDGGATFTKSSAAGLPTK
GNIRFAAVPGHEGDIWLAGGETNSTYGMWRSTDSGATFTRITAVDEGDVV
GFGKPAPGRSYPAVYTSSKINGVRGIFRSDDAGTTWVRINDDQHQWAWTG
AAITGDPDVYGRVYIGTNGRGVIVGDLDGPPPQPTEEPTEEPSTPPTEEP
TEEPTEEPSTPPTEEPPGDAACAVSYQVLNEWGGGFQGEVTITNTGDTPI
NGWELTWTFPDNQQITQAWNTQLTQSGAKVTARDAGWNSTIAPGGTASFG
FLGSPAPGSKPTEFTLNGTPCSAAG
--------------------------------------------------
Sequence ID: 83
Sequence Length: 540
Sequence Type: Protein
Organism: Phanerochaete chrysosporium
Class of Enzyme: Exoglucanase/Cellobiohydrolase I
CAZy Family: GH7
Enzyme Classification Number: 3.2.1.-
Accession Number: CAA82761
MFRPAALLAFTCLAMVSGQQAGTNTAENHPQLQSQQCTTSGGCKPLSTKV
VLDSNWRWVHSTSGYTNCYTGNEWDTSLCPDGKTCAANCALDGADYSGTY
GITSTGTALTLKFVTGSNVGSRVYLMADDTHYQLLKLLNQEFTFDVDMSN
LPCGLNGALYLSAMDADGGMSKYPGNKAGAKYGTGYCDSQCPKDIKFING
EANVGNWTETGSNTGTGSYGTCCSEMDIWEANNDAAAFTPHPCTTTGQTR
CSGDDCARNTGLCDGDGCDFNSFRMGDKTFLGKGMTVDTSKPFTVVTQFL
TNDNTSTGTLSEIRRIYIQNGKVIQNSVANIPGVDPVNSITDNFCAQQKT
AFGDTNWFAQKGGLKQMGEALGNGMVLALSIWDDHAANMLWLDSDYPTDK
DPSAPGVARGTCATTSGVPSDVESQVPNSQVVFSNIKFGDIGSTFSGTSS
PNPPGGSTTSSPVTTSPTPPPTGPTVPQWGQCGGIGYSGSTTCASPYTCH
VLNPCESILSLQRSSNADQYLQTTRSATKRRLDTALQPRK
--------------------------------------------------
Sequence ID: 84
Sequence Length: 837
Sequence Type: Protein
Organism: Clostridium thermocellum
Class of Enzyme: Xylanase
CAZy Family: GH10
Enzyme Classification Number: 3.2.1.8
Accession Number: YP_001038374
MSRKLFSVLLVGLMLMTSLLVTISSTSAASLPTMPPSGYDQVRNGVPRGQ
VVNISYFSTATNSTRPARVYLPPGYSKDKKYSVLYLLHGIGGSENDWFEG
GGRANVIADNLIAEGKIKPLIIVTPNTNAAGPGIADGYENFTKDLLNSLI
PYIESNYSVYTDREHRAIAGLSMGGGQSFNIGLTNLDKFAYIGPISAAPN
TYPNERLFPDGGKAAREKLKLLFIACGTNDSLIGFGQRVHEYCVANNINH
VYWLIQGGGHDFNVWKPGLWNFLQMADEAGLTRDGNTPVPTPSPKPANTR
IEAEDYDGINSSSIEIIGVPPEGGRGIGYITSGDYLVYKSIDFGNGATSF
KAKVANANTSNIELRLNGPNGTLIGTLSVKSTGDWNTYEEQTCSISKVTG
INDLYLVFKGPVNIDWFTFGVESSSTGLGDLNGDGNINSSDLQALKRHLL
GISPLTGEALLRADVNRSGKVDSTDYSVLKRYILRIITEFPGQGDVQTPN
PSVTPTQTPIPTISGNALRDYAEARGIKIGTCVNYPFYNNSDPTYNSILQ
REFSMVVCENEMKFDALQPRQNVFDFSKGDQLLAFAERNGMQMRGHTLIW
HNQNPSWLTNGNWNRDSLLAVMKNHITTVMTHYKGKIVEWDVANECMDDS
GNGLRSSIWRNVIGQDYLDYAFRYAREADPDALLFYNDYNIEDLGPKSNA
VFNMIKSMKERGVPIDGVGFQCHFINGMSPEYLASIDQNIKRYAEIGVIV
SFTEIDIRIPQSENPATAFQVQANNYKELMKICLANPNCNTFVMWGFTDK
YTWIPGTFPGYGNPLIYDSNYNPKPAYNAIKEALMGY

In some embodiments, a cell wall-modifying enzyme polypeptide provided herein is characterized by at least one enzymatic activity. In some embodiments, the activity is an activity listed in Table 2.

TABLE 2
Representative enzyme activities of provided
cell wall-modifying enzyme polypeptides

| Enzyme Activity | Classification | CAZy family |
|---|---|---|
| Endoglucanase | EC 3.2.1.4 | GH5 |
| Reducing end specific | EC 3.2.1.— | GH7 |
| exoglucanase | | |
| Non-reducing end specific | EC 3.2.1.91 | GH48 |
| exoglucanase | | |
| Xylanase | EC 3.2.1.8 | GH10 |
| Ferulic Acid Esterase | EC 3.1.1.73 | CE1 |
| Alpha L | EC 3.2.1.55 | GH51, 62 |
| arabinofuranosidase | | |
| Licheninase | EC 3.2.1.73/4 | GH12, 16 |
| Laminarinase | EC 3.2.1.39 | GH16 |
| Acetylxylan esterase | EC 3.1.1.72 | CE6 |
| Pectinmethylesterase | EC 3.1.1.11 | CE8 |
| Endopolygalacturonase | EC 3.2.1.15 | GH28 |
| Rhamnogalacturonan lyase | EC 4.2.2.— EC 4.2.2.— | PL4 |
| Beta-xylosidase | EC 3.2.1.37 | GH3 |
| Endoxyloglucanse | EC 3.2.1.151 | GH74 |
| Endoarabinase | EC 3.2.1.99 | GH43 |
| Exopolygalacturonase | EC 3.2.1.—/82/67 | GH28 |
| Endogalactanase | EC 3.2.1.89 | GH53 |
| Exooligoxylanase | EC 3.2.1.156 | GH8 |
| Pectin lyase | EC 4.2.2.10 | PL1 |
| Pectate lyase | EC 4.2.2.2 | PL1 |
| Alpha-L-rhamnosidase | EC 3.2.1.40 | GH78 |
| Pectin acetylesterase | EC 3.1.1.— | CE10, 12 |
| Beta-1,3(4)-glucanase | EC 3.2.1.6 | GH16 |
| Beta-glucosidase | EC 3.2.1.21 | GH1 |
| Glururonoyl esterase | EC 3.1.1.— | CE15 |
| Beta-1,3-xylanase | EC 3.2.1.32 | GH26 |
| Endomannanase | EC 3.2.1.78 | GH26 |
| Glucuronoarabinoxylan | EC 3.2.1.8/136 | GH5 |
| endo-1,4-betaxylanase | | |
| Beta-Glucan | EC 3.2.1.74 | GH1 |
| Glucohydrolase | | |

In some embodiments, the cell wall-modifying enzyme polypeptide has cellulase activity. In some embodiments, the cell wall-modifying enzyme polypeptide has an activity selected from the group consisting of feruloyl esterase (also known as ferulic acid esterase), xylanase, alpha-L-arabinofuranosidase, endogalactanase, acetylxylan esterase, beta-xylosidase, xyloglucanase, glucuronoyl esterase, endo-1,5-alpha-L-arabinosidase, pectin methylesterase, endopolygalacturonase, exopolygalacturonase, pectin lyase, pectate lyase, rhamnogalacturonan lyase, pectin acetylesterase, alpha-L-rhamnosidase, mannanase, exoglucanase, glucan glycohydrolase, licheninase, laminarinase, beta-(1,3)-(1,4)-glucanase and beta-glucosidase activity. Such activities may be similar to that of other enzyme polypeptides, including those known in the art that are classified by an EC class and/or listed in enzyme databases (such as CaZY, www.cazy.org, which lists carbohydrate-active enzymes).

Activity of cell wall-modifying enzyme polypeptides can be characterized by one or more activity assays, including ones known in the art. Generally, extracts (e.g., of plants that have been transformed to express one or more cell wall-modifying enzyme polypeptides) are incubated with a substrate such as (methylumbelliferyl cellobioside (MUC), 4-methylumbelliferyl p-trimethylammonio-cinnamate chloride (MUTMAC), etc.) and one or more cleavage product(s) is/are measured and taken as an indication of enzyme activity. (See, for example, Examples 2, 9, and 10).

In some embodiments, the cell wall-modifying enzyme polypeptide modifies a plant cell wall component. In many such embodiments, the cell wall-modifying enzyme polypeptide modifies the plant cell wall component in such a way that the plant biomass is more amenable to processing steps (e.g., enzymatic digestion). For example, cell wall-modifying enzyme polypeptides may modify plant cell wall components in such a way as to allow increased digestability, increased hydrolysis, and/or increased sugar yields.

In some embodiments, modifying comprises cleavage and/or hydrolysis of the plant cell wall component. Examples of plant cell wall components that may be modified include, but are not limited to, xylans, xylan side chains, glucuronoarabinoxylans, xyloglucans, mixed-linkage glucans, pectins, pectates, rhamnogalacturonans, rhamnogalacturonan side chains, lignin, cellulose, mannans, galactans, arabinans, oligosaccharides derived from cell wall polysaccharides, and combinations thereof.

In some embodiments, the cell wall-modifying enzyme polypeptide disrupts an interaction in the plant biomass such as a covalent linkage, an ionic bonding interaction, a hydrogen bonding interaction, or a combination thereof. Examples of linkages that may be disrupted include, but are not limited to, hemicellulose-cellulose-lignin, hemicellulose-cellulose-pectin, hemicellulose-diferululate-hemicellulose, hemicellulose-ferulate-lignin, mixed beta-D-glucan-cellulose, mixed-beta-D-glucan-hemicellulose, pectin-ferulate-lignin linkages, and combinations thereof. In some embodiments, disrupting comprises hydrolyzing a linkage, such as a feruloyl ester linkage.

### II. Lignocellulolytic Enzyme Polypeptides

In some embodiments, one or more cell wall-modifying enzyme polypeptides are used in combination with one or more lignocellulolytic enzyme polypeptides. It will be understood by those of ordinary skill in the art that at least some of the cell wall-modifying enzyme polypeptides described in the section above may also be classified as “lignocellulolytic enzyme polypeptides.” This section is intended to provide an overview of several broad classes of lignocellulolytic enzyme polypeptides and describe, as non-limiting examples, certain lignocellulolytic enzyme polypeptides that can be used in combination with the cell wall-modifying enzyme polypeptides described in the above section.

Suitable lignocellulolytic enzyme polypeptides include enzymes that are involved in the disruption and/or degradation of lignocellulose. Lignocellulolytic enzyme polypeptides include, but are not limited to, cellulases, hemicellulases and ligninases. Representative examples of lignocellulolytic enzyme polypeptides are presented in Table 3.

TABLE 3
Examples of lignocellulolytic enzyme polypeptides

| | | | GenBank |
|---|---|---|---|
| Gene | Microbial | Amino Acid Sequence of Exemplary | Accession |
| name | species | Lignocellulolytic Enzyme Polypeptide | Number |
| E1 | Acidothermus | AGGGYWHTSGREILDANNVPVRIAGINWFGFETCNYVVHGLWSRDYRS | AAA75477 |
| | cellulolyticus | MLDQIKSLGYNTIRLPYSDDILKPGTMPNSINFYQMNQDLQGLTSLQV | |
| | | MDKIVAYAGQIGLRIILDRHRPDCSGQSALWYTSSVSEATWISDLQAL | |
| | | AQRYKGNPTVVGFDLHNEPHDPACWGCGDPSIDWRLAAERAGNAVLSV | |
| | | NPNLLIFVEGVQSYNGDSYWWGGNLQGAGQYPVVLNVPNRLVYSAHDY | |
| | | ATSVYPQTWFSDPTFPNNMPGIWNKNWGYLFNQNIAPVWLGEFGTTLQ | |
| | | STTDQTWLKTLVQYLRPTAQYGADSFQWTFWSWNPDSGDTGGILKDDW | |
| | | QTVDTVKDGYLAPIKSSIFDPVG | |
| gux1 | Acidothermus | MGAPGLRRRLRAGIVSAAALGSLVSGLVAVAPVAHAAVTLKAQYKNND | ABK52390.1 |
| | cellulolyticus | SAPSDNQIKPGLQLVNTGSSSVDLSTVTVRYWFTRDGGSSTLVYNCDW | |
| | | AAMGCGNIRASFGSVNPATPTADTYLQLSFTGGTLAAGGSTGEIQNRV | |
| | | NKSDWSNFDETNDYSYGTNTTFQDWTKVTVYVNGVLVWGTEPSGATAS | |
| | | PSASATPSPSSSPTTSPSSSPSPSSSPTPTPSSSSPPPSSNDPYIQRF | |
| | | LTMYNKIHDPANGYFSPQGIPYHSVETLIVEAPDYGHETTSEAYSFWL | |
| | | WLEATYGAVTGNWTPFNNAWTTMETYMIPQHADQPNNASYNPNSPASY | |
| | | APEEPLPSMYPVAIDSSVPVGHDPLAAELQSTYGTPDIYGMHWLADVD | |
| | | NIYGYGDSPGGGCELGPSAKGVSYINTFQRGSQESVWETVTQPTCDNG | |
| | | KYGGAHGYVDLFIQGSTPPQWKYTDAPDADARAVQAAYWAYTWASAQG | |
| | | KASAIAPTIAKAAKLGDYLRYSLFDKYFKQVGNCYPASSCPGATGRQS | |
| | | ETYLIGWYYAWGGSSQGWAWRIGDGAAHFGYQNPLAAWAMSNVTPLIP | |
| | | LSPTAKSDWAASLQRQLEFYQWLQSAEGAIAGGATNSWNGNYGTPPAG | |
| | | DSTFYGMAYDWEPVYHDPPSNNWFGFQAWSMERVAEYYYVTGDPKAKA | |
| | | LLDKWVAVWKPNVTTGASWSIPSNLSWSGQPDTWNPSNPGTNANLHVT | |
| | | ITSSGQDVGVAAALAKTLEYYAAKSGDTASRDLAKGLLDSIWNNDQDS | |
| | | LGVSTPETRTDYSRFTQVYDPTTGDGLYIPSGWTGTMPNGDQIKPGAT | |
| | | FLSIRSWYTKDPQWSKVQAYLNGGPAPTFNYHRFWAESDFAMANADFG | |
| | | MLFPSGSPSPTPSPTPTSSPSPTPSSSPTPSPSPSPTGDTTPPSVPTG | |
| | | LQVTGTTTSSVSLSWTASTDNVGVAHYNVYRNGTLVGQPTATSFTDTG | |
| | | LAAGTSYTYTVAAVDAAGNTSAQSSPVTATTASPSPSPSPSPTPTSSP | |
| | | SPTPSPTPSPTSTSGASCTATYVVNSDWGSGFTTTVTVTNTGTRATSG | |
| | | WTVTWSFAGNQTVTNYWNTALTQSGKSVTAKNLSYNNVIQPGQSTTFG | |
| | | FNGSYSGTNTAPTLSCTASZ | |
| Xy1E | Acidothermus | MGHHAMRRMVTSASVVGVATLAAATVLITGGIAHAASTLKQGAEANGR | ABK51955.1 |
| | cellulolyticus | YFGVSASVNTLNNSAAANLVATQFDMLTPENEMKWDTVESSRGSFNFG | |
| | | PGDQIVAFATAHNMRVRGHNLVWHSQLPGWVSSLPLSQVQSAMESHIT | |
| | | AEVTHYKGKIYAWDVVNEPFDDSGNLRTDVFYQAMGAGYIADALRTAH | |
| | | AADPNAKLYLNDYNIEGINAKSDAMYNLIKQLKSQGVPIDGVGFESHF | |
| | | IVGQVPSTLQQNMQRFADLGVDVAITELDDRMPTPPSQQNLNQQATDD | |
| | | ANVVKACLAVARCVGITQWDVSDADSWVPGTFSGQGAATMFDSNLQPK | |
| | | PAFTAVLNALSASASVSPSPSPSPSPSPSPSPSPSPSPSPSPSPSPSP | |
| | | SSSPVSGGVKVQYKNNDSAPGDNQIKPGLQVVNTGSSSVDLSTVTVRY | |
| | | WFTRDGGSSTLVYNCDWAVMGCGNIRASFGSVNPATPTADTYLQLSFT | |
| | | GGTLPAGGSTGEIQSRVNKSDWSNFTETNDYSYGTNTTFQDWSKVTVY | |
| | | VNGRLVWGTEPSGTSPSPTPSPSPTPSPSPSPSPSPSPSPSPSPSPSP | |
| | | SSSPSSGCVASMRVDSSWPGGFTATVTVSNTGGVSTSGWQVGWSWPSG | |
| | | DSLVNAWNAVVSVTGTSVRAVNASYNGVIPAGGSTTFGFQANGTPGTP | |
| | | TFTCTTSADLZ | |
| aviIII | Acidothermus | MAATTQPYTWSNVAIGGGGFVDGIVFNEGAPGILYVRTDIGGMYRWDA | ABK52391.1 |
| | cellulolyticus | ANGRWIPLLDWVGWNNWGYNGVVSIAADPINTNKVWAAVGMYTNSWDP | |
| | | NDGAILRSSDQGATWQITPLPFKLGGNMPGRGMGERLAVDPNNDNILY | |
| | | FGAPSGKGLWRSTDSGATWSQMTNFPDVGTYIANPTDTTGYQSDIQGV | |
| | | VWVAFDKSSSSLGQASKTIFVGVADPNNPVFWSRDGGATWQAVPGAPT | |
| | | GFIPHKGVFDPVNHVLYIATSNTGGPYDGSSGDVWKFSVTSGTWTRIS | |
| | | PVPSTDTANDYFGYSGLTIDRQHPNTIMVATQISWWPDTIIFRSTDGG | |
| | | ATWTRIWDWTSYPNRSLRYVLDISAEPWLTFGVQPNPPVPSPKLGWMD | |
| | | EAMAIDPFNSDRMLYGTGATLYATNDLTKWDSGGQIHIAPMVKGLEET | |
| | | AVNDLISPPSGAPLISALGDLGGFTHADVTAVPSTIFTSPVFTTGTSV | |
| | | DYAELNPSIIVRAGSFDPSSQPNDRHVAFSTDGGKNWFQGSEPGGVTT | |
| | | GGTVAASADGSRFVWAPGDPGQPVVYAVGFGNSWAASQGVPANAQIRS | |
| | | DRVNPKTFYALSNGTFYRSTDGGVTFQPVAAGLPSSGAVGVMFHAVPG | |
| | | KEGDLWLAASSGLYHSTNGGSSWSAITGVSSAVNVGFGKSAPGSSYPA | |
| | | VFVVGTIGGVTGAYRSDDGGTTWVRINDDQHQYGNWGQAITGDPRIYG | |
| | | RVYIGTNGRGIVYGDIAGAPSGSPSPSVSPSASPSLSPSPSPSSSPSP | |
| | | SPSPSSSPSSSPSPSPSPSPSPSRSPSPSASPSPSSSPSPSSSPSSSP | |
| | | SPTPSSSPVSGGVKVQYKNNDSAPGDNQIKPGLQVVNTGSSSVDLSTV | |
| | | TVRYWFTRDGGSSTLVYNCDWAAIGCGNIRASFGSVNPATPTADTYLQ | |
| | | LSFTGGTLAAGGSTGEIQNRVNKSDWSNFTETNDYSYGTNTVFQDWSK | |
| | | VTVYVNGRLVWGTEPSGTSPSPTPSPSPTPSPSPSPSPGGDVTPPSVP | |
| | | TGVVVTGVSGSSVSLAWNASTDNVGVAHYNVYRNGVLVGQPTVTSFTD | |
| | | TGLAAGTAYTYTVAAVDAAGNTSAPSTPVTATTTSPSPSPSPTPSPTP | |
| | | SPTPSPSPSPSLSPSPSPSPSPSPSPSLSPSPSTSPSPSPSPTPSPSS | |
| | | SGVGCRATYVVNSDWGSGFTATVTVTNTGSRATSGWTVAWSFGGNQTV | |
| | | TNYWNTLLTQSGASVTATNLSYNNVIQPGQSTTFGFNATYAGTNTPPT | |
| | | PTCTTNSD | |
| Xy1E | Acidothermus | MGHHAMRRMVTSASVVGVATLAAATVLITGGIAHAASTLKQGAEANGR | ABK51955.1 |
| | cellulolyticus | YFGVSASVNTLNNSAAANLVATQFDMLTPENEMKWDTVESSRGSFNFG | |
| | | PGDQIVAFATAHNMRVRGHNLVWHSQLPGWVSSLPLSQVQSAMESHIT | |
| | | AEVTHYKGKIYAWDVVNEPFDDSGNLRTDVFYQAMGAGYIADALRTAH | |
| | | AADPNAKLYLNDYNIEGINAKSDAMYNLIKQLKSQGVPIDGVGFESHF | |
| | | IVGQVPSTLQQNMQRFADLGVDVAITELDDRMPTPPSQQNLNQQATDD | |
| | | ANVVKACLAVARCVGITQWDVSDADSWVPGTFSGQGAATMFDSNLQPK | |
| | | PAFTAVLNALSASASVSPSPSPSPSPSPSPSPSPSPSPSPSPSPSPSP | |
| | | SSSPVSGGVKVQYKNNDSAPGDNQIKPGLQVVNTGSSSVDLSTVTVRY | |
| | | WFTRDGGSSTLVYNCDWAVMGCGNIRASFGSVNPATPTADTYLQLSFT | |
| | | GGTLPAGGSTGEIQSRVNKSDWSNFTETNDYSYGTNTTFQDWSKVTVY | |
| | | VNGRLVWGTEPSGTSPSPTPSPSPTPSPSPSPSPSPSPSPSPSPSPSP | |
| | | SSSPSSGCVASMRVDSSWPGGFTATVTVSNTGGVSTSGWQVGWSWPSG | |
| | | DSLVNAWNAVVSVTGTSVRAVNASYNGVIPAGGSTTFGFQANGTPGTP | |
| | | TFTCTTSADLZ | |
| aviIII | Acidothermus | MAATTQPYTWSNVAIGGGGFVDGIVFNEGAPGILYVRTDIGGMYRWDA | ABK52391.1 |
| | cellulolyticus | ANGRWIPLLDWVGWNNWGYNGVVSIAADPINTNKVWAAVGMYTNSWDP | |
| | | NDGAILRSSDQGATWQITPLPFKLGGNMPGRGMGERLAVDPNNDNILY | |
| | | FGAPSGKGLWRSTDSGATWSQMTNFPDVGTYIANPTDTTGYQSDIQGV | |
| | | VWVAFDKSSSSLGQASKTIFVGVADPNNPVFWSRDGGATWQAVPGAPT | |
| | | GFIPHKGVFDPVNHVLYIATSNTGGPYDGSSGDVWKFSVTSGTWTRIS | |
| | | PVPSTDTANDYFGYSGLTIDRQHPNTIMVATQISWWPDTIIFRSTDGG | |
| | | ATWTRIWDWTSYPNRSLRYVLDISAEPWLTFGVQPNPPVPSPKLGWMD | |
| | | EAMAIDPFNSDRMLYGTGATLYATNDLTKWDSGGQIHIAPMVKGLEET | |
| | | AVNDLISPPSGAPLISALGDLGGFTHADVTAVPSTIFTSPVFTTGTSV | |
| | | DYAELNPSIIVRAGSFDPSSQPNDRHVAFSTDGGKNWFQGSEPGGVTT | |
| | | GGTVAASADGSRFVWAPGDPGQPVVYAVGFGNSWAASQGVPANAQIRS | |
| | | DRVNPKTFYALSNGTFYRSTDGGVTFQPVAAGLPSSGAVGVMFHAVPG | |
| | | KEGDLWLAASSGLYHSTNGGSSWSAITGVSSAVNVGFGKSAPGSSYPA | |
| | | VFVVGTIGGVTGAYRSDDGGTTWVRINDDQHQYGNWGQAITGDPRIYG | |
| | | RVYIGTNGRGIVYGDIAGAPSGSPSPSVSPSASPSLSPSPSPSSSPSP | |
| | | SPSPSSSPSSSPSPSPSPSPSPSRSPSPSASPSPSSSPSPSSSPSSSP | |
| | | SPTPSSSPVSGGVKVQYKNNDSAPGDNQIKPGLQVVNTGSSSVDLSTV | |
| | | TVRYWFTRDGGSSTLVYNCDWAAIGCGNIRASFGSVNPATPTADTYLQ | |
| | | LSFTGGTLAAGGSTGEIQNRVNKSDWSNFTETNDYSYGTNTVFQDWSK | |
| | | VTVYVNGRLVWGTEPSGTSPSPTPSPSPTPSPSPSPSPGGDVTPPSVP | |
| | | TGVVVTGVSGSSVSLAWNASTDNVGVAHYNVYRNGVLVGQPTVTSFTD | |
| | | TGLAAGTAYTYTVAAVDAAGNTSAPSTPVTATTTSPSPSPSPTPSPTP | |
| | | SPTPSPSPSPSLSPSPSPSPSPSPSPSLSPSPSTSPSPSPSPTPSPSS | |
| | | SGVGCRATYVVNSDWGSGFTATVTVTNTGSRATSGWTVAWSFGGNQTV | |
| | | TNYWNTLLTQSGASVTATNLSYNNVIQPGQSTTFGFNATYAGTNTPPT | |
| | | PTCTTNSD | |
| cbhE | Talaromyces | MDPQQAGTATAENHPPLTWQECTAPGSCTTQNGAVVLDANWRWVHDVN | AAL33602.2 |
| | emersonii | GYTNCYTGNTWDPTYCPDDETCAQNCALDGADYEGTYGVTSSGSSLKL | |
| | | NFVTGSNVGSRLYLLQDDSTYQIFKLLNREFSFDVDVSNLPCGLNGAL | |
| | | YFVAMDADGGVSKYPNNKAGAKYGTGYCDSQCPRDLKFIDGEANVEGW | |
| | | QPSSNNANTGIGDHGSCCAEMDVWEANSISNAVTPHPCDTPGQTMCSG | |
| | | DDCGGTYSNDRYAGTCDPDGCDFNPYRMGNTSFYGPGKIIDTTKPFTV | |
| | | VTQFLTDDGTDTGTLSEIKRFYIQNSNVIPQPNSDISGVTGNSITTEF | |
| | | CTAQKQAFGDTDDFSQHGGLAKMGAAMQQGMVLVMSLDDYAAQMLWLD | |
| | | SDYPTDADPTTPGIARGTCPTDSGVPSDVESQSPNSYVTYSNIKFGPI | |
| | | NSTFTASGD | |

### A—Cellulases

Cellulases are enzyme polypeptides involved in cellulose degradation. Cellulase enzyme polypeptides are classified on the basis of their mode of action. There are two basic kinds of cellulases: the endocellulases, which cleave the polymer chains internally; and the exocellulases, which cleave from the reducing and non-reducing ends of molecules generated by the action of endocellulases. Cellulases include cellobiohydrolases, endoglucanases, and β-D-glucosidases. Endoglucanases randomly attack the amorphous regions of cellulose substrate, yielding mainly higher oligomers. Cellulobiohydrolases are exocellulases which hydrolyze crystalline cellulose and release cellobiose (glucose dimer). Both types of enzymes hydrolyze-1,4-glycosidic bonds. β-D-glucosidases or cellulobiase converts oligosaccharides and cellubiose to glucose. Beta-glucan glucohydrolase hydrolyzes oligosaccharides to glucose.

According to the present invention, plants may be engineered to comprise a gene encoding a cellulase enzyme polypeptide. Alternatively, plants may be engineered to comprise more than one gene encoding a cellulase enzyme polypeptide. For example, plants may be engineered to comprise one or more genes encoding a cellulase of the cellubiohydrolase class, one or more genes encoding a cellulase of the endoglucanase class, and/or one or more genes encoding a cellulase of the β-D-glucosidase class.

Examples of endoglucanase genes that can be used in the present invention can be obtained from Aspergillus aculeatus (U.S. Pat. No. 6,623,949; WO 94/14953), Aspergillus kawachii (U.S. Pat. No. 6,623,949), Aspergillus oryzae (Kitamoto et al., Appl. Microbiol. Biotechnol., 1996, 46: 538-544; U.S. Pat. No. 6,635,465), Aspergillus nidulans (Lockington et al., Fungal Genet. Biol., 2002, 37: 190-196), Cellulomonas fimi (Wong et al., Gene, 1986, 44: 315-324), Bacillus subtilis (MacKay et al., Nucleic Acids Res., 1986, 14: 9159-9170), Cellulomonas pachnodae (Cazemier et al., Appl. Microbiol. Biotechnol., 1999, 52: 232-239), Fusarium equiseti (Goedegebuur et al., Curr. Genet., 2002, 41: 89-98), Fusarium oxysporum (Hagen et al., Gene, 1994, 150: 163-167; Sheppard et al., Gene, 1994, 150: 163-167), Humicola insolens (U.S. Pat. No. 5,912,157; Davies et al., Biochem J., 2000, 348: 201-207), Hypocrea jecorina (Penttila et al., Gene, 1986, 45: 253-263), Humicola grisea (Goedegebuur et al., Curr. Genet., 2002, 41: 89-98), Micromonospora cellulolyticum (Lin et al., J. Ind. Microbiol., 1994, 13: 344-350), Myceliophthora thermophila (U.S. Pat. No. 5,912,157), Rhizopus oryzae (Moriya et al., J. Bacteriol., 2003, 185: 1749-1756), Trichoderma reesei (Saloheimo et al., Mol. Microbiol., 1994, 13: 219-228), and Trichoderma viride (Kwon et al., Biosci. Biotechnol. Biochem., 1999, 63: 1714-1720; Goedegebuur et al., Curr. Genet., 2002, 41: 89-98).

In certain embodiments, plants are engineered to comprise the endo-1,4-β-glucanase E1 gene (GenBank Accession No. U33212, See Table 1). This gene was isolated from the thermophilic bacterium Acidothermus cellulolyticus. Acidothermus cellulolyticus has been characterized with the ability to hydrolyze and degrade plant cellulose. The cellulase complex produced by A. cellulolyticus is known to contain several different thermostable cellulase enzymes with maximal activities at temperatures of 75° C. to 83° C. These cellulases are resistant to inhibition from cellobiose, an end product of the reactions catalyzed by endo- and exo-cellulases.

The E1 endo-1,4-β-glucanase is described in detail in U.S. Pat. No. 5,275,944. This endoglucanase demonstrates a temperature optimum of 83° C. and a specific activity of 40 μmol glucose release from carboxymethylcellulose/min/mg protein. This E1 endoglucanase was further identified as having an isoelectric pH of 6.7 and a molecular weight of 81,000 Daltons by SDS polyacrylamide gel electrophoresis. It is synthesized as a precursor with a signal peptide that directs it to the export pathway in bacteria. The mature enzyme polypeptide is 521 amino acids (aa) in length. The crystal structure of the catalytic domain of about 40 kD (358 aa) has been described (J. Sakon et al., Biochem., 1996, 35: 10648-10660). Its pro/thr/ser-rich linker is 60 aa, and the cellulose binding domain (CBD) is 104 aa. The properties of the cellulose binding domain that confer its function are not well-characterized. Plant expression of the E1 gene has been reported (see for example, M. T. Ziegler et al., Mol. Breeding, 2000, 6: 37-46; Z. Dai et al., Mol. Breeding, 2000, 6: 277-285; Z. Dai et al., Transg. Res., 2000, 9: 43-54; and T. Ziegelhoffer et al., Mol. Breeding, 2001, 8: 147-158).

Examples of cellobiohydrolase genes that can be used in the present invention can be obtained from Acidothermus cellulolyticus, Acremonium cellulolyticus (U.S. Pat. No. 6,127,160), Agaricus bisporus (Chow et al., Appl. Environ. Microbiol., 1994, 60: 2779-2785), Aspergillus aculeatus (Takada et al., J. Ferment. Bioeng., 1998, 85: 1-9), Aspergillus niger (Gielkens et al., Appl. Environ. Microbiol., 65: 1999, 4340-4345), Aspergillus oryzae (Kitamoto et al., Appl. Microbiol. Biotechnol., 1996, 46: 538-544), Athelia rolfsii (EMBL accession No. AB103461), Chaetomium thermophilum (EMBL accession Nos. AX657571 and CQ838150), Cullulomonas fimi (Meinke et al., Mol. Microbiol., 1994, 12: 413-422), Emericella nidulans (Lockington et al., Fungal Genet. Biol., 2002, 37: 190-196), Fusarium oxysporum (Hagen et al., Gene, 1994, 150: 163-167), Geotrichum sp. 128 (EMBL accession No. AB089343), Humicola grisea (de Oliviera and Radford, Nucleic Acids Res., 1990, 18: 668; Takashima et al., J. Biochem., 1998, 124: 717-725), Humicola nigrescens (EMBL accession No. AX657571), Hypocrea koningii (Teeri et al., Gene, 1987, 51: 43-52), Mycelioptera thermophila (EMBL accession No. AX657599), Neocallimastix patriciarum (Denman et al., Appl. Environ. Microbiol., 1996, 62: 1889-1896), Phanerochaete chrysosporium (Tempelaars et al., Appl. Environ. Microbiol., 1994, 60: 4387-4393), Thermobifida fusca (Zhang, Biochemistry, 1995, 34: 3386-3395), Trichoderma reesei (Terri et al., BioTechnology, 1983, 1: 696-699; Chen et al., BioTechnology, 1987, 5: 274-278), and Trichoderma viride (EMBL accession Nos. A4368686 and A4368688).

Examples of β-D-glucosidase genes that can be used in the present invention can be obtained from Aspergillus aculeatus (Kawaguchi et al., Gene, 1996, 173: 287-288), Aspergillus kawachi (Iwashita et al., Appl. Environ. Microbiol., 1999, 65: 5546-5553), Aspergillus oryzae (WO 2002/095014), Cellulomonas biazotea (Wong et al., Gene, 1998, 207: 79-86), Penicillium funiculosum (WO 200478919), Saccharomycopsis fibuligera (Machida et al., Appl. Environ. Microbiol., 1988, 54: 3147-3155), Schizosaccharomyces pombe (Wood et al., Nature, 2002, 415: 871-880), and Trichoderma reesei (Barnett et al., BioTechnology, 1991, 9: 562-567).

Other examples of cellulases that can be used in accordance with the present invention include family 48 glycoside hydrolases such as gux1 from Acidothermus cellulolyticus, avicelases such as aviIII from Acidothermus cellulolyticus, and cbhE from Talaromyces emersonii. (See Table 1.)

Transgene expression of cellulases in plants for the conversion of cellulose to glucose has been reported (see, for example, Y. Jin Cai et al., Appl. Environ. Microbiol., 1999, 65: 553-559; C. R. Sanchez et al., Revista de Microbiologica, 1999, 30: 310-314; R. Cohen et al., Appl. Environ., 2995, 71: 2412-2417; Z. Dai et al., Transg. Res., 2005, 14: 627-543).

### B—Hemicellulases

Hemicellulases are enzyme polypeptides that are involved in hemicellulose degradation. Hemicellulases include xylanases, arabinofuranosidases, acetyl xylan esterases, ferulic acid esterases, xyloglucanases, β-glucanases, β-xylosidases, glucuronidases, mannanases, galactanases, and arabinases. Similar to cellulase enzyme polypeptides, hemicellulases are classified on the basis of their mode of action: the endo-acting hemicellulases attack internal bonds within the polysaccharide chain; the exo-acting hemicellulases act progressively from either the reducing or non-reducing end of polysaccharide chains.

According to the present invention, plants may be engineered to comprise a gene encoding a hemicellulase enzyme polypeptide. Alternatively, plants may be engineered to comprise more than one gene encoding a hemicellulase enzyme polypeptide. For example, plants may be engineered to comprise one or more genes encoding a hemicellulase of the xylanase class, one or more genes encoding a hemicellulase of the arabinofuranosidase class, one or more genes encoding a hemicellulase of the acetyl xylan esterase class, one or more genes encoding a hemicellulase of the glucuronidase class, one or more genes encoding a hemicellulase of the mannanase class, one or more genes encoding a hemicellulase of the galactanase class, and/or one or more genes encoding a hemicellulase of the arabinase class.

Examples of endo-acting hemicellulases include endoarabinanase, endoarabinogalactanase, endoglucanase, endomannanase, endoxylanase, and feraxan endoxylanase. Examples of exo-acting hemicellulases include α-L-arabinosidase, β-L-arabinosidase, α-1,2-L-fucosidase, α-D-galactosidase, β-D-galactosidase, β-D-glucosidase, β-D-glucuronidase, β-D-mannosidase, β-D-xylosidase, exo-glucosidase, exo-mannobiohydrolase, exo-mannanase, exo-xylanase, xylan α-glucuronidase, and coniferin β-glucosidase.

Hemicellulase genes can be obtained from any suitable source, including fungal and bacterial organisms, such as Aspergillus, Disporotrichum, Penicillium, Neurospora, Fusarium, Trichoderma, Humicola, Thermomyces, and Bacillus. Examples of hemicellulases that can be used in the present invention can be obtained from Acidothermus cellulolyticus, Acidobacterium capsulatum (Inagaki et al., Biosci. Biotechnol. Biochem., 1998, 62: 1061-1067), Agaricus bisporus (De Groot et al., J. Mol. Biol., 1998, 277: 273-284), Aspergillus aculeatus (U.S. Pat. No. 6,197,564; U.S. Pat. No. 5,693,518), Aspergillus kawachii (Ito et al., Biosci. Biotechnol. Biochem., 1992, 56: 906-912), Aspergillus niger (EMBL accession No. AF108944), Magnaporthe grisea (Wu et al., Mol. Plant. Microbe Interact., 1995, 8: 506-514), Penicillium chrysogenum (Haas et al., Gene, 1993, 126: 237-242), Talaromyces emersonii (WO 02/24926), and Trichoderma reesei (EMBL accession Nos. X69573, X69574, and AY281369).

In certain embodiments, plants are engineered to comprise the A. cellulolyticus endoxylanase xylE (see the Examples section).

### C—Ligninases

Ligninases are enzyme polypeptides that are involved in the degradation of lignin. Lignin-degrading enzyme polypeptides include, but are not limited to, lignin peroxidases, manganese-dependent peroxidases, hybrid peroxidases (which exhibit combined properties of lignin peroxidases and manganese-dependent peroxidases), and laccases. Hydrogen peroxide, required as co-substrate by the peroxidases, can be generated by glucose oxidase, aryl alcohol oxidase, and/or lignin peroxidase-activated glyoxal oxidase.

According to the present invention, plants may be engineered to comprise a gene encoding a ligninase enzyme polypeptide. Alternatively, plants may be engineered to comprise more than one gene encoding a ligninase enzyme polypeptide. For example, plants may be engineered to comprise one or more genes encoding a ligninase of the lignin peroxidase class, one or more genes encoding a ligninase of the manganese-dependent peroxidase class, one or more genes encoding a ligninase of the hybrid peroxidase class, and/or one or more genes encoding a ligninase of the laccase class.

Lignin-degrading genes may be obtained from Acidothermus cellulolyticus, Bjerkandera adusta, Ceriporiopsis subvermispora (see WO 02/079400), Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride.

Examples of genes encoding ligninases that can be used in the invention can be obtained from Bjerkandera adusta (WO 2001/098469), Ceriporiopsis subvermispora (Conesa et al., J. Biotechnol., 2002, 93: 143-158), Cantharellus cibariusi (Ng et al., Biochem. and Biophys. Res. Comm., 2004, 313: 37-41), Coprinus cinereus (WO 97/008325; Conesa et al., J. Biotechnol., 2002, 93: 143-158), Lentinula edodes (Nagai et al., Applied Microbiol. and Biotechnol., 2002, 60: 327-335, 2002), Melanocarpus albomyces (Kiiskinen et al., FEBS Letters, 2004, 576: 251-255, 2004), Myceliophthora thermophila (WO 95/006815), Phanerochaete chrysosporium (Conesa et al., J. Biotechnol., 2002, 93: 143-158; Martinez, Enz, Microb, Technol, 2002, 30: 425-444), Phlebia radiata (Conesa et al., J. Biotechnol., 2002, 93: 143-158), Pleurotus eryngii (Conesa et al., J. Biotechnol., 2002, 93: 143-158), Polyporus pinsitus (WO 96/000290), Rigidoporus lignosus (Garavaglia et al., J. of Mol. Biol., 2004, 342: 1519-1531), Rhizoctonia solani (WO 96/007988), Scytalidium thermophilum (WO 95/033837), Tricholoma giganteum (Wang et al., Biochem. Biophys. Res. Comm., 2004, 315: 450-454), and Trametes versicolor (Conesa et al., J. Biotechnol., 2002, 93: 143-158).

For example, plants may be engineered to comprise one or more lignin peroxidases. Genes encoding lignin peroxidases may be obtained from Phanerochaete chrysosporium or Phlebia radiata. Lignin-peroxidases are glycosylated heme proteins (MW 38 to 46 kDa) which are dependent on hydrogen peroxide for activity and catalyze the oxidative cleavage of lignin polymer. At least six (6) heme proteins (H1, H2, H6, H7, H8 and H10) with lignin peroxidase activity have been identified Phanerochaete chrysosporium in strain BKMF-1767. In certain embodiments, plants are engineered to comprise the white rot filamentous Phanerochaete chrysosporium ligninase (CGL5) (H. A. de Boer et al., Gene, 1988, 69(2): 369) (see the Examples section).

### D—Other Lignocellulolytic Enzyme Polypeptides

In addition to cellulases, hemicellulases and ligninases, lignocellulolytic enzyme polypeptides that can be used in the practice of the present invention also include enzymes that degrade pectic substances or phenolic acids such as ferulic acid. Pectic substances are composed of homogalacturonan (or pectin), rhamno-galacturonan, and xylogalacturonan. Enzymes that degrade homogalacturonan include pectate lyase, pectin lyase, polygalacturonase, pectin acetyl esterase, and pectin methyl esterase. Enzymes that degrade rhamnogalacturonan include alpha-arabinofuranosidase, beta-galactosidase, galactanase, arabinanase, alpha-arabinofuranosidase, rhamnogalacturonase, rhamnogalacturonan lyase, and rhamnogalacturonan acetyl esterase. Enzymes that degrade xylogalacturonan include xylogalacturonosidase, xylogalacturonase, and rhamnogalacturonan lyase.

Phenolic acids include ferulic acid, which functions in the plant cell wall to cross-link cell wall components together. For example, ferulic acid may cross-link lignin to hemicellulose, cellulose to lignin, and/or hemicellulose polymers to each other. Ferulic acid esterases cleave ferulic acid, disrupting the cross linkages.

Other enzymes that may enhance or promote lignocellulose disruption and/or degradation include, but are not limited to, amylases (e.g., alpha amylase and glucoamylase), esterases, lipases, phospholipases, phytases, proteases, and peroxidases.

### E—Combinations of Lignocellulolytic Enzyme Polypeptides

According to the present invention, plants may be engineered to comprise a gene encoding a lignocellulolytic enzyme polypeptide, e.g., a cellulase enzyme polypeptide, a hemicellulase enzyme polypeptide, or a ligninase enzyme polypeptide. Alternatively, plants may be engineered to comprise two or more genes encoding lignocellulolytic enzyme polypeptides, e.g., enzymes from different classes of cellulases, enzymes from different classes of hemicellulases, enzymes from different classes of ligninases, or any combinations thereof. For example, combinations of genes may be selected to provide efficient degradation of one component of lignocellulose (e.g., cellulose, hemicellulose, or lignin). Alternatively, combinations of genes may be selected to provide efficient degradation of the lignocellulosic material.

In certain embodiments, genes are optimized for the substrate (e.g., cellulose, hemicellulase, lignin or whole lignocellulosic material) in a particular plant (e.g., corn, tobacco, switchgrass). Tissue from one plant species is likely to be physically and/or chemically different from tissue from another plant species. Selection of genes or combinations of genes to achieve efficient degradation of a given plant tissue is within the skill of artisans in the art.

In some embodiments, combinations of genes are selected to provide for synergistic enzymes activity (i.e., genes are selected such that the interaction between distinguishable enzymes or enzyme activities results in the total activity of the enzymes taken together being greater than the sum of the effects of the individual activities).

Efficient lignocellulolytic activity may be achieved by production of two or more enzymes in a single transgenic plant. As mentioned above, plants may be transformed to express more than one enzyme, for example, by employing the use of multiple gene constructs encoding each of the selected enzymes or a single construct comprising multiple nucleotide sequences encoding each of the selected enzymes. Alternatively, individual transgenic plants, each stably transformed to express a given enzyme, may be crossed by methods known in the art (e.g., pollination, hand detassling, cytoplasmic male sterility, and the like) to obtain a resulting plant that can produce all the enzymes of the individual starting plants.

Alternatively or additionally, efficient lignocellulolytic activity may be achieved by production of two or more lignocellulolytic enzyme polypeptides in separate plants. For example, three separate lines of plants (e.g., corn), one expressing one or more enzymes of the cellulase class, another expressing one or more enzymes of the hemicellulase class and the third one expressing one or more enzymes of the ligninase class, may be developed and grown simultaneously. The desired “blend” of enzymes produced may be achieved by simply changing the seed ratio, taking into account farm climate and soil type, which are expected to influence enzyme yields in plants.

Other advantages of this approach include, but are not limited to, increased plant health (which is known to be adversely affected as the number of introduced genes increases), simpler transformations procedures and great flexibility in incorporating the desired traits in commercial plant varieties for large-scale production.

### G—Thermophilic and Thermostable Enzyme Polypeptides

It may be sometimes desirable to use transgenic plants expressing thermophilic and/or thermostable enzyme polypeptides. For example, enzyme polypeptides whose optimal range of temperature for activity (thermophilic enzyme polypeptides) may be expressed in transgenic plants in accordance with the invention. Without wishing to be bound by any particular theory, the limited activity or absence of activity during growth of the plant (at moderate or low temperatures, at which the enzyme polypeptide is less active) may be beneficial to the health of the plant. Alternatively or additionally, and without wishing to be bound by any particular theory, such enzyme polypeptides may facilitate increased hydrolysis because of their high activity at high temperature conditions commonly used in the processing of cellulosic biomass.

In some embodiments, the present invention provides a transgenic plant, the genome of which is augmented with a recombinant polynucleotide encoding at least one lignocellulolytic enzyme polypeptide that exhibits low activity at a temperature below about 60° C., below about 50° C., below about 40° C., or below about 30° C. In some embodiments, the present invention provides a transgenic plant, the genome of which is augmented with a recombinant polynucleotide encoding at least one lignocellulolytic enzyme polypeptide that exhibits high activity at a temperature above about 50° C., above about 60° C., above about 70° C., above about 80° C., or above about 90° C.

In some embodiments, the present invention provides a transgenic plant, the genome of which is augmented with a recombinant polynucleotide encoding at least one lignocellulolytic enzyme polypeptide that is or is homologous to a lignocellulolytic enzyme polypeptide found in a thermophilic microorganism (e.g., bacterium, fungus, etc.). In some such embodiments, the thermophilic organism is a bacterium that is a member of a genus selected from the group consisting of Aeropyrum, Acidilobus, Acidothermus, Aciduliprofundum, Anaerocellum, Archaeoglobus, Aspergillus, Bacillus, Caldibacillus, Caldicellulosiruptor, Caldithrix, Cellulomonas, Chaetomium, Chloroflexus, Clostridium, Cyanidium, Deferribacter, Desulfotomaculum, Desulfurella, Desulfurococcus, Fervidobacterium, Geobacillus, Geothermobacterium, Humicola, Ignicoccus, Marinitoga, Methanocaldococcus, Methanococcus, Methanopyrus, Methanosarcina, Methanothermobacter, Nautilia, Pyrobaculum, Pyrococcus, Pyrodictium, Rhizomucor, Rhodothermus, Staphylothermus, Scylatidium, Spirochaeta, Sulfolobus, Talaromyces, Thermoascus, Thermobifida, Thermococcus, Thermodesulfobacterium, Thermodesulfovibrio, Thermomicrobium, Thermoplasma, Thermoproteus, Thermothrix, Thermotoga, Thermus, and Thiobacillus; in some such embodiments, the thermophilic microorganism is a bacterium that is a member of a species selected from the group consisting of Acidothermus cellulolyticus, Pyrococcus furiosus, and Talaromyces emersonii.

### III. Nucleic Acid Constructs

Nucleic acid constructs to be used in the practice of the present invention generally encompass expression cassettes for expression in the plant of interest. The cassette generally includes 5′ and 3′ regulatory sequences operably linked to a nucleotide sequence encoding a cell wall modifying-enzyme polypeptide (e.g., one whose amino acid sequence is listed in Table 1).

### Expression Cassettes

Techniques used to isolate or clone a gene encoding an enzyme (e.g., a cell wall-modifying enzyme polypeptide) are known in the art and include isolation from genomic DNA, preparation from cDNA, or a combination thereof. The cloning of a gene from such genomic DNA, can be effected, e.g., by using polymerase chain reaction (PCR) or antibody screening or expression libraries to detect cloned DNA fragments with shared structural features (Innis et al., “PCR: A Guide to Method and Application”, 1990, Academic Press: New York). Other nucleic acid amplification procedures such as ligase chain reaction (LCR), ligated activated transcription (LAT) and nucleotide sequence-based amplification (NASBA) may be used.

The expression cassette will generally include in the 5′-3′ direction of transcription, a transcriptional and translational initiation region, a coding sequence for a cell wall-modifying enzyme polypeptide, and a transcriptional and translational termination region functional in plants. The transcriptional initiation region, i.e., the promoter, can be native or analogous (i.e., found in the native plant) or foreign or heterologous (i.e., not found in the native plant) to the plant host. Additionally, the promoter can be the natural sequence or alternatively a synthetic sequence.

In certain embodiments, the promoter is a constitutive plant promoter, i.e., an unregulated promoter that allows continual expression of a gene associated with it. Examples of plant promoters include, but are not limited to, the 35S cauliflower mosaic virus (CaMV) promoter, a promoter of nopaline synthase, and a promoter of octopine synthase. Examples of other constitutive promoters used in plants are the 19S promoter and promoters from genes encoding actin and ubiquitin. Promoters may be obtained from genomic DNA by using polymerase chain reaction (PCR), and then cloned into the construct.

The constitutive promoter may allow expression of an associated gene throughout the life of a plant. In some embodiments, the cell wall-modifying enzyme polypeptide is produced throughout the life of the plant. In some embodiments, the cell wall-modifying enzyme polypeptide is active through the life of the plant. Alternatively or additionally, a constitutive promoter may allow expression of an associated gene in all or a majority of plant tissues. In some embodiments, the cell wall-modifying enzyme polypeptide is present in all plant tissues during the life of the plant.

Other sequences that can be present in nucleic acid constructs are sequences that enhance gene expression such as intron sequences and leader sequences. Examples of introns that have been reported to enhance expression include, but are not limited to, the introns of the Maize Adh1 gene and introns of the Maize bronze1 gene (J. Callis et. al., Genes Develop. 1987, 1: 1183-1200). Examples of non-translated leader sequences that are known to enhance expression include, but are not limited to, leader sequences from Tobacco Mosaic Virus (TMV, the “omegasequence”), Maize Chlorotic Mottle Virus (MCMV), and Alfalfa Mosaic Virus (AlMV) (see, for example, D. R. Gallie et al., Nucl. Acids Res. 1987, 15: 8693-8711; J. M. Skuzeski et. al., Plant Mol. Biol. 1990, 15: 65-79).

The transcriptional and translational termination region can be native with the transcription initiation region, can be native with the operably linked polynucleotide sequence of interest, or can be derived from another source. Convenient termination regions are available from the T₁-plasmid of A. tumefaciens, such as the octopine synthase and nopaline synthase termination regions (An et al., Plant Cell, 1989, 1: 115-122; Guerineau et al., Mol. Gen. Genet. 1991, 262: 141-144; Proudfoot, Cell, 1991, 64: 671-674; Sanfacon et al., Genes Dev. 1991, 5: 141-149; Mogen et al., Plant Cell, 1990, 2:1261-1272; Munroe et al., Gene, 1990, 91:151-158; Ballas et al., Nucleic Acids Res., 1989, 17: 7891-7903; and Joshi et al., Nucleic Acid Res., 1987, 15: 9627-9639).

Where appropriate, the gene(s) or polynucleotide sequence(s) encoding the enzyme(s) of interest may be modified to include codons that are optimized for expression in the transformed plant (Campbell and Gowri, Plant Physiol., 1990, 92: 1-11; Murray et al., Nucleic Acids Res., 1989, 17: 477-498; Wada et al., Nucl. Acids Res., 1990, 18: 2367, and U.S. Pat. Nos. 5,096,825; 5,380,831; 5,436,391; 5,625,136, 5,670,356 and 5,874,304). Codon optimized sequences are synthetic sequences, and preferably encode the identical polypeptide (or an enzymatically active fragment of a full length polypeptide which has substantially the same activity as the full length polypeptide) encoded by the non-codon optimized parent polynucleotide which encodes a cell wall-modifying enzyme polypeptide.

### Other Polynucleotide Sequences

Optional components of nucleic acid constructs include one or more marker genes. Marker genes are genes that impart a distinct phenotype to cells expressing the marker gene and thus allow transformed cells to be distinguished from cells that do not have the marker. Such genes may encode either a selectable or screenable marker. The characteristic phenotype allows the identification of cells, groups of cells, tissues, organs, plant parts or whole plants containing the construct. Many examples of suitable marker genes are known in the art. The marker may also confer additional benefit(s) to the transgenic plant such as herbicide resistance, insect resistance, disease resistance, and increased tolerance to environmental stress (e.g., drought).

Alternatively, a marker gene can provide some other visibly reactive response (e.g., may cause a distinctive appearance such as color or growth pattern relative to plants or plant cells not expressing the selectable marker gene in the presence of some substance, either as applied directly to the plant or plant cells or as present in the plant or plant cell growth media). It is now well known in the art that transcriptional activators of anthocyanin biosynthesis, operably linked to a suitable promoter in a construct, have widespread utility as non-phytotoxic markers for plant cell transformation.

Examples of markers that provide resistance to herbicides include, but are not limited to, the bar gene from Streptomyces hygroscopicus encoding phosphinothricin acetylase (PAT), which confers resistance to the herbicide glufosinate; mutant genes which encode resistance to imidazalinone or sulfonylurea such as genes encoding mutant form of the ALS and AHAS enzyme (Lee at al., EMBO J., 1988, 7: 1241; Miki et al., Theor. Appl. Genet., 1990, 80: 449; and U.S. Pat. No. 5,773,702); genes which confer resistance to glycophosphate such as mutant forms of EPSP synthase and aroA; resistance to L-phosphinothricin such as the glutamine synthetase genes; resistance to glufosinate such as the phosphinothricin acetyl transferase (PAT and bar) gene; and resistance to phenoxy propionic acids and cyclohexones such as the ACCAse inhibitor-encoding genes (Marshall et al., Theor. Appl. Genet., 1992, 83: 435).

Examples of genes which confer resistance to pests or disease include, but are not limited to, genes encoding a Bacillus thuringiensis protein such as the delta-endotoxin (U.S. Pat. No. 6,100,456); genes encoding lectins (Van Damme et al., Plant Mol. Biol., 1994, 24: 825); genes encoding vitamin-binding proteins such as avidin and avidin homologs which can be used as larvicides against insect pests; genes encoding protease or amylase inhibitors, such as the rice cysteine proteinase inhibitor (Abe et al., J. Biol. Chem., 1987, 262: 16793) and the tobacco proteinase inhibitor I (Hubb et al., Plant Mol. Biol., 1993, 21: 985); genes encoding insect-specific hormones or pheromones such as ecdysteroid and juvenile hormone, and variants thereof, mimetics based thereon, or an antagonists or agonists thereof; genes encoding insect-specific peptides or neuropeptides which, upon expression, disrupts the physiology of the pest; genes encoding insect-specific venom such as that produced by a wasp, snake, etc.; genes encoding enzymes responsible for the accumulation of monoterpenes, sesquiterpenes, asteroid, hydroxamic acid, phenylpropanoid derivative or other non-protein molecule with insecticidal activity; genes encoding enzymes involved in the modification of a biologically active molecule (U.S. Pat. No. 5,539,095); genes encoding peptides which stimulate signal transduction; genes encoding hydrophobic moment peptides such as derivatives of Tachyplesin which inhibit fungal pathogens; genes encoding a membrane permease, a channel former or channel blocker (Jaynes et al., Plant Sci., 1993, 89: 43); genes encoding a viral invasive protein or complex toxin derived therefrom (Beachy et al., Ann. Rev. Phytopathol., 1990, 28: 451); genes encoding an insect-specific antibody or antitoxin or a virus-specific antibody (Tavladoraki et al., Nature, 1993, 366: 469); and genes encoding a developmental-arrestive protein produced by a plant, pathogen or parasite which prevents disease.

Examples of genes which confer resistance to environmental stress include, but are not limited to, mtld and HVA1, which are genes that confer resistance to environmental stress factors; rd29A and rd19B, which are genes of Arabidopsis thaliana that encode hydrophilic proteins which are induced in response to dehydration, low temperature, salt stress, or exposure to abscisic acid and enable the plant to tolerate the stress (Yamaguchi-Shinozaki et al., Plant Cell, 1994, 6: 251-264). Other genes contemplated can be found in U.S. Pat. Nos. 5,296,462 and 5,356,816.

### Tissue-Specific Expression

In certain embodiments, cell wall-modifying enzyme polypeptide expression is targeted to specific tissues of the transgenic plant such that the cell wall-modifying enzyme is present in only some plant tissues during the life of the plant. For example, tissue specific expression may be performed to preferentially express enzymes in leaves and stems rather than grain or seed (which can reduce concerns about human consumption of genetically modified organism (GMOs)). Tissue-specific expression has other benefits including targeted expression of enzyme(s) to the appropriate substrate.

Tissue specific expression may be functionally accomplished by introducing a constitutively expressed gene in combination with an antisense gene that is expressed only in those tissues where the gene product (e.g., cell wall-modifying enzyme polypeptide) is not desired. For example, a gene coding for a cell wall-modifying enzyme polypeptide may be introduced such that it is expression in all tissues using the 35S promoter from Cauliflower Mosaic Virus. Expression of an antisense transcript of the gene in maize kernel, using for example a zein promoter, would prevent accumulation of the cell wall-modifying enzyme polypeptide in seed. Hence the enzyme encoded by the introduced gene would be present in all tissues except the kernel.

Moreover, several tissue-specific regulated genes and/or promoters have been reported in plants. Some reported tissue-specific genes include the genes encoding the seed storage proteins (such as napin, cruciferin, β-conglycinin, and phaseolin) zein or oil body proteins (such as oleosin), or genes involved in fatty acid biosynthesis (including acyl carrier protein, stearoyl-ACP desaturase, and fatty acid desaturases (fad 2-1)), and other genes expressed during embryo development, such as Bce4 (Kridl et al., Seed Science Research, 1991, 1: 209). Examples of tissue-specific promoters, which have been described include the lectin (Vodkin, Prog. Clin. Biol. Res., 1983, 138: 87; Lindstrom et al., Der. Genet., 1990, 11: 160), corn alcohol dehydrogenase 1 (Dennis et al., Nucleic Acids Res., 1984, 12: 983), corn light harvesting complex (Bansal et al., Proc. Natl. Acad. Sci. USA, 1992, 89: 3654), corn heat shock protein, pea small subunit RuBP carboxylase, Ti plasmid mannopine synthase, Ti plasmid nopaline synthase, petunia chalcone isomerase (van Tunen et al., EMBO J., 1988, 7:125), bean glycine rich protein 1 (Keller et al., Genes Dev., 1989, 3: 1639), truncated CaMV 35s (Odell et al., Nature, 1985, 313: 810), potato patatin (Wenzler et al., Plant Mol. Biol., 1989, 13: 347), root cell (Yamamoto et al., Nucleic Acids Res., 1990, 18: 7449), maize zein (Reina et al., Nucleic Acids Res., 1990, 18: 6425; Kriz et al., Mol. Gen. Genet., 1987, 207: 90; Wandelt et al., Nucleic Acids Res., 1989, 17 2354), PEPCase, R gene complex-associated promoters (Chandler et al., Plant Cell, 1989, 1: 1175), and chalcone synthase promoters (Franken et al., EMBO J., 1991, 10: 2605). Particularly useful for seed-specific expression is the pea vicilin promoter (Czako et al., Mol. Gen. Genet., 1992, 235: 33).

### Subcellular Specific Expression

In some embodiments, cell wall-modifying enzyme polypeptide expression is targeted to specific cellular compartments or organelles, such as, for example, the cytosol, the vacuole, the nucleus, the endoplasmic reticulum, the cell wall, the mitochondria, the apoplast, the peroxisomes, plastids, or combinations thereof. In some embodiments of the invention, the cell wall-modifying enzyme polypeptide is expressed in one or more subcellular compartments or organelles, for example, the cell wall and/or endoplasmic reticulum, during the life of the plant.

Directing the cell wall-modifying enzyme polypeptide to a specific cell compartment or organelle may allow the enzyme to be localized such that it will not come into contact with the substrate during plant growth. The enzyme would not act until it is allowed to contact its substrate, e.g., following physical disruption of the cell integrity by milling.

Targeting expression of a cell wall-modifying enzyme polypeptide to the cell wall (as in the apoplast) can help overcome the difficulty of mixing hydrophobic cellulose and hydrophilic enzymes that make it hard to achieve efficient hydrolysis with external enzymes.

In some embodiments, the invention provides plants engineered to express a cell wall-modifying enzyme polypeptide (or more than one cell wall-modifying enzyme polypeptide) in more than one subcellular compartments or organelles. By using promoters targeted at different locations in the plant cell, one can increase the total enzyme produced in the plant. Thus, for example, using an apoplast promoter with the E1 gene, and a chloroplast promoter with the E1 gene, in a plant would increase total production of E1 compared to a single promoter/E1 construct in the plant. Furthermore, by using promoters targeted at different locations in the plant in the case of expression of multiple cell wall-modifying enzyme polypeptides, one can minimize in vivo (pre-processing) deconstruction of the cell wall that occurs when multiple synergistic enzymes are present in a cell. For example, combining an endoglucanase with an apoplast promoter, a hemicellulase with a vacuole promoter, and an exoglucanase with a chloroplast promoter, sequesters each enzyme in a different part of the cell and achieves the advantages listed above. This method circumvents the limit on enzyme mass that can be expressed in a single organelle or location of the cell.

The localization of a nuclear-encoded protein (e.g., enzyme polypeptide) within the cell is known to be determined by the amino acid sequence of the protein. The protein localization can be altered by modifying the nucleotide sequence that encodes the protein in such a manner as to alter the protein's amino acid sequence. The polynucleotide sequences encoding ligno-cellulolytic enzymes can be altered to redirect the cellular localization of the encoded enzymes by any suitable method (see, e.g., Dai et al., Trans. Res., 2005, 14: 627, the entire contents of which are herein incorporated by reference). In some embodiments of the invention, protein localization is altered by fusing a sequence encoding a signal peptide to the sequence encoding the enzyme polypeptide. Signal peptides that may be used in accordance with the invention include a secretion signal from sea anemone equistatin (which allows localization to apoplasts) and secretion signals comprising the KDEL motif (which allows localization to endoplasmic reticulum).

### Expression Vectors

Nucleic acid constructs according to the present invention may be cloned into a vector, such as, for example, a plasmid. Vectors suitable for transforming plant cells include, but are not limited to, Ti plasmids from Agrobacterium tumefaciens (J. Darnell, H. F. Lodish and D. Baltimore, “Molecular Cell Biology”, 2^{nd} Ed., 1990, Scientific American Books: New York), a plasmid containing a β-glucuronidase gene and a cauliflower mosaic virus (CaMV) promoter plus a leader sequence from alfalfa mosaic virus (J. C. Sanford et al., Plant Mol. Biol. 1993, 22: 751-765) or a plasmid containing a bar gene cloned downstream from a CaMV 35S promoter and a tobacco mosaic virus (TMV) leader. Other plasmids may additionally contain introns, such as that derived from alcohol dehydrogenase (Adh1), or other DNA sequences. The size of the vector is not a limiting factor.

For constructs intended to be used in Agrobacterium-mediated transformation, the plasmid may contain an origin of replication that allows it to replicate in Agrobacterium and a high copy number origin of replication functional in E. coli. This permits facile production and testing of transgenes in E. coli prior to transfer to Agrobacterium for subsequent introduction in plants. Resistance genes can be carried on the vector, one for selection in bacteria, for example, streptomycin, and another that will function in plants, for example, a gene encoding kanamycin resistance or herbicide resistance. Also present on the vector are restriction endonuclease sites for the addition of one or more transgenes and directional T-DNA border sequences which, when recognized by the transfer functions of Agrobacterium, delimit the DNA region that will be transferred to the plant.

Methods of preparation of nucleic acid constructs and expression vectors are well known in the art and can be found described in several textbooks such as, for example, J. Sambrook, E. F. Fritsch and T. Maniatis, “Molecular Cloning: A Laboratory Manual”, 1989, Cold Spring Harbor Laboratory: Cold Spring Harbor, and T. J. Silhavy, M. L. Berman, and L. W. Enquist, “Experiments with Gene Fusions”, 1984, Cold Spring Harbor Laboratory: Cold Spring Harbor; F. M. Ausubel et al., “Current Protocols in Molecular Biology”, 1989, John Wiley & Sons: New York.

Additional desirable properties of the transgenic plants may include, but are not limited to, ability to adapt for growth in various climates and soil conditions; well studied genetic model system; incorporation of bioconfinement features such as male (or total) sterile flowers; incorporation of phytoremediation features such as contaminant hyperaccumulation, greater biomass, or promotion of contaminant-degrading mycorrhizae.

### IV. Transgenic Plants

In some embodiments, the present invention provides novel transgenic plants that express one or more enzyme polypeptides. In some embodiments, provided transgenic plants express one or more cell wall-modifying enzyme polypeptides. In some embodiments, provided transgenic plants express one or more lignocellulolytic enzyme polypeptides.

Nucleic acid constructs, such as those described above, can be used to transform any plant including monocots and dicots. In some embodiments, plants are green field plants. In some embodiments, provided are transgenic plants, the genome of which are augmented with: a recombinant polynucleotide encoding at least one enzyme polypeptide linked to a promoter sequence, wherein the polynucleotide is optimized for expression in the plant, wherein the at least one enzyme polypeptide has at least 85% sequence identity to at least one of SEQ ID NO.: 1 to 84.

In other embodiments, plants are grown specifically for “biomass energy” and/or phytoremediation. Examples of suitable plants for use in the methods of the present invention include, but are not limited to, alfalfa, bamboo, barley, canola, corn, cotton, cottonwood (e.g., Populus deltoides), eucalyptus, miscanthus, poplar, pine (pinus sp.), potato, rape, rice, soy, sorghum, sugar beet, sugarcane, sunflower, sweetgum, switchgrass, tobacco, turf grass, wheat, and willow. Using transformation methods, genetically modified plants, plant cells, plant tissue, seeds, and the like can be obtained.

Transformation according to the present invention may be performed by any suitable method. In certain embodiments, transformation comprises steps of introducing a nucleic acid construct, as described above, into a plant cell or protoplast to obtain a stably transformed plant cell or protoplast; and regenerating a whole plant from the stably transformed plant cell or protoplast.

### Cell Transformation

Delivery or introduction of a nucleic acid construct into eukaryotic cells may be accomplished using any of a variety of methods. The method used for the transformation is not critical to the instant invention. Suitable techniques include, but are not limited to, non-biological methods, such as microinjection, microprojectile bombardment, electroporation, induced uptake, and aerosol beam injection, as well as biological methods such as direct DNA uptake, liposomes and Agrobacterium-mediated transformation. Any combinations of the above methods that provide for efficient transformation of plant cells or protoplasts may also be used in the practice of the invention.

Methods of introduction of nucleic acid constructs into plant cells or protoplasts have been described. See, for example, “Methods for Plant Molecular Biology”, Weissbach and Weissbach (Eds.), 1989, Academic Press, Inc; “Plant Cell, Tissue and Organ Culture: Fundamental Methods”, 1995, Springer-Verlag: Berlin, Germany; and U.S. Pat. Nos. 4,945,050; 5,036,006; 5,100,792; 5,240,855; 5,302,523; 5,322,783; 5,324,646; 5,384,253; 5,464,765; 5,538,877; 5,538,880; 5,550,318; 5,563,055; and 5,591,616).

In particular, electroporation has frequently been used to transform plant cells (see, for example, U.S. Pat. No. 5,384,253). This method is generally performed using friable tissues (such as a suspension culture of cells or embryogenic callus) or target recipient cells from immature embryos or other organized tissue that have been rendered more susceptible to transformation by electroporation by exposing them to pectin-degrading enzymes or by mechanically wounding them in a controlled manner. Intact cells of maize (see, for example, K. D'Halluin et al., Plant cell, 1992, 4: 1495-1505; C. A. Rhodes et al., Methods Mol. Biol. 1995, 55: 121-131; and U.S. Pat. No. 5,384,253), wheat, tomato, soybean, and tobacco have been transformed by electroporation. As reviewed, for example, by G. W. Bates (Methods Mol. Biol. 1999, 111: 359-366), electroporation can also be used to transform protoplasts.

Another method of transformation is microprojectile bombardment (see, for example, U.S. Pat. Nos. 5,538,880; 5,550,318; and 5,610,042; and WO 94/09699). In this method, nucleic acids are delivered to living cells by coating or precipitating the nucleic acids onto a particle or microprojectile (for example tungsten, platinum or gold), and propelling the coated microprojectile into the living cell. Microprojectile bombardment techniques are widely applicable, and may be used to transform virtually any monocotyledonous or dicotyledonous plant species (see, for example, U.S. Pat. Nos. 5,036,006; 5,302,523; 5,322,783 and 5,563,055; WO 95/06128; A. Ritala et al., Plant Mol. Biol. 1994, 24: 317-325; L. A. Hengens et al., Plant Mol. Biol. 1993, 23: 643-669; L. A. Hengens et al., Plant Mol. Biol. 1993, 22: 1101-1127; C. M. Buising and R. M. Benbow, Mol. Gen. Genet. 1994, 243: 71-81; C. Singsit et al., Transgenic Res. 1997, 6: 169-176).

The use of Agrobacterium-mediated transformation of plant cells is well known in the art (see, for example, U.S. Pat. No. 5,563,055). This method has long been used in the transformation of dicotyledonous plants, including Arabidopsis and tobacco, and has recently also become applicable to monocotyledonous plants, such as rice, wheat, barley and maize (see, for example, U.S. Pat. No. 5,591,616). In plant strains where Agrobacterium-mediated transformation is efficient, it is often the method of choice because of the facile and defined nature of the gene transfer. Agrobacterium-mediated transformation of plant cells is carried out in two phases. First, the steps of cloning and DNA modifications are performed in E. coli, and then the plasmid containing the gene construct of interest is transferred by heat shock treatment into Agrobacterium, and the resulting Agrobacterium strain is used to transform plant cells. In some embodiments, Agrobacterium infiltrates plant leaves. In some embodiments, the bacterial strain Agrobacterium tumefaciens is used to transform plant cells.

Transformation of plant protoplasts can be achieved using methods based on calcium phosphate precipitation, polyethylene glycol treatment, electroporation, and combinations of these treatments (see, e.g., I. Potrykus et al., Mol. Gen. Genet. 1985, 199: 169-177; M. E. Fromm et al., Nature, 1986, 31: 791-793; J. Callis et al., Genes Dev. 1987, 1: 1183-1200; S. Omirulleh et al., Plant Mol. Biol. 1993, 21: 415-428).

Alternative methods of plant cell transformation, which have been reviewed, for example, by M. Rakoczy-Trojanowska (Cell Mol. Biol. Lett. 2002, 7: 849-858), can also be used in the practice of the present invention.

The successful delivery of the nucleic acid construct into the host plant cell or protoplast may be preliminarily evaluated visually. Selection of stably transformed plant cells can be performed, for example, by introducing into the cell, a nucleic acid construct comprising a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Examples of herbicides which may be used include phosphinothricin and glyphosate. Potentially transformed cells then are exposed to the selective agent. Cells where the resistance-conferring gene has been integrated and expressed at sufficient levels to permit cell survival will generally be present in the population of surviving cells.

Alternatively, host cells comprising a nucleic acid sequence of the invention and which express its gene product may be identified and selected by a variety of procedures, including, DNA-DNA or DNA-RNA hybridization and protein bioassay or immunoassay techniques such as membrane, solution or chip-based technologies for the detection and/or quantification of nucleic acid or protein.

Plant cells are available from a wide range of sources including the American Type Culture Collection (Rockland, Md.), or from any of a number of seed companies including, for example, A. Atlee Burpee Seed Co. (Warminster, Pa.), Park Seed Co. (Greenwood, S.C.), Johnny Seed Co. (Albion, Me.), or Northrup King Seeds (Hartsville, S.C.). Descriptions and sources of useful host cells are also found in I. K. Vasil, “Cell Culture and Somatic Cell Genetics of Plants”, Vol. I, II and II; 1984, Laboratory Procedures and Their Applications Academic Press: New York; R. A. Dixon et al., “Plant Cell Culture—A Practical Approach”, 1985, IRL Press: Oxford University; and Green et al., “Plant Tissue and Cell Culture”, 1987, Academic Press: New York.

Plant cells or protoplasts stably transformed according to the present invention are provided herein.

### Plant Regeneration

In plants, every cell is capable of regenerating into a mature plant, and in addition contributing to the germ line such that subsequent generations of the plant will contain the transgene of interest. Stably transformed cells may be grown into plants according to conventional ways (see, for example, McCormick et al., Plant Cell Reports, 1986, 5: 81-84). Plant regeneration from cultured protoplasts has been described, for example by Evans et al., “Handbook of Plant Cell Cultures”, Vol. 1, 1983, MacMilan Publishing Co: New York; and I. R. Vasil (Ed.), “Cell Culture and Somatic Cell Genetics of Plants”, Vol. I (1984) and Vol. II (1986), Acad. Press: Orlando.

Means for regeneration vary from species to species of plants, but generally a suspension of transformed protoplasts or a Petri plate containing transformed explants is first provided. Callus tissue is formed and shoots may be induced from callus and subsequently roots. Alternatively, somatic embryo formation can be induced in the callus tissue. These somatic embryos germinate as natural embryos to form plants. The culture media will generally contain various amino acids and plant hormones, such as auxin and cytokinins. Glutamic acid and proline may also be added to the medium. Efficient regeneration generally depends on the medium, on the genotype, and on the history of the culture.

Regeneration from transformed individual cells to obtain transgenic whole plants has been shown to be possible for a large number of plants. For example, regeneration has been demonstrated for dicots (such as apple; Malus pumila; blackberry, Rubus; Blackberry/raspberry hybrid, Rubus; red raspberry, Rubus; carrot; Daucus carota; cauliflower; Brassica oleracea; celery; Apium graveolens; cucumber; Cucumis sativus; eggplant; Solanum melongena; lettuce; Lactuca sativa; potato; Solanum tuberosum; rape; Brassica napus; soybean (wild); Glycine canescens; strawberry; Fragaria x ananassa; tomato; Lycopersicon esculentum; walnut; Juglans regia; melon; Cucumis melo; grape; Vitis vinifera; and mango; Mangifera indica) as well as for monocots (such as rice; Ojyza sativa; rye, Secale cereale; and Maize).

Primary transgenic plants may then be grown using conventional methods. Various techniques for plant cultivation are well known in the art. Plants can be grown in soil, or alternatively can be grown hydroponically (see, for example, U.S. Pat. Nos. 5,364,451; 5,393,426; and 5,785,735). Primary transgenic plants may be either pollinated with the same transformed strain or with a different strain and the resulting hybrid having the desired phenotypic characteristics identified and selected. Two or more generations may be grown to ensure that the subject phenotypic characteristics is stably maintained and inherited and then seeds are harvested to ensure that the desired phenotype or other property has been achieved.

As is well known in the art, plants may be grown in different media such as soil, growth solution or water.

Selection of plants that have been transformed with the construct may be performed by any suitable method, for example, with northern blot, Southern blot, herbicide resistance screening, antibiotic resistance screening or any combinations of these or other methods. The Southern blot and northern blot techniques, which test for the presence (in a plant tissue) of a nucleic acid sequence of interest and of its corresponding RNA, respectively, are standard methods (see, for example, Sambrook & Russell, “Molecular Cloning”, 2001, Cold Spring Harbor Laboratory Press: Cold Spring Harbor).

### V. Compositions of Matter

In one aspect, provided are compositions of matter that be used, among other things, in cost-effective methods for processing lignocellulolic biomass.

In many embodiments, provided compositions of matter comprise plant biomass and at least one cell wall-modifying enzyme polypeptide as described herein. In some embodiments, the at least one cell wall-modifying enzyme polypeptide has at least 85% amino acid sequence identity to at least one of SEQ ID NO: 1 to 84. In some embodiments, the cell wall-modifying enzyme polypeptide has at least 85% amino acid sequence identity to a sequence selected from the group consisting of SEQ ID NO: 1, SEQ ID NO: 2, SEQ ID NO: 11, SEQ ID NO: 40, SEQ ID NO: 78, and SEQ ID NO: 80. (See, for example, Example 4.)

“Plant biomass” as used herein refers to biomass that includes a plurality of components found in plant, such as lignin, cellulose, hemicellulose, beta-glucans, homogalacturonans, rhamnogalacturonans, droxycinnamic acids, polyphenolic acids, and proteins. Plant biomass may be obtained, for example, from a transgenic plant expressing at least one cell wall-modifying enzyme polypeptide as described herein. Plant biomass may be obtained from any part of a plant, including, but not limited to, leaves, stems, seeds, and combinations thereof.

In some embodiments, the plant biomass comprises biomass from a monocotyledonous plant. The monocotyledonous plant may be selected from the group consisting of maize, sorghum, switchgrass, miscanthus, sugarcane, wheat, rice, rye, turfgrass, and millet. In some embodiments, the plant biomass comprises biomass from a dicotyledonous plant. The dicotyledonous plant may be selected from the group consisting of tobacco, potato, soybean, canola, sunflower, alfalfa, cotton and poplar, eucalyptus, pine, sweetgum, and cottonwood (e.g., Populus deltoides).

Compositions of matter provided in the present invention include compositions in which the plant biomass has undergone one or more processes, yet still retains a plurality of components found in plant. For example, compositions of matter may comprise plant biomass that has been stored and/or ensiled. Alternatively or additionally, compostions of matter of the present invention may comprise plant biomass that has been tempered as defined herein. For example, plant biomass may be tempered by incubating at a temperature such as 37° C. for a period of time such as 24 h.

In some embodiments, activity of the cell wall-modifying enzyme polypeptide is engaged by post-harvest processing of the plant biomass. Examples of such post-harvest processing include, but are not limited to, ensilage, thermochemical bioprocessing, processing in the digestive tract of a mammal, and combinations thereof.

### VI. Antibodies, Arrays, and Plates

In some aspects, provided are reagents and materials that have, among other things, diagnostic and research applications. For example, these reagents and materials may be used to detect and/or assess levels of expression (e.g., gene expression and/or protein expression) of cell wall-modifying enzyme polypeptides. Transgenic plants used for commercial purposes (such as biofuel production), may for example, be screened and/or evaluated using the reagents and materials described herein.

### Antibodies

In one aspect, provided are antibodies that bind to cell wall-modifying enzyme polypeptides. In some embodiments, antibodies are isolated antibodies, e.g., separated from one or more components with which they are normally naturally associated. Such antibodies may be useful, for example in a variety of assays for detecting protein expression of cell wall-modifying enzyme polypeptides.

In some embodiments, provided are antibodies that bind specifically to a feruloyl esterase polypeptide. In some such embodiments, the feruloyl esterase polypeptide has an amino acid sequence at least 85% identical to SEQ ID NO: 2. (See Example 3.)

In some embodiments provided are antibodies that bind specifically to an exoglucanase polypeptide. In some such embodiments, the exoglucanase polypeptide has an amino acid sequence at least 85% identical to SEQ ID NO: 40. (See Example 3.)

In some embodiments, provided antibodies are monoclonal antibodies. In some embodiments, provided antibodies are polyclonal antibodies. Methods of producing polyclonal antibodies are known in the art. For example, a cell wall-modifying enzyme polypeptide, or a peptide derived therefrom, may be injected into an animal (such as a rabbit, chicken, goat, donkey, etc.) to generate polyclonal antibodies against the enzyme polypeptide.

In some embodiments, provided antibodies are monoclonal antibodies. Methods of producing monoclonal antibodies are known in the art. For example, monoclonal antibodies may be generated by fusing antibody-producing B cells (generated, for example, by immunizing an animal with a cell wall-modifying enzyme polypeptide are peptide therefrom) with multiple myeloma cells that cannot produce their own antibodies to generate hybridoma cells. Such hybridoma cells can be grown and screened for antibodies against the antigen of interest and then desired hybridoma cell lines can be grown to produce many copies of the desired monoclonal antibody. In some embodiments, such hybridoma cell lines are injected into a laboratory animal (e.g., a mouse) to form one or more tumors. The desired antibodies can then be collected from such mice, for example from ascites fluid.

### Arrays

In one aspect, provided are arrays that may be used, for example, to detect expression of one or more genes encoding cell wall-modifying enzyme polypeptides. In many embodiments, provides arrays comprise a solid substrate with a surface and a plurality of genetic probes for cell wall modifying enzyme polypeptides, each immobilized to a discrete spot on the surface of the substrate to form an array. In some embodiments, the plurality of genetic probes comprises at least ten different oligonucleotides, each oligonucleotide comprising at least ten consecutive nucleotides from a nucleic acid encoding a polypeptide have a sequence of one of SEQ ID NO: 1 to 84.

Methods of making and using arrays are well known in the art (see, for example, S. Kern and G. M. Hampton, Biotechniques, 1997, 23:120-124; M. Schummer et al., Biotechniques, 1997, 23:1087-1092; S. Solinas-Toldo et al., Genes, Chromosomes & Cancer, 1997, 20: 399-407; M. Johnston, Curr. Biol. 1998, 8: R171-R174; D. D. Bowtell, Nature Gen. 1999, Supp. 21:25-32; S. J. Watson and H. Akil, Biol Psychiatry. 1999, 45: 533-543; W. M. Freeman et al., Biotechniques. 2000, 29: 1042-1046 and 1048-1055; D. J. Lockhart and E. A. Winzeler, Nature, 2000, 405: 827-836; M. Cuzin, Transfus. Clin. Biol. 2001, 8:291-296; P. P. Zarrinkar et al., Genome Res. 2001, 11: 1256-1261; M. Gabig and G. Wegrzyn, Acta Biochim. Pol. 2001, 48: 615-622; and V. G. Cheung et al., Nature, 2001, 40: 953-958; see also, for example, U.S. Pat. Nos. 5,143,854; 5,434,049; 5,556,752; 5,632,957; 5,700,637; 5,744,305; 5,770,456; 5,800,992; 5,807,522; 5,830,645; 5,856,174; 5,959,098; 5,965,452; 6,013,440; 6,022,963; 6,045,996; 6,048,695; 6,054,270; 6,258,606; 6,261,776; 6,277,489; 6,277,628; 6,365,349; 6,387,626; 6,458,584; 6,503,711; 6,516,276; 6,521,465; 6,558,907; 6,562,565; 6,576,424; 6,587,579; 6,589,726; 6,594,432; 6,599,693; 6,600,031; and 6,613,893).

Substrate surfaces suitable for use in the present invention can be made of any of a variety of rigid, semi-rigid or flexible materials that allow direct or indirect attachment (i.e., immobilization) of genetic probes to the substrate surface. Suitable materials include, but are not limited to: cellulose (see, for example, U.S. Pat. No. 5,068,269), cellulose acetate (see, for example, U.S. Pat. No. 6,048,457), nitrocellulose, glass (see, for example, U.S. Pat. No. 5,843,767), quartz or other crystalline substrates such as gallium arsenide, silicones (see, for example, U.S. Pat. No. 6,096,817), various plastics and plastic copolymers (see, for example, U.S. Pat. Nos. 4,355,153; 4,652,613; and 6,024,872), various membranes and gels (see, for example, U.S. Pat. No. 5,795,557), and paramagnetic or supramagnetic microparticles (see, for example, U.S. Pat. No. 5,939,261). When fluorescence is to be detected, arrays comprising cyclo-olefin polymers may in some embodiments be used (see, for example, U.S. Pat. No. 6,063,338). Other materials that may be used include, but are not limit metals, resins, polymers, ceramic, graphite, etc. In some embodiments, substrates comprise a material selected from the group consisting of metals, resins, polymers, ceramic, glass, graphite, and combinations thereof.

Substrate surfaces may be in any of a variety of forms, for example, flow cells, microelectrodes, beads, gels, plates, slides, capillary tubes, etc.

Presence of reactive functional chemical groups (such as, for example, hydroxyl, carboxyl, amino groups and the like) on the material can be exploited to directly or indirectly attach genetic probes to the substrate surface. Methods for immobilizing genetic probes to substrate surfaces to form an array are well-known in the art.

More than one copy of each genetic probe may be spotted on the array (for example, in duplicate or in triplicate). This arrangement may, for example, allow assessment of the reproducibility of the results obtained. Related genetic probes may also be grouped in probe elements on an array. For example, a probe element may include a plurality of related genetic probes of different lengths but comprising substantially the same sequence. Alternatively, a probe element may include a plurality of related genetic probes that are fragments of different lengths resulting from digestion of more than one copy of a cloned piece of DNA. A probe element may also include a plurality of related genetic probes that are identical fragments except for the presence of a single base pair mismatch. An array may contain a plurality of probe elements. Probe elements on an array may be arranged on the substrate surface at different densities.

Genetic probes may be long cDNA sequences (500 to 5,000 bases long) or shorter sequences (for example, 20-80-mer oligonucleotides). The sequences of the genetic probes are those for which gene expression levels information is desired. Additionally or alternatively, the array may comprise nucleic acid sequences of unknown significance or location. Genetic probes may be used as positive or negative controls (for example, the array may contain perfect match sequences as well as single base pair mismatch sequences to adjust for non-specific hybridization).

Techniques for the preparation and manipulation of genetic probes are well-known in the art (see, for example, J. Sambrook et al., “Molecular Cloning: A Laboratory Manual”, 1989, 2nd Ed., Cold Spring Harbour Laboratory Press: New York, N.Y.; “PCR Protocols: A Guide to Methods and Applications”, 1990, M. A. Innis (Ed.), Academic Press: New York, N.Y.; P. Tijssen “Hybridization with Nucleic Acid Probes—Laboratory Techniques in Biochemistry and Molecular Biology (Parts I and II)”, 1993, Elsevier Science; “PCR Strategies”, 1995, M. A. Innis (Ed.), Academic Press: New York, N.Y.; and “Short Protocols in Molecular Biology”, 2002, F. M. Ausubel (Ed.), 5th Ed., John Wiley & Sons).

Long cDNA sequences may be obtained and manipulated by cloning into various vehicles. They may be screened and re-cloned or amplified from any source of genomic DNA. Genetic probes may be derived from genomic clones including mammalian and human artificial chromosomes (MACs and HACs, respectively, which can contain inserts from ˜5 to 400 kilobases (kb)), satellite artificial chromosomes or satellite DNA-based artificial chromosomes (SATACs), yeast artificial chromosomes (YACs; 0.2-1 Mb in size), bacterial artificial chromosomes (BACs; up to 300 kb); P1 artificial chromosomes (PACs; 70-100 kb) and the like.

Genetic probes may also be obtained and manipulated by cloning into other cloning vehicles such as, for example, recombinant viruses, cosmids, or plasmids (see, for example, U.S. Pat. Nos. 5,266,489; 5,288,641 and 5,501,979).

In some embodiments, genetic probes are synthesized in vitro by chemical techniques well-known in the art and then immobilized on arrays. Such methods are especially suitable for obtaining genetic probes comprising short sequences such as oligonucleotides and have been described in scientific articles as well as in patents (see, for example, S. A. Narang et al., Meth. Enzymol. 1979, 68: 90-98; E. L. Brown et al., Meth. Enzymol. 1979, 68: 109-151; E. S. Belousov et al., Nucleic Acids Res. 1997, 25: 3440-3444; D. Guschin et al., Anal. Biochem. 1997, 250: 203-211; M. J. Blommers et al., Biochemistry, 1994, 33: 7886-7896; and K. Frenkel et al., Free Radic. Biol. Med. 1995, 19: 373-380; see also for example, U.S. Pat. No. 4,458,066).

For example, oligonucleotides may be prepared using an automated, solid-phase procedure based on the phosphoramidite approach. In such a method, each nucleotide is individually added to the 5-end of the growing oligonucleotide chain, which is attached at the 3′-end to a solid support. The added nucleotides are in the form of trivalent 3′ phosphoramidites that are protected from polymerization by a dimethoxytrityl (or DMT) group at the 5-position. After base-induced phosphoramidite coupling, mild oxidation to give a pentavalent phosphotriester intermediate and DMT removal provides a new site for oligonucleotide elongation. The oligonucleotides are then cleaved off the solid support, and the phosphodiester and exocyclic amino groups are deprotected with ammonium hydroxide. These syntheses may be performed on commercial oligo synthesizers such as the Perkin Elmer/Applied Biosystems Division DNA synthesizer.

Methods of attachment (or immobilization) of oligonucleotides on substrate supports have been described (see, for example, U. Maskos and E. M. Southern, Nucleic Acids Res. 1992, 20: 1679-1684; R. S. Matson et al., Anal. Biochem. 1995, 224; 110-116; R. J. Lipshutz et al., Nat. Genet. 1999, 21: 20-24; Y. H. Rogers et al., Anal. Biochem. 1999, 266: 23-30; M. A. Podyminogin et al., Nucleic Acids Res. 2001, 29: 5090-5098; Y. Belosludtsev et al., Anal. Biochem. 2001, 292: 250-256).

Oligonucleotide-based arrays have also been prepared by synthesis in situ using a combination of photolithography and oligonucleotide chemistry (see, for example, A. C. Pease et al., Proc. Natl. Acad. Sci. USA 1994, 91: 5022-5026; D. J. Lockhart et al., Nature Biotech. 1996, 14: 1675-1680; S. Singh-Gasson et al., Nat. Biotechn. 1999, 17: 974-978; M. C. Pirrung et al., Org. Lett. 2001, 3: 1105-1108; G. H. McGall et al., Methods Mol. Biol. 2001, 170; 71-101; A. D. Barone et al., Nucleosides Nucleotides Nucleic Acids, 2001, 20: 525-531; J. H. Butler et al., J. Am. Chem. Soc. 2001, 123: 8887-8894; E. F. Nuwaysir et al., Genome Res. 2002, 12: 1749-1755). The chemistry for light-directed oligonucleotide synthesis using photolabile protected 2′-deoxynucleoside phosphoramites has been developed by Affymetrix Inc. (Santa Clara, Calif.) and is well known in the art (see, for example, U.S. Pat. Nos. 5,424,186 and 6,582,908).

### Plates

In one aspect, provided are plates that may be used, for example, in ELISAs (enzyme-linked immunosorbent assays) to detect and/or quantitate protein expression of cell wall-modifying enzyme polypeptides and/or expression of antibodies against cell wall modifying enzymes. In many embodiments, such plates comprise a solid substrate with a surface, and a peptide immobilized to the surface. In some embodiments, the peptide comprises at least six consecutive amino acids from a polypeptide having a sequence of one of SEQ ID NO: 1 to 84.

Suitable materials for such plates are known in the art and may include those materials described above for arrays. In some embodiments, plates are made of plastics and/or copolymers.

### VII. Uses of Inventive Transgenic Plants and Compositions of Matter

Transgenic plants, plant parts, and compositions of matter disclosed herein may be used advantageously in a variety of applications. More specifically, the present invention, which involves genetically engineering plants for both increased biomass and expression of cell wall-modifying enzyme polypeptides, results in downstream process innovations and/or improvements in a variety of applications including ethanol production, phytoremediation and hydrogen production.

In some embodiments, provided are methods comprising steps of: pretreating a plant part under conditions to promote accessibility of celluloses within the lignocellulosic biomass; and treating the pretreated plant part under conditions that promote hydrolysis of cellulose to fermentable sugars, wherein the plant part is obtained from at least one transgenic plant, the genome of which is augmented with: a recombinant polynucleotide encoding at least one enzyme polypeptide operably linked to a promoter sequence, wherein the polynucleotide is optimized for expression in the plant and wherein the at least one enzyme polypeptide has at least 85% sequence identity to at least one of SEQ ID NO.: 1 to 84.

### A—Ethanol Production

Plants transformed according to the present invention provide a means of increasing ethanol yields, reducing pretreatment costs by reducing acid/heat pretreatment requirements for saccharification of biomass; and/or reducing other plant production and processing costs, such as by allowing multi-applications and isolation of commercially valuable by-products.

Plant Culture. As already mentioned above, farmers can grow different transgenic plants of the present invention (e.g., different variety of transgenic corn, each expressing a cell wall-modifying enzyme polypeptide or a combination of enzyme polypeptides) simultaneously, achieving the desired “blend” of enzyme polypeptides produced by changing the seed ratio.

Plant Harvest. Transgenic plants of the present invention can be harvested as known in the art. For example, current techniques may cut corn stover at the same time as the grain is harvested, but leave the stover lying in the field for later collection. However, dirt collected by the stover can interfere with ethanol production from lignocellulosic material. The present invention provides a method in which transgenic plants are cut, collected, stored, and transported so as to minimize soil contact. In addition to minimizing interference from dirt with ethanol production, this method can result in reduction in harvest and transportation costs.

Tempering. Inventive methods include a tempering phase that conditions the biomass for pretreatment and hydrolysis. Tempering may facilitate reducing severity of pretreatment conditions to achieve a desired glucan conversion yield and/or improving hydrolysis and glucan conversion after treatment. For example, a typical yield from biomass that has been pretreated under standard pretreatment conditions (e.g., 1% sulfuric acid, 170° C., for 10 minutes) is at least 80% glucan conversion. When tempered as described herein, the same typical yield may be achieved under less severe pretreatment conditions and/or with reduced amounts of externally applied enzymes. Less severe pretreatment conditions may comprise, for example, reduced acid concentrations, lower incubation temperatures, and/or shorter pretreatment times.

In some embodiments, when tempered as described herein and using the same pretreatment conditions, typical yield may be increased above at least 80% glucan conversion.

Without wishing to be bound by any particular theory, tempering may facilitate such improvements by, for example, allowing activation of endoplant enzyme polypeptides after harvest, increasing susceptibility of lignin and hemicellulose to traditional pretreatment, and/or increasing accessibility of polysaccharides (e.g., cellulose).

A variety of techniques for tempering may be used. In some embodiments, tempering comprises increasing the temperature of the biomass to activate thermophilic enzymes. Increasing the temperature to activate thermophilic enzymes may be achieved, for example, by one or more of ensilement, grinding, pelleting, and warm water suspension/slurries. In some embodiments, tempering comprises disrupting cell walls. Cell wall disruption may be achieved, for example, by sonication and/or liquid extraction to release enzyme polypeptides from sequestered locations in the plant (which may allow further activation and/or extraction to be added back after pretreatment). In some embodiments, tempering comprises adding accessory enzyme polypeptides during an incubation period before pretreatment. Such accessory enzyme polypeptides may weaken cross linking and improve accessibility of the biomass to embedded glucanases or xylanases. In some embodiments, tempering comprises incubating the biomass in a particular set of conditions (e.g., a particular temperature, particular pH, and/or particular moisture conditions). Such incubations may in some embodiments increase susceptibility to various glucanases and/or accessory enzyme polypeptides present in the plant tissues or added to the sample. For example, samples may be tempered as a liquid slurry (e.g., comprising about 10% to about 30% total solids) under conditions favorable to activate cell wall-modifying enzymes. In some embodiments, samples are tempered as a liquid slurry for about 1 to about 48 hours. In some embodiments, conditions favorable to activate cell wall-modifying enzymes comprise a pH of about 4 to about 7 and a temperature of about 25° C. to about 100° C. Alternatively or additionally, samples may be tempered as a lower moisture ensilement (e.g., about 40% to about 60% total solids) under anaerobic conditions. In some embodiments, samples are ensiled for about 21 days to several months.

In some embodiments, tempering is integrated with other processes such as one or more of harvest, storage, and transportation of biomass. For example, biomass can be ensiled under conditions that condition the biomass for subsequent pretreatment and hydrolysis; that is, storage and tempering are combined. In some embodiments, during ensilement of biomass, temperatures are increased in the ensiled material such that thermally active embedded enzymes are activated. Ensilement conditions may allow preservation of biomass while providing sufficient time for enzyme polypeptides to affect characteristics of the biomass (such as, for example, amenability to pretreatment and improvement of subsequent hydrolysis).

In some embodiments, the tempering phase precedes entirely the pretreatment phase. In some embodiments, the tempering phase overlaps with the pretreatment phase.

In some embodiments as described herein, transgenic plants express more than one cell wall-modifying enzyme polypeptide. In some such embodiments, it may be desirable to activate enzyme polypeptides sequentially. It may be desirable to do so, for example, if the efficiency of endoplant enzymes is a function of the sequence in which they are activated. For example, beta-glucosidases may be most efficient after endo- and exoglucanases have cleaved cellulose into dimers, and cellulases and hemicellulases may be more efficient when accessory enzymes have reduced cross-linkages between cellulose, hemicellulose, and lignin. Accordingly, in some embodiments, cellulases might be activated after ferulic acid esterases (FAEs) have had the opportunity to cleave ferulate-polysaccharide-lignin complexes, or after other accessory enzymes have had the opportunity to cleave cellulose-hemicellulose cross linkages.

Sequential activation could be attained, for example, by using enzymes with different peak temperature and/or pH optima. Increasing temperature continually or stepwise (e.g., during a tempering step), could thereby allow activation of enzyme polypeptides with lower temperature optima first. For example, a wound-induced promoter could be used to produce a non-thermostable enzyme polypeptide after harvesting that breaks lingin cross-links and leads to cell death, before increasing temperature during tempering to activate a thermostable cellulase in the biomass.

In some embodiments as described herein, cell wall-modifying enzyme polypeptides are specifically targeted to organelles and/or plant parts. In some embodiments, cell wall-modifying enzyme polypeptides are specifically targeted to seeds. Cell wall hydrolyzing enzymes in the grain could improve yields of fermentable sugars by targeting the cellulose and hemicellulose in the grain bran and fiber, or could loosen or weaken the outer layers of the grain kernel, making it easier to mill. Starch in corn grain is often processed to produce ethanol, but significant quantities of cellulose and hemicellulose from the bran and fiber are not used. In some embodiments, incorporating a tempering step prior to starch hydrolysis (e.g., of transgenic corn grain), endogenous enzymes can act on the fiber and bran and increase the yield of fermentable sugars. In some embodiments, dry seed (e.g., dry wheat) is tempered by soaking in water at a slightly elevated temperature for several hours before further processing. Such a tempering step may decrease the energy required for milling and increase the quality and eventual yield. Endogenous enzymes in the grain may also provide additional benefits.

In some embodiments, tempering comprises externally applying an amount of at least one cell wall-modifying enzyme polypeptide. External application of cell wall-modifying enzyme polypeptides is discussed in more detail in the “Saccharification” section.

In some embodiments, the seed or grain of a transgenic plant is tempered.

Pretreatment. Conventional methods include physical, chemical, and/or biological pretreatments. For example, physical pretreatment techniques can include one or more of various types of milling, crushing, irradiation, steaming/steam explosion, and hydrothermolysis. Chemical pretreatment techniques can include acid, alkaline, organic solvent, ammonia, sulfur dioxide, carbon dioxide, and pH-controlled hydrothermolysis. Biological pretreatment techniques can involve applying lignin-solubilizing microorganisms (T.-A. Hsu, “Handbook on Bioethanol: Production and Utilization”, C. E. Wyman (Ed.), 1996, Taylor & Francis: Washington, D.C., 179-212; P. Ghosh and A. Singh, A., Adv. Appl. Microbiol., 1993, 39: 295-333; J. D. McMillan, in “Enzymatic Conversion of Biomass for Fuels Production”, M. Himmel et al., (Eds.), 1994, Chapter 15, ACS Symposium Series 566, American Chemical Society: B. Hahn-Hagerdal, Enz. Microb. Tech., 1996, 18: 312-331; and L. Vallander and K. E. L. Eriksson, Adv. Biochem. Eng./Biotechnol., 1990, 42: 63-95). The purpose of the pretreatment step is to break down the lignin and carbohydrate structure to make the cellulose fraction accessible to cellulolytic enzymes.

Simultaneous use of transgenic plants that express one or more cellulases, one or more hemicellulases and/or one or more ligninases according to the present invention reduces or eliminates expensive grinding of the biomass, reduces or eliminates the need for heat and strong acid required to strip lignin and hemicellulose away from cellulose before hydrolyzing the cellulose.

In some embodiments, lignocellulosic biomass of plant parts obtained from inventive transgenic plants is more easily hydrolyzable than that of non-transgenic plants. Thus, the extent and/or severity of pretreatment required to achieve a particular level of hydrolysis is reduced. Therefore, the present invention in some embodiments provides improvements over existing pretreatment methods. Such improvements may include one or more of: reduction of biomass grinding, elimination of biomass grinding, reduction of the pretreatment temperature, elimination of heat in the pretreatment, reduction of the strength of acid in the pretreatment step, elimination of acid in the pretreatment step, and any combination thereof.

In some embodiments, lower temperatures of pretreatment may be used to achieve a desired level of hydrolysis. In some embodiments, pretreating is performed at temperatures below about 175° C., below about 145° C., or below about 115° C. For example, under some conditions, the yield of hydrolysis products from lignocellulosic biomass from transgenic plant parts pretreated at about 140° C. is comparable to the yield of hydrolysis products from non-transgenic plant parts pretreated at about 170° C. Under some conditions, the yield of hydrolysis products from lignocellulosic biomass from transgenic plant parts pretreated at about 170° C. is above about 60%, above about 70%, above about 80%, or above about 90% of theoretical yields. Under some conditions, the yield of hydrolysis products from lignocellulosic biomass from transgenic plant parts pretreated at about 140° C. is above about 60%, above about 70%, or above about 80% of theoretical yields. Under some conditions, the yield of hydrolysis products from lignocellulosic biomass from transgenic plant parts pretreated at about 110° C. is above about 40%, above about 50%, or above about 60% of theoretical yields. Such yields from transgenic plant parts can represent an increase of up to about 20% of yields from non-transgenic plant parts.

In some embodiments, such improvements are observed in inventive transgenic plants expressing a cell wall-modifying enzyme polypeptide at a level less than about 0.5%, less than about 0.4%, less than about 0.3%, less than about 0.2%, or less than about 0.1% of total soluble protein. Without wishing to be bound by any particular theory, the inventors propose that low levels of enzyme expression may facilitate modifying the cell wall, possibly by nicking cellulose or hemicellulose strands. Such modification of the cell wall may make the biomass more susceptible to pretreatment. Thus, biomass from inventive transgenic plants expressing low levels of cell wall-modifying enzymes may require less pretreatment, and/or pretreatment in less severe conditions.

In certain embodiments, the pretreated material is used for saccharification without further manipulation. In other embodiments, it may be desired to process the plant tissue so as to produce an extract comprising the cell wall-modifying enzyme polypeptide(s). In this case, the extraction is carried out in the presence of components known in the art to favor extraction of active enzymes from plant tissue and/or to enhance the degradation of cell-wall polysaccharides in the lignocellulosic biomass. Such components include, but are not limited to, salts, chelators, detergents, antioxidants, polyvinylpyrrolidone (PVP), and polyvinylpolypyrrolidone (PVPP). The remaining plant tissue may then be submitted to a pretreatment process.

### Saccharification.

In saccharification (or enzymatic hydrolysis), lignocellulose is converted into fermentable sugars (i.e. glucose monomers) by cell wall-modifying enzyme polypeptides present in the pretreated material. If desired, external cellulolytic enzyme polypeptides (i.e., enzymes not produced by the transgenic plants being processed) may be added to this mixture. Extracts comprising cell wall-modifying enzyme polypeptides obtained as described above can be added back to the lignocellulosic biomass before saccharification. Here again, external cellulolytic enzyme polypeptides may be added to the saccharification reaction mixture.

In some embodiments, the amount of externally applied enzyme polypeptide that is required to achieve a particular level of hydrolysis of lignocellulosic biomass from inventive transgenic plants is reduced as compared to the amount required to achieve a similar level of hydrolysis of lignocellulosic biomass from non-transgenic plants. For example, in some embodiments, processing transgenic lignocellulosic biomass in the presence of as low as 15 mg externally applied cellulase per gram of biomass (15 mg/g) yields a similar level of hydrolysis as processing non-transgenic lignocellulosic biomass in the presence of 100 mg/g cellulase. This represents a reduction of almost 90% of cellulases needed for hydrolysis can be achieved when processing biomass from inventive transgenic plants. Such a reduction in externally applied cellulases used can represent significant cost savings.

In some embodiments, a mixture of enzyme polypeptides each having different enzyme activities (e.g., exoglucanase, endoglucanase, hemi-cellulase, beta-glucosidase, and combinations thereof), and/or an enzyme polypeptide having more than one enzyme activity (e.g., exoglucanase, endoglucanase, hemi-cellulase, beta-glucosidase, and combinations thereof) is added during a “treatment” step to promote saccharification. Without wishing to be bound by any particular theory, such combinations of enzyme activity, whether through the activity of an enzyme complex or other mixture of enzymes, may allow a greater degree of hydrolysis than can be achieved with a single enzyme activity alone. Commercially available enzyme complexes that can be employed in the practice of the invention include, but are not limited to, Accellerase™ 1000 (Genencor), which contains multiple enzyme activities, mainly exoglucanase, endoglucanase, hemi-cellulase, and beta-glucosidase.

Saccharification is generally performed in stirred-tank reactors or fermentors under controlled pH, temperature, and mixing conditions. A saccharification step may last up to 200 hours. Saccharification may be carried out at temperatures from about 30° C. to about 65° C., in particular around 50° C., and at a pH in the range of between about 4 and about 5, in particular, around pH 4.5. Saccharification can be performed on the whole pretreated material.

The present Applicants have shown that adding cellulases to E1-transformed plants increases total glucose production compared to adding cellulases to non-transgenic plants, which suggests that simply using transgenic E1 plants with current external cellulase techniques can substantially increase ethanol yields. The experiment also indicates that adding cellulases to E1 plants increases total glucose production compared to adding cellulases to non-transgenic plants. This is an important result since it suggests that simply using transgenic E1 plants with current external cellulase techniques can substantially increase ethanol yields in the presence or absence of pretreatment processes.

Fermentation. In the fermentation step, sugars, released from the lignocellulose as a result of the pretreatment and enzymatic hydrolysis steps, are fermented to one or more organic substances, e.g., ethanol, by a fermenting microorganism, such as yeasts and/or bacteria. The fermentation can also be carried out simultaneously with the enzymatic hydrolysis in the same vessels, again under controlled pH, temperature and mixing conditions. When saccharification and fermentation are performed simultaneously in the same vessel, the process is generally termed simultaneous saccharification and fermentation or SSF.

Fermenting microorganisms and methods for their use in ethanol production are known in the art (Sheehan, “The Road to Bioethanol: A Strategic Perspective of the US Department of Energy's National Ethanol Program” In: “Glycosyl Hydrolases For Biomass Conversion”, ACS Symposium Series 769, 2001, American Chemical Society: Washington, D.C.). Existing ethanol production methods that utilize corn grain as the biomass typically involve the use of yeast, particularly strains of Saccharomyces cerevisiae. Such strains can be utilized in the methods of the invention. While such strains may be preferred for the production of ethanol from glucose that is derived from the degradation of cellulose and/or starch, the methods of the present invention do not depend on the use of a particular microorganism, or of a strain thereof, or of any particular combination of said microorganisms and said strains.

Yeast or other microorganisms are typically added to the hydrolysate and the fermentation is allowed to proceed for 24-96 hours, such as 35-60 hours. The temperature of fermentation is typically between 26-40° C., such as 32° C., and at a pH between 3 and 6, such as about pH 4-5.

A fermentation stimulator may be used to further improve the fermentation process, in particular, the performance of the fermenting microorganism, such as, rate enhancement and ethanol yield. Fermentation stimulators for growth include vitamins and minerals. Examples of vitamins include multivitamin, biotin, pantothenate, nicotinic acid, meso-inositol, thiamine, pyridoxine, para-aminobenzoic acid, folic acid, riboflavin, and vitamins A, B, C, D, and E (Alfenore et al., “Improving ethanol production and viability of Saccharomyces cerevisiae by a vitamin feeding strategy during fed-batch process”, 2002, Springer-Verlag). Examples of minerals include minerals and mineral salts that can supply nutrients comprising phosphate, potassium, manganese, sulfur, calcium, iron, zinc, magnesium and copper.

Recovery. Following fermentation (or SSF), the mash is distilled to extract the ethanol. Ethanol with a purity greater than 96 vol. % can be obtained.

By-Products. The hydrolysis process of lignocellulosic raw material also releases by-products such as weak acids, furans, and phenolic compounds, which are inhibitory to the fermentation process. Removing such by-products may enhance fermentation. In particular, lignin and lignin breakdown products such as phenols, produced by enzymatic activity and by other processing activities, from the saccharified cellulosic biomass is likely to be important to speeding up fermentation and maintaining optimum viscosity.

Thus, in another aspect, the present invention provides methods of speeding up fermentation which comprise removing, from the hydrolysate, products of the enzymatic process that cannot be fermented. Such products comprise, but are not limited to, lignin, lignin breakdown products, phenols, and furans. In certain embodiments, products of the enzymatic process that cannot be fermented can be separated and used subsequently. For example, the products can be burned to provide heat required in some steps of the ethanol production such as saccharification, fermentation, and ethanol distillation, thereby reducing costs by reducing the need for current external energy sources such as natural gas. Alternatively, such by-products may have commercial value. For example, phenols can find applications as chemical intermediates for a wide variety of applications, ranging from plastics to pharmaceuticals and agricultural chemicals. Phenol condensed to with aldehydes (e.g., methanol) make resinous compounds, which are the basis of plastics which are used in electrical equipment and as bonding agents in manufacturing wood products such as plywood and medium density fiberboard (MDF).

Separation of by-products from the hydrolysate can be done using a variety of chemical and physical techniques that rely on the different chemical and physical properties of the by-products (e.g., lignin and phenols). Such techniques include, but are not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, distillation, or extraction.

Some of the hydrolysis by-products, such as phenols, or fermentation/processing products, such as methanol, can be used as ethanol denaturants. Currently about 5% gasoline is added immediately to distilled ethanol as a denaturant under the Bureau of Alcohol, Tobacco and Firearms regulations, to prevent unauthorized non-fuel use. This requires shipping gasoline to the ethanol production plant, then shipping the gas back with the ethanol to the refinery. The gas also impedes the use of ethanol-optimized engines that make use of ethanol's higher compression ratio and higher octane to improve performance. Using transgenic plant derived phenols and/or methanol as denaturants in lieu of gasoline can reduce costs and increase automotive engine design alternatives.

Reducing Lignin Content. Another way of reducing lignin and lignin breakdown products that are not fermentable in hydrolysate is to reduce lignin content in transgenic plant of the present invention. Such methods have been developed and can be used to modify the inventive plants (see, for example, U.S. Pat. Nos. 6,441,272 and 6,969,784, U.S. Pat. Appln. No. 2003-0172395, US and PCT publication No. WO 00/71670).

Combined Starch Hydrolysis and Cellulolytic Material Hydrolysis. The transgenic plants and plant parts disclosed herein can be used in methods involving combined hydrolysis of starch and of cellulosic material for increased ethanol yields. In addition to providing enhanced yields of ethanol, these methods can be performed in existing starch-based ethanol processing facilities.

Starch is a glucose polymer that is easily hydrolyzed to individual glucose molecules for fermentation. Starch hydrolysis may be performed in the presence of an amylolytic microorganism or enzymes such as amylase enzymes. In certain embodiments of the invention, starch hydrolysis is performed in the presence of at least one amylase enzyme. Examples of suitable amylase enzymes include α-amylase (which randomly cleaves the α(1-4)glycosidic linkages of amylose to yield dextrin, maltose or glucose molecules) and glucoamylase (which cleaves the α(1-4) and α(1-6)glycosidic linkages of amylose and amylopectin to yield glucose).

In the inventive methods, hydrolysis of starch and hydrolysis of cellulosic material can be performed simultaneously (i.e., at the same time) under identical conditions (e.g., under conditions commonly used for starch hydrolysis). Alternatively, the hydrolytic reactions can be performed sequentially (e.g., hydrolysis of lignocellulose can be performed prior to hydrolysis of starch). When starch and cellulosic material are hydrolyzed simultaneously, the conditions are preferably selected to promote starch degradation and to activate cell wall-modifying enzyme polypeptide(s) for the degradation of lignocellulose. Factors that can be varied to optimize such conditions include physical processing of the plants or plant parts, and reaction conditions such as pH, temperature, viscosity, processing times, and addition of amylase enzymes for starch hydrolysis.

The inventive methods may use transgenic plants (or plant parts) alone or a mixture of non-transgenic plants (or plant parts) and plants (or plant parts) transformed according to the present invention. Suitable plants include any plants that can be employed in starch-based ethanol production (e.g., corn, wheat, potato, cassaya, etc). For example, the present inventive methods may be used to increase ethanol yields from corn grains.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

### Example 1

### Identification and Isolation of Cell Wall Modifying Enzymes

The present Example describes identification and isolation of enzyme polypeptides that may be useful in breaking down cellulosic biomass. Based on strategies as outlined below, enzyme polypeptides disclosed in the present Example are expected to enhance breakdown of plant cell wall structures, which may comprise networks of hemicellulose, lignin, and pectin.

### Materials and Methods

### Identification of Enzyme Classes of Interest

The inventors have strategized identifying enzymes classes of interest by examining cellulases and xylanases, which are core enzymes to break cellulose and hemicellulose into fermentable sugars. Beyond those core enzyme polypeptides, the inventors have also examined enzyme polypeptides that hydrolyze chemical bonds that link adjacent polymer strands (e.g., hemicellulose to lignin), as these are the major physical bases for cell wall recalcitrance. The inventors have further examined enzyme polypeptides that remove chemical side chains to improve catalytic efficiency (i.e., acetyl xylan esterase removes acetyl groups from the xylan backbone of hemicellulose and subsequently makes that deacetylated xylan a more reactive substrate for xylanase enzymes). Another class of enzymes examined by the inventors are enzyme polypeptides that relieve product feedback inhibition. (For example, beta-glucosidases, by virtue of cleaving cellobiose to 2 molecules of glucose remove the ability of cellobiose to inhibit cellulases).

Some enzymes (e.g., those hydrolyzing pectins) were selected as likely candidates to improve the processing of dicot plant biomass (e.g., poplar) that have relatively high amounts of pectin compared to monocots. Some highly specialized enzymes, such as EcGXX glucuronoxylanase, were selected because they have a stricter substrate specificity than generic xylanases. EcGXX may have less toxic effect when expressed in plants than a less specific xylanase and the glucuronyl side chains that EcGXX requires are involved in crosslinking adjacent polymer strands, thus they specifically target regions of hemicellulose that are involved in recalcitrance.

Using strategies outlined above, microbial organisms that efficiently degrade lignocellulosic biomass were identified by reviewing published scientific reports (Lee et al. (2005) Nuc Acids Res. 33:577-586; Weiner et al. (2008) PLoS Genet. 4:e10000087.; and Martinez et al. (2008) Nat Biotech., 26-36-560, the contents of each of which are herein incorporated by reference in their entirety). Comparative genomic screening was performed by analyzing the genomes of several lignocellulose-degrading microbes in the public CAZy database (http://www.cazy.org/geno/acc_geno.html; Cantrarel et al., 2008) and expression frequencies of various cell wall modifying enzyme classes were plotted to nominally identify classes of enzyme polypeptides that may be useful for degradation of lignocellulosic biomass.

### Identification of Enzyme Candidates Representing Enzyme Classes of Interest

Amino acid sequences for individual enzyme polypeptide candidates (SEQ ID NO: 1 to SEQ ID NO:84) representing identified enzyme classes were retrieved by manually mining the CAZy database and links therein (http://www.cazy.org/index.html) and by directed keyword searching of the PubMed literature database (http://www.ncbi.nlm.nih.gov/sites/entrez).

### Results

Approximately 29 classes of enzyme polypeptides were identified for further study as polypeptides that may be useful in breaking down cellulosic biomass, in particular, cell wall components. These enzyme classes included cellulase and include, but are not limited to, feruloyl esterases, xylanases, alpha-L-arabinofuranosidases, endogalactanases, acetylxylan esterases, beta-xylosidase, xyloglucanases, glucuronoyl esterases, endo-1,5-alpha-L-arabinosidases, pectin methylesterases, endopolygalacturonases, exopolygalacturonases, pectin lyases, pectate lyases, rhamnogalacturonan lyases, pectin acetylesterases, alpha-L-rhamnosidases, mannanases, exoglucanases, licheninases, laminarinases, beta-(1,3)-(1,4)-glucanases and beta-glucosidases. (See Table 2.) From within these enzyme polypeptide classes, amino acid sequences of at least 84 enzyme polypeptides of interest were determined. This list of potentially cell-wall modifying enzyme polypeptides and their polypeptide sequences facilitates further studies, including those described in Examples 3-9.

### Example 2

### Recombinant Protein Expression, Purification, and Characterization

The present Example demonstrates successful expression, purification, and characterization of a variety of recombinant enzyme polypeptides having cell wall-modifying activity, including an exoglucanase (CBH-E), feruloyl esterases (NcFAE and PfFAE), a beta-glucan glucohydrolase (TnGGH), a glucuronoxylan xylanase (EcGXX), and an acetyl xylan esterase (FsAXE).

Materials and methods
Cloning into pHAT Bacterial Expression Vectors

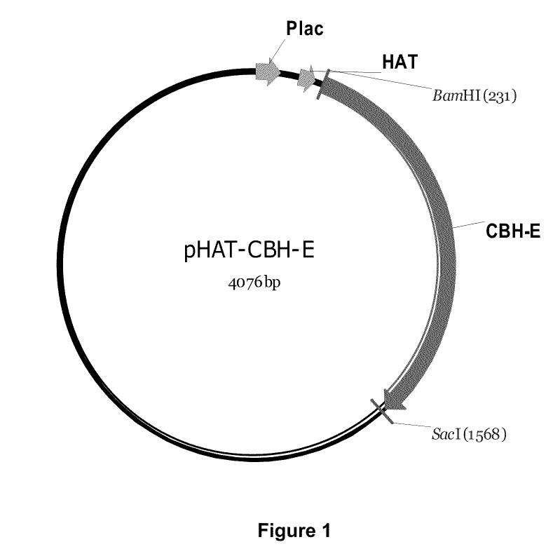

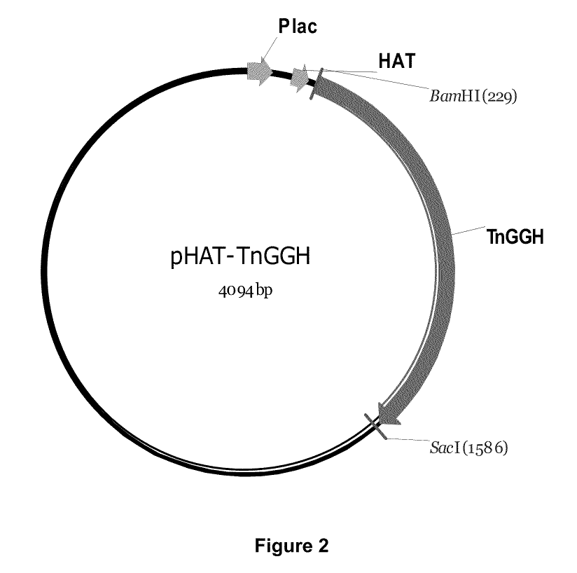

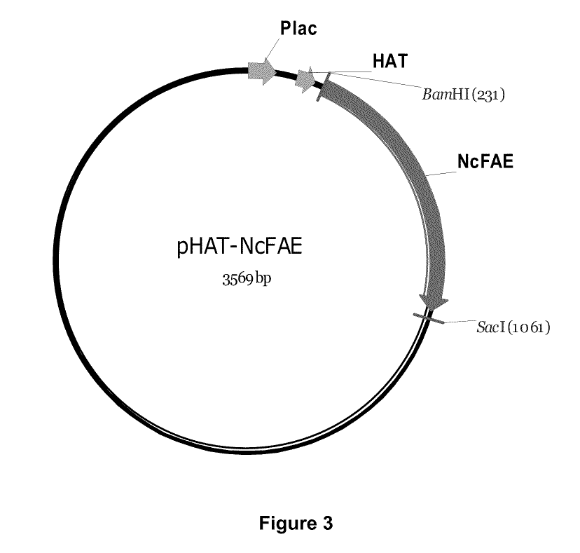

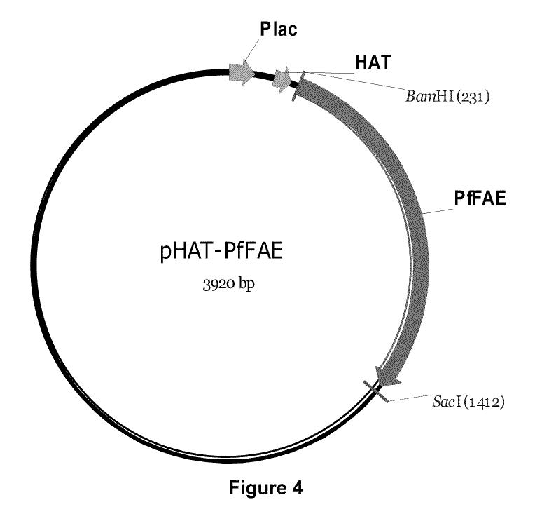

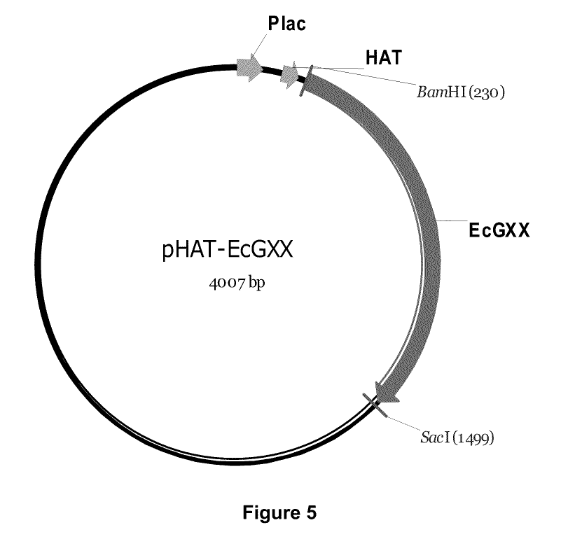

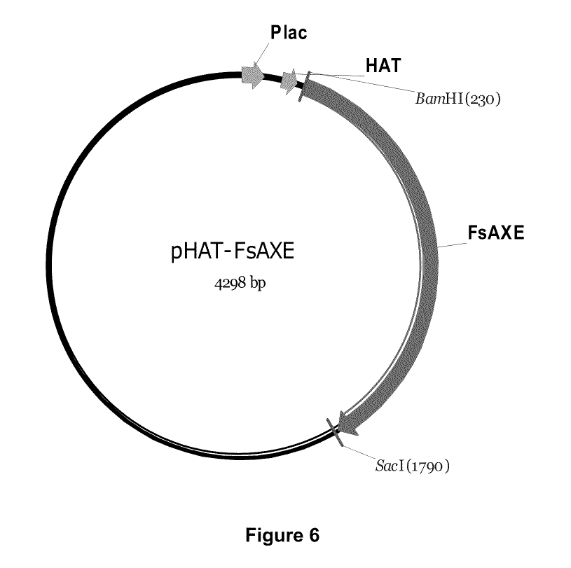

Codon-optimized genes of interest (GOI; SEQ ID NOs: 85-90) encoding enzyme polypeptides were first cloned into Impact Vector 1.2 to add a SacI restriction enzyme site at the 3′ end of the coding region. Genes were digested with BamHI/SacI enzymes and were cloned into pHAT vector series 10/11/12 (Clontech, Mountain View, Calif.) depending upon the translational frame (FIGS. 1-6). Recombinant DNA clones were further transformed into BL21 bacterial cells for protein expression.

### Expression and Purification of HAT-Tagged Fusion Proteins

Transformed E. coli cells were grown in LB media containing 100 μg/ml carbenicillin and 25 μg/ml chloramphenacol. When growth media reached an optical density of 0.6 at A600, IPTG was added to a final concentration of 1 mM to chemically induce recombinant bacterial expression of HAT-tagged enzymes. After adding IPTG, induced cells were incubated for 3 hours, harvested by centrifugation, and lysed by a combination of lysozyme treatment and sonication. Clarified supernatants were prepared by centrifugation and were subsequently incubated with agarose-conjugated cobalt metal ion affinity chromatography resin. Following extensive rinsing of immobilized metal affinity chromatography resin, tightly bound HAT-tagged fusion proteins were eluted with buffer containing imidazole.

### Characterization of Purified HAT-Tagged Fusion Proteins

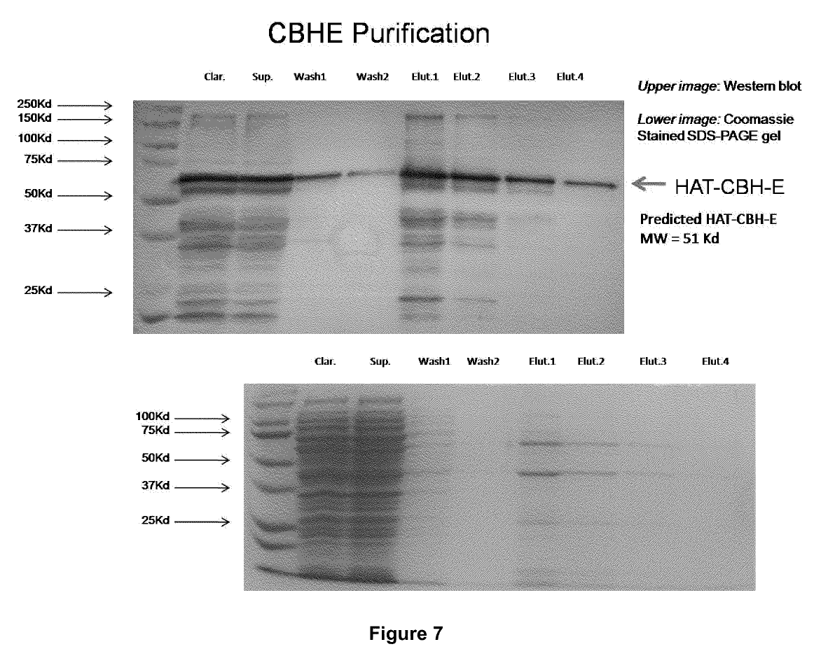

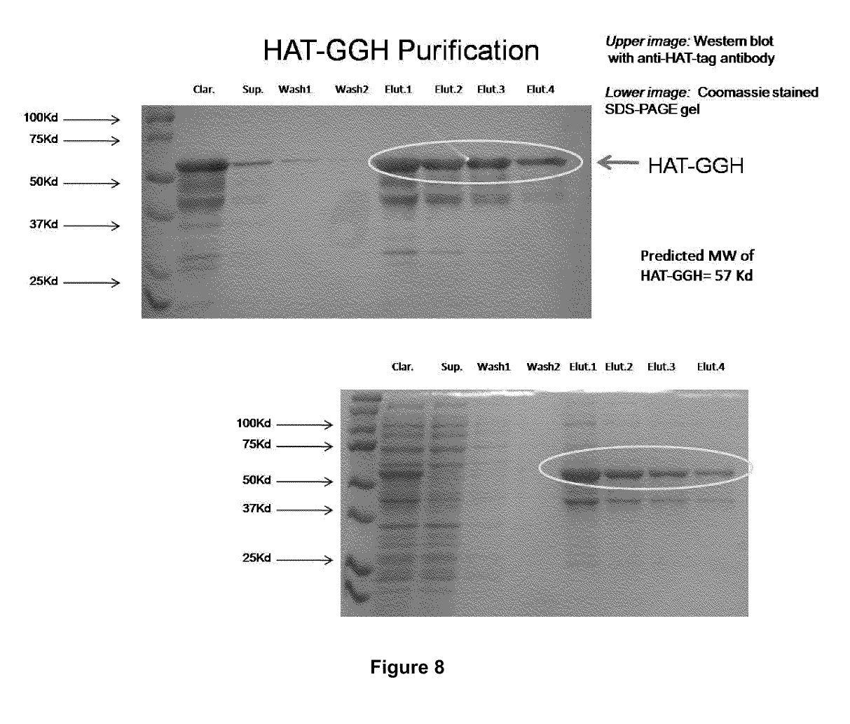

Purified proteins were resolved on 10% SDS-PAGE gels and stained with Coomassie Brilliant Blue dye (lower images in FIGS. 7 and 8) or transferred to PVDF membranes and subsequently immunoblotted with a commercial anti-HAT-tag primary antibody and appropriate horseradish peroxidase (HRP)-labeled secondary antibody. Immunoreactive bands were visualized using an HRP-catalyzed reaction that converts a non-colored substrate into a purple-colored precipitate in situ (upper images in FIGS. 7 and 8).

Measurement of Enzyme Activity Associated with HAT-Purified Proteins

Cellulase activity of purified recombinant HAT-tagged-exoglucanase (CBH-E; SEQ ID NO.: 40; Tuohy et al. (2002) Biochim Biophys Acta. 1596:366-380 (the contents of which are herein incorporated by reference in their entirety)) was determined by incubating the protein with a reaction mixture containing 50 mM sodium acetate, pH 5.0 and substrate (100 μM 4-methylumbelliferyl cellobioside (MUC)) and incubating the sample at 65° C. for a period of time ranging from 1 to 24 hours. At the end of the incubation period, an equal volume of 1M sodium carbonate was added, an aliquot of the mixture was transferred to a black 96-well plate, and release of 4-methylumbelliferone (4-MU) was measured with a fluorescent plate reader (excitation wavelength, 355 nm; emission wavelength, 450 nm).

Cellulase activity of purified recombinant HAT-tagged-cellulase (GGH; SEQ ID NO.:80; Yernool et al. (2000) J Bacteriol. 182:5172-5179 (the contents of which are herein incorporated by reference in their entirety)) was measuring by incubating the protein with a reaction mixture containing 50 mM sodium acetate, pH 5.0 and substrate (250 μM 4-nitrophenyl-β-D-glucopyranoside (pNPG)) and incubating the sample at 90° C. for 1 hour. At the end of the incubation period, an equal volume of 1M sodium carbonate was added and absorbance at 405 nm was measured to detect the release of p-nitrophenol.

### Results

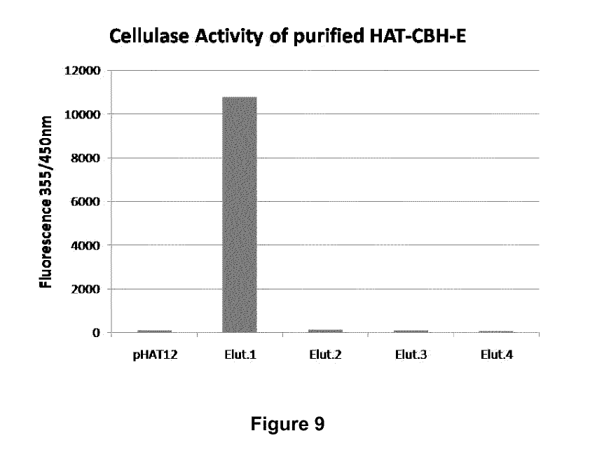

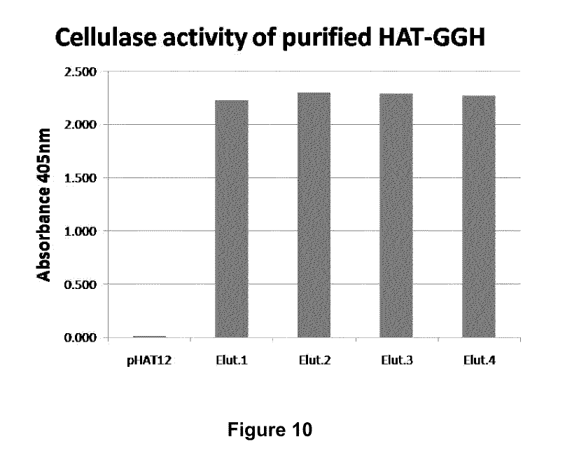

As shown in FIGS. 7 and 8, HAT-tagged enzyme polypeptides were successfully produced and purified. For HAT-CBH-E, the majority of cellulase activity eluted in the first fraction containing imidazole (“Elut. 1” in FIG. 9). No activity was detected in a control sample obtained from bacteria transformed with a vector lacking the exoglucanase GOI insert (“pHAT12” in FIG. 9). For HAT-GGH, cellulase activity was high in all four fractions containing imidazole (“Elut. 1-4” in FIG. 10), and no activity was detected in a control sample obtained from bacteria transformed with the empty vector pHAT12.

These results demonstrate successful production and purification of enzyme polypeptides having cellulase activity.

### Example 3

### Production of Polyclonal Antibodies Against Cell Wall-Modifying Enzyme Polypeptides

The present Example demonstrates successful generation of antibodies against two kinds of cell wall-modifying enzyme polypeptides, a feruloyl esterase and an exoglucanase. Such antibodies are useful for, among other things, purifying and/or detecting such enzyme polypeptides.

### Materials and Methods

Regions of potentially high antigenicity in a feruloyl esterase (NcFAE; SEQ ID NO: 2; Crepin et al. (2004) Appl Microbiol Biotech. 63:567-570 (the contents of which are herein incorporated by reference in their entirety) and an exoglucanase (CBH-E; SEQ ID NO: 40) were identified by analyzing their amino acid sequences. Peptide antigens shown in Table 4 were synthesized and conjugated to a carrier protein (keyhole limpet hemocyanin) and a pair of antigens (one unique to feruloyl esterase and one unique exoglucanase) were separately injected into rabbits.

TABLE 4
Sequences of peptide antigens used to generate
polyclonal antibodies to enzyme polypeptides

| Amino acid sequence | | |
|---|---|---|
| of enzyme poly- | | Corresponding |
| peptide from | | region |
| which antigen | Peptide antigen | of enzyme |
| was derived | sequence¹ | polypeptide² |
| See SEQ ID NO: 2 | CNPSQRDPGQNDPFA | 265-278 |
| See SEQ ID NO: 2 | CRYTVRLPDNYNQNNPY | 53-68 |
| See SEQ ID NO: 40 | CYPNNKAGAKYGTGY | 173-186 |
| See SEQ ID NO: 40 | CNPYRMGNTSFYGPGK | 279-293 |

¹An extra cysteine residue was added to each peptide to facilitate conjugation to the carrier protein. The underlined portion of each sequence refers to the portion derived from the enzyme polypeptide.
²Residues numbers refer to positions in the full-length enzyme polypeptide (as listed in the Sequence Listing) that match the underlined portion of the sequences listed in this Table.

Rabbits received three subsequent booster immunizations, one week apart each, with the same antigens with which they were initially injected. Following the terminal bleed, antibodies were purified using a protein A affinity chromatography column. Affinity-purified antibodies were analyzed by ELISA. Antibody titer was measured based on reactivity against peptide coated on ELISA plates.

### Results

Antibodies were successfully produced against a feruloyl esterase polypeptide (NcFAE; SEQ ID NO: 2) and an exoglucanase polypeptide (CBH-E; SEQ ID NO: 40) by injecting antigenic peptides derived from the enzyme polypeptides into rabbits. As measured by ELISA, antibody titers of affinity-purified antibodies were greater than 1:10,000.

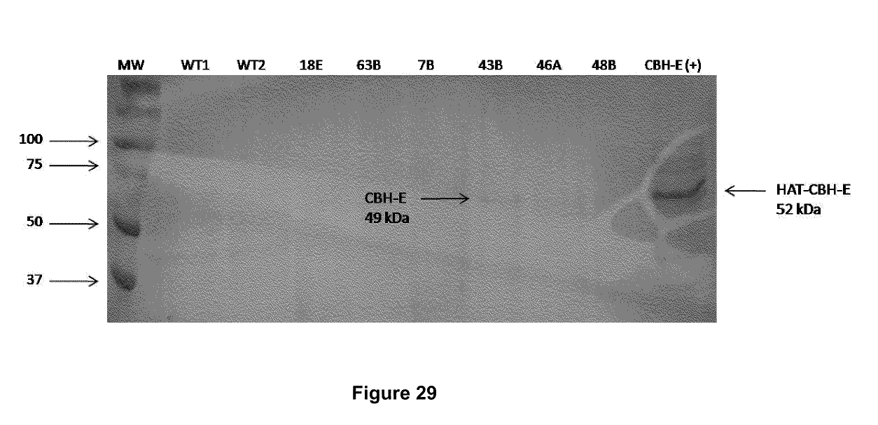

The results demonstrated that anti-CBH-E antibody produced in this Example detected HAT-tagged and native CBH-E. (See FIG. 29.)

### Example 4

### Gene Synthesis and Expression Vector Construction for Expression in Plants

Example 2 demonstrated expression of cell wall-modifying enzyme polypeptides in bacteria. In the present Example, reagents were generated for expressing cell wall-modifying enzyme polypeptides in plants. Codon-optimized genes encoding enzyme polypeptides were synthesized, and plant expression vectors containing these genes and apoplast-targeting sequences were constructed.

### Gene Codon Optimization and Synthesis

Amino acid sequences listed as SEQ ID NOs: 1, 2, 11, 40, 78, and 80 were back-translated into nucleotide sequences (SEQ ID NOs: 85-90, shown in Table 5). Codons for individual amino acid residues were optimized by altering, as necessary, to substitute rare codons for codons of high relative abundance in corn, rice, or dicot plant species. Nucleic acids having these codon-optimized gene sequences for genes of interest (GOI) were synthesized chemically using a commercial vendor.

TABLE 5
Codon-optimized nucleotide sequences encoding
cell wall-modifying enzyme polypeptides

| SEQ ID | |
|---|---|
| NO: | Nucleotide sequence (restriction |
| (Gene) | enzyme site in bold underline) |
| SEQ ID | Corresponding amino acid sequence: SEQ ID |
| NO.: 85 | NO: 1 |
| (CBH-E) | CCATGGATCCACAGCAAGCGGGTACGGCCACCGCGGAGAACC |
| | ATCCCCCCCTTACGTGGCAAGAATGCACCGCCCCCGGATCGT |
| | GCACTACTCAAAATGGCGCTGTGGTTCTCGATGCTAACTGGC |
| | GGTGGGTTCACGATGTTAATGGTTACACTAACTGCTATACAG |
| | GCAATACATGGGACCCGACCTACTGCCTGACGACGAGACTTG |
| | CGCCCAGAACTGCGCACTTGATGGTGCGGATTATGAAGGAAC |
| | GTACGGAGTCACCTCCTCCGGCTCTTCCCTTAAGCTTAATTT |
| | CGTGACAGGCAGCAATGTGGGATCAAGGCTCTATCTGCTCCA |
| | GGACGATTCTACCTACCAAATATTCAAGCTCCTCAACAGAGA |
| | ATTTTCCTTCGACGTCGACGTTTCTAATCTCCCTTGTGGCCT |
| | CAATGGTGCACTCTATTTCGTAGCCATGGACGCAGACGGCGG |
| | AGTCTCGAAATACCCAAACAACAAGGCTGGTGCTAAGTATGG |
| | TACGGGATACTGCGATAGCCAGTGTCCACGCGATCTTAAATT |
| | TATTGACGGTGAAGCAAACGTAGAAGGTTGGCAGCCATCATC |
| | TAACAACGCAAACACAGGTATCGGCGATCACGGCAGCTGTTG |
| | TGCTGAAATGGACGTCTGGGAAGCAAACTCAATATCCAATGC |
| | GGTTACCCCCCATCCTTGCGATACCCCAGGTCAGACGATGTG |
| | CTCTGGAGACGATTGTGGTGGAACCTACTCGAATGACCGCTA |
| | TGCCGGCACCTGCGATCCAGATGGATGCGACTTCAATCCCTA |
| | CCGCATGGGTAATACCTCATTCTACGGCCCCGGAAAAATAAT |
| | TGACACCACGAAGCCTTTCACTGTAGTAACTCAATTTTTGAC |
| | TGACGACGGAACAGACACCGGTACCCTGTCCGAGATCAAAAG |
| | ATTCTACATCCAGAATTCAAACGTCATCCCTCAACCTAATAG |
| | CGACATATCAGGCGTGACCGGTAACTCGATAACAACTGAGTT |
| | TTGCACAGCCCAGAAACAAGCGTTCGGCGACACAGACGATTT |
| | CTCCCAACACGGAGGCCTGGCAAAAATGGGAGCTGCGATGCA |
| | ACAAGGCATGGTACTCGTGATGAGTCTTTGGGATGATTATGC |
| | TGCGCAAATGCTTTGGCTGGATTCCGATTATCCGACAGATGC |
| | AGACCCAACAACCCCAGGAATAGCTAGAGGCACCTGCCCAAC |
| | TGATTCAGGCGTACCGAGCGATGTCGAAAGCCAGTCTCCTAA |
| | TTCTTACGTTACATACTCCAATATTAAGTTCGGACCAATTAA |
| | CTCTACATTCACGGCCTCAGGAGATCT |
| SEQ ID | Corresponding amino acid sequence: SEQ ID |
| NO.: 86 | NO: 2 |
| (NcFAE) | CCATGGATCCAGCTCCCTCCTCCGGCTGCGGAAAAGGACCAA |
| | CTCTGCGCAACGGCCAAACGGTGACAACAAATATTAACGGCA |
| | AGAGTAGGAGATACACCGTGAGGTTGCCGGATAACTACAATC |
| | AGAACAACCCATACCGCCTGATATTCCTCTGGCATCCGCTCG |
| | GATCTTCCATGCAGAAGATCATCCAGGGCGAGGACCCCAACA |
| | GAGGCGGCGTCCTGCCTTACTACGGCCTGCCGCCGCTCGATA |
| | CATCCAAGTCAGCCATCTATGTGGTTCCGGATGGATTGAACG |
| | CGGGCTGGGCGAATCAGAACGGAGAGGACGTCTCATTCTTTG |
| | ATAACATCTTGCAAACCGTGTCAGACGGTCTGTGTATCGACA |
| | CAAATCTTGTGTTCAGCACCGGCTTCAGCTACGGAGGGGGCA |
| | TGTCTTTCTCCCTTGCCTGCAGCCGCGCGAACAAGGTGCGCG |
| | CTGTCGCCGTGATTAGTGGTGCACAGCTCTCCGGGTGCGCAG |
| | GCGGAAACGACCCGGTGGCGTACTACGCTCAGCACGGTACCA |
| | GCGACGGCGTCCTTAATGTGGCGATGGGCCGCCAGCTCCGGG |
| | ACAGGTTCGTCAGGAACAACGGCTGCCAGCCCGCCAATGGCG |
| | AGGTGCAGCCAGGCAGTGGAGGAAGGAGCACCCGCGTCGAAT |
| | ACCAAGGTTGTCAGCAAGGCAAGGATGTGGTGTGGGTCGTTC |
| | ACGGCGGGGACCACAACCCATCCCAAAGGGACCCCGGTCAGA |
| | ATGACCCGTTCGCTCCTAGGAACACCTGGGAATTTTTCAGTC |
| | GCTTCAACTAAGGCGCGCCAGATCT |
| SEQ ID | Corresponding amino acid sequence: SEQ ID |
| NO.: 87 | NO: 11 |
| (PfFAE) | CCATGGATCCAGAGCAGACCCAAACACAGACACTTGAGTCGA |
| | ACAGCCCGACTCAAACCACAACCACGACCAGCCCTCAAATCA |
| | CTGTGACTTTCATTGTCTCAGTCCCCGAATACACCCCTGAGA |
| | ATGACTCTATCTATATCGCGGGCGACTTCAACAACTGGAATC |
| | CGAAGGATGAAAGATACAAGCTGGTGAAGCTGCCGGACGGGA |
| | GGTGGAAGATTACTCTCACCTTCCCTTACGGTAAGACCATCC |
| | AGTTCAAGTTCACGCGCGGCTCCTGGGAGACGGTGGAGAAGG |
| | GCATCAACGGCGAGGAGATCCCGAACCGCAGATTTACGTTCA |
| | CGAAGAGCGGCACCTATGAATTTAAGGTTCACAATTGGAGAG |
| | ATTTTGTGGAAAAGAATGTGAAGCACACAATCACCGGCAACG |
| | TGATCACTTTCGAGATGTTCATCCCACAGCTCAACACCACAA |
| | GGAGAATCTGGATCTATCTCCCACCGGACTACAACTACTCAA |
| | CCAAGCGCTACCCGGTGCTCTACATGTTCGATGGCCAGAATC |
| | TGTTCGATGCGGCAACATCTTTCGCTGGGGAGTGGGGAGTGG |
| | ACGAAGCGCTTGAGAAGCTTTACAAGGAAAAGAATTTCTCCA |
| | TTATTGTTGTCGGCATTGATAACGGCGGCGACAGGCGCATTG |
| | ATGAGTATGCCCCTTGGGTTAACCGGGATTACAGAAGGGGTG |
| | GACTGGGAAACGCCACCGTCAAGTTCATAGTCGAGACGCTGA |
| | AGCCTTACATTGACGCGCACTACAGGACAGACCCCGAAAAGA |
| | CCGGTATCATGGGAAGCAGCCTGGGAGGCCTGATGGCTATAT |
| | ATGCCGGTTTCTCTTATCCGGAAGTGTTCAGGTACGTAGGCG |
| | CCATGTCGAGTGCCTTCTGGTTTAACCCGGAAATTTATGATT |
| | TCGTTCGCGAGGCCAAGAAGGGCCCAGAGAAGATTTATATCG |
| | ACTGGGGTACCAACGAAGGCCGCAACCCGAAGGCGTTCAGCG |
| | AGAGTAACGAGAAAATGGTCAAGATCCTCAAAGAGAAGGGGT |
| | ACCGCGAGGAGTTCAACCTCAAGGTCGTGATCGATAAAGGAG |
| | GGCTGCACAACGAGTATTACTGGGGAAAGAGATTCCCTCAGG |
| | CCGTGTTGTGGCTCTTCGAGGAGTAAGGCGCGCCAGATCTGA |
| | GCTC |
| SEQ ID | Corresponding amino acid sequence: SEQ ID |
| NO.: 88 | NO: 40 |
| (TnGGH) | CCATGGATCCTAAGAAGTTCCCGGAGGGCTTTCTCTGGGGCG |
| | TGGCGACCGCCAGCTACCAGATCGAGGGCTCCCCACTCGCCG |
| | ATGGCGCAGGCATGTCCATCTGGCACACCTTCAGTCACACGC |
| | CGGGCAATGTCAAGAACGGTGACACCGGCGACGTGGCTTGCG |
| | ACCACTACAACCGCTGGAAGGAGGACATCGAGATCATAGAGA |
| | AGATCGGCGCCAAGGCCTACAGGTTTTCCATCTCCTGGCCAA |
| | GGATACTCCCGGAGGGAACCGGCAAGGTCAACCAGAAGGGCC |
| | TCGACTTTTACAACCGGATCATTGACACCCTCCTGGAGAAGA |
| | ACATCACCCCGTTCATCACCATCTACCACTGGGATCTCCCCT |
| | TTTCCCTTCAGCTCAAGGGCGGCTGGGCCAACAGGGACATCG |
| | CTGATTGGTTCGCCGAGTATTCCCGCGTGCTCTTCGAGAACT |
| | TCGGCGACAGAGTGAAGCACTGGATCACCCTCAACGAGCCGT |
| | GGGTGGTGGCCATCGTTGGCCACCTCTACGGCGTGCACGCCC |
| | CAGGCATGAAGGATATATACGTGGCTTTCCACACCGTGCACA |
| | ATCTCCTTAGGGCCCACGCGAAGAGCGTGAAGGTGTTTAGGG |
| | AAACCGTGAAGGACGGCAAGATCGGCATTGTGTTCAACAATG |
| | GCTACTTCGAGCCGGCTTCCGAGAGGGAAGAGGACATCAGGG |
| | CCGCCAGGTTTATGCACCAGTTCAATAACTACCCGCTGTTTC |
| | TCAACCCGATATACAGGGGCGAGTACCCGGACCTCGTGCTTG |
| | AGTTCGCCAGGGAATACCTGCCCAGGAACTACGAGGATGACA |
| | TGGAGGAAATCAAGCAGGAGATTGACTTCGTGGGCCTCAACT |
| | ACTACAGTGGCCACATGGTGAAGTACGATCCGAACTCCCCAG |
| | CCAGGGTGTCCTTCGTGGAGAGGAACCTCCCAAAGACCGCTA |
| | TGGGCTGGGAGATCGTTCCGGAGGGCATATACTGGATTCTCA |
| | AGGGCGTGAAGGAGGAGTACAACCCGCAGGAGGTGTATATCA |
| | CCGAGAACGGCGCTGCCTTCGACGATGTTGTGTCCGAGGGCG |
| | GTAAAGTGCACGACCAGAACAGGATCGACTACTTGCGAGCCC |
| | ATATTGAGCAGGTCTGGAGGGCAATTCAGGATGGCGTTCCGC |
| | TCAAGGGGTACTTCGTGTGGTCCCTGCTCGACAATTTTGAGT |
| | GGGCCGAGGGCTACTCCAAGAGGTTCGGCATCGTTTACGTGG |
| | ACTACAACACCCAGAAGAGGATCATTAAGGACTCCGGCTACT |
| | GGTACAGTAACGGCATCAAAAACAACGGCCTCACCGACTAAG |
| | GCGCGCCAGATCTGAGCTC |
| SEQ ID | Corresponding amino acid sequence: SEQ ID |
| NO.: 89 | NO: 78 |
| (EcGXX) | CCATGGCAAACGGCAACGTGTCCCTCTGGGTGAGGCACTGCC |
| | TCCACGCAGCACTCTTCGTGTCCGCAACCGCAGGCTCCTTCT |
| | CCGTGTACGCCGACACCGTGAAGATCGACGCCAACGTGAACT |
| | ACCAGATCATCCAGGGCTTCGGCGGCATGTCCGGCGTGGGCT |
| | GGATCAACGACCTCACCACCGAGCAGATCAACACCGCCTACG |
| | GCTCCGGCGTGGGCCAGATCGGCCTCTCCATCATGAGGGTGA |
| | GGATCGACCCGGACTCCTCCAAGTGGAACATCCAGCTCCCGT |
| | CCGCCAGGCAGGCCGTGTCCCTCGGAGCAAAGATCATGGCAA |
| | CCCCGTGGTCCCCACCAGCCTACATGAAGTCCAACAACTCCC |
| | TCATCAACGGCGGCAGGCTCCTCCCGGCCAACTACTCCGCCT |
| | ACACCTCCCACCTCCTCGACTTCTCCAAGTACATGCAGACCA |
| | ACGGCGCCCCGCTCTACGCCATCTCCATCCAGAACGAGCCGG |
| | ACTGGAAGCCGGACTACGAGTCCTGCGAGTGGTCCGGCGACG |
| | AGTTCAAGTCCTACCTCAAGTCCCAGGGCTCCAAGTTCGGCT |
| | CCCTCAAGGTCATCGTGGCAGAGTCCCTCGGCTTCAACCCAG |
| | CACTCACCGACCCGGTGCTCAAGGACTCCGACGCCTCCAAGT |
| | ATGTGAGCATTATCGGAGGACACCTCTACGGAACCACCCCAA |
| | AGCCATACCCACTCGCACAGAACGCAGGCAAGCAGCTCTGGA |
| | TGACCGAGCACTACGTGGACTCCAAGCAGTCCGCCAACAACT |
| | GGACCTCCGCCATCGAAGTGGGCACCGAGCTGAACGCCAGCA |
| | TGGTGTCCAACTACTCCGCCTACGTGTGGTGGTACATCAGGA |
| | GGTCCTATGGCCTCCTCACCGAGGACGGCAAGGTGTCCAAGA |
| | GGGGCTACGTGATGTCCCAGTACGCCAGGTTCGTGAGGCCGG |
| | GCGCCCTCAGAATCCAGGCCACCGAGAACCCGCAGTCCAACG |
| | TGCACCTCACCGCCTACAAGAACACCGACGGAAAGATGGTCA |
| | TCGTGGCCGTGAACACCAACGACTCCGACCAGATGCTCTCCC |
| | TCAACATCTCCAACGCCAACGTGACCAAGTTCGAGAAGTACT |
| | CCACCTCCGCCTCCCTCAACGTGGAGTACGGAGGCTCCTCCC |
| | AGGTGGACTCCTCCGGCAAGGCAACCGTGTGGCTCAACCCAC |
| | TCTCCGTGACCACCTTCGTGTCCAAGTCAGATCTC |
| SEQ ID | Corresponding amino acid sequence: SEQ ID |
| NO.: 90 | NO: 80 |
| (FsAXE) | CCATGGCAGCCCCGGACCCGAACTTCCACATCTACATCGCCT |
| | ACGGCCAGTCCAACATGGAGGGCAACGCCAGGAACTTCACCG |
| | ACGTGGACAAGAAGGAGCACCCGAGGGTGAAGATGTTCGCAA |
| | CCACCTCCTGCCCGTCCCTCGGAAGGCCAACCGTGGGAGAGA |
| | TGTACCCAGCAGTGCCACCAATGTTCAAGTGCGGAGAGGGAC |
| | TCTCCGTGGCAGACTGGTTCGGAAGGCACATGGCAGACTCCC |
| | TCCCAAACGTGACCATCGGCATCATCCCAGTGGCACAGGGAG |
| | GCACCTCCATCAGGCTCTTCGACCCGGACGACTACAAGAACT |
| | ACCTCAACTCCGCCGAGTCCTGGCTCAAGAACGGCGCCAAGG |
| | CCTACGGCGACGACGGCAACGCTATGGGAAGGATCATCGAGG |
| | TGGCCAAGAAGGCCCAGGAGAAGGGCGTGATCAAGGGCATCA |
| | TCTTCCACCAGGGCGAGACCGACGGCGGCATGTCCAACTGGG |
| | AGCAGATCGTGAAGAAGACCTACGAGTACATGCTCAAGCAGC |
| | TCGGCCTCAACGCAGAGGAGACCCCATTCGTGGCAGGAGAGA |
| | TGGTGGACGGAGGCTCCTGCGCAGGCTTCTCCTCCAGGGTGA |
| | GGGGCCTCTCCAAGTACATCGCCAACTTCGGCGTGGCCTCCT |
| | CCAAGGGCTACGGCTCCAAGGGCGACGGCCTCCACTTCACCG |
| | TGGAGGGCTACAGGGGCATGGGCCTCCGCTACGCCCAGCAGA |
| | TGCTCAAGCTCATCAACGTGGCACCAGTGGACCCGGTGCCAC |
| | AGGAGCCGTTCAAGGGTGCTCCAATCGCAATCCCAGGCAAGG |
| | TGGAGGTGGAGGACTTCGACAAGCCGGGCATCGGCAAGAACG |
| | AGGACGGCACCTCCAACGCCTCCTACTCCGACGAGGACTCCG |
| | AGAACCACGGCGACTCCGACTACAGGAAGGACACCGGAGTGG |
| | ACCTCTACAAGGCAGGCGACGGAGTGGCACTCGGATACACCC |
| | AGACCGGAGAGTGGCTGGAGTACACCGTGGACGTGAAGGCCG |
| | ACGGCGAGTACAACATCGACGCCTCCGTGGCCGCCGGCAACT |
| | CCACCTCCGCCTTCAAGCTCTACATCGACGAGAAGGCCATCA |
| | CCGACGACGTGTCCGTGCCGCAGACCGCCGACAACTCCTGGG |
| | ACACCTACAAGACCATCTCCGTGAAGGAGAAGGTGACCCTCA |
| | AGGCCGGCAAGCACGTGCTCAAGCTGGAGATCACCGCCAACT |
| | ACGTGAACATCGACTGGATTCAGTTCTCCGAGCCGAAGAAGG |
| | AGGACCCGCCGTCCGCCATCGCCAAGGTGAGGTTCGACATGA |
| | CCGAGGCCGAGTCCAACTTCTCCGTGTACTCCATGCAGGGCC |
| | AGAAGCTCGGCACCTTCACCGCCAAGGGCATGGCCGACGCCA |
| | TGAACCTCGTGAAGACCGACGCCAAGCTCAGGAAGCAGGCCA |
| | AGGGCGTGTTCTTCGTGAGGAAGGAGGGCGCCAAGCTCATGT |
| | CCAAGAAGGTGGTGGTGTTCGAGTCAGATCTC |

### Cloning of Plant Transformation Vectors

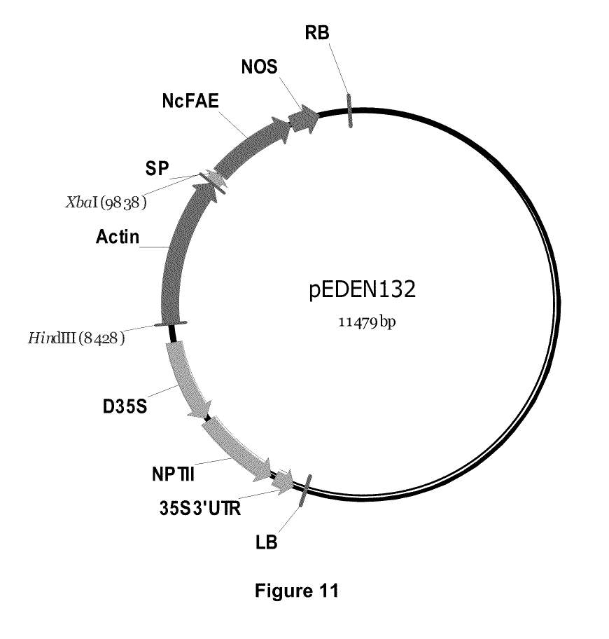

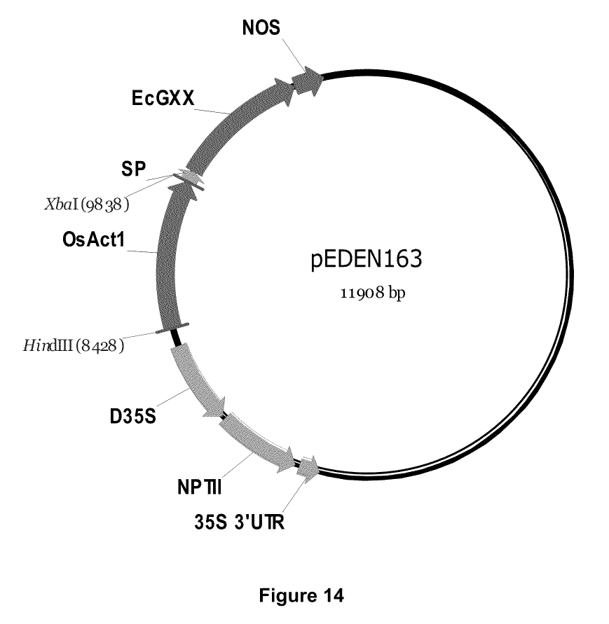

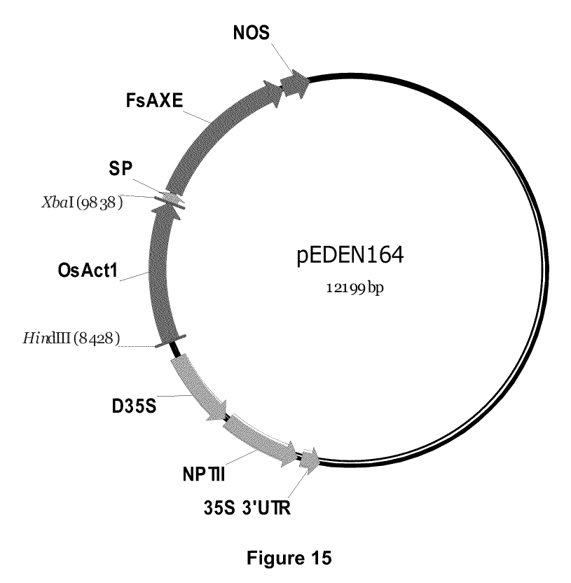

Appropriate restriction enzyme sites were engineered at the ends of gene-coding regions, and the specified DNA was synthesized by a commercial vendor. Nucleic acids were digested with BamHI/BglII enzymes and were cloned into Impact Vector 1.2 so as to create in-frame fusions with an N-terminal apoplast targeting signal peptide. The rice Actin (OsAct1) promoter or 35S promoter was cloned into the resulting vector as a HindIII/XbaI fragment to drive corresponding genes of interest. Finally, gene cassettes comprising OsAct1-GOI or 35S-GOI were cloned into pPZPY112 to create transformation-ready binary vectors (FIGS. 11-17 for plasmid maps).

Plant expression vectors generated in the present Example were used to induce expression in a variety of plants, including corn, poplar, and tobacco, as described in Examples 5-10.

### Example 5

### Stable Transformation of Corn to Express Enzyme Polypeptides

In the present Example, corn plants were stably transformed to express cell wall-modifying enzyme polypeptides.

### Materials and Methods

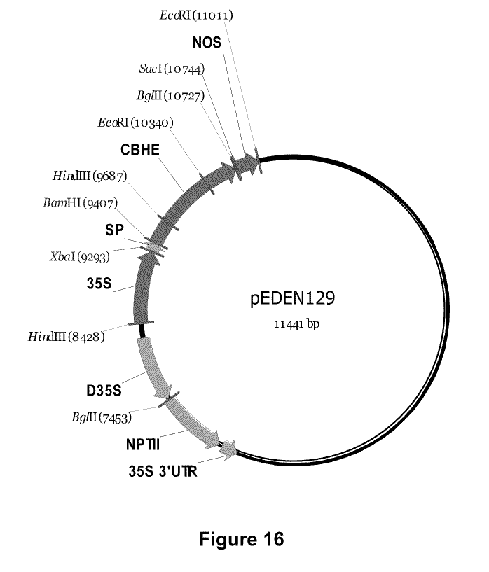

Stable transformation of corn was performed according to a protocol using immature embryos of the Hi-II corn genotype. (See FIG. 16.) The protocol developed by Frame et al. ((2002) Plant Physiol. 129:13-22) (the contents of which are herein incorporated by reference in their entirety) was modified to adapt to a selection strategy using paromomycin and a neomycin phosphotransferase II (NPTII) gene (Prakash et al., (2008) Transgenic Res. 17:695-704). Immature embryos were isolated and infected with Agrobacterium containing the expression construct. Infected embryos were co-cultivated with Agrobacterium for 3 days in the dark. After co-cultivation, infected embryos were moved to selection medium containing paromomycin (100 mg/L) and incubated in an incubator at 27° C. in the dark in a plant tissue culture chamber. Resistant Type II calli induced from immature embryos were selected for 8 weeks at 27° C. in the dark with 200 mg/L of paromomycin. After 4 rounds of selection, proliferated embryogenic calli were sub-cultured into somatic embryo maturation medium for two weeks at 27° C. in dark. Matured somatic embryos were subcultured on regeneration medium for another two weeks under light at 27° C. (16 h/8 h light/dark cycle, Conviron TC26; tissue culture chamber). Green and elongated somatic embryos that emerged in 2-4 weeks were transferred to basic nutrient medium for further elongation and rooting in magenta boxes and grown at 27° C. under a 16 h/8 h light/dark photoperiod. Plantlets with well-established roots were transferred to soil and acclimatized in a plant growth chamber (Conviron, Adaptis A1000). After molecular characterization for transgene integration plants were moved to green house and grown to maturity.

### Results

Corn plants were successfully transformed with expression vectors encoding cell wall-modifying enzyme polypeptides, including a feruloyl esterase polypeptide and an exoglucanse polypeptide. Characterization of transformed plants, including analyses of cellulase activity and impact on digestibility of plant tissue, is described in Example 6.

### Example 6

### Characterization of Corn Plants Stably Transformed with a Construct for Expressing a Feruloyl Esterase

Corn plants were stably transformed with expression vectors as described in Example 4. The present Example presents experimental results characterizing corn plants that had been transformed with expression vectors for a feruloyl esterase.

### Materials and Methods

### Screening of Transgenic Plants

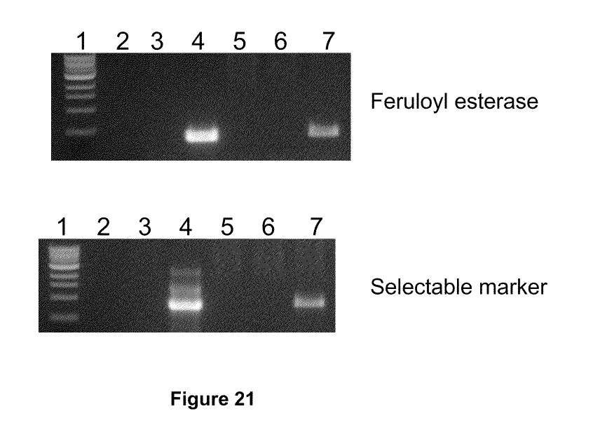

Corn plants were transformed with pEDEN132 (FIG. 11), an expression vector encoding a feruloyl esterase, and selected for paromomycin resistance as described in Example 4. Paromomycin-selected plants were screened by PCR for presence of NcFAE (a Neurospora crassa feruloyl esterase whose amino acid sequence is listed as SEQ ID NO: 2) and npt II (the selectable marker) genes, using NcFAE and npt II primers as listed in Table 6. Plants for which positive signals for NcFAE and the selectable marker were detected by PCR (see FIG. 21 for an example) were chosen for further study.

TABLE 6
Nucleotide sequences of primers used for PCR
analysis

| | | SEQ | |
|---|---|---|---|
| Primer | Primer | ID | |
| Name | ID | NO: | Primer Sequence (5′ to 3′) |
| Feruloyl | ES463 | 90 | ACG GCC AAA CGG TGA CAA CAA A |
| esterase | | | |
| (NcFAE)- | | | |
| forward | | | |
| Feruloyl | ES464 | 91 | AGC CGG TGC TGA ACA CAA GAT T |
| esterase | | | |
| (NcFAE)- | | | |
| reverse | | | |
| Exoglu- | ES455 | 92 | ACA GGC AGC AAT GTG GGA TCA A |
| canase | | | |
| (CBH-E)- | | | |
| forward | | | |
| Exoglu- | ES456 | 93 | TGT TGC ATC GCA GCT CCC ATT T |
| canase | | | |
| (CBH-E)- | | | |
| reverse | | | |
| Promoter-SM | ES531 | 94 | TTC ATT TCA TTT GGA GAG GAC A |
| (D35S- | | | |
| nptII)- | | | |
| forward | | | |
| Promoter-SM | ES532 | 95 | CAA GCT CTT CAG CAA TAT CAC G |
| (D35S- | | | |
| nptII)- | | | |
| reverse | | | |

### Measurement of Feruloyl Esterase Activity of Transgenic Corn

Leaves harvested from individual corn plants were flash frozen with liquid nitrogen and ground using a mortar and pestle. Duplicate five milligram samples of ground corn material were incubated with a reaction mixture containing 50 mM sodium acetate pH 5.0 and substrate (250 mM 4-methylumbelliferyl p-trimethylammonio-cinnamate chloride (MUTMAC)) at 37° C. for 2 h to determine feruloyl esterase activity. After the incubation period, 0.5 volumes of 1M Tris pH 7.5 were added to each sample and an aliquot of each sample was used to measure fluorescence (excitation wavelength 355 nm; emission wavelength 450 nm) with a plate reader.

### Tempering, Enzyme Digestion, Sugar Yield Analysis, and Digestibility Assays

Composite corn stover samples were obtained by combining several samples that had feruloyl esterase activity. A control composite corn stover sample was similarly obtained by combining samples that had no detectable feruloyl esterase activity. Extractive compounds were removed from composite samples using a standard ethanol-acetone extraction procedure and dried to completeness in a fume hood. The tare weight of empty sample tubes was recorded and ground material (˜50 mg) was transferred to each tube. Dry weights of each sample plus tube was recorded. Samples were treated according to their experimental group.

Half of the feruloyl esterase (SEQ ID NO: 2) and half of the control samples formed the “Tempered” group and were reconstituted in sodium acetate buffer, pH 5.0 containing 0.02% sodium azide and incubated at 37° C. for 24 h. The other half of the samples, the “Not Treated” group, were kept in their dry state. Samples in the Tempered group were centrifuged following tempering and the supernatant was discarded. Samples in the Tempered and Not Treated groups were reconstituted in buffer (sodium acetate pH 5.0, 5 mM CaCl₂, and 0.02% sodium azide) containing 8 mg Novozymes Celluclast 1.5 L/g of starting dry weight and 0.2 units of Novozymes 188 β-glucosidase. Samples were incubated at 50° C. for 24 h. A sample of the supernatant was then analyzed for reducing sugars using the DNS assay, and solids were rinsed extensively with water to remove hydrolyzed materials liberated during the 24 h hydrolysis period.

After enzyme digestion, samples were dried to completeness in a dehydrator, and the final dry weight of the sample plus tube was recorded. The amount of mass lost during enzyme digestion was determined by subtracting the final sample weight from the starting weight. The digestibility of a sample was determined by calculating percentage of mass lost during the in vitro dry matter digestibility (IVDMD) procedure. Data were graphed and analyzed by one-way Analysis of Variance (ANOVA) with post-hoc testing using the Tukey method.

### Xylanase Treatment

Triplicate 5 milligram samples of ethanol-acetone extracted (as described above) feruloyl esterase-expressing and control corn biomass were incubated for 30 minutes at 50° C. in buffer (50 mM sodium acetate pH 5.0) containing 0, 0.1, or 1 unit of Trichoderma viride beta-xylanase M1 (Megazyme). Reducing sugars were then determined using the DNS reagent and absorbance at 540 nm was measured using a plate reader.

### Results

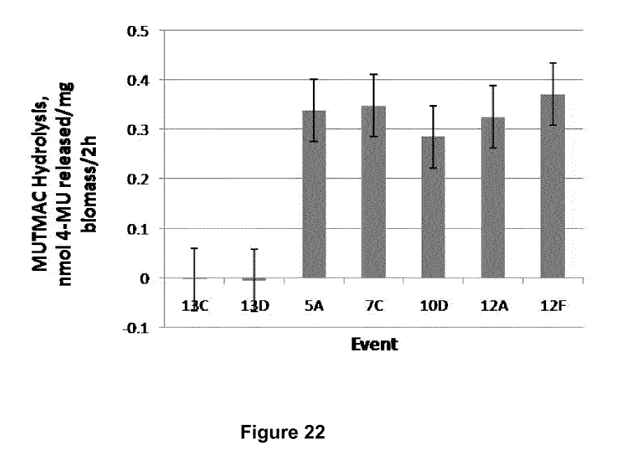

Corn plants identified as bearing feruloyl esterase and selection marker genes were examined for feruloyl esterase activity. As depicted in FIG. 22, feruloyl esterase activity was detected in a number of samples from different transformation events. Five samples from four different transformation events (5A, 7C, 10D, 12A, and 12F) showed high feruloyl esterase activity, whereas two samples (siblings 13C and 13D from transformation event 13) showed no activity. A composite sample of feruloyl esterase transgenic biomass was prepared by thoroughly mixing samples 5A, 7C, 10D, 12A, and 12F. Ground biomass from several event 13 siblings was thoroughly mixed to use as a composite control sample. These composite samples were used for further analyses.

Samples were divided into two groups: the “tempered” group, which were incubated at 37° C. for 24 h before digestion, and the “not treated” group. Without wishing to be bound by any particular theory, it is contemplated that such an incubation step may improve sugar yield by allowing enzymes in the biomass to become activated.

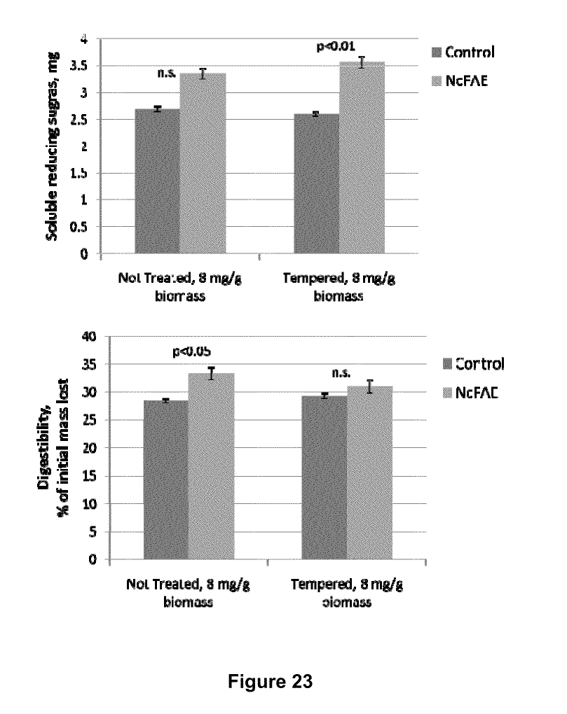

To examine whether transgenic expression of feruloyl esterase had any effect on sugar yield, samples were inciated in an enzyme mixture and the resulting supernatant analyzed for reducing sugars using a DNS (3,5-dinitrosalicylic acid) assay. As shown in the right side of the upper panel of FIG. 23, pre-incubating the samples in a tempering” step led to a significant increase in the amount of reducing sugars released from feruloyl esterase-expressing stover as compared to the control (non-feruloyl esterase-expressing stover) sample. A smaller increase was also observed in the “not treated” group, but this increase did not reach statistical significance in this experiment.

Without wishing to be bound by any particular theory, it is contemplated that the tempering step may have allowed increased sugar yield from feruloyl esterase-expressing stover by allowing feruloyl esterase to hydrolyze cell wall substrates before digestion by externally added enzyme.

To examine whether transgenic expression of feruloyl esterase had any effect on digestibility of corn stover, an in vitro dry matter digestibility (IVDMD) assay was used. In this assay, the percentage of mass lost during enzyme digestion is used as an indication of digestibility. As shown in the lower panel of FIG. 23, among the “not treated” samples, feruloyl esterase-transgenic stover was more digestible than non-transgenic stover (p<0.05). The difference in digestibility observed between transgenic stover and non-transgenic stover among the tempered samples did not reach statistical significance.

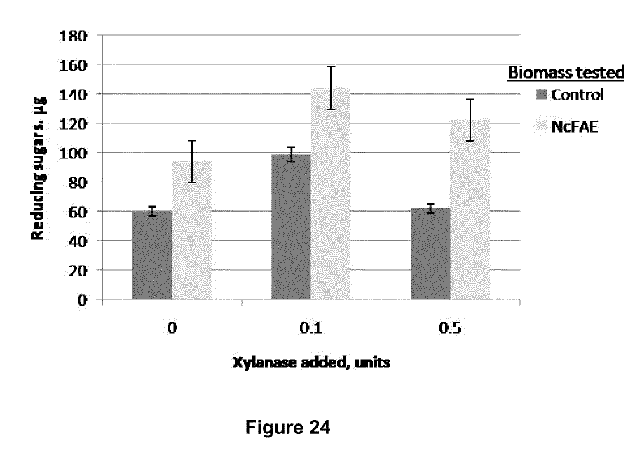

To examine whether sugar yields could be enhanced further by using externally added xylanase, feruloyl esterase-expressing biomass was incubated in xylanase. As shown in FIG. 24, corn biomass expressing feruloyl esterase produces significantly more reducing sugars in the presence of xylanase compared non-transgenic control corn biomass treated with xylanase.

These results indicate that feruloyl esterase improves the digestibility of hemicellulose by externally added enzymes such as xylanase. Without wishing to be bound by any particular theory, it is contemplated that such improvement was observed because feruloyl esterases hydrolyze diferulate ester linkages. Ester bonds link cinnamic acids to hemicellulose, and both diferulate and monoferulate esters are known to impair the accessibility of xylanases to the xylan backbone of hemicellulose.

### Example 7

### Characterization of Corn Plants Stably Transformed with a Construct for Expressing an Exoglucanase

Corn plants were stably transformed with expression vectors as described in Example 4. The present Example presents experimental results characterizing corn plants that had been transformed with expression vectors for an exoglucanase.

### Materials and Methods

### Screening of Transgenic Plants

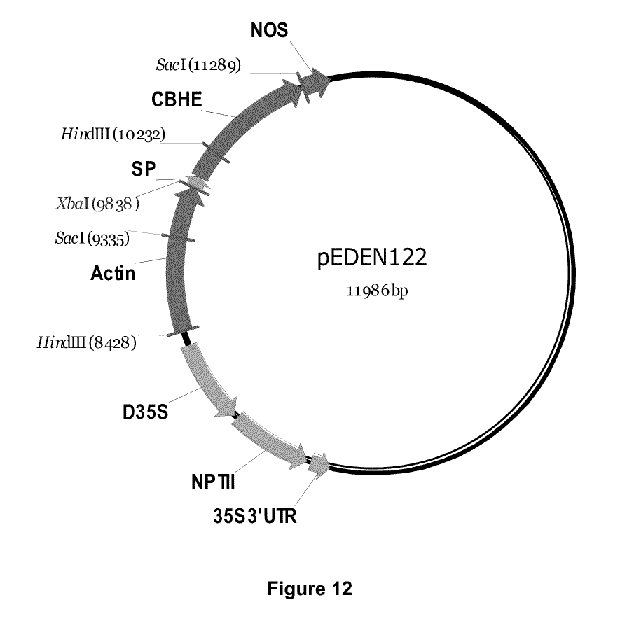

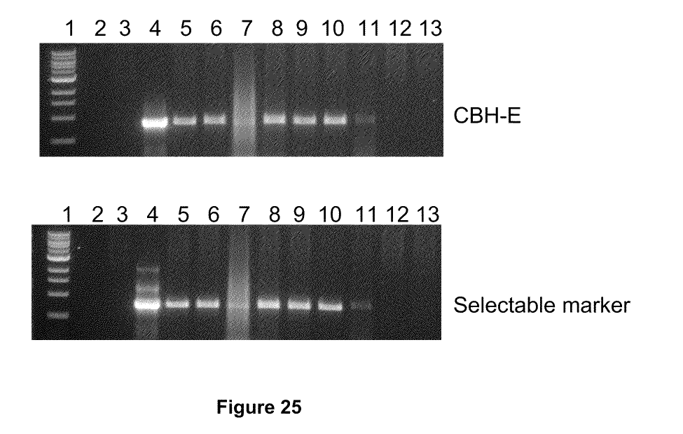

Corn plants were transformed with pEDEN122 (FIG. 12), an expression vector encoding CBH-E (an exoglucanase expressed by Talaromyces emorsonii) and selected for paromomycin resistance as described in Example 4. Paromomycin-selected plants were screened by PCR for presence of CBH-E (whose amino acid sequence is listed as SEQ ID NO: 40) and npt II (the selectable marker) genes, using CBH-E and npt II primers as listed in Table 6. (See Example 8. Plants for which positive signals for CBH-E and the selectable marker were detected by PCR (see FIG. 25) were chosen for further study.

### Tempering, Enzyme Digestion, Sugar Yield Analysis, and Digestibility Assays

Extractive compounds were removed from exoglucanase-expressing and control corn stover composite samples using a standard ethanol-acetone extraction procedure and dried to completeness in a fume hood. The tare weight of empty sample tubes was recorded and then ground material from exoglucanase-expressing and control biomass (˜50 mg) was transferred to each tube. The dry weight of the sample plus the tube was recorded. Samples were then treated according to their experimental group. Half of the exoglucanase-expressing and control samples, the “Pretreated” group, were reconstituted in 100 mM sulfuric acid and heated at 120° C. for 10 minutes followed by neutralization with 0.5 N sodium hydroxide. The second half of the samples, the “Not Treated” group, were kept in their dry state. Following neutralization, samples in the Pretreated group were centrifuged and the supernatant was discarded. Samples in the Pretreated and Not Treated groups were reconstituted in buffer (sodium acetate pH 5.0, 5 mM CaCl₂, and 0.02% sodium azide) containing either 0.4 mg or 8 mg Novozymes Celluclast 1.5 L/g of starting dry weight and 0.2 units of Novozymes 188 β-glucosidase. The samples were incubated at 50° C. for 24 h after which time the solids rinsed extensively with water to remove hydrolyzed materials liberated during the 24 h hydrolysis period. Samples were dried to completeness in a dehydrator and the final dry weight of the sample plus tube recorded. The amount of mass lost during the enzyme digestion was determined by subtracting the final sample weight from the starting weight. The digestibility of a sample was determined by calculating percentage of mass lost during the in vitro dry matter digestibility (IVDMD) procedure. Data were graphed and analyzed by one-way Analysis of Variance (ANOVA) with post-hoc testing using the Tukey method.

### Results

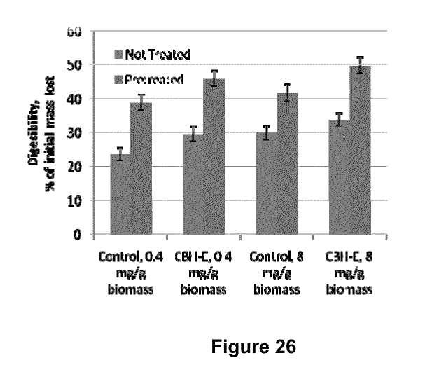

Corn plants identified as bearing CBH-E glucanase and selection marker genes were identified by PCR (see FIG. 25) and analyzed for digestibility using an in vitro dry matter digestibility (IVDMD) assay. As seen in FIG. 26, the group of samples pretreated with dilute acid (“pretreated”) exhibited significantly increased digestibility relative to samples in the “not treated” group. Hydrolysis of pretreated exoglucanase-expressing corn material with either a low (0.4 mg/g) or high (8 mg/g) concentration of commercial cellulase cocktail (Novozymes Celluclast 1.5 L) exhibited substantially greater digestibility than pretreated control corn material (FIG. 26). Furthermore, pretreated exoglucanase corn material hydrolyzed with a low dose (0.4 mg/g) of Celluclast 1.5 L had a significantly greater digestibility than pretreated control corn material hydrolyzed with a high dose (8 mg/g) of Celluclast 1.5 L, indicating that exoglucanase-expressing corn material can achieve efficient biomass conversion yields using much lower levels of exogenous enzymes.

### Example 8

### Stable Transformation of Poplar to Express Enzyme Polypeptides

In the present Example, poplar plants were stably transformed to express cell wall-modifying enzyme polypeptides.

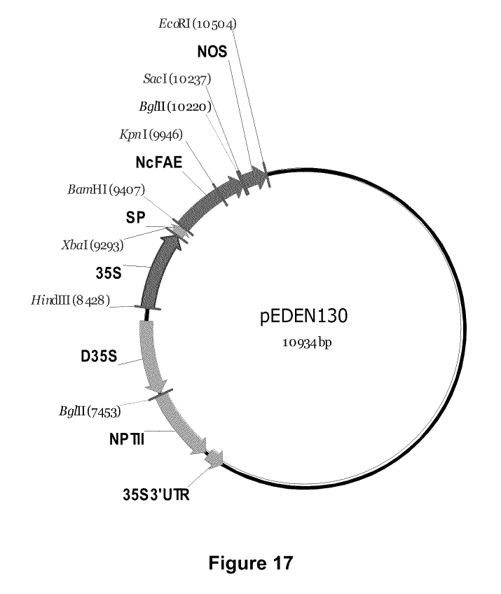

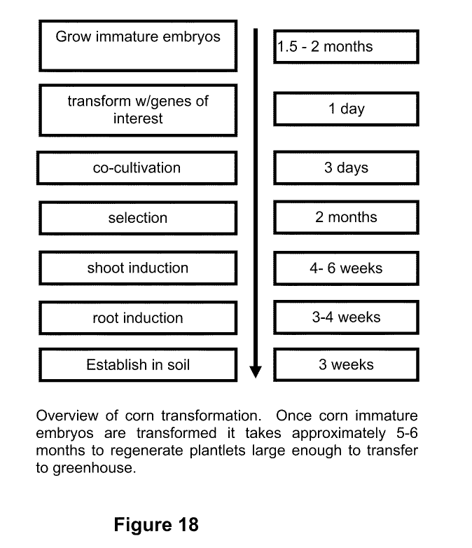

Plant transformation vectors (pEDEN 129 (FIG. 16) for CBH-E expression and pEDEN130 (FIG. 17) for NcFAE expression) were transformed into agrobacterium strain AGL1. Poplar leaf explants generated from material grown at Edenspace were transformed accordingly. Established stable lines of hybrid poplar (Populus alba x P. tremuloides) grown in a laboratory setting in sterile Magenta boxes were used as a stable source of transformable material. Micro-cuttings from shoot and leaf tissue were transferred to hormone-free MS medium in Magenta boxes and grown at 25° C. for 16 h in the light. Explants from forty to fifty day-old, in vitro-grown poplar plantlets were used for transformations.

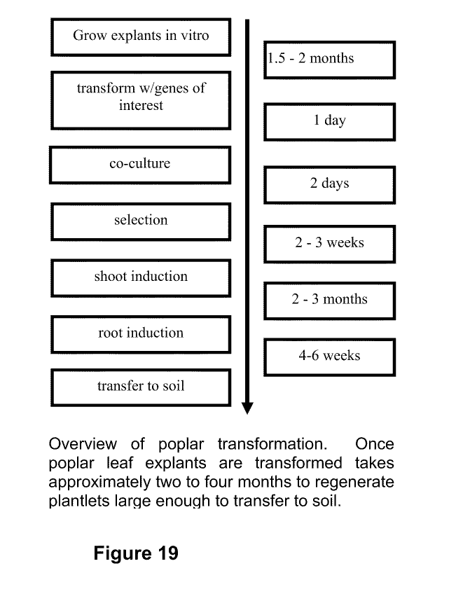

Poplar leaf explants were genetically transformed by a method outlined in FIG. 19 and described below. Leaf discs were wounded with multiple fine cuts and inoculated by swirling in a suspension of Agrobacterium containing an expression construct containing a selectable marker and a gene of interest. Inoculated explants were co-cultivated on callus induction medium in darkness for 2 days and then moved to selection media containing 100 mg/L of kanamycin to induce callus formation and begin selection of transformed cells. Calli were then transferred to shoot induction medium and placed in the light. Calli were subcultured every 3-4 weeks under selection until the calli formed clear shoot tissue. Regenerated shoots were transferred onto rooting medium, and after approximately thirty days healthy plantlets were transplanted into soil.

### Results

Poplar plants were successfully genetically transformed with expression vectors encoding cell wall-modifying enzyme polypeptides. Characterization of transformed plants, including analyses of cellulase activity and impact on digestibility of plant tissue, is described in Example 9.

### Example 9

### Characterization of Poplar Plants Stably Transformed with Constructs Expressing Exoglucanase or Feruloyl Esterase

In the present Example, poplar plants stably transformed with expression vectors for exoglucanase and for feruloyl esterase were characterized by enzyme polypeptide activity and digestibility assays.

### Materials and Methods

### Determination of Exoglucanase and Feruloyl Esterase Activity in Poplar Leaf Extracts

Leaves were collected from a series of independent transformation events regenerated from poplar explants transformed with pEDEN129 (exoglucanase construct comprising SEQ ID NO: 85) see FIG. 16) or pEDEN130 (feruloyl esterase construct comprising SEQ ID NO: 86; see FIG. 17). Harvested leaves were ground in buffer (50 mM MES pH 5.6, 2 mM dithiothreitol, 1 mM EDTA, 1× protease inhibitor cocktail, 0.1% Triton X-100) using a mortar and pestle and the concentration of soluble proteins was determined using the Bradford reagent according to manufacturer's instructions (Bio-Rad).

Exoglucanase activity of leaf extracts from poplar plants transformed with pEDEN129 was measured by incubating ˜10 μg of extracted plant proteins, in duplicate, in a reaction mixture containing 50 mM sodium acetate, pH 5.0 and substrate (250 μM 4-methylumbelliferyl cellobioside (MUC)) at 65° C. for 24 h. At the end of the incubation period, an equal volume of 1 M sodium carbonate was added, and an aliquot of each sample was used to measure fluorescence (excitation wavelength 355 nm; emission wavelength 450 nm) with a plate reader.

Feruloyl esterase activity of leaf extracts from poplar plants transformed with pEDEN130 was measured by incubating duplicate samples containing ˜10 μg of extracted protein with a reaction mixture containing 50 mM sodium acetate pH 5.0 and substrate (250 μM 4-methylumbelliferyl p-trimethylammonio-cinnamate chloride (MUTMAC)) at 37° C. for 2 h to determine feruloyl esterase activity. After the incubation period, 0.5 volumes of 1 M Tris pH 7.5 were added to each sample and an aliquot of each sample was used to measure fluorescence (excitation wavelength 355 nm; emission wavelength 450 nm) with a plate reader.

### Characterization of Exoglucanase Protein Expression in Poplar Leaf Extracts

Leaves from several independent transformation events regenerated from poplar explants transformed with pEDEN129 were harvested and ground in buffer (50 mM MES pH 5.6, 2 mM dithiothreitol, 1 mM EDTA, 1× protease inhibitor cocktail, 0.1% Triton X-100) using a mortar and pestle and the concentration of soluble proteins was determined using the Bradford reagent according to manufacturer's instructions (Bio-Rad).

Extracted proteins were resolved on 10% SDS-PAGE gels, transferred to PVDF membranes, and subsequently immunoblotted with a polyclonal anti-CBH-E primary antibody (described in Example 3) and an appropriate horseradish peroxidase (HRP)-labeled secondary antibody. Immunoreactive bands were visualized using an HRP-catalyzed reaction that converts a non-colored substrate into a purple colored precipitate in situ.

### Results

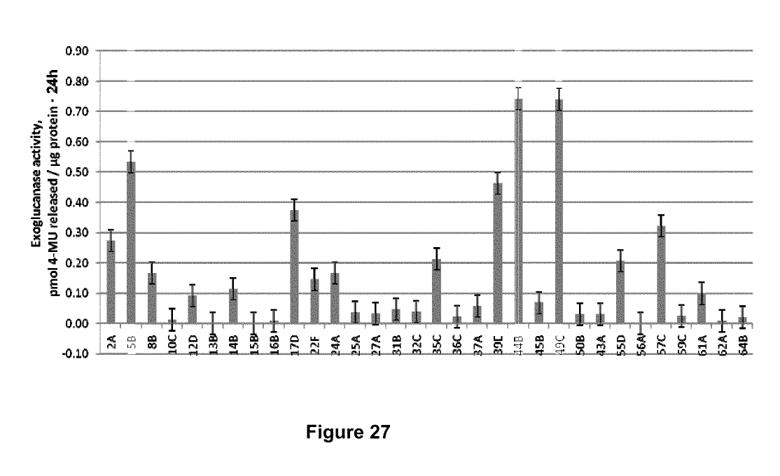

Poplar plants stably transformed with expression vectors for exoglucanase were characterized for exoglucanase activity. As shown in FIG. 27, extracts from plants generated from a number of independent transformation events (2A, 5B, 17D, 35C, 39E, 44B, 49C, 55D, 57D) had significantly elevated levels of exoglucanase activity, whereas extracts from plants generated from other events had intermediate to no detectable exoglucanase activity.

Poplar plants stably transformed to express exoglucanase were also characterized for protein expression by Western blot using polyclonal anti-CBH-E antibody produced as described in Example 3. As shown in FIG. 29, signals were detected for HAT-tagged recombinant CBH-E protein (positive control; CBH-E(+)) as well as non-tagged CBH-E in poplar transformation event 43B.

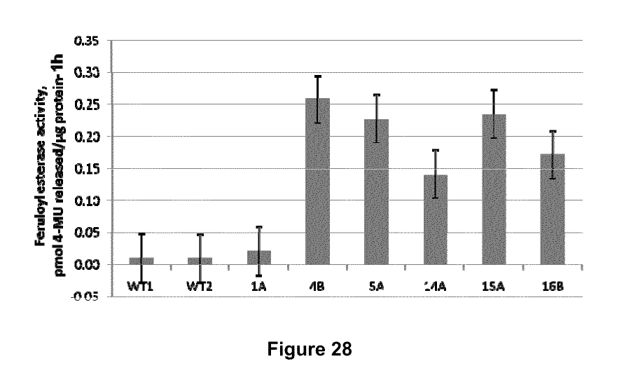

Poplar plants were also stably transformed with expression vectors to express feruloyl esterase (see Example 8) and were characterized for feruloyl esterase activity. As depicted in FIG. 28, extracts from plants generated from a number of independent transformation events 4B, 5A, 14A, 15A, and 16B had significantly elevated levels of feruloyl esterase activity, whereas feruloyl esterase activity in extracts from a plant generated from another event (1A) was similar to that of wild-type, non-transformed poplar leaf extracts (WT1 and WT2).

These results demonstrate successful transformation of poplar plants with expression vectors encoding cell wall modifying enzyme polypeptides having exoglucanase or feruloyl esterase activity.

### Example 10

### Transient Enzyme Polypeptide Expression in Tobacco

The present Example demonstrates successful expression of cell wall-modifying enzyme polypeptides by Agrobacterium-mediated transient expression of tobacco. Expressed enzyme polypeptides were tested and demonstrated to have cellulase activity.

### Materials and Methods

### Transient Protein Expression

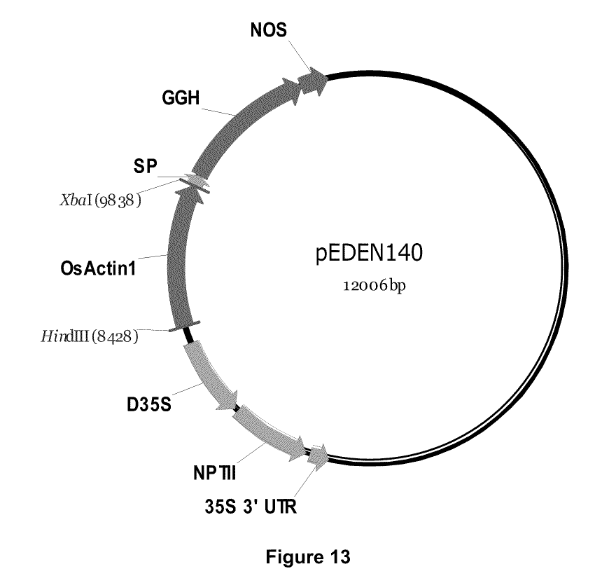

The pEDEN140 plasmid, containing an expression construct encoding GGH, was transformed into Agrobacterium tumefaciens (var. AGL-1) via electroporation and selected for on media supplemented with spectinomycin. Transformed Agrobacteria containing pEDEN140 (which encodes GGH, a beta-glucan glucohydrolase; see FIG. 13 and SEQ ID NO: 80) was resuspended in infiltration media (50 mM MES, 2 mM Na₃PO₄, 0.5% glucose, and 100 μM acetosyringone) and then used to infiltrate Nicotiana benthamiana plants. Undersides of leaves from 7-8 week plants were infiltrated with Agrobacterium suspensions to induce transient protein expression. Multiple leaves were infiltrated with Agrobacteria harboring the transformation construct. As a negative control, a single leaf was infiltrated with media alone. Infiltrated plants were placed in a growth chamber for 48 h before they were harvested for analyses.

### Determination of Cellulase Activity in Infiltrated Tobacco Leaf Extracts

Harvested leaves were ground in buffer (50 mM MES pH 5.6, 2 mM dithiothreitol, 1 mM EDTA, 1× protease inhibitor cocktail, 0.1% Triton X-100) using a mortar and pestle and concentrations of soluble proteins were determined using the Bradford reagent according to manufacturer's instructions (Bio-Rad). Cellulase activity of tobacco leaf extracts infiltrated with media only (control) or with Agrobacteria transformed with pEDEN140 (FIG. 13) was measured by incubating plant extracts in a reaction mixture containing 50 mM sodium acetate, pH 5.0 and substrate (100 μM 4-methylumbelliferyl cellobioside (MUC)) at 65° C. or 95° C. for 30 min. At the end of the incubation period, an equal volume of 1M sodium carbonate was added and the fluorescence of the released 4-methylumbelliferone (4-MU) was measured in a fluorometer.

### Results

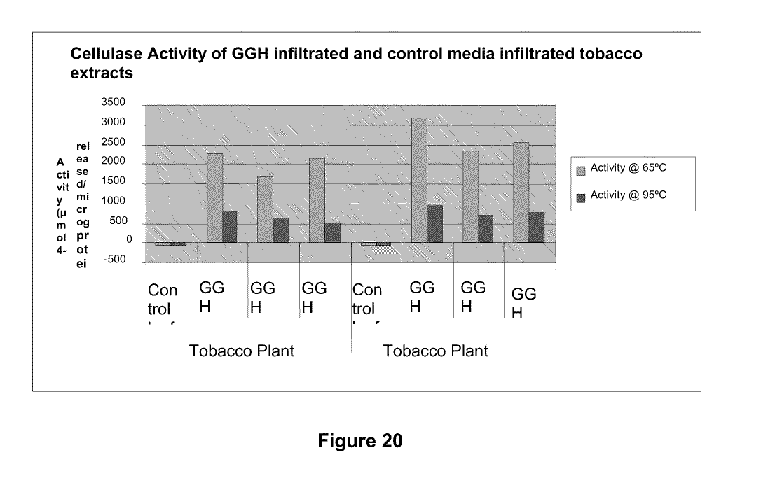

Tobacco leaves were successfully induced to express a cell wall-modifying enzyme polypeptide (GGH) using Agrobacterium-mediated transformation. As shown in FIG. 20, extracts from the tobacco leaves infiltrated with Agrobacteria harboring the expression construct displayed strong cellulase activity at 65° C. and detectable, but lower levels of cellulase activity at 95° C. Extracts from control leaves (e.g., those infiltrated with media alone) had no detectable activity at either 65° C. or 95° C.

These results demonstrate successful induction of cell wall-modifying enzyme polypeptide expression in tobacco, a commercially relevant plant.

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims.

1. A composition of matter comprising plant biomass and at least one enzyme polypeptide having at least 85% amino acid sequence identity to at least one of SEQ ID NO: 1 to 84.

2. The composition of claim 1, wherein the enzyme polypeptide has at least 85% amino acid sequence identity to a sequence selected from the group consisting of SEQ ID NO: 1, SEQ ID NO: 2, SEQ ID NO: 11, SEQ ID NO: 40, SEQ ID NO: 78, and SEQ ID NO: 80.

3. The composition of claim 2, wherein the enzyme polypeptide is encoded by a nucleotide sequence selected from the group consisting of SEQ ID NO: 85, SEQ ID NO: 86, SEQ ID NO: 87, SEQ ID NO: 88, SEQ ID NO: 89, and SEQ ID NO: 90.

4. The composition of claim 1, wherein the enzyme polypeptide has an activity selected from the group consisting of feruloyl esterase, xylanase, alpha-L-arabinofuranosidase, endogalactanase, acetylxylan esterase, beta-xylosidase, xyloglucanase, glucuronoyl esterase, endo-1,5-alpha-L-arabinosidase, pectin methylesterase, endopolygalacturonase, exopolygalacturonase, pectin lyase, pectate lyase, rhamnogalacturonan lyase, pectin acetylesterase, alpha-L-rhamnosidase, mannanase, exoglucanase, endoglucanase, cellulase, licheninase, laminarinase, beta-(1,3)-(1,4)-glucanase, beta-glucan glucohyrdolase, and beta-glucosidase activity.

5. The composition of claim 4, wherein the enzyme polypeptide has feruloyl esterase activity.

6. The composition of claim 4, wherein the enzyme polypeptide has exoglucanase activity.

7. The composition of claim 4, wherein the activity of the enzyme polypeptide is engaged by post-harvest processing of the plant biomass.

8. The composition of claim 7, wherein the post-harvest processing is selected from the group consisting of ensilage, thermochemical bioprocessing, processing in the digestive tract of a mammal, and combinations thereof.

9. The composition of claim 1, wherein the enzyme polypeptide modifies a plant cell wall component selected from the group consisting of xylans, xylan side chains, glucuronoarabinoxylans, xyloglucans, mixed-linkage glucans, pectins, pectates, rhamnogalacturonans, rhamnogalacturonan side chains, lignin, cellulose, mannans, galactans, arabinans, oligosaccharides derived from cell wall polysaccharides, and combinations thereof.

10. The composition of claim 9, wherein the enzyme polypeptide hydrolyzes the plant cell wall component.

11. The composition of claim 1, wherein the enzyme polypeptide hydrolyzes a linkage within plant cell wall.

12. The composition of claim 11, wherein the linkage is a feruloyl ester linkage.

13. The composition of claim 1, wherein the enzyme polypeptide hydrolyzes an interaction in the plant biomass selected from the group consisting of covalent linkages, ionic bonding interactions, and hydrogen bonding interactions.

14. The composition of claim 13, wherein the interaction comprises hemicellulose-cellulose-lignin, hemicellulose-cellulose-pectin, hemicellulosediferululate-hemicellulose, hemicellulose-ferulate-lignin, mixed beta-D-glucan-cellulose, mixed-beta-D-glucan-hemicellulose, or pectin-ferulate-lignin linkages.

15. The composition of claim 1, wherein the plant biomass comprises biomass from a monocotyledonous plant.

16. The composition of claim 11, wherein the monocotyledonous plant is selected from the group consisting of maize, sorghum, switchgrass, miscanthus, sugarcane, wheat, rice, rye, turfgrass, and millet.

17. The composition of claim 1, wherein the plant biomass comprises biomass from a dicotyledonous plant.

18. The composition of claim 17, wherein the dicotyledonous plant is selected from the group consisting of tobacco, potato, soybean, canola, sunflower, alfalfa, cotton and poplar, eucalyptus, pine, sweetgum, and cottonwood.

19. The composition of claim 1, wherein the plant biomass is obtained from a plant part selected from the group consisting of leaves, stems, seeds, and combinations thereof.

20. The composition of claim 1, further comprising an enzyme polypeptide not having at least 85% amino acid sequence identity to any of SEQ ID NO: 1 to 84.

21. The composition of claim 20, wherein the enzyme polypeptide is selected from the group consisting of a cellulase polypeptide, a hemicellulase polypeptide, a ligninase polypeptide, and combinations thereof.

22. A transgenic plant, the genome of which is augmented with:
a recombinant polynucleotide encoding at least one enzyme polypeptide linked to a promoter sequence,
wherein the polynucleotide is optimized for expression in the plant, wherein the at least one enzyme polypeptide has at least 85% sequence identity to at least one of SEQ ID NO.: 1 to 84.

23-46. (canceled)

47. An expression vector comprising a nucleic acid encoding an enzyme polypeptide having at least 85% amino acid sequence identity to at least one of SEQ ID NO: 1 to 84.

48-50. (canceled)

51. A transformed cell comprising a nucleic acid encoding an enzyme polypeptide having at least 85% amino acid sequence identity to at least one of SEQ ID NO: 1 to 84.

52-53. (canceled)

54. A method comprising steps of:
pretreating a plant part under conditions to promote accessibility of celluloses within the lignocellulosic biomass; and
treating the pretreated plant part under conditions that promote hydrolysis of cellulose to fermentable sugars,
wherein the plant part is obtained from at least one transgenic plant, the genome of which is augmented with:
a recombinant polynucleotide encoding at least one enzyme polypeptide operably linked to a promoter sequence, wherein the polynucleotide is optimized for expression in the plant and wherein the at least one enzyme polypeptide has at least 85% sequence identity to at least one of SEQ ID NO.: 1 to 84.

55. An isolated antibody that binds specifically to a feruloyl esterase polypeptide.

56. (canceled)

57. An isolated antibody that binds specifically to an exoglucanase polypeptide.

58-60. (canceled)

61. An array comprising a solid substrate, the substrate having a surface, and a plurality of genetic probes
wherein each genetic probe is immobilized to a discrete spot on the surface of the substrate to form an array, and
wherein the plurality of genetic probes comprises at least ten different oligonucleotides, each oligonucleotide comprising at least ten consecutive nucleotides from a nucleic acid encoding a polypeptide have a sequence of one of SEQ ID NO: 1 to 84.

62-64. (canceled)

65. A plate comprising a solid substrate, the substrate having a surface, and a peptide immobilized to the surface, wherein the peptide comprises at least six consecutive amino acids from a polypeptide having a sequence of one of SEQ ID NO: 1 to 84.
